(19) United States
(12) Patent Application Publication
(10) Pub. No.: US 2021/0340225 A1
(43) Pub. Date: Nov. 4, 2021

(54) SARS-COV-2 (SARS2, COVID-19) ANTIBODIES

(71) Applicants: Harbour Antibodies BV, Rotterdam (NL); Universiteit Utrecht Holding BV, Ultrecht (NL)

(72) Inventors: Franklin Gerardus Grosveld, Rotterdam (NL); Dubravka Drabek, Rotterdam (NL); Rien van Haperen, Rotterdam (NL); Berend Jan Bosch, Utrecht (NL); Juliette Fedry, Utrecht (NL); Daniel L. Hurdiss, Utrecht (NL); Thijs Kuiken, Rotterdam (NL); Batholomeus Leonardus Haagmans, Rotterdam (NL); Barry Hubertus Gerardus Rockx, Rotterdam (NL)

(21) Appl. No.: 17/194,076

(22) Filed: Mar. 5, 2021

(30) Foreign Application Priority Data
Mar. 12, 2020 (GB) 2003632.3
Mar. 25, 2020 (GB) 2004340.2
Aug. 20, 2020 (GB) 2013024.1
Sep. 25, 2020 (GB) 2015240.1
Feb. 8, 2021 (GB) 2101720.7

(51) Int. Cl. C07K 16/10; A61P 31/14

(52) U.S. Cl. CPC C07K 16/10; C07K 2317/76; C07K 2317/565; A61P 31/14

(57) ABSTRACT

The invention relates to antibodies and antigen-binding fragments thereof that recognize SARS-Cov-2 spike proteins (SARS2-S). In some embodiments, the antibodies bind to SARS2-S with high affinity and/or inhibit SARS-Cov-2 infection of human cells. In some embodiments, the antibodies provide a means of preventing, treating or ameliorating SARS2 infection. In some embodiments, the antibodies are used in diagnostic assays (e.g. serodiagnostic assays for SARS2).

### RELATED APPLICATIONS

This application claims priority to GB Patent Application No. 2003632.3, filed on Mar. 12, 2020; GB Patent Application No. 2004340.2 filed on Mar. 25, 2020; GB Patent Application No. 2013024.1, filed on Aug. 20, 2020; GB Patent Application No. 2015240.1, filed on Sep. 25, 2020; and GB Patent Application No. 2101720.7, filed on Feb. 8, 2021. The entire contents of each of the foregoing applications are hereby incorporated by reference in their entirety.

### SEQUENCE LISTING

The application contains a Sequence Listing which has been filed electronically in ASCII format and is hereby incorporated by reference in its entirety. Said ASCII copy, created on May 14, 2021, is named “A103017 1570US P079509WO_Sequence listing_05-14-2021.txt” and is 89,989 bytes in size.

All documents cited herein are incorporated by reference in their entirety.

### TECHNICAL FIELD

The invention relates to antibodies and antigen-binding fragments thereof that recognize the spike (S) protein of SARS-Cov-2 coronavirus (Covid-19), hereafter called SARS2 or SARS-CoV-2.

### BACKGROUND

Coronavirus disease 19 (COVID-19) is caused by the zoonotic severe acute respiratory syndrome coronavirus 2 (SARS-CoV-2). The virus infects humans and human-to-human transmission is efficient, it occurs at close contact and via airway expelled micro-droplets. It is a lower airway infection with fever, coughing and shortness of breath as the primary symptoms and leads in ±2% of cases to a lethal pneumonia. Elderly people and patients with an impaired immune system are particularly vulnerable. (the WHO, in 2020). The first case was documented in Wuhan China late 2019 and has since spread to other parts of Asia, Africa, the Americas and Europe (https://coronavirus.jhu.edu/; https://www.who.int/emergencies/diseases/novel-coronavirus-2019/situation-reports). Vaccines and targeted therapeutics are currently lacking to protect from disease. SARS-CoV-2 belongs to the Sarbecovirus subgenus along with another zoonotic coronavirus SARS-CoV1 (originally termed SARS-CoV; hereafter referred to as SARS-1 or SARS-CoV-1) that emerged in 2002/2003 displaying a ˜10% fatality rate.

Neutralizing epitopes for another coronavirus MERS-CoV and SARS-CoV1 have recently been described (see Widjaja et al. (2019) Emerging Microbes & Infection 8(1):516-530).

There is an immediate need for neutralizing antibodies that recognize the SARS-CoV2 spike protein (SARS2-S).

### SUMMARY OF THE INVENTION

The invention provides an antibody that binds to SARS-Cov-2 coronavirus (SARS2) spike protein (SARS2-S).

In some embodiments, the antibody is capable of inhibiting the infection of human cells by

SARS2. In some embodiments, the antibody binds to the S1 subunit of SARS2-S. In some embodiments, the antibody binds to the S1_{A}, S1_{B}, S1_{C} or S1_{D} subunit of SARS2-S. In some embodiments, the antibody binds to the S1_{B} subunit of SARS2-S and inhibits the binding of SARS2-S to human angiotensin-2 (ACE2). In some embodiments, the antibody binds to the S1_{B} subunit of SARS2-S, but does not inhibit the binding of SARS2-S to human angiotensin-2 (ACE2).

In some embodiments, the antibody binds to the same epitope as an antibody comprising a heavy chain variable region of the amino acid sequence of SEQ ID NO: 10 and a light chain variable region of the amino acid sequence of SEQ ID NO: 9. In some embodiments, the antibody competes for binding to SARS2-S with an antibody comprising a heavy chain variable region of the amino acid sequence of SEQ ID NO: 10 and a light chain variable region of the amino acid sequence of SEQ ID NO: 9.

In some embodiments, the antibody comprises complementarity determining regions (CDRs) with the sequences of:
i. SEQ ID NO: 56 for CDR1 of the heavy chain;
ii. SEQ ID NO: 57 for CDR2 of the heavy chain;
iii. SEQ ID NO: 58 for CDR3 of the heavy chain;
iv. SEQ ID NO: 53 for CDR1 of the light chain;
v. SEQ ID NO: 54 for CDR2 of the light chain; and
vi. SEQ ID NO: 55 for CDR3 of the light chain.

In some embodiments, the antibody comprises a heavy chain variable region of the amino acid sequence of SEQ ID NO: 10 and a light chain variable region of the amino acid sequence of SEQ ID NO: 9.

In some embodiments, the antibody binds to the same epitope as an antibody comprising a heavy chain variable region of the amino acid sequence of SEQ ID NO: 6 and a light chain variable region of the amino acid sequence of SEQ ID NO: 5. In some embodiments, the antibody competes for binding to SARS2-S with an antibody comprising a heavy chain variable region of the amino acid sequence of SEQ ID NO: 6 and a light chain variable region of the amino acid sequence of SEQ ID NO: 5.

In some embodiments, the antibody comprises complementarity determining regions (CDRs) with the sequences of:
i. SEQ ID NO: 44 for CDR1 of the heavy chain;
ii. SEQ ID NO: 45 for CDR2 of the heavy chain;
iii. SEQ ID NO: 46 for CDR3 of the heavy chain;
iv. SEQ ID NO: 41 for CDR1 of the light chain;
v. SEQ ID NO: 42 for CDR2 of the light chain; and
vi. SEQ ID NO: 43 for CDR3 of the light chain

In some embodiments, the antibody comprises a heavy chain variable region of the amino acid sequence of SEQ ID NO: 6 and a light chain variable region of the amino acid sequence of SEQ ID NO: 5.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69.

The invention further provides a combination of antibodies of the invention.

The invention further provides an isolated nucleic acid encoding the antibody of the invention.

The invention further provides a vector comprising the nucleic acid of the invention.

The invention further provides a host cell comprising the vector of the invention.

The invention further provides a pharmaceutical composition comprising the antibody of the invention, or the combination of antibodies of the invention, and a pharmaceutically acceptable carrier.

The invention further provides an antibody of the invention, or a combination of antibodies of the invention, for use in therapy. In some embodiments, the therapy is preventing, treating or ameliorating coronavirus infection, optionally betacoronavirus infection, such as SARS2 infection.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,

for use in preventing, treating or ameliorating SARS2 infection in a human subject, wherein the antibody prevents, treats or ameliorates:

(a) SARS2-induced pneumonia, optionally severe SARS2-induced pneumonia, and/or
(b) SARS2-induced weight loss, and/or
(c) SARS2-induced lung inflammation, and/or
(d) SARS2 replication, optionally SARS2 replication in the lower respiratory tract, and/or
(e) SARS2-induced lung lesions, optionally SARS2-induced gross lesions in the lung.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating SARS2 infection in a human subject, wherein the antibody prevents, treats or ameliorates:
   (a) SARS2-induced pneumonia, optionally severe SARS2-induced pneumonia, and/or
   (b) SARS2-induced weight loss, and/or
   (c) SARS2-induced lung inflammation, and/or
   (d) SARS2 replication, optionally SARS2 replication in the lower respiratory tract, and/or
   (e) SARS2-induced lung lesions, optionally SARS2-induced gross lesions in the lung.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66,
   for use in preventing, treating or ameliorating SARS2 infection in a human subject, wherein the antibody prevents, treats or ameliorates:
   (a) SARS2-induced pneumonia, optionally severe SARS2-induced pneumonia, and/or
   (b) SARS2-induced weight loss, and/or
   (c) SARS2-induced lung inflammation, and/or
   (d) SARS2 replication, optionally SARS2 replication in the lower respiratory tract, and/or
   (e) SARS2-induced lung lesions, optionally SARS2-induced gross lesions in the lung.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69,
   for use in preventing, treating or ameliorating SARS2 infection in a human subject, wherein the antibody prevents, treats or ameliorates:
   (a) SARS2-induced pneumonia, optionally severe SARS2-induced pneumonia, and/or
   (b) SARS2-induced weight loss, and/or
   (c) SARS2-induced lung inflammation, and/or
   (d) SARS2 replication, optionally SARS2 replication in the lower respiratory tract, and/or
   (e) SARS2-induced lung lesions, optionally SARS2-induced gross lesions in the lung.

### DESCRIPTION OF THE DRAWINGS

FIG. 1. Immunisation of H2L2 transgenic mice to generate Antibodies containing human heavy and light chain variable regions. Six mice were immunized with the S domain of OC43, MERS and SARS1 which each contained the S domain coupled to a trimerisation sequence (GCN4, Oshaben et al 2012 or T4, Krammer et al 2012) and a strep tag, resulting in OC43-Secto-GCN4-ST, SARS1-Secto-GCN4-ST and MERS-Secto-T4-ST (see Widjaja et al. (2019) Emerging Microbes & Infections 8(1):516-530; PCT/EP2020/054521). The synthesis of the different proteins is described in the Methods section and the immunisation was carried out in the order OC43, followed 2 weeks later by SARS1, followed 2 weeks later by MERS. In each round the mice were immunized with 25 μg of S protein per mouse. This procedure was repeated once followed by a final boost of each mouse with a mix of the three proteins (10 μg each). A standard Elisa assay was carried out to test which serum of which mice contained antibodies recognizing the S proteins using a Rift valley Fever virus Strep tagged protein as a control.

FIG. 2. Identification of monoclonal H2L2 antibodies targeting the OC43, MERS, SARS1-CoV spike protein. B-cells were collected from the immunized mice and a standard protocol was used to generate antibody producing hybridomas. Each of the hybridomas was cloned and tested for the production of antibodies potentially binding the S protein of each virus and strep tag reacting antibodies were ruled out using a non-related RVFV-ST protein (such as in mouse 503 recognizing the ST tag).

FIG. 3. ELISA cross-reactivity of antibody-containing supernatants of SARS-S H2L2 hybridomas towards SARS2-S1. SARS-S targeting hybridomas were developed by conventional hybridoma technology from immunized H2L2 transgenic mice (Harbour Biomed), as described before (see Widjaja et al. (2019) Emerging Microbes & Infections 8(1):516-530; PCT/EP2020/054521). These mice—carrying genes encoding the heavy and light chain human immunoglobulin repertoire—were sequentially immunized with 2-week intervals with trimeric spike protein ectodomains (S_{ecto}) of three human coronaviruses from the betacoronavirus genus in the following order: 1. HCoV-OC43-S_{ecto}, 2. SARS-CoV-S_{ecto}, 3. MERS-CoV-S_{ecto}, 4. HCoV-OC43-S_{ecto}, 5. SARS-CoV-S_{ecto}, 6. MERS-COV-S_{ecto}. Four days after the last immunization, splenocytes and lymph node lymphocytes were harvested and hybridomas were generated. Antibodies in the cell supernatants were tested for ELISA-reactivity against SARS-S_{ecto}, SARS-S1, SARS-S1_{A} and SARS2-S1. Of the 51 hybridoma supernatants that reacted with SARS-S_{ecto} only, 23 reacted with SARS-S1_{A}, 22 with SARS-S1 but not SARS-S1_{A}, 6 with SARS-S_{ecto} but not SARS-S1. Four of the 51 SARS-S_{ecto} hybridoma supernatants reacted with SARS2-S1 (see column on the right). The table displays ELISA-signal intensities (OD_{450 nm} values) of hybridoma supernatants for the different antigens.

FIGS. 4A-4D. Sequences of four anti-SARS2-S antibodies. Variable region and CDR sequences of the: (FIG. 4A) 65h9, (FIG. 4B) 52d9, (FIG. 4C) 47d11 and (FIG. 4D) 49f1 antibodies.

FIG. 5. The 47D11 antibody binds to SARS-CoV and SARS-CoV-2 spike proteins. Binding of 47D11 to HEK-293T cells expressing GFP-tagged spike proteins of SARS-CoV and SARS-CoV-2 detected by immunofluorescence assay. The human mAb 7.7G6 targeting the MERS-CoV S1_{B} spike domain was taken along as a negative control, cell nuclei in the overlay images are visualized with DAPI.

FIGS. 6A-6F. The 47D11 antibody neutralizes SARS-CoV and SARS-CoV-2. Antibody-mediated neutralization of infection of luciferase-encoding VSV particles pseudotyped with spike proteins of SARS-CoV and SARS-CoV-2. Pseudotyped VSV particles pre-incubated with antibodies at indicated concentrations (see methods) were used to infect VeroE6 cells and luciferase activities in cell lysates were determined at 24 h post transduction to calculate infection (%) relative to non-antibody-treated controls. The average±SD from at least two independent experiments performed is shown. Iso-CTRL: irrelevant isotype monoclonal antibody. Antibody-mediated neutralization of SARS-CoV and SARS-CoV-2 infection on VeroE6 cells. The experiment was performed with triplicate samples, the average±SD is shown.

FIG. 7. The neutralizing 47D11 monoclonal antibody binds the receptor binding domain of SARS-CoV and SARS-CoV-2 spike proteins without eliminating S1_{B}/ACE2 receptor interaction. Interference of antibodies with binding of the S-S1_{B} of SARS-CoV and SARS-CoV-2 to cell surface ACE2-GFP analysed by flow cytometry. Prior to cell binding, S1_{B} was mixed with mAb (mAbs 47D11, 35F4, 43C6, 7.7G6, in H2L2 format) with indicated specificity in a mAb:S1_{B} molar ratio of 8:1 (see FIG. 8 for an extensive analysis using different mAb:S1_{B} molar ratio's). Cells are analysed for (ACE2-)GFP expression (x-axis) and S1_{B} binding (y-axis). Percentages of cells that scored negative, single positive, or double positive are shown in each quadrant.

FIGS. 8A-8C. 47D11 does not prevent binding of SARS-S1_{B} and SARS2-S1_{B} to ACE2-expressing cells. Human HEK-293T cells expressing human ACE2-GFP proteins (see Methods) were detached and fixed with 2% PFA, incubated with a fixed amount of human Fc-tagged S1_{B} domain of SARS-S or SARS2-S that was preincubated for 1 h with mAb (mAbs 47D11, 35F4, 43C6, 7.7G6, in H2L2 format) at the indicated mAb:S1_{B} molar ratios, and analysed by flow cytometry using a Alexa Fluor 594-conjugated secondary antibody targeting the human Fc tag. Cells are analysed for GFP expression (x-axis, GFP signal) and antibody binding (y-axis, Alexa 594 signal). Percentages of cells that scored negative, single positive, or double positive are shown in each quadrant. Binding controls include PBS-treated cells (mock), treatment of cells with SARS-S1_{B} and SARS2-S1_{B} in the absence of antibody, and cells treated with antibodies only. The experiment was performed twice, data from a representative experiment are shown.

FIG. 9. ELISA-based receptor binding inhibition assay. Recombinant soluble human ACE2 was coated on NUNC Maxisorp plates (Thermo Scientific) at 4° C. overnight. Plates were washed three times with PBS containing 0.05% Tween-20 and blocked with 3% BSA in PBS containing 0.1% Tween-20 at room temperature for 2 hours. Recombinant S_{ecto} and S1_{B} of SARS-S or SARS2-S (300 ng) and serially diluted mAbs (mAbs 47D11, 35F4, 43C6, 7.7G6, in H2L2 format) were mixed for 1 h at RT, added to the plate for 1 hour at room temperature, after which the plates were washed three times. Binding to ACE2 was detected using HRP-conjugated StrepMAb (IBA) that recognizes the C-terminal Streptag on the S_{ecto} and S1_{B} proteins.

FIG. 10. Cell-cell fusion inhibition assay. VeroE6 cells were seeded with density of 10⁵ cells per ml. After reaching 70˜80% confluency, cells were transfected with plasmids encoding full length SARS-S, SARS2-S and MERS-S-C-terminally fused to GFP—using Lipofectamine 2000 (Invitrogen). The furin recognition site in the SARS2-S was mutated (R⁶⁸²RAR to A⁶⁸²AAR) to inhibit cleavage of the protein by endogenous furin and allow trypsin-induced syncytia formation. Two days post transfection, cells were pretreated DMEM only or DMEM with 20 μg/ml mAbs for 1 h and subsequently treated with DMEM with 15 μg/ml trypsin (to activate the spike fusion function) in the absence or presence of 20 μg/ml mAbs (47D11 crossreactive SARS-S and SARS2-S, 35F4 reactive to SARS-S, 7.7G6 reactive to MERS-S). After incubation at 37° C. for 2 hrs, the cells were fixed with 2% PFA in PBS for 20 min at room temperature and stained for nuclei with 4,6-diamidino-2-phenylindole (DAPI). Cells expressing the S-GFP proteins were detected by fluorescence microscopy and S-mediated cell-cell fusion was observed by the formation of (fluorescent) multi-nucleated syncytia. The fluorescence images were recorded using a Leica SpeII confocal microscope. The experiment was performed twice, data from a representative experiment are shown.

FIG. 11. Divergence in surface residues in MB of SARS-CoV and SARS-CoV-2. Upper panel: Structure of the SARS-CoV spike protein S1_{B} RBD in complex with human ACE2 receptor (PDB: 2AJF). ACE2 (wheat colour) is visualized in ribbon presentation. The S1_{B} core domain (blue) and subdomain (orange) are displayed in surface presentation using PyMOL, and are visualized with the same colors in the linear diagram of the spike protein above, with positions of the 51 and S2 subunits, the S ectodomain (S_{ecto}), the 51 domains S1_{A-D} and the transmembrane Domain™ indicated. Lower panel: Similar as panel above with surface residues on S1_{B} of SARS-CoV that are at variance with SARS-CoV-2 colorored in white.

FIG. 12. Protein sequence alignment of the MB receptor binding domain (RBD) of the SARS-CoV and SARS-CoV-2 spike proteins by ClustalW. Numbering denotes the residue position in the full-length spike protein of SARS-CoV (Genbank: AAP13441.1) and SARS-CoV-2 (Genbank: QHD43416.1). Asterisks (*) indicated fully conserved residues, the colon symbol (:) indicates conservation between groups of very similar properties, and the period symbol (.) indicates conservation between groups of weakly similar properties. Sequences corresponding to the S1_{B} receptor binding core domain and the receptor binding subdomain are colored in blue and orange, respectively. The fourteen residues that are involved in binding of SARS-CoV S1_{B} to human ACE2 are highlighted in grey.

FIGS. 13A and 13B. The 49f1 antibody. The 49f1 antibody having the V_{H} domain of SEQ ID NO: 6 and the V_{L} domain of: (FIG. 13A) SEQ ID NO: 5 (FIG. 13B) Virus neutralization assay for the 49f1 antibody having the VH domain of SEQ ID NO: 6 and the V_{L} domain of SEQ ID NO: 5 (sup. 42-2) or SEQ ID NO: 47 (sup. 42-3).

FIGS. 14A and 14B. The neutralizing 47D11 monoclonal antibody binds the receptor binding domain of SARS-CoV and SARS-CoV-2 spike proteins. ELISA binding curves of 47D11 to S_{ecto} (left panel; FIG. 14A) or S1_{A} and S1_{B} (RBD) (right panel; FIG. 14B) of SARS-S and SARS2-S coated at equimolar concentrations. The average±SD from at least two independent experiments performed is shown.

FIG. 15. ELISA binding curve of the anti-StrepMAb (IBA) antibody to Strep-tagged spike antigens to corroborate equimolar ELISA plate coating of SARS-S_{ecto}/SARS2-S_{ecto} (top-left panel), SARS-S1_{B}/SARS2-S1_{B} (top-right panel) and SARS-S1_{A}/SARS2-S1_{A} (bottom-left panel) antigens used in FIG. 14.

FIG. 16. Binding kinetics of 47D11 to the S ectodomain and MB of SARS-CoV and SARS-CoV-2. Binding kinetics of 47D11 to immobilized recombinant SARS-S_{ecto}, SARS2-S_{ecto}, SARS-S1_{B} and SARS2-S1_{B} was measured using biolayer interferometry at 25° C., as described previously (see Widjaja et al. (2019) Emerging Microbes & Infections 8(1):516-530; PCT/EP2020/054521). Kinetic binding assay was performed by loading 47D11 mAb at optimal concentration (42 nM) on anti-human Fc biosensor for 10 mins. Antigen association step was performed by incubating the sensor with a range of concentrations of the recombinant spike ectodomain (1600-800-400-200-100-50-25 nM) for 10 min, followed by a dissociation step in PBS for 60 min. The kinetics constants were calculated using 1:1 Langmuir binding model on Fortebio Data Analysis 7.0 software.

FIG. 17. ELISA reactivity data for supernatants after transfection.

FIGS. 18A-1811. Effect of prophylactic neutralizing antibody treatment on weight loss and virus replication following SARS-CoV-2 infection in hamsters. FIG. 18A. Body weights of hamsters treated with antibodies were measured at indicated days after inoculation with SARS-CoV-2. SARS-CoV-2 viral RNA (FIGS. 18B, 18C, 18E and 18G) or infectious virus (FIGS. 18D, 18F and 18H) was detected in throat (FIG. 18B), nasal washes (FIGS. 18C and 18D), lung (FIGS. 18E and 18F) and nasal turbinates (FIGS. 18G and 18H). The mean % of starting weight, the mean copy number or the mean infectious titer is shown, error bars represent the standard error of mean. n=4. *=P<0.01 and +=P<0.05, ANOVA compared to SARS-CoV-2 inoculated, untreated animals.

FIGS. 19A-19F. Gross pathological examination of the lungs of SARS-CoV-2 infected hamsters. Foci (arrowheads) of pulmonary consolidation in untreated SARS-CoV-2 infected animals (FIG. 19A) and animals treated with control MAb (FIG. 19D) or low dose plasma (FIG. 19F). Protection against pulmonary lesions in hamsters treated with MAb 47D11 (FIG. 19C) and high dose plasma (FIG. 19E), similar to mock infected animals (FIG. 19B). Images are from representative animals of each treatment group.

FIGS. 20A-20D. Histopathological changes and virus antigen expression in nasal turbinates of hamsters after challenge with SARS-CoV-2. In the nasal turbinate of a sham-inoculated hamster (left column), the nasal cavity is empty and the histology of the olfactory mucosa is normal (FIG. 20A). In a serial section, there is no SARS-CoV-2 antigen expression (FIG. 20C). In the nasal turbinate of a non-treated SARS-CoV-2-inoculated hamster (FIGS. 20B and 20D), the nasal cavity is filled with edema fluid mixed with inflammatory cells and debris and the olfactory mucosa is infiltrated by neutrophils (FIG. 20B). A serial section of this tissue shows SARS-CoV-2 antigen expression in many olfactory mucosal cells, as well as in cells in the lumen (FIG. 20C).

FIGS. 21A-21H. Histopathological changes and virus antigen expression in bronchioles and alveoli of hamsters after challenge with SARS-CoV-2. In the lung of a non-treated SARS-CoV-2-inoculated hamster (FIGS. 21A and 21B), the alveoli are flooded by edema fluid (FIG. 21B), fibrin, sloughed epithelial cells, cell debris, neutrophils, mononuclear cells, and erythrocytes, obfuscating the histological architecture, and there are a few inflammatory cells in the lumen and epithelium of a bronchiole (FIG. 21A), and in the perivascular space around an adjacent blood vessel. In the lung of a sham inoculated hamster (FIGS. 21C and 21D), the lumina of the alveoli are empty (FIG. 21D), the alveolar walls are thin, and there are no inflammatory cells in or around the walls of the bronchiole and adjacent blood vessel (FIG. 21C). The lung of a non-treated SARS-CoV-2-inoculated hamster shows SARS-CoV-2 antigen expression in multiple (FIG. 21E) or solitary (FIG. 21F) bronchiolar epithelial cells, type I pneumocytes (FIG. 21G), and type II pneumocytes (FIG. 21H, arrowheads).

FIG. 22. Effect of preventive treatment with MAb or high dose convalescent plasma on severity of pneumonia and level of virus antigen expression in lung parenchyma of hamsters after challenge with SARS-CoV-2. Comparison of extent of histopathological changes (HE) and virus antigen expression (IHC) at four days after SARS-CoV-2 inoculation at low magnification (two left columns) and high magnification (two right columns) in hamsters treated 24 hours before virus inoculation with neutralizing antibodies (second, third and fourth rows) compared to no treatment before SARS-CoV-2 inoculation (first row) and sham inoculation (fifth row).

FIGS. 23A and 23B. Quantitative assessment of histopathological changes and virus antigen expression. Percentage of inflamed lung tissue (FIG. 23A) and percentage of lung tissue expressing SARS-CoV-2 antigen (FIG. 23B) estimated by microscopic examination in different groups of hamsters at four days after SARS-CoV-2 inoculation. Individual (symbols) and mean (horizontal lines) percentages are shown. Error bars represent the standard error of mean. n=4. *=P<0.01 and +=P<0.05, ANOVA compared to SARS-CoV-2 inoculated, untreated animals.

FIGS. 24A and 24B. 47D11 has differing conformational selectivity for the SARS-CoV and SARS-CoV-2 spike. FIG. 24A) Surface rendering of the fully closed SARS-CoV spike bound to three 47D11 antibody Fab fragments, shown as two orthogonal views. FIG. 24B) Surface rendering of the partially open SARS-CoV-2 spike in complex with two 47D11 antibody Fab fragments, shown as two orthogonal views. For clarity, only the Fab variable region is shown.

FIGS. 25A-25F. FIG. 25A) Top view of the 47D11 bound SARS-CoV-2 spike. The spike protomers are coloured light grey, and the 47D11 heavy and light chain are coloured dark grey. Glycans, and the N-terminal domain, are omitted for clarity and only the Fab variable region is shown. The superposed structure of the partially open apo SARS-CoV-2 spike (PDB ID: 6ZGG) is shown as a silhouette. FIG. 25B) Zoomed in view of the boxed region in panel A. FIG. 25C) Zoomed in view of the SARS-CoV-2 open RBD and adjacent NTD. The overlaid 47D11 Fab is coloured white and the N343 glycan is shown in ball-and-stick representation and coloured dark grey. FIG. 25D) Top view of the 47D11 bound SARS-CoV-2 spike coloured as shown in panel A. The superposed structure of the closed apo SARS-CoV spike (PDB ID: 5XLR) is shown as a silhouette. FIG. 25E) Zoomed in view of the boxed region in panel D, showing a putative salt bridge between the 47D11 variable light chain and the RBD loop. FIG. 25F) ELISA-binding curves of 47D11 binding to wildtype and loop swapped spike ectodomains.

FIGS. 26A-26K. FIG. 26A) Ribbon diagram of the SARS2-S receptor-binding domain (RBD) in complex with the 47D11 antibody Fab fragment. For comparison, residues 1-84 of the RBD bound ACE2 (PDB ID: 6M0J) are shown as a silhouette. FIG. 26B) Close-up view of the 47D11 epitope with the hydrophobic pocket residues shown as sticks and coloured dark grey. The N343 glycan is shown in ball-and-stick representation and coloured light grey. For clarity, only the core pentasaccharide is shown. FIG. 26C) Slice through the surface rendered 47D11 bound SARS2-S RBD. D) Equivalent view as shown in panel C for the apo RBD (PDB ID:6VYB). FIG. 26E) Relative surface binding of 47D11 and (FIG. 26F) ACE2, (G) CR3022 and (FIG. 26H) an anti-FLAG antibody to full-length SARS-CoV-2 spike epitope mutants, determined by fluorescence-activated cell sorting. FIG. 26I) Antibody-mediated neutralization of infection of luciferase-encoding VSV particles pseudotyped with wild-type, V367A or V367F SARS2-S. FIG. 26J) Surface representation of the 47D11 bound SARS2-S RBD coloured according to mean mutation effect on expression (darker grey indicates more constrained). The Fab is shown as a ribbon diagram. FIG. 26K) As shown in E for the 5309 bound SARS-CoV-2 RBD.

FIG. 27. Surface rendering of the SARS2 RBD, coloured according to the mean mutation effect on expression (darker grey indicates more constrained) (Starr et al. (2020) Cell doi:10.1016/j.ce11.2020.08.012), with binding positions of 47D11, CR3022 (PDB ID: 6W41) and VHH-72 (PDB ID: 6WAQ) overlaid.

FIG. 28. Multiple sequence alignment of the RBD residues from SARS1, SARS2 and 11 SARS-like viruses. The sequence alignment was performed using Clustal Omega (Sievers et al. (2011) Mol. Syst. Biol. 7:539) and the image was generated by ESPript 3.01 (Robert et al. (2014) Nucleic Acids Res. 42:W320-4). The secondary structure assignment, based on the SARS2 RBD, is shown.

FIGS. 29A-29C. FIG. 29A) Surface representation of the 47D11 bound RBD coloured according to sequence conservation across SARS-CoV-1, SARS-CoV-2 and 13 SARS-like viruses (FIG. 28). The 47D11 Fab variable chains are shown as a ribbon diagram and coloured grey. Heavy chain residues W102 and F103 are shown as sticks. For comparison, residues 1-84 of the RBD bound ACE2 (PDB ID: 6M0J) are shown as a silhouette. FIG. 29B) ELISA-binding curves of 47D11 to the S1B domain of SARS1, SARS2, WIV16, HKU3-3 and HKU9-3. The average±SD from two independent experiments with technical duplicates is shown. FIG. 29C) Aligned RBD sequences of SARS-CoV-2, SARS-CoV-1, WIV16, HKU3-3 and HKU9-3. Key residues in the 47D11 epitope are indicated by red arrows.

FIGS. 30A-30D. FIG. 30A) Superposed atomic coordinates for the 47D11 bound SARS and SARS2 RBD, coloured cyan and pink, respectively. FIG. 30B) The SARS2 RBD shown as a surface representation and coloured according to the Kyte-Doolittle scale. The 47D11 Fab fragment is shown as a ribbon diagram, with the light chain shown semi-transparent. FIG. 30C) Zoomed in view of the hydrophobic pocket for the 47D11 bound SARS2 RBD (dark grey) overlaid with the equivalent region from an apo structure (PDB ID: 6VYB). FIG. 30D) Ribbon diagram of the 47D11 bound RBD with the 55 Å³ solvent accessible cavity, generated using CASTp 3.0, shown as grey spheres (Tian et al. (2018) Nucleic Acids Res. 46:W363-W367).

FIGS. 31A-31F. Proposed mechanism of 47D11 binding to SARS-CoV and SARS-CoV-2 FIG. 31A) The Spike protein in solution exists in equilibrium between the closed and the partially open conformation. The S_{1A} (NTD) domains are colored in light grey at the three corners of the complex, the S_{1B} domains are in grey and the rest of the spike protein is in dark gray. For clarity, the letter «o» is added on the S_{1B} domain shown at the bottom of the complex when in it is in the open conformation. FIG. 31B) The 47D11 Fab is added and binding of a first Fab on the closed S_{1B} depicted towards the top of the complex, right next to the open S_{1B} at the bottom of the complex is favored (47D11 epitope is more accessible). FIG. 31C) A second 47D11 Fab binds the second closed beige S_{1B} domain. In the case of SARS-CoV it is favored by the formation of an extra salt bridge between this second Fab and the previous S_{1B} distal loop (Fab LC R18/S D463). FIG. 31D) For SARS-CoV-2 the lowermost S_{1B} is stabilized in the open conformation, likely by hydrophobic interactions formed by the F486 of the distal loop with the open S_{1B}. No 47D11 Fab can bind to this open third S_{1B} as this would result in severe clashes with the neighbouring S_{1A}. This is the final conformation stabilized by 47D11 for SARS-CoV-2 S. FIG. 31E) for SARS-CoV S, an equilibrium can exist in the form with 2 bound 47D11 Fabs, where the lowermost S_{1B} domain could open and close. The closed form is stabilised by the formation of the extra salt bridge. FIG. 31F) When the lowermost S_{1B} domain is closed, a third 47D11 Fab can bind on it. This is the final conformation stabilised by the 47D11 Fab for SARS-CoV. In both SARS-CoV and SARS-CoV-2 the 47D11 Fab prevents the possibility of fully opening the 3 S_{1B} domains (as 2 or 3 of them are bound by 47D11 and cannot fully open anymore because of severe clashes with the next S_{1A}). They may be able to bind ACE2 in a partially open conformation that would impair the timely release of the fusogenic S₂ domain and thereby prevent viral fusion.

FIGS. 32A-32D. FIG. 32A) Gold-standard Fourier shell correlation (FSC) curve generated from the independent half maps contributing to the 3.8 Å global resolution density map of the SARS-CoV spike in complex with the 47D11 antibody Fab fragment. FIG. 32B) Local resolution filtered EM density map for the C3 refined SARS-CoV spike:47D11 complex, coloured according to local resolution which was calculated in Relion3.1. FIG. 32C) Gold-standard Fourier shell correlation (FSC) curve generated from the independent half maps contributing to the 4 Å global resolution density map of the SARS-CoV-2 spike in complex with the 47D11 antibody Fab fragment. FIG. 32D) Local resolution filtered EM density map for the C3 refined SARS-CoV-2 spike:47D11 complex, coloured according to local resolution which was calculated in Relion3.1.

FIGS. 33A-33F. 47D11 binds specifically to the RBD in down conformation and prevents their full compaction. FIG. 33A) Top view of the 47D11 bound SARS2 spike, shown as a ribbon diagram. The 47D11-bound spike protomers are colored in light grey, grey and dark grey, and the 47D11 heavy and light chain are shown semi-transparent and colored grey. Glycans, and the N-terminal domain, are omitted for clarity and only the Fab variable region is shown. The superposed structure of the partially open apo SARS2 spike (PDB ID: 6ZGG) is colored black. FIG. 33B) Zoomed in view of the boxed region in panel A. The region encompassing residues 470-490, used for the loop swap experiments, is indicated with scissors. FIG. 33C) Zoomed in view of the SARS2 “up” RBD and adjacent NTD, shown in cartoon representation. The overlaid 47D11 Fab is shown as a silhouette and the N343 glycan is shown in ball-and-stick representation and colored grey. The inset panel shows a zoomed in view of the clash between the NTD residue V171 and the 47D11 heavy chain. FIG. 33D) Top view of the 47D11 bound SARS spike colored as shown in panel A. The superposed structure of the closed apo SARS spike (PDB ID: 5XLR) is colored black. FIG. 33E) Zoomed in view of the boxed region in panel D, showing a putative salt bridge between the 47D11 variable light chain and the RBD loop. The region encompassing residues 457-477, used for the loop swap experiments, is indicated with scissors. FIG. 33F) ELISA-binding curves of 47D11 binding to wildtype, loop swapped and D463A spike ectodomains.

FIGS. 34A-34G. The 47D11 epitope comprises a mutationally constrained hydrophobic pocket which is normally shielded by glycan N343. FIG. 34A) Close-up view of the 47D11 epitope with the hydrophobic pocket residues shown as sticks and colored dark blue. The N343 glycan is shown in ball-and-stick representation and colored tan. For clarity, only the core pentasaccharide is shown. FIG. 34B) Slice through the surface rendered 47D11 bound SARS2-S RBD. The helix encompassing residues 365-370 is shown in darker grey. FIG. 34C) Equivalent view as shown in panel C for the apo RBD (PDB ID:6VYB). FIG. 34D) Relative binding of 47D11, (FIG. 34E) ACE2, (FIG. 34F) CR3022 and (FIG. 34G) 49F1 to cell surface expressed SARS2-S, determined by fluorescence-activated cell sorting. The data were analyzed by the unpaired, two-tailed, Student's t test using GraphPad Prism 7.0. A P value <0.05 was considered significant. *P<0.05, **P<0.01, ***P<0.0001.

FIGS. 35A and 35B. 47D11 neutralizes pseudoviruses with RBM mutations from the currently circulating SARS-CoV-2 variants. FIG. 35A) Surface representation of the 47D11 bound RBD highlighting the localization of the RBD mutations found in recently emerging SARS_CoV-2 variants. FIG. 35B) 47D11-mediated neutralization of infection of luciferase-encoding VSV particles pseudotyped with SARS-CoV-2 S mutants. The average±SD from two independent experiments is shown.

FIGS. 36A-36D. The 47D11 epitope is conserved in SARS-like viruses. FIG. 36A) Phylogenetic tree of SARS-like viruses RBD used to assess 47D11 binding. FIG. 36B) Surface representation of the 47D11 bound RBD colored according to sequence conservation across SARS-CoV, SARS-CoV-2 and 11 SARS-like viruses (FIG. 41). The 47D11 Fab variable chains are shown as a ribbon diagram and colored grey. Heavy chain residues W102 and F103 are shown as sticks. For comparison, residues 1-84 of the RBD bound ACE2 (PDB ID: 6M0J) are shown as a silhouette. FIG. 36C) 47D11-mediated neutralization of infection of luciferase-encoding VSV particles pseudotyped with WIV16-S. An anti-Strep-tag human monoclonal antibody was used as an antibody isotype control. The average±SD from two independent experiments is shown. FIG. 36D) Aligned RBD sequences of SARS-CoV-2, SARS-CoV, WIV16, HKU3-3 and HKU9-3. Key residues in the 47D11 epitope are indicated by arrows.

FIGS. 37A-37D. FIG. 37A) Gold-standard Fourier shell correlation (FSC) curve generated from the independent half maps contributing to the 3.8 Å global resolution density map of the SARS-CoV spike in complex with the 47D11 antibody Fab fragment. FIG. 37B) Angular distribution plot of the final C3 refined SARS-CoV spike EM density map. FIG. 37C) Gold-standard FSC curve generated from the independent half maps contributing to the 4 Å global resolution density map of the SARS-CoV-2 spike in complex with the 47D11 antibody Fab fragment. FIG. 37D) Angular distribution plot of the final C1 refined SARS-CoV-2 spike EM density map.

FIGS. 38A-38G. Proposed mechanism of 47D11 binding to SARS-CoV and SARS-CoV-2 FIG. 38A) In solution, the spike protein, exists in equilibrium between the closed and the partially open conformation. The S1A (NTD) domains are colored in light gray, the S1B domains are in salmon, beige and blue, and the rest of the spike protein is in dark gray. For clarity, the letter “o” is added on the blue S1B domain when in the open conformation. FIG. 38B) Initially, a 47D11 Fab binds to the closed S1B, located counterclockwise of the open S1B, where the 47D11 epitope is more accessible. FIG. 38C) A second 47D11 Fab binds to the remaining closed S1B domain. For SARS-CoV-2 the open S1B is likely stabilized in this conformation by hydrophobic interactions with F486 located on the adjacent, 47D11-bound, closed S1B. FIG. 38D) Closure of the remaining open S1B is likely prevent by clashes with the first bound Fab and the N234 glycan. The 47D11 Fab is unable to bind to the open S1B due to clashes with the N331 glycan and the neighboring S1A (see FIG. 33C). This is the final conformation stabilized by 47D11 for the SARS-CoV-2 Spike. FIG. 38E) For SARS-CoV S, an equilibrium can exist between the form with 2 bound 47D11 Fabs, where the blue S1B domain could be open or closed. FIG. 38F) The closed form is stabilized by the formation of a salt bridge between R18 on the 47D11 light chain and D463 on the S1B distal loop. FIG. 38G) When the blue S1B domain is closed, a third 47D11 Fab can bind to it. This is the final conformation stabilized by the 47D11 Fab for SARS-CoV.

FIGS. 39A-39F. FIG. 39A) EM density for the 47D11 epitope region in SARS-S and FIG. 39B) SARS2-S. FIG. 39C) Superposed atomic coordinates for the 47D11 bound SARS-S and SARS2-S RBD, colored dark grey and light grey, respectively. FIG. 39D) The SARS2-S RBD shown as a surface representation and colored according to the Kyte-Doolittle scale. The 47D11 Fab fragment is shown as a ribbon diagram, with the light chain shown semi-transparent. FIG. 39E) Zoomed in view of the hydrophobic pocket for the 47D11 bound SARS2 RBD overlaid with the equivalent region from an apo RBD (PDB ID: 6VYB) and linoleic acid-bound RBD (PDB ID: 6ZB4). FIG. 39F) Ribbon diagram of the 47D11 bound RBD with the 55 Å³ solvent accessible cavity, generated using CASTp 3.0, shown as grey spheres.

FIGS. 40A and 40B. ELISA-based EC50 for wt or D463A SARS-S ectodomains binding to (FIG. 40A) 47D11 or (FIG. 40B) ACE2.

FIGS. 41A and 41B. FIG. 41A) Surface rendering of the SARS2-S RBD, colored according to the mean mutation effect on expression (dark grey indicates more constrained) (3), with binding positions of 47D11, 5309 (PDB ID: 6WPS), H104 (PDB ID: 7CAH), CR3022 (PDB ID: 6W41) and VHH-72 (PDB ID: 6WAQ) overlaid. FIG. 41B) Superposed atomic coordinates for the 47D11-bound SARS2-S RBD, and two adjacent S2M11-bound RBDs (PDB ID: 7K43). For clarity, only the antibody heavy chains are shown.

FIG. 42. Multiple sequence alignment of the RBD residues from SARS-CoV, SARS-CoV-2 and 11 SARS-like viruses. The sequence alignment was performed using Clustal Omega and the image was generated by ESPript 3.01. The secondary structure assignment, based on the full length SARS-CoV-2 spike (PDB ID: 6XR8), is shown. Key residues in the 47D11 epitope are indicated by asterisks.

FIG. 43. Single-particle cryo-EM image processing workflow for the SARS-CoV:47D11 complex.

FIG. 44. Single-particle cryo-EM image processing workflow for the SARS-CoV-2:47D11 complex.

FIGS. 45A-45C. (FIG. 45A) 47D11, (FIG. 45B) ACE2, and (FIG. 45C) anti-FLAG antibody binding to wild-type and three mutant SARS2-S proteins (E484K, N501Y and K417N).

FIGS. 46A-46D. FIG. 46A) 47D11-mediated neutralization of infection of luciferase-encoding VSV particles pseudotyped with SARS-CoV-2 S mutants. Binding of (FIG. 46B) 47D11, (FIG. 46C) ACE2 and (FIG. 46FD) anti-FLAG antibody to wild-type and five mutant SARS2-S proteins (N439K, E484K, F490S, Q493R and S494P). Each of the SARS2-S proteins carried a C-terminally appended FLAG-tag epitope.

FIGS. 47A and 47B. ELISA reactivity data for 49F1 showing that this antibody binds to the ectodomain and 51 domain of SARS-S and SARS2-S. These data also show that 49F1 does not bind to the S1_{A} or S1_{B} domains.

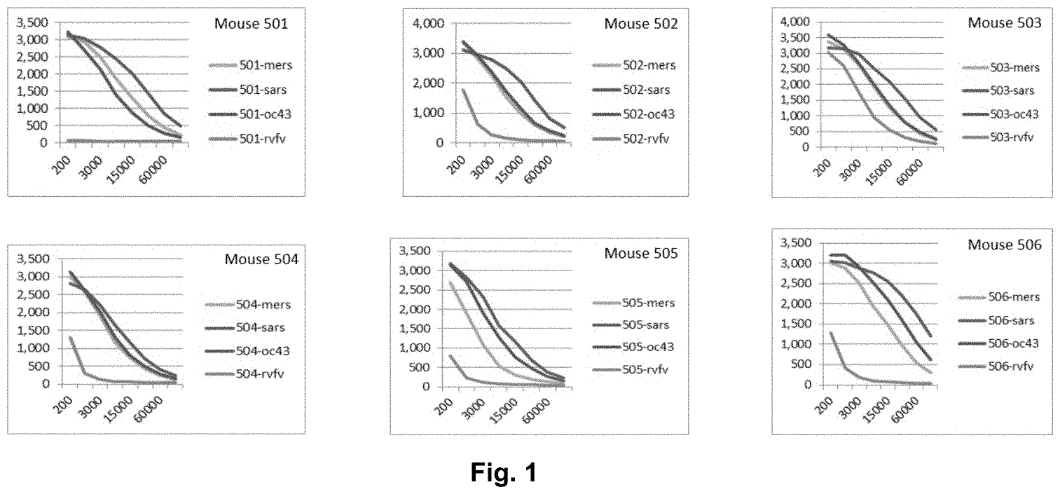

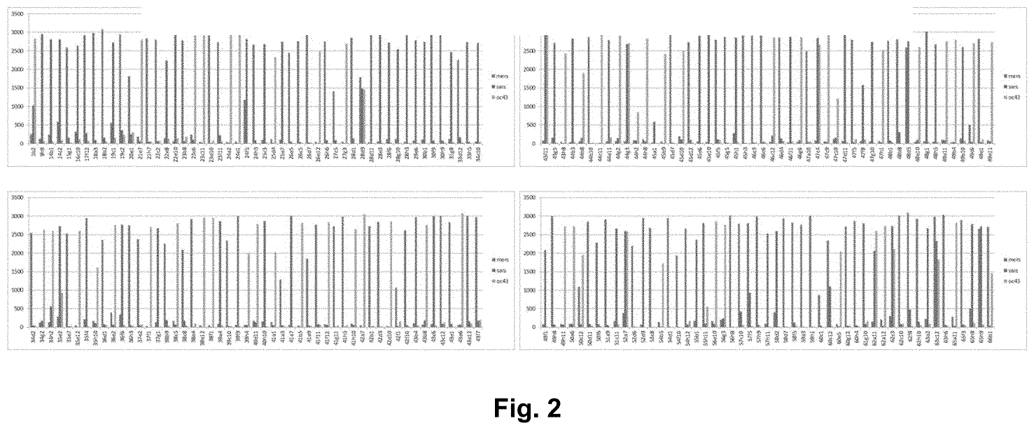

### SEQUENCE LISTING

| | VL domain | VH domain | CDRL1-3 | CDRH1-3 | VL domain | VH domain |
|---|---|---|---|---|---|---|
| Clone | (protein) | (protein) | (protein) | (protein) | (DNA) | (DNA) |
| 65h9 | SEQ ID NO: | SEQ ID NO: | SEQ ID NOs: | SEQ ID NOs: | SEQ ID NO: | SEQ ID NO: |
| | 1 | 2 | 29-31 | 32-34 | 15 | 16 |
| 52d9 | SEQ ID NO: | SEQ ID NO: | SEQ ID NOs: | SEQ ID NOs: | SEQ ID NO: | SEQ ID NO: |
| | 3 | 4 | 35-37 | 38-40 | 17 | 18 |
| 49f1 | SEQ ID NO: | SEQ ID NO: | SEQ ID NOs: | SEQ ID NOs: | SEQ ID NO: | SEQ ID NO: |
| | 5 | 6 | 41-43 | 44-46 | 19 | 20 |
| 47d11 | SEQ ID NO: | SEQ ID NO: | SEQ ID NOs: | SEQ ID NOs: | SEQ ID NO: | SEQ ID NO: |
| | 9 | 10 | 53-55 | 56-58 | 23 | 24 |

| SEQ ID NO | Description | Protein or DNA |
|---|---|---|
| 7 | 52d9 Q3 variant VH domain | Protein |
| 8 | SARS-CoV-2 S | Protein |
| 11 | VK 3-15 germline sequence | Protein |
| 12 | VH 4-59 germline sequence | Protein |
| 13 | VK 1-39 germline sequence | Protein |
| 14 | VH 1-69 germline sequence | Protein |
| 21 | SARS-CoV-2 S RBD | Protein |
| 22 | SARS-CoV-2 S RBD Orange | Protein |
| 25 | SARS-CoV S | Protein |
| 26 | SARS-CoV S RBD | Protein |
| 27 | SARS-CoV S RBD Orange | Protein |
| 47 | 49f1 v2 VL domain | Protein |
| 48 | 49f1 v3 VL domain | Protein |
| 49 | 49f1 v2 VL domain | DNA |
| 50 | 49f1 v3 VL domain | DNA |
| 52 | VK 1-5*03 germline sequence | Protein |
| 59-61 | 49f1 v2 CDRL1-3 | Protein |
| 62-64 | 49f1 v3 CDRL1-3 | Protein |
| 65 | 47d11 heavy chain | Protein |
| 66 | 47d11 light chain | Protein |
| 67 | SARS2-S V367F | Protein |
| 68 | SARS2-S1B V367F | Protein |
| 69 | 47d11 heavy chain | DNA |
| 70 | 47d11 light chain | DNA |
| 71 | SARS-CoV-2 S (GenBank: QHD43416.1) | Protein |
| 72 | 49F1 v2 light chain (kappa) | DNA |
| 73 | 49F1 v2 light chain (kappa) | Protein |
| 74 | 49F1 heavy chain (IgG1) | DNA |
| 75 | 49F1 heavy chain (IgG1) | Protein |

### DETAILED DESCRIPTION OF THE INVENTION

### Anti-SARS2-S Antibodies

The invention provides an antibody that binds to SARS2. In some embodiments, the antibody binds to the spike protein of SARS2 (SARS2-S). In some embodiments, the antibody binds to the S1 subunit of SARS2-S. In some embodiments, the antibody binds to the S1_{B} subunit of SARS2-S. In some embodiments, the antibody binds to the S1_{A} subunit of SARS2-S. In some embodiments, the antibody binds to the S1_{C} subunit of SARS2-S. In some embodiments, the antibody binds to the S1_{D} subunit of SARS2-S. In some embodiments, the antibody binds to the S2 subunit of SARS2-S.

In some embodiments, the antibody binds to a SARS2-S protein having the sequence of SEQ ID NO: 8. In some embodiments, the antibody binds to a SARS2-S protein having the sequence of SEQ ID NO: 71. SEQ ID NO: 71 is the SARS2-S protein sequence at GenBank: QHD43416.1. In some embodiments, the antibody binds to the SARS2-S protein at one or more (e.g. two or more, three or more, four or more, or five or more) of the residues in the sequence of SEQ NO: 21. In some embodiments, the antibody binds to binds to the SARS2-S protein at an epitope within the sequence of SEQ NO: 21. In some embodiments, the antibody binds to the SARS2-S protein at one or more (e.g. two or more, three or more, four or more, or five or more) of the residues in the sequence of SEQ ID NO: 22. In some embodiments, the antibody binds to binds to the SARS2-S protein at an epitope within the sequence of SEQ NO: 22.

In some embodiments, the antibody is additionally capable of binding to a SARS1-S protein having the sequence of SEQ ID NO: 25. In some embodiments, the antibody binds to the SARS1-S protein at one or more (e.g. two or more, three or more, four or more, or five or more) of the residues in the sequence of SEQ NO: 26. In some embodiments, the antibody binds to binds to the SARS1-S protein at an epitope within the sequence of SEQ NO: 26. In some embodiments, the antibody binds to the SARS1-S protein at one or more (e.g. two or more, three or more, four or more, or five or more) of the residues in the sequence of SEQ ID NO: 27. In some embodiments, the antibody binds to binds to the SARS1-S protein at an epitope within the sequence of SEQ NO: 27.

In some embodiments, the antibody binds to the S1 subunit of SARS2-S and inhibits the binding of SARS2-S to human angiotensin converting enzyme 2 receptor (ACE2). In some embodiments, the antibody binds to the S1_{B} subunit of SARS2-S and inhibits the binding of SARS2-S to ACE2. In some embodiments, the antibody binds to the S1_{B} subunit of SARS2-S, but does not inhibit the binding of SARS2-S to ACE2.

In some embodiments, the antibody binds to the S1 subunit of SARS2-S and inhibits SARS2 infections of human cells. In some embodiments, the antibody binds to the S1_{A} subunit of SARS2-S and inhibits SARS2 infections of human cells. In some embodiments, the antibody binds to the S1_{B} subunit of SARS2-S and inhibits SARS2 infections of human cells. In some embodiments, the antibody binds to the S2 subunit of SARS2-S and inhibits SARS2 infections of human cells.

In some embodiments, the antibody binds to the S1 subunit of SARS2-S and inactivates SARS2-S (e.g. through antibody-induced destabilization of the prefusion structure of SARS2-S). In some embodiments, the antibody binds to the S1_{B} subunit of SARS2-S and inactivates SARS2-S (e.g. through antibody-induced destabilization of the prefusion structure of SARS2-S). In some embodiments, the antibody binds to the S1_{A} subunit of SARS2-S and inactivates SARS2-S (e.g. through antibody-induced destabilization of the prefusion structure of SARS2-S). In some embodiments, the antibody binds to the S2 subunit of SARS2-S and inactivates SARS2-S (e.g. through antibody-induced destabilization of the prefusion structure of SARS2-S).

The Examples show that the 47d11 antibody binds to SARS2-S1, as well as SARS-S_{octo} and SARS-S1. The 47d11 antibody does not bind to SARS-S1_{A}. The 47d11 antibody impairs SARS-S and SARS2-S mediated syncytia formation. The 47d11 antibody also potently neutralizes SARS1 and SARS2 infections of human cells. The 47d11 antibody is also shown in Example 5 to protect against weight loss and to significantly reduce pneumonia and pulmonary virus replication in an animal model of severe SARS-CoV-2 pneumonia. The 47d11 antibody binds to receptor binding domain mutants including K417N, E484K and N501Y, as well as N439K, F490S, Q493R and S494P. Similar functional properties can be expected to be associated with the antibodies defined below which share structural and binding characteristics with the 47d11 antibody.

The invention provides an anti-SARS2-S antibody that binds to SARS2-S having the sequence of SEQ ID NO: 71, wherein the antibody binds to an epitope comprising one or more (e.g. two, three, four, five, six, seven or more) of residues F338, F342, N343, Y365, V367, L368, F374 and W436 of SEQ ID NO: 71. In some embodiments, the antibody is an IgG (e.g. an IgG1) that comprises two heavy and two light chains.

The invention provides an anti-SARS2-S antibody that binds to SARS2-S having the sequence of SEQ ID NO: 71, wherein the antibody binds to an epitope comprising residues F338, F342, N343, Y365, V367, L368, F374 and W436 of SEQ ID NO: 71. In some embodiments, the antibody is an IgG (e.g. an IgG1) that comprises two heavy and two light chains.

The invention provides an anti-SARS2-S antibody that binds to SARS2-S having the sequence of SEQ ID NO: 71, wherein the antibody binds to an epitope consisting of residues F338, F342, N343, Y365, V367, L368, F374 and W436 of SEQ ID NO: 71. In some embodiments, the antibody is an IgG (e.g. an IgG1) that comprises two heavy and two light chains.

The invention provides an anti-SARS2-S antibody that binds to SARS2-S having the sequence of SEQ ID NO: 8, wherein the antibody binds to an epitope comprising one or more (e.g. two, three, four, five, six, seven or more) of residues F268, F272, N273, Y295, V297, L298, F304 and W366 of SEQ ID NO: 8. In some embodiments, the antibody is an IgG (e.g. an IgG1) that comprises two heavy and two light chains.

The invention provides an anti-SARS2-S antibody that binds to SARS2-S having the sequence of SEQ ID NO: 8, wherein the antibody binds to an epitope comprising residues F268, F272, N273, Y295, V297, L298, F304 and W366 of SEQ ID NO: 8. In some embodiments, the antibody is an IgG (e.g. an IgG1) that comprises two heavy and two light chains.

The invention provides an anti-SARS2-S antibody that binds to SARS2-S having the sequence of SEQ ID NO: 8, wherein the antibody binds to an epitope consisting of residues F268, F272, N273, Y295, V297, L298, F304 and W366 of SEQ ID NO: 8. In some embodiments, the antibody is an IgG (e.g. an IgG1) that comprises two heavy and two light chains.

The invention provides an anti-SARS2-S antibody that binds to SARS2-S having the sequence of SEQ ID NO: 21, wherein the antibody binds to an epitope comprising one or more (e.g. two, three, four, five, six, seven or more) of residues F3, F7, N8, Y30, V32, L33, F39 and W101 of SEQ ID NO: 21. In some embodiments, the antibody is an IgG (e.g. an IgG1) that comprises two heavy and two light chains.

The invention provides an anti-SARS2-S antibody that binds to SARS2-S having the sequence of SEQ ID NO: 21, wherein the antibody binds to an epitope comprising residues F3, F7, N8, Y30, V32, L33, F39 and W101 of SEQ ID NO: 21. In some embodiments, the antibody is an IgG (e.g. an IgG1) that comprises two heavy and two light chains.

The invention provides an anti-SARS2-S antibody that binds to SARS2-S having the sequence of SEQ ID NO: 21, wherein the antibody binds to an epitope consisting of residues F3, F7, N8, Y30, V32, L33, F39 and W101 of SEQ ID NO: 21. In some embodiments, the antibody is an IgG (e.g. an IgG1) that comprises two heavy and two light chains.

The invention provides an anti-SARS2-S antibody that binds to SARS2-S having the sequence of SEQ ID NO: 67, wherein the antibody binds to an epitope comprising one or more (e.g. two, three, four, five, six, seven or more) of residues F268, F272, N273, Y295, V297, L298, F304 and W366 of SEQ ID NO: 67. In some embodiments, the antibody is an IgG (e.g. an IgG1) that comprises two heavy and two light chains.

The invention provides an anti-SARS2-S antibody that binds to SARS2-S having the sequence of SEQ ID NO: 67, wherein the antibody binds to an epitope comprising residues F268, F272, N273, Y295, V297, L298, F304 and W366 of SEQ ID NO: 67. In some embodiments, the antibody is an IgG (e.g. an IgG1) that comprises two heavy and two light chains.

The invention provides an anti-SARS2-S antibody that binds to SARS2-S having the sequence of SEQ ID NO: 67, wherein the antibody binds to an epitope consisting of residues F268, F272, N273, Y295, V297, L298, F304 and W366 of SEQ ID NO: 67. In some embodiments, the antibody is an IgG (e.g. an IgG1) that comprises two heavy and two light chains.

The invention provides an anti-SARS2-S antibody that binds to SARS2-S having the sequence of SEQ ID NO: 68, wherein the antibody binds to an epitope comprising one or more (e.g. two, three, four, five, six, seven or more) of residues F3, F7, N8, Y30, V32, L33, F39 and W101 of SEQ ID NO: 68. In some embodiments, the antibody is an IgG (e.g. an IgG1) that comprises two heavy and two light chains.

The invention provides an anti-SARS2-S antibody that binds to SARS2-S having the sequence of SEQ ID NO: 68, wherein the antibody binds to an epitope comprising residues F3, F7, N8, Y30, V32, L33, F39 and W101 of SEQ ID NO: 68. In some embodiments, the antibody is an IgG (e.g. an IgG1) that comprises two heavy and two light chains.

The invention provides an anti-SARS2-S antibody that binds to SARS2-S having the sequence of SEQ ID NO: 68, wherein the antibody binds to an epitope consisting of residues F3, F7, N8, Y30, V32, L33, F39 and W101 of SEQ ID NO: 68. In some embodiments, the antibody is an IgG (e.g. an IgG1) that comprises two heavy and two light chains. The invention provides an anti-SARS2-S antibody that binds to SARS2-S comprising the K417N mutation.

The invention provides an anti-SARS2-S antibody that binds to SARS2-S comprising the E484K mutation.

The invention provides an anti-SARS2-S antibody that binds to SARS2-S comprising the N501Y.

The invention further provides an anti-SARS2-S antibody that bind to SARS2-S comprising one or more of the K417N, E484K and N501Y mutations.

The invention provides an anti-SARS2-S antibody that binds to SARS2-S having the sequence of SEQ ID NO: 71 with a single substitution, wherein the substitution is K417N.

The invention provides an anti-SARS2-S antibody that binds to SARS2-S having the sequence of SEQ ID NO: 71 with a single substitution, wherein the substitution is E484K.

The invention provides an anti-SARS2-S antibody that binds to SARS2-S having the sequence of SEQ ID NO: 71 with a single substitution, wherein the substitution is N501Y.

The invention provides an anti-SARS2-S antibody that binds to SARS2-S having the sequence of SEQ ID NO: 71 with two substitutions, wherein the substitutions are K417N and E484K.

The invention provides an anti-SARS2-S antibody that binds to SARS2-S having the sequence of SEQ ID NO: 71 with two substitutions, wherein the substitutions are K417N and N501Y.

The invention provides an anti-SARS2-S antibody that binds to SARS2-S having the sequence of SEQ ID NO: 71 with three substitutions, wherein the substitutions are K417N, E484K and N501Y.

Accordingly, the invention provides an anti-SARS2-S antibody that binds to:
(a) SARS2-S having the sequence of SEQ ID NO: 71,
(b) SARS2-S having the sequence of SEQ ID NO: 71 with a single substitution, wherein the substitution is K417N,
(c) SARS2-S having the sequence of SEQ ID NO: 71 with a single substitution, wherein the substitution is E484K, and
(d) SARS2-S having the sequence of SEQ ID NO: 71 with a single substitution, wherein the substitution is N501Y.

The invention further provides an anti-SARS2-S antibody that:
(i) binds to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K, and
   (d) SARS2-S having the sequence of SEQ ID NO: 71 with a single substitution, wherein the substitution is N501Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 71 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K, and
   (d) SARS2-S having the sequence of SEQ ID NO: 71 with a single substitution, wherein the substitution is N501Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the antibody is an IgG (e.g. an IgG1) that comprises two heavy and two light chains.

In some embodiments, the presence or absence of binding is determined by flow cytometry.

The invention further provides an anti-SARS2-S antibody that:
(i) binds to:
   (a) SARS2-S having the sequence of SEQ ID NO: 71,
   (b) SARS2-S having the sequence of SEQ ID NO: 71 with a single substitution, wherein the substitution is K347N,
   (c) SARS2-S having the sequence of SEQ ID NO: 71 with a single substitution, wherein the substitution is E414K, and
   (d) SARS2-S having the sequence of SEQ ID NO: 71 with a single substitution, wherein the substitution is N431Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 71 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K, and
   (d) SARS2-S having the sequence of SEQ ID NO: 71 with a single substitution, wherein the substitution is N501Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the antibody is an IgG (e.g. an IgG1) that comprises two heavy and two light chains.

In some embodiments, the presence or absence of binding is determined by flow cytometry, for example using cells expressing the SARS2-S protein.

The invention further provides an anti-SARS2-S antibody that bind to SARS2-S comprising one or more of the N439K, F490S, Q493R and S494P mutations.

The invention provides an anti-SARS2-S antibody that binds to SARS2-S having the sequence of SEQ ID NO: 71 with a single substitution, wherein the substitution is N439K.

The invention provides an anti-SARS2-S antibody that binds to SARS2-S having the sequence of SEQ ID NO: 71 with a single substitution, wherein the substitution is F490S.

The invention provides an anti-SARS2-S antibody that binds to SARS2-S having the sequence of SEQ ID NO: 71 with a single substitution, wherein the substitution is Q493R.

The invention provides an anti-SARS2-S antibody that binds to SARS2-S having the sequence of SEQ ID NO: 71 with a single substitution, wherein the substitution is S494P.

Accordingly, the invention provides an anti-SARS2-S antibody that binds to:
(a) SARS2-S having the sequence of SEQ ID NO: 71,
(b) SARS2-S having the sequence of SEQ ID NO: 71 with a single substitution, wherein the substitution is K417N,
(c) SARS2-S having the sequence of SEQ ID NO: 71 with a single substitution, wherein the substitution is E484K,
(d) SARS2-S having the sequence of SEQ ID NO: 71 with a single substitution, wherein the substitution is N501Y,
(e) SARS2-S having the sequence of SEQ ID NO: 71 with a single substitution, wherein the substitution is N439K,
(f) SARS2-S having the sequence of SEQ ID NO: 71 with a single substitution, wherein the substitution is Q493R, and
(g) SARS2-S having the sequence of SEQ ID NO: 71 with a single substitution, wherein the substitution is S494P.

The invention further provides an anti-SARS2-S antibody that:
(i) binds to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (g) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 71 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (g) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the antibody is an IgG (e.g. an IgG1) that comprises two heavy and two light chains.

In some embodiments, the presence or absence of binding is determined by flow cytometry.

The invention further provides an anti-SARS2-S antibody that:
(i) binds to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (a) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 71 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (g) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the antibody is an IgG (e.g. an IgG1) that comprises two heavy and two light chains.

In some embodiments, the presence or absence of binding is determined by flow cytometry, for example using cells expressing the SARS2-S protein.

The invention provides an anti-SARS2-S antibody that binds to SARS2-S having the sequence of SEQ ID NO: 8 with a single substitution, wherein the substitution is K347N.

The invention provides an anti-SARS2-S antibody that binds to SARS2-S having the sequence of SEQ ID NO: 8 with a single substitution, wherein the substitution is E414K.

The invention provides an anti-SARS2-S antibody that binds to SARS2-S having the sequence of SEQ ID NO: 8 with a single substitution, wherein the substitution is the N431Y.

The invention provides an anti-SARS2-S antibody that binds to SARS2-S having the sequence of SEQ ID NO: 8 with two substitutions, wherein the substitutions are K347N and N431Y.

The invention provides an anti-SARS2-S antibody that binds to SARS2-S having the sequence of SEQ ID NO: 8 with two substitutions, wherein the substitutions are E414K and N431Y.

The invention provides an anti-SARS2-S antibody that binds to SARS2-S having the sequence of SEQ ID NO: 8 with two substitutions, wherein the substitutions are K347N and E414K.

The invention provides an anti-SARS2-S antibody that binds to SARS2-S having the sequence of SEQ ID NO: 8 with three substitutions, wherein the substitutions are K347N, E414K and N431Y.

Accordingly, the invention provides an anti-SARS2-S antibody that binds to:
(a) SARS2-S having the sequence of SEQ ID NO: 8,
(b) SARS2-S having the sequence of SEQ ID NO: 8 with a single substitution, wherein the substitution is K347N,
(c) SARS2-S having the sequence of SEQ ID NO: 8 with a single substitution, wherein the substitution is E414K, and
(d) SARS2-S having the sequence of SEQ ID NO: 8 with a single substitution, wherein the substitution is N431Y.

The invention further provides an anti-SARS2-S antibody that:
(i) binds to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A, and
   (c) SARS2-S having the sequence of SEQ ID NO: 8 with a single substitution, wherein the substitution is L298A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 8 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A, and
   (c) SARS2-S having the sequence of SEQ ID NO: 8 with a single substitution, wherein the substitution is L298A.

In some embodiments, the antibody is an IgG (e.g. an IgG1) that comprises two heavy and two light chains.

In some embodiments, the presence or absence of binding is determined by flow cytometry.

The invention further provides an anti-SARS2-S antibody that:
(i) binds to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 8 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A, and
   (g) SARS2-S having the sequence of SEQ ID NO: 8 with a single substitution, wherein the substitution is W366A.

In some embodiments, the antibody is an IgG (e.g. an IgG1) that comprises two heavy and two light chains.

In some embodiments, the presence or absence of binding is determined by flow cytometry, for example using cells expressing the SARS2-S protein.

The invention further provides an anti-SARS2-S antibody that bind to SARS2-S comprising one or more of the N439K, F490S, Q493R and S494P mutations.

The invention provides an anti-SARS2-S antibody that binds to SARS2-S having the sequence of SEQ ID NO: 8 with a single substitution, wherein the substitution is N369K.

The invention provides an anti-SARS2-S antibody that binds to SARS2-S having the sequence of SEQ ID NO: 8 with a single substitution, wherein the substitution is F420S.

The invention provides an anti-SARS2-S antibody that binds to SARS2-S having the sequence of SEQ ID NO: 8 with a single substitution, wherein the substitution is Q423R.

The invention provides an anti-SARS2-S antibody that binds to SARS2-S having the sequence of SEQ ID NO: 8 with a single substitution, wherein the substitution is S424P.

Accordingly, the invention provides an anti-SARS2-S antibody that binds to:
(a) SARS2-S having the sequence of SEQ ID NO: 8,
(b) SARS2-S having the sequence of SEQ ID NO: 8 with a single substitution, wherein the substitution is K347N,
(c) SARS2-S having the sequence of SEQ ID NO: 8 with a single substitution, wherein the substitution is E414K,
(d) SARS2-S having the sequence of SEQ ID NO: 8 with a single substitution, wherein the substitution is N431Y,
(e) SARS2-S having the sequence of SEQ ID NO: 8 with a single substitution, wherein the substitution is N369K,
(f) SARS2-S having the sequence of SEQ ID NO: 8 with a single substitution, wherein the substitution is Q423R, and
(d) SARS2-S having the sequence of SEQ ID NO: 8 with a single substitution, wherein the substitution is S424P.

The invention further provides an anti-SARS2-S antibody that:
(i) binds to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K,
   (d) SARS2-S having the sequence of SEQ ID NO: 8 with a single substitution, wherein the substitution is N431Y,
   (e) SARS2-S having the sequence of SEQ ID NO: 8 with a single substitution, wherein the substitution is N369K,
   (f) SARS2-S having the sequence of SEQ ID NO: 8 with a single substitution, wherein the substitution is Q423R, and
   (g) SARS2-S having the sequence of SEQ ID NO: 8 with a single substitution, wherein the substitution is S424P; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A, and
   (c) SARS2-S having the sequence of SEQ ID NO: 8 with a single substitution, wherein the substitution is L298A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 8 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K,
   (d) SARS2-S having the sequence of SEQ ID NO: 8 with a single substitution, wherein the substitution is N431Y,
   (e) SARS2-S having the sequence of SEQ ID NO: 8 with a single substitution, wherein the substitution is N369K,
   (f) SARS2-S having the sequence of SEQ ID NO: 8 with a single substitution, wherein the substitution is Q423R, and
   (g) SARS2-S having the sequence of SEQ ID NO: 8 with a single substitution, wherein the substitution is S424P; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A, and
   (c) SARS2-S having the sequence of SEQ ID NO: 8 with a single substitution, wherein the substitution is L298A.

In some embodiments, the antibody is an IgG (e.g. an IgG1) that comprises two heavy and two light chains.

In some embodiments, the presence or absence of binding is determined by flow cytometry.

The invention further provides an anti-SARS2-S antibody that:
(i) binds to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y,
   (e) SARS2-S having the sequence of SEQ ID NO: 8 with a single substitution, wherein the substitution is N369K,
   (f) SARS2-S having the sequence of SEQ ID NO: 8 with a single substitution, wherein the substitution is Q423R, and
   (g) SARS2-S having the sequence of SEQ ID NO: 8 with a single substitution, wherein the substitution is S424P; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the antibody is an IgG (e.g. an IgG1) that comprises two heavy and two light chains.

In some embodiments, the presence or absence of binding is determined by flow cytometry.

In some embodiments, the antibody binds to SARS2-S having the sequence of SEQ ID NO: 8 with a K_{D} of 10⁻⁸ M or less, 10⁻⁹ M or less, or 10⁻¹⁰ M or less, as determined by surface plasmon resonance.

The invention further provides an anti-SARS2-S antibody that:
(i) binds with a K_{D} of 10⁻⁸ M or less, as determined by surface plasmon resonance, to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K,
   (d) SARS2-S having the sequence of SEQ ID NO: 8 with a single substitution, wherein the substitution is N431Y,
   (e) SARS2-S having the sequence of SEQ ID NO: 8 with a single substitution, wherein the substitution is N369K,
   (f) SARS2-S having the sequence of SEQ ID NO: 8 with a single substitution, wherein the substitution is Q423R, and
   (g) SARS2-S having the sequence of SEQ ID NO: 8 with a single substitution, wherein the substitution is S424P; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A, and
   (g) SARS2-S having the sequence of SEQ ID NO: 8 with a single substitution, wherein the substitution is W366A.

In some embodiments, the antibody is an IgG (e.g. an IgG1) that comprises two heavy and two light chains.

In some embodiments, the presence or absence of binding is determined by flow cytometry, for example using cells expressing the SARS2-S protein.

The invention further provides an anti-SARS2-S antibody that binds specifically to the closed conformation of S1_{B} of SARS2-S.

In some embodiments, the antibody binds to S1_{B} distal to the ACE2 binding site.

In some embodiments, the antibody stabilises the N343 glycan of SARS2-S in an upright conformation.

In some embodiments, the antibody stabilises the N343 glycan of SARS2-S in an upright conformation, thereby exposing a hydrophobic pocket to which the antibody binds via one or more aromatic residues in one or more of its CDRs.

In some embodiments, the antibody binds to S1_{B} distal to the ACE2 binding site, and stabilises the N343 glycan of SARS2-S in an upright conformation, thereby exposing a hydrophobic pocket to which the antibody binds via one or more aromatic residues in one or more of its CDRs.

In some embodiments, the antibody stabilises the N343 glycan of SARS2-S in an upright conformation, thereby exposing a hydrophobic pocket to which the antibody binds via one or more aromatic residues in a CDRH3 loop.

In some embodiments, the antibody binds to S1_{B} distal to the ACE2 binding site, and stabilises the N343 glycan of SARS2-S in an upright conformation, thereby exposing a hydrophobic pocket to which the antibody binds via one or more aromatic residues in a CDRH3 loop.

In some embodiments, the antibody stabilises the N343 glycan of SARS2-S in an upright conformation, thereby exposing a hydrophobic pocket to which the antibody binds via two aromatic residues in a CDRH3 loop.

In some embodiments, the antibody binds to S1_{B} distal to the ACE2 binding site, and stabilises the N343 glycan of SARS2-S in an upright conformation, thereby exposing a hydrophobic pocket to which the antibody binds via two aromatic residues in a CDRH3 loop.

The invention further provides an anti-SARS2-S antibody that binds specifically to the partially open conformation of a SARS2-S trimer.

The invention further provides an anti-SARS2-S antibody that binds specifically to: (i) the partially open conformation of a SARS2-S trimer and (ii) the closed conformation of a SARS1-S trimer.

In some embodiments, when the antibody binds to SARS1-S, when the antibody binds to SARS1-S, the antibody stabilises the N330 glycan of SARS1-S in an upright conformation.

In some embodiments, when the antibody binds to SARS1-S, the antibody forms a stabilising salt bridge with the receptor binding ridge of SARS1-S.

In some embodiments, when the antibody binds to SARS1-S, a light chain variable domain of the antibody forms a stabilising salt bridge with the receptor binding ridge of SARS1-S.

In some embodiments, when the antibody binds to SARS1-S, when the antibody binds to SARS1-S, the antibody stabilises the N330 glycan of SARS1-S in an upright conformation, and the antibody forms a stabilising salt bridge with the receptor binding ridge of SARS1-S.

In some embodiments, when the antibody binds to SARS1-S, when the antibody binds to SARS1-S, the antibody stabilises the N330 glycan of SARS1-S in an upright conformation, and a light chain variable domain of the antibody forms a stabilising salt bridge with the receptor binding ridge of SARS1-S.

In some embodiments, when the antibody binds to SARS1-S, when the antibody binds to SARS1-S, the antibody stabilises the N330 glycan of SARS1-S in an upright conformation, and a light chain variable domain of the antibody forms a stabilising salt bridge with the receptor binding ridge of SARS1-S, wherein the stabilising salt bridge is formed between R18 of the light chain variable domain and D463 of SARS1-S.

The invention further provides an anti-SARS2-S antibody that:
(a) wherein the antibody binds to S1_{B} distal to the ACE2 binding site, wherein the antibody binds to a S1_{B} in closed conformation within the partially open form of a SARS2-S trimer, wherein the antibody stabilises the N343 glycan of SARS2-S in an upright conformation, thereby exposing a hydrophobic pocket to which the antibody binds via one or more (e.g. two) aromatic residues in a CDRH3 loop, and
(b) wherein the antibody binds to S1_{B} of SARS1-S within the closed form of a SARS1-S trimer, wherein the antibody stabilises the N330 glycan of SARS2-S in an upright conformation, thereby exposing a hydrophobic pocket to which the antibody binds via one or more (e.g. two) aromatic residues in a CDRH3 loop, and wherein a light chain variable domain of the antibody forms a stabilising salt bridge with the receptor binding ridge of SARS1-S.

In some embodiments, the antibody specifically binds to the S1_{B} domain of WIV16.

In some embodiments, the antibody specifically binds to the S1_{B} domain of WIV16 with similar binding affinity to the S1_{B} domain of SARS1-S and SARS2-S.

In some embodiments, the antibody does not bind to the S1_{B} domain of HKU3.3 or the S1_{B} domain of HKU9.3.

In some embodiments, the antibody binds to a mutated S1_{B} domain of HKU3.3, wherein DK has been mutated to GE to align with G4 and E5 in the sequence of SEQ ID NO: 21.

Exemplary screening methods for identifying further antibodies with the properties set out above may involve:
(i) positive screening for binding to SARS1-S1_{B}, SARS2-S1_{B}, WIV16-S1_{B}, and/or mutant HKU3.3-S_{1B} in which DK has been mutated to GE to align with G4 and E5 in the sequence of SEQ ID NO: 21, and/or
(ii) negative screening for binding to SARS1-S1A, SARS2-S1A, and/or mutant SARS2-S1_{B} having the sequence of SEQ ID NO: 21 with any one of the following substitutions: F3A, F7A, NBA, Y30A, L33A, F39A and W101A.

Exemplary screening methods may also involve assessing competition with 47D11 for binding to SARS1-S1_{B}, SARS2-S1_{B}, WIV16-S1_{B}, and/or mutant HKU3.3-S1_{B} in which DK has been mutated to GE to align with G4 and E5 in the sequence of SEQ ID NO: 21.

Exemplary screening methods may also involve negative screening for competitive inhibition with ACE2 for binding to SARS2-S1_{B}.

Sequence analysis of the CDRL3 and CDRH3 may also be used to identify whether there are hydrophobic aromatic residues (e.g. W or F) present.

The invention provides an anti-SARS2-S antibody that binds to the same epitope as an antibody comprising a light chain variable region of the amino acid sequence of SEQ ID NO: 9 and a heavy chain variable region of the amino acid sequence of SEQ ID NO: 10.

The invention further provides an anti-SARS2-S antibody that binds to the same epitope as a heavy chain-only antibody comprising a heavy chain variable region of the amino acid sequence of SEQ ID NO: 10.

The invention further provides an anti-SARS2-S antibody that competes for binding to SARS2-S with an antibody comprising a light chain variable region of the amino acid sequence of SEQ ID NO: 9 and a heavy chain variable region of the amino acid sequence of SEQ ID NO: 10.

The invention further provides an anti-SARS2-S antibody that competes for binding to SARS2-S with a heavy chain-only antibody comprising a heavy chain variable region of the amino acid sequence of SEQ ID NO: 10.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain.

In some embodiments, the antibody comprises a light chain variable region of the amino acid sequence of SEQ ID NO: 9.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 9.

In some embodiments, the antibody comprises a heavy chain variable region of the amino acid sequence of SEQ ID NO: 10.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 10.

In some embodiments, the antibody comprises a light chain variable region of the amino acid sequence of SEQ ID NO: 9 and a heavy chain variable region of the amino acid sequence of SEQ ID NO: 10.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 9 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 10.

The invention further provides an anti-SARS2-S antibody comprising complementarity determining regions (CDRs) with:
i. a sequence that is at least 90% identical to SEQ ID NO: 53 for CDR1 of the light chain;
ii. a sequence that is at least 90% identical to SEQ ID NO: 54 for CDR2 of the light chain;
iii. a sequence that is at least 90% identical to SEQ ID NO: 55 for CDR3 of the light chain;
iv. a sequence that is at least 90% identical to SEQ ID NO: 56 for CDR1 of the heavy chain;
v. a sequence that is at least 90% identical to SEQ ID NO: 57 for CDR2 of the heavy chain; and
vi. a sequence that is at least 90% identical to SEQ ID NO: 58 for CDR3 of the heavy chain,
   wherein the antibody competes for binding to SARS2-S with an antibody comprising a light chain variable region of the amino acid sequence of SEQ ID NO: 9 and a heavy chain variable region of the amino acid sequence of SEQ ID NO: 10.

The invention further provides an anti-SARS2-S antibody comprising complementarity determining regions (CDRs) with:
i. a sequence that is at least 95% identical to SEQ ID NO: 53 for CDR1 of the light chain;
ii. a sequence that is at least 95% identical to SEQ ID NO: 54 for CDR2 of the light chain;
iii. a sequence that is at least 95% identical to SEQ ID NO: 55 for CDR3 of the light chain;
iv. a sequence that is at least 95% identical to SEQ ID NO: 56 for CDR1 of the heavy chain;
v. a sequence that is at least 95% identical to SEQ ID NO: 57 for CDR2 of the heavy chain; and
vi. a sequence that is at least 95% identical to SEQ ID NO: 58 for CDR3 of the heavy chain,
   wherein the antibody competes for binding to SARS2-S with an antibody comprising a light chain variable region of the amino acid sequence of SEQ ID NO: 9 and a heavy chain variable region of the amino acid sequence of SEQ ID NO: 10.

The invention further provides an anti-SARS2-S antibody comprising complementarity determining regions (CDRs) with:
i. a sequence that is at least 70% identical to SEQ ID NO: 53 for CDR1 of the light chain;
ii. a sequence that is at least 70% identical to SEQ ID NO: 54 for CDR2 of the light chain;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is W, Y or F,
   X₇ is P,
   X₈ is L, G, A, V or I, and
   X₉ is T, S, C, U or M;
iv. a sequence that is at least 70% identical to SEQ ID NO: 56 for CDR1 of the heavy chain;
v. a sequence that is at least 70% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W, F or Y,
   X₈ is F, W or Y,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

The invention further provides an anti-SARS2-S antibody comprising complementarity determining regions (CDRs) with:
i. a sequence that is at least 80% identical to SEQ ID NO: 53 for CDR1 of the light chain;
ii. a sequence that is at least 80% identical to SEQ ID NO: 54 for CDR2 of the light chain;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is W, Y or F,
   X₇ is P,
   X₈ is L, G, A, V or I, and
   X₉ is T, S, C, U or M;
iv. a sequence that is at least 80% identical to SEQ ID NO: 56 for CDR1 of the heavy chain;
v. a sequence that is at least 80% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W, F or Y,
   X₈ is F, W or Y,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

The invention further provides an anti-SARS2-S antibody comprising complementarity determining regions (CDRs) with:
i. a sequence that is at least 90% identical to SEQ ID NO: 53 for CDR1 of the light chain;
ii. a sequence that is at least 90% identical to SEQ ID NO: 54 for CDR2 of the light chain;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is W, Y or F,
   X₇ is P,
   X₈ is L, G, A, V or I, and
   X₉ is T, S, C, U or M;
iv. a sequence that is at least 90% identical to SEQ ID NO: 56 for CDR1 of the heavy chain;
v. a sequence that is at least 90% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W, F or Y,
   X₈ is F, W or Y,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

The invention further provides an anti-SARS2-S antibody comprising complementarity determining regions (CDRs) with:
i. a sequence that is at least 70% identical to SEQ ID NO: 53 for CDR1 of the light chain;
ii. a sequence that is at least 70% identical to SEQ ID NO: 54 for CDR2 of the light chain;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is W,
   X₇ is P,
   X₈ is L, G, A, V or I, and
   X₉ is T, S, C, U or M;
iv. a sequence that is at least 70% identical to SEQ ID NO: 56 for CDR1 of the heavy chain;
v. a sequence that is at least 70% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W,
   X₈ is F,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

The invention further provides an anti-SARS2-S antibody comprising complementarity determining regions (CDRs) with:
i. a sequence that is at least 80% identical to SEQ ID NO: 53 for CDR1 of the light chain;
ii. a sequence that is at least 80% identical to SEQ ID NO: 54 for CDR2 of the light chain;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is
   X₇ is P,
   X₈ is L, G, A, V or I, and
   X₉ is T, S, C, U or M;
iv. a sequence that is at least 80% identical to SEQ ID NO: 56 for CDR1 of the heavy chain;
v. a sequence that is at least 80% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W,
   X₈ is F,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

The invention further provides an anti-SARS2-S antibody comprising complementarity determining regions (CDRs) with:
i. a sequence that is at least 90% identical to SEQ ID NO: 53 for CDR1 of the light chain;
ii. a sequence that is at least 90% identical to SEQ ID NO: 54 for CDR2 of the light chain;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is
   X₇ is P,
   X₈ is L, G, A, V or I, and
   X₉ is T, S, C, U or M;
iv. a sequence that is at least 90% identical to SEQ ID NO: 56 for CDR1 of the heavy chain;
v. a sequence that is at least 90% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W,
   X₈ is F,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

The invention further provides an anti-SARS2-S antibody comprising complementarity determining regions (CDRs) with:
i. a sequence comprising or consisting of X₁X₂X₃X₄X₅X₆ for CDR1 of the light chain, wherein:
   X₁ is Q, D, E or N,
   X₂ is S, C, U, T or M,
   X₃ is V, G, A, L or I,
   X₄ is S, C, U, T or M,
   X₅ is S, C, U, T or M, and
   X₆ is S, C, U, T or M;
ii. a sequence comprising or consisting of X₁X₂X₃ for CDR2 of the light chain, wherein:
   X₁ is G, A, V, L or I,
   X₂ is A, G, V, L or I, and
   X₃ is S, C, U, T or M;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is W, Y or F,
   X₇ is P,
   X₈ is L, G, A, V or I, and
   X₉ is T, S, C, U or M;
iv. a sequence that is comprising or consisting of X₁X₂X₃X₄X₅X₆X₇X₈ for CDR1 of the heavy chain, wherein:
   X₁ is G, A, V, L or I,
   X₂ is G, A, V, L or I,
   X₃ is S, C, U, T or M,
   X₄ is I, G, A, V or L,
   X₅ is S, C, U, T or M,
   X₆ is S, C, U, T or M,
   X₇ is H, K or R, and
   X₈ is Y, F or W;
v. a sequence that is comprising or consisting of X₁X₂X₃X₄X₅X₆X₇ for CDR2 of the heavy chain, wherein:
   X₁ is G, A, V, L or I,
   X₂ is Y, F or W,
   X₃ is Y, F or W,
   X₄ is S, C, U, T or M,
   X₅ is G, A, V, L or I,
   X₆ is S, C, U, T or M, and
   X₇ is T, S, C, U or M; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W, F or Y,
   X₈ is F, W or Y,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

The invention further provides an anti-SARS2-S antibody comprising complementarity determining regions (CDRs) with:
i. a sequence comprising or consisting of X₁X₂X₃X₄X₅X₆ for CDR1 of the light chain, wherein:
   X₁ is Q, D, E or N,
   X₂ is S, C, U, T or M,
   X₃ is V, G, A, L or I,
   X₄ is S, C, U, T or M,
   X₅ is S, C, U, T or M, and
   X₆ is S, C, U, T or M;
ii. a sequence comprising or consisting of X₁X₂X₃ for CDR2 of the light chain, wherein:
   X₁ is G, A, V, L or I,
   X₂ is A, G, V, L or I, and
   X₃ is S, C, U, T or M;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is W,
   X₇ 1 S P,
   X₈ is L, G, A, V or I, and
   X₉ is T, S, C, U or M;
iv. a sequence that is comprising or consisting of X₁X₂X₃X₄X₅X₆X₇X₈ for CDR1 of the heavy chain, wherein:
   X₁ is G, A, V, L or I,
   X₂ is G, A, V, L or I,
   X₃ is S, C, U, T or M,
   X₄ is I, G, A, V or L,
   X₅ is S, C, U, T or M,
   X₆ is S, C, U, T or M,
   X₇ is H, K or R, and
   X₈ is Y, F or W;
v. a sequence that is comprising or consisting of X₁X₂X₃X₄X₅X₆X₇ for CDR2 of the heavy chain, wherein:
   X₁ is G, A, V, L or I,
   X₂ is Y, F or W,
   X₃ is Y, F or W,
   X₄ is S, C, U, T or M,
   X₅ is G, A, V, L or I,
   X₆ is S, C, U, T or M, and
   X₇ is T, S, C, U or M; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W,
   X₈ is F,
   X₉ is G, A, V, L or I,
   X₁₀ is Q, D, E or N,
   X₁₁ is P,
   X₁₂ is G, A, V or L,
   X₁₃ is F, W or Y,
   X₁₄ is Q, D, E or N, and
   X₁₅ is I, G, A, V or L.

In some embodiments, the anti-SARS2 antibody binds to SARS2-S1_{B} having the sequence of SEQ ID NO: 21 and does not bind to mutant SARS2-S1_{B} having the sequence of SEQ ID NO: 21 with any one of the following substitutions: F3A, F7A, NBA, Y30A, L33A, F39A and W101A.

The Examples show that the 65h9 antibody binds to SARS2-S1, as well as SARS-S_{ecto} and SARS-S1. The 65h9 antibody does not bind to SARS-S1_{A}. Similar functional properties can be expected to be associated with the antibodies defined below which share structural and binding characteristics with the 65h9 antibody.

The invention provides an anti-SARS2-S antibody that binds to the same epitope as an antibody comprising a light chain variable region of the amino acid sequence of SEQ ID NO: 1 and a heavy chain variable region of the amino acid sequence of SEQ ID NO: 2.

The invention further provides an anti-SARS2-S antibody that binds to the same epitope as a heavy chain-only antibody comprising a heavy chain variable region of the amino acid sequence of SEQ ID NO: 2.

The invention further provides an anti-SARS2-S antibody that competes for binding to SARS2-S with an antibody comprising a light chain variable region of the amino acid sequence of SEQ ID NO: 1 and a heavy chain variable region of the amino acid sequence of SEQ ID NO: 2.

The invention further provides an anti-SARS2-S antibody that competes for binding to SARS2-S with a heavy chain-only antibody comprising a heavy chain variable region of the amino acid sequence of SEQ ID NO: 2.

The invention further provides an anti-SARS2-S antibody comprising complementarity determining regions (CDRs) with the sequences of:
i. SEQ ID NO: 29 for CDR1 of the light chain;
ii. SEQ ID NO: 30 for CDR2 of the light chain;
iii. SEQ ID NO: 31 for CDR3 of the light chain;
iv. SEQ ID NO: 32 for CDR1 of the heavy chain;
v. SEQ ID NO: 33 for CDR2 of the heavy chain; and
vi. SEQ ID NO: 34 for CDR3 of the heavy chain.

In some embodiments, the antibody comprises a light chain variable region of the amino acid sequence of SEQ ID NO: 1.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 1.

In some embodiments, the antibody comprises a heavy chain variable region of the amino acid sequence of SEQ ID NO: 2.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 2.

In some embodiments, the antibody comprises a light chain variable region of the amino acid sequence of SEQ ID NO: 1 and a heavy chain variable region of the amino acid sequence of SEQ ID NO: 2.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 1 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 2.

The invention further provides an anti-SARS2-S antibody comprising complementarity determining regions (CDRs) with:
i. a sequence that is at least 90% identical to SEQ ID NO: 29 for CDR1 of the light chain;
ii. a sequence that is at least 90% identical to SEQ ID NO: 30 for CDR2 of the light chain;
iii. a sequence that is at least 90% identical to SEQ ID NO: 31 for CDR3 of the light chain;
iv. a sequence that is at least 90% identical to SEQ ID NO: 32 for CDR1 of the heavy chain;
v. a sequence that is at least 90% identical to SEQ ID NO: 33 for CDR2 of the heavy chain; and
vi. a sequence that is at least 90% identical to SEQ ID NO: 34 for CDR3 of the heavy chain,
   wherein the antibody competes for binding to SARS2-S with an antibody comprising a light chain variable region of the amino acid sequence of SEQ ID NO: 1 and a heavy chain variable region of the amino acid sequence of SEQ ID NO: 2.

The invention further provides an anti-SARS2-S antibody comprising complementarity determining regions (CDRs) with:
i. a sequence that is at least 95% identical to SEQ ID NO: 29 for CDR1 of the light chain;
ii. a sequence that is at least 95% identical to SEQ ID NO: 30 for CDR2 of the light chain;
iii. a sequence that is at least 95% identical to SEQ ID NO: 31 for CDR3 of the light chain;
iv. a sequence that is at least 95% identical to SEQ ID NO: 32 for CDR1 of the heavy chain;
v. a sequence that is at least 95% identical to SEQ ID NO: 33 for CDR2 of the heavy chain; and
vi. a sequence that is at least 95% identical to SEQ ID NO: 34 for CDR3 of the heavy chain,
   wherein the antibody competes for binding to SARS2-S with an antibody comprising a light chain variable region of the amino acid sequence of SEQ ID NO: 1 and a heavy chain variable region of the amino acid sequence of SEQ ID NO: 2.

The Examples show that the 52d9 antibody binds to SARS2-S1, as well as SARS-S_{ecto}, SARS-S1 and SARS-S1_{A}. Similar functional properties can be expected to be associated with the antibodies defined below which share structural and binding characteristics with the 52d9 antibody.

The invention provides an anti-SARS2-S antibody that binds to the same epitope as an antibody comprising a light chain variable region of the amino acid sequence of SEQ ID NO: 3 and a heavy chain variable region of the amino acid sequence of SEQ ID NO: 4.

The invention further provides an anti-SARS2-S antibody that binds to the same epitope as a heavy chain-only antibody comprising a heavy chain variable region of the amino acid sequence of SEQ ID NO: 4.

The invention further provides an anti-SARS2-S antibody that competes for binding to SARS2-S with an antibody comprising a light chain variable region of the amino acid sequence of SEQ ID NO: 3 and a heavy chain variable region of the amino acid sequence of SEQ ID NO: 4.

The invention further provides an anti-SARS2-S antibody that competes for binding to SARS2-S with a heavy chain-only antibody comprising a heavy chain variable region of the amino acid sequence of SEQ ID NO: 4.

The invention further provides an anti-SARS2-S antibody comprising complementarity determining regions (CDRs) with the sequences of:
i. SEQ ID NO: 35 for CDR1 of the light chain;
ii. SEQ ID NO: 36 for CDR2 of the light chain;
iii. SEQ ID NO: 37 for CDR3 of the light chain;
iv. SEQ ID NO: 38 for CDR1 of the heavy chain;
v. SEQ ID NO: 39 for CDR2 of the heavy chain; and
vi. SEQ ID NO: 40 for CDR3 of the heavy chain.

In some embodiments, the antibody comprises a light chain variable region of the amino acid sequence of SEQ ID NO: 3.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 3.

In some embodiments, the antibody comprises a heavy chain variable region of the amino acid sequence of SEQ ID NO: 4.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 4.

In some embodiments, the antibody comprises a light chain variable region of the amino acid sequence of SEQ ID NO: 3 and a heavy chain variable region of the amino acid sequence of SEQ ID NO: 4.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 3 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 4.

The invention further provides an anti-SARS2-S antibody comprising complementarity determining regions (CDRs) with:
i. a sequence that is at least 90% identical to SEQ ID NO: 35 for CDR1 of the light chain;
ii. a sequence that is at least 90% identical to SEQ ID NO: 36 for CDR2 of the light chain;
iii. a sequence that is at least 90% identical to SEQ ID NO: 37 for CDR3 of the light chain;
iv. a sequence that is at least 90% identical to SEQ ID NO: 38 for CDR1 of the heavy chain;
v. a sequence that is at least 90% identical to SEQ ID NO: 39 for CDR2 of the heavy chain; and
vi. a sequence that is at least 90% identical to SEQ ID NO: 40 for CDR3 of the heavy chain,
   wherein the antibody competes for binding to SARS2-S with an antibody comprising a light chain variable region of the amino acid sequence of SEQ ID NO: 3 and a heavy chain variable region of the amino acid sequence of SEQ ID NO: 4.

The invention further provides an anti-SARS2-S antibody comprising complementarity determining regions (CDRs) with:
i. a sequence that is at least 95% identical to SEQ ID NO: 35 for CDR1 of the light chain;
ii. a sequence that is at least 95% identical to SEQ ID NO: 36 for CDR2 of the light chain;
iii. a sequence that is at least 95% identical to SEQ ID NO: 37 for CDR3 of the light chain;
iv. a sequence that is at least 95% identical to SEQ ID NO: 38 for CDR1 of the heavy chain;
v. a sequence that is at least 95% identical to SEQ ID NO: 39 for CDR2 of the heavy chain; and
vi. a sequence that is at least 95% identical to SEQ ID NO: 40 for CDR3 of the heavy chain,
   wherein the antibody competes for binding to SARS2-S with an antibody comprising a light chain variable region of the amino acid sequence of SEQ ID NO: 3 and a heavy chain variable region of the amino acid sequence of SEQ ID NO: 4.

A variant of the 52d9 antibody has been produced in which the third amino acid of the VH domain is glutamine instead of histidine (see SEQ ID NO: 7).

Accordingly, invention provides an anti-SARS2-S antibody that binds to the same epitope as an antibody comprising a light chain variable region of the amino acid sequence of SEQ ID NO: 3 and a heavy chain variable region of the amino acid sequence of SEQ ID NO: 7.

The invention further provides an anti-SARS2-S antibody that binds to the same epitope as a heavy chain-only antibody comprising a heavy chain variable region of the amino acid sequence of SEQ ID NO: 7.

The invention further provides an anti-SARS2-S antibody that competes for binding to SARS2-S with an antibody comprising a light chain variable region of the amino acid sequence of SEQ ID NO: 3 and a heavy chain variable region of the amino acid sequence of SEQ ID NO: 7.

The invention further provides an anti-SARS2-S antibody that competes for binding to SARS2-S with a heavy chain-only antibody comprising a heavy chain variable region of the amino acid sequence of SEQ ID NO: 7.

In some embodiments, the antibody comprises a heavy chain variable region of the amino acid sequence of SEQ ID NO: 7.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 7.

In some embodiments, the antibody comprises a light chain variable region of the amino acid sequence of SEQ ID NO: 3 and a heavy chain variable region of the amino acid sequence of SEQ ID NO: 7.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 3 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 7.

The invention further provides an anti-SARS2-S antibody comprising complementarity determining regions (CDRs) with:
i. a sequence that is at least 90% identical to SEQ ID NO: 35 for CDR1 of the light chain;
ii. a sequence that is at least 90% identical to SEQ ID NO: 36 for CDR2 of the light chain;
iii. a sequence that is at least 90% identical to SEQ ID NO: 37 for CDR3 of the light chain;
iv. a sequence that is at least 90% identical to SEQ ID NO: 38 for CDR1 of the heavy chain;
v. a sequence that is at least 90% identical to SEQ ID NO: 39 for CDR2 of the heavy chain; and
vi. a sequence that is at least 90% identical to SEQ ID NO: 40 for CDR3 of the heavy chain,
   wherein the antibody competes for binding to SARS2-S with an antibody comprising a light chain variable region of the amino acid sequence of SEQ ID NO: 3 and a heavy chain variable region of the amino acid sequence of SEQ ID NO: 7.

The invention further provides an anti-SARS2-S antibody comprising complementarity determining regions (CDRs) with:
i. a sequence that is at least 95% identical to SEQ ID NO: 35 for CDR1 of the light chain;
ii. a sequence that is at least 95% identical to SEQ ID NO: 36 for CDR2 of the light chain;
iii. a sequence that is at least 95% identical to SEQ ID NO: 37 for CDR3 of the light chain;
iv. a sequence that is at least 95% identical to SEQ ID NO: 38 for CDR1 of the heavy chain;
v. a sequence that is at least 95% identical to SEQ ID NO: 39 for CDR2 of the heavy chain; and
vi. a sequence that is at least 95% identical to SEQ ID NO: 40 for CDR3 of the heavy chain,
   wherein the antibody competes for binding to SARS2-S with an antibody comprising a light chain variable region of the amino acid sequence of SEQ ID NO: 3 and a heavy chain variable region of the amino acid sequence of SEQ ID NO: 7.

The Examples show that the 49f1 antibody binds to SARS2-S1, as well as SARS2-S_{ecto}, SARS-S_{ecto} and SARS-S1. The 49f1 antibody does not bind to SARS-S1A, SARS2-S1A, SARS-S1_{B} or SARS2-S1_{B}. Without wishing to be bound by any theory, this may mean that 49F1 binds to Sic or S1_{D} of SARS1 and SARS2. Alternatively, 49F1 may bind to an epitope that comprises residues from more than one S1 domain. Similar functional properties can be expected to be associated with the antibodies defined below which share structural and binding characteristics with the 49f1 antibody.

The invention provides an anti-SARS2-S antibody that binds to the same epitope as an antibody comprising a light chain variable region of the amino acid sequence of SEQ ID NO: 5 and a heavy chain variable region of the amino acid sequence of SEQ ID NO: 6.

The invention further provides an anti-SARS2-S antibody that binds to the same epitope as a heavy chain-only antibody comprising a heavy chain variable region of the amino acid sequence of SEQ ID NO: 6.

The invention further provides an anti-SARS2-S antibody that competes for binding to SARS2-S with an antibody comprising a light chain variable region of the amino acid sequence of SEQ ID NO: 5 and a heavy chain variable region of the amino acid sequence of SEQ ID NO: 6.

The invention further provides an anti-SARS2-S antibody that competes for binding to SARS2-S with a heavy chain-only antibody comprising a heavy chain variable region of the amino acid sequence of SEQ ID NO: 6.

The invention further provides an anti-SARS2-S antibody comprising complementarity determining regions (CDRs) with the sequences of:
i. SEQ ID NO: 41 for CDR1 of the light chain;
ii. SEQ ID NO: 42 for CDR2 of the light chain;
iii. SEQ ID NO: 43 for CDR3 of the light chain;
iv. SEQ ID NO: 44 for CDR1 of the heavy chain;
v. SEQ ID NO: 45 for CDR2 of the heavy chain; and
vi. SEQ ID NO: 46 for CDR3 of the heavy chain.

In some embodiments, the antibody comprises a light chain variable region of the amino acid sequence of SEQ ID NO: 5.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 5.

In some embodiments, the antibody comprises a heavy chain variable region of the amino acid sequence of SEQ ID NO: 6.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6.

In some embodiments, the antibody comprises a light chain variable region of the amino acid sequence of SEQ ID NO: 5 and a heavy chain variable region of the amino acid sequence of SEQ ID NO: 6.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 5 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6.

The invention further provides an anti-SARS2-S antibody comprising complementarity determining regions (CDRs) with:
i. a sequence that is at least 90% identical to SEQ ID NO: 41 for CDR1 of the light chain;
ii. a sequence that is at least 90% identical to SEQ ID NO: 42 for CDR2 of the light chain;
iii. a sequence that is at least 90% identical to SEQ ID NO: 43 for CDR3 of the light chain;
iv. a sequence that is at least 90% identical to SEQ ID NO: 44 for CDR1 of the heavy chain;
v. a sequence that is at least 90% identical to SEQ ID NO: 45 for CDR2 of the heavy chain; and
vi. a sequence that is at least 90% identical to SEQ ID NO: 46 for CDR3 of the heavy chain,
   wherein the antibody competes for binding to SARS2-S with an antibody comprising a light chain variable region of the amino acid sequence of SEQ ID NO: 5 and a heavy chain variable region of the amino acid sequence of SEQ ID NO: 6.

The invention further provides an anti-SARS2-S antibody comprising complementarity determining regions (CDRs) with:
i. a sequence that is at least 95% identical to SEQ ID NO: 41 for CDR1 of the light chain;
ii. a sequence that is at least 95% identical to SEQ ID NO: 42 for CDR2 of the light chain;
iii. a sequence that is at least 95% identical to SEQ ID NO: 43 for CDR3 of the light chain;
iv. a sequence that is at least 95% identical to SEQ ID NO: 44 for CDR1 of the heavy chain;
v. a sequence that is at least 95% identical to SEQ ID NO: 45 for CDR2 of the heavy chain; and
vi. a sequence that is at least 95% identical to SEQ ID NO: 46 for CDR3 of the heavy chain,
   wherein the antibody competes for binding to SARS2-S with an antibody comprising a light chain variable region of the amino acid sequence of SEQ ID NO: 5 and a heavy chain variable region of the amino acid sequence of SEQ ID NO: 6.

The V_{H} domain of the 49f1 antibody can bind to either of the alternative V_{L} domains of SEQ ID NOs: 47 and 48.

Accordingly, the invention provides an anti-SARS2-S antibody that binds to the same epitope as an antibody comprising a light chain variable region of the amino acid sequence of SEQ ID NO: 47 and a heavy chain variable region of the amino acid sequence of SEQ ID NO: 6. The invention further provides an anti-SARS2-S antibody that binds to the same epitope as an antibody comprising a light chain variable region of the amino acid sequence of SEQ ID NO: 48 and a heavy chain variable region of the amino acid sequence of SEQ ID NO: 6.

The invention further provides an anti-SARS2-S antibody that competes for binding to SARS2-S with an antibody comprising a light chain variable region of the amino acid sequence of SEQ ID NO: 47 and a heavy chain variable region of the amino acid sequence of SEQ ID NO: 6. The invention further provides an anti-SARS2-S antibody that competes for binding to SARS2-S with an antibody comprising a light chain variable region of the amino acid sequence of SEQ ID NO: 48 and a heavy chain variable region of the amino acid sequence of SEQ ID NO: 6.

The invention further provides an anti-SARS2-S antibody comprising complementarity determining regions (CDRs) with the sequences of:
i. SEQ ID NO: 59 for CDR1 of the light chain;
ii. SEQ ID NO: 60 for CDR2 of the light chain;
iii. SEQ ID NO: 61 for CDR3 of the light chain;
iv. SEQ ID NO: 44 for CDR1 of the heavy chain;
v. SEQ ID NO: 45 for CDR2 of the heavy chain; and
vi. SEQ ID NO: 46 for CDR3 of the heavy chain.

The invention further provides an anti-SARS2-S antibody comprising complementarity determining regions (CDRs) with the sequences of:
i. SEQ ID NO: 62 for CDR1 of the light chain;
ii. SEQ ID NO: 63 for CDR2 of the light chain;
iii. SEQ ID NO: 64 for CDR3 of the light chain;
iv. SEQ ID NO: 44 for CDR1 of the heavy chain;
v. SEQ ID NO: 45 for CDR2 of the heavy chain; and
vi. SEQ ID NO: 46 for CDR3 of the heavy chain.

In some embodiments, the antibody comprises a light chain variable region of the amino acid sequence of SEQ ID NO: 47. In some embodiments, the antibody comprises a light chain variable region of the amino acid sequence of SEQ ID NO: 48.

In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 47. In some embodiments, the antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 48.

In some embodiments, the antibody comprises a light chain variable region of the amino acid sequence of SEQ ID NO: 47 and a heavy chain variable region of the amino acid sequence of SEQ ID NO: 6. In some embodiments, the antibody comprises a light chain variable region of the amino acid sequence of SEQ ID NO: 48 and a heavy chain variable region of the amino acid sequence of SEQ ID NO: 6.

In some embodiments, the antibody comprises a light chain variable region of the amino acid sequence of SEQ ID NO: 47 and a heavy chain variable region of the amino acid sequence of SEQ ID NO: 6, wherein the antibody is of IgG1, IgG2, IgG3 or IgG4 isotype.

In some embodiments, the antibody is an IgG1 antibody comprising a light chain variable region of the amino acid sequence of SEQ ID NO: 47 and a heavy chain variable region of the amino acid sequence of SEQ ID NO: 6, wherein the antibody comprises an Fc containing Ser239Asp, Ala330Leu and Ile332Glu modifications relative to the wild-type IgG1 sequence. Without wishing to be bound by any theory, the DLE modifications advantageously enhance ADCC and ADCP via increased FcγRIIIa affinity and low binding to inhibitory FcγRIIb.

In some embodiments, the antibody is a human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 75, with DLE modifications (Ser239Asp, Ala330Leu and Ile332Glu)
and wherein each light chain comprises the amino acid sequence of SEQ ID NO: 73.

In some embodiments, the antibody is a human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 75, with DLE modifications (Ser239Asp, Ala330Leu and Ile332Glu according to Kabat numbering)
and wherein each light chain comprises the amino acid sequence of SEQ ID NO: 73.

In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 47 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6. In some embodiments, the antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 48 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6.

The invention further provides an anti-SARS2-S antibody comprising complementarity determining regions (CDRs) with:
i. a sequence that is at least 90% identical to SEQ ID NO: 59 for CDR1 of the light chain;
ii. a sequence that is at least 90% identical to SEQ ID NO: 60 for CDR2 of the light chain;
iii. a sequence that is at least 90% identical to SEQ ID NO: 61 for CDR3 of the light chain;
iv. a sequence that is at least 90% identical to SEQ ID NO: 44 for CDR1 of the heavy chain;
v. a sequence that is at least 90% identical to SEQ ID NO: 45 for CDR2 of the heavy chain; and
vi. a sequence that is at least 90% identical to SEQ ID NO: 46 for CDR3 of the heavy chain,
   wherein the antibody competes for binding to SARS2-S with an antibody comprising a light chain variable region of the amino acid sequence of SEQ ID NO: 47 and a heavy chain variable region of the amino acid sequence of SEQ ID NO: 6.

The invention further provides an anti-SARS2-S antibody comprising complementarity determining regions (CDRs) with:
i. a sequence that is at least 90% identical to SEQ ID NO: 62 for CDR1 of the light chain;
ii. a sequence that is at least 90% identical to SEQ ID NO: 63 for CDR2 of the light chain;
iii. a sequence that is at least 90% identical to SEQ ID NO: 64 for CDR3 of the light chain;
iv. a sequence that is at least 90% identical to SEQ ID NO: 44 for CDR1 of the heavy chain;
v. a sequence that is at least 90% identical to SEQ ID NO: 45 for CDR2 of the heavy chain; and
vi. a sequence that is at least 90% identical to SEQ ID NO: 46 for CDR3 of the heavy chain,
   wherein the antibody competes for binding to SARS2-S with an antibody comprising a light chain variable region of the amino acid sequence of SEQ ID NO: 48 and a heavy chain variable region of the amino acid sequence of SEQ ID NO: 6.

The invention further provides an anti-SARS2-S antibody comprising complementarity determining regions (CDRs) with:
i. a sequence that is at least 95% identical to SEQ ID NO: 59 for CDR1 of the light chain;
ii. a sequence that is at least 95% identical to SEQ ID NO: 60 for CDR2 of the light chain;
iii. a sequence that is at least 95% identical to SEQ ID NO: 61 for CDR3 of the light chain;
iv. a sequence that is at least 95% identical to SEQ ID NO: 44 for CDR1 of the heavy chain;
v. a sequence that is at least 95% identical to SEQ ID NO: 45 for CDR2 of the heavy chain; and
vi. a sequence that is at least 95% identical to SEQ ID NO: 46 for CDR3 of the heavy chain,
   wherein the antibody competes for binding to SARS2-S with an antibody comprising a light chain variable region of the amino acid sequence of SEQ ID NO: 47 and a heavy chain variable region of the amino acid sequence of SEQ ID NO: 6.

The invention further provides an anti-SARS2-S antibody comprising complementarity determining regions (CDRs) with:
i. a sequence that is at least 95% identical to SEQ ID NO: 62 for CDR1 of the light chain;
ii. a sequence that is at least 95% identical to SEQ ID NO: 63 for CDR2 of the light chain;
iii. a sequence that is at least 95% identical to SEQ ID NO: 64 for CDR3 of the light chain;
iv. a sequence that is at least 95% identical to SEQ ID NO: 44 for CDR1 of the heavy chain;
v. a sequence that is at least 95% identical to SEQ ID NO: 45 for CDR2 of the heavy chain; and
vi. a sequence that is at least 95% identical to SEQ ID NO: 46 for CDR3 of the heavy chain,
   wherein the antibody competes for binding to SARS2-S with an antibody comprising a light chain variable region of the amino acid sequence of SEQ ID NO: 48 and a heavy chain variable region of the amino acid sequence of SEQ ID NO: 6.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 75, and wherein each light chain comprises the amino acid sequence of SEQ ID NO: 73.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 74,
and wherein each light chain is encoded by the nucleic acid sequence of SEQ ID NO: 72.

The section below specifies features that are combinable with each of the previous embodiments, as appropriate.

In some embodiments, the antibody binds to SARS2-S with a K_{D} of 10⁻⁷ M or less, 10⁻⁸M or less, 10⁻⁹M or less, or 10¹⁰ M or less. In some embodiments, antibody binding affinity is determined using an Octet® RED96 system (ForteBio, Inc.). For example, a Flag-tagged 51 domain or a Flag-tagged S2 domain may be immobilized to an anti-Flag biosensor and incubated with varying concentrations of the antibody in solution, binding data are then collected. In some embodiments, antibody binding affinity is determined by surface plasmon resonance.

In some embodiments, the antibody is capable of inhibiting the interaction between SARS2-S and the human ACE2 protein by at least 20%, at least 30%, at least 40%, at least 50%, at least 60%, at least 70%, at least 80%, at least 90%, at least 95%, at least 99% or 100%. In some embodiments, the capability for inhibiting the interaction between SARS2-S and the human ACE2 protein is measured in a blocking ELISA assay.

In some embodiments, the antibody is capable of neutralizing SARS2 infectivity of human host cells by more than 50%, by more than 60%, by more than 70%, by more than 80%, by more than 90%, by more than 95%, by more than 99% or by 100%. In some embodiments, the 50% inhibitory concentration (IC₅₀) value of the antibody for neutralizing SARS2 infectivity is less than 100 μg/ml, less than 50 μg/ml, less than 40 μg/ml, less than 30 μg/ml, less than 20 μg/ml, less than 15 μg/ml, less than 10 μg/ml, less than 5 μg/ml, less than 1 μg/ml, less than 0.1 μg/ml, less than 0.01 μg/ml, less than 0.001 μg/ml or less than 0.0001 μg/ml. In some embodiments, the neutralizing capability of anti-SARS2-S antibodies is measured in a virus-like particle (VLP) neutralization assay (Tang et al. (2012) PNAS 111(19): E2018-E2026).

In some embodiments, the antibody is capable of interfering with SARS2-S protein-mediated membrane fusion. The S2 domain is thought to be responsible for mediating membrane fusion. Accordingly, antibodies that bind to the S2 domain may be capable of interfering with interfering with SARS2-S protein-mediated membrane fusion. To test this property, a SARS2-S driven cell-cell fusion assay may be used (e.g. an assay which uses a GFP-tagged SARS2 spike protein that has a mutated the furin cleavage site at the S1/S2 junction). Inhibition of the formation of syncytia in this assay indicates that the test antibody is capable of interfering with SARS2 S protein-mediated membrane fusion.

In some embodiments, whether a test antibody competes with a reference antibody for binding to SARS2-S is determined using an in vitro binding competition assay. For example, a Flag-tagged S1 domain or a Flag-tagged S2 domain may be immobilized to an anti-Flag biosensor, the association of the reference antibody to the immobilized Flag-tagged S1 or S2 domain is then measured (e.g. using the Octet® RED96 system, ForteBio, Inc.) and then the degree of additional binding is assessed by exposing the immobilized Flag-tagged S1 or S2 domain to the test antibody in the presence of the reference antibody.

In some embodiments, the anti-SARS2-S antibody recognizes SARS2 and one or more additional beta coronaviruses. For example, in some embodiments the anti-SARS2-S antibody recognizes: (i) SARS2 and MERS-CoV, (ii) SARS2 and mouse hepatitis virus (MHV), (iii) SARS2 and SARS1, (iv) SARS2, MERS-CoV and MHV, (v) SARS2, MERS-CoV and SARS1, (vi) SARS2, MHV and SARS1, or (iv) SARS2, MERS-CoV, MHV and SARS1.

In some embodiments, the anti-SARS2-S antibody is a heavy chain-only antibody.

### Combinations of Anti-SARS2-S Antibodies

The invention provides a combination of two or more (e.g. three or more, four or more, or five or more, six or more, or seven or more) anti-SARS2-S antibodies, wherein the antibodies bind to different epitopes on the SARS2-S protein. In some embodiments, the antibodies in the combination target non-overlapping epitopes. Advantageously, antibody combinations targeting non-overlapping epitopes may act synergistically resulting in lower dosage and may mitigate risk of immune escape.

S1_{B} binding by 47D11 further away from the receptor binding interface explains its inability to compromise spike-receptor interaction and opens possibilities for combination treatments with non-competing, potent neutralizing antibodies that target the receptor binding subdomain.

The invention provides a combination of anti-SARS2-S antibodies comprising: (i) a first anti-SARS2-S antibody that binds to the closed conformation of SARS2-S1_{B}, and (ii) a second anti-SARS2-S antibody that binds to the open conformation of SARS2-S1_{B}.

The invention provides a combination of anti-SARS2-S antibodies comprising: (i) a first anti-SARS2-S antibody that binds to the closed conformation of SARS2-S1_{B} within the partially open form of a SARS2-S trimer, and (ii) a second anti-SARS2-S antibody that binds to the open conformation of SARS2-S1_{B}.

The invention provides a combination of anti-SARS2-S antibodies comprising: (i) a first anti-SARS2-S antibody that binds to the closed conformation of SARS2-S1_{B} within the partially open form of a SARS2-S trimer, and (ii) a second anti-SARS2-S antibody that binds to the open conformation of SARS2-S1_{B} within the partially open form of a SARS2-S trimer.

The invention provides a combination of anti-SARS2-S antibodies comprising: (i) a first anti-SARS2-S antibody that binds to the S1_{B} subunit, and (ii) a second anti anti-SARS2-S antibody that binds to the S1_{A} subunit.

The invention provides a combination of anti-SARS2-S antibodies comprising: (i) a first anti-SARS2-S antibody that binds to the S1_{B} subunit, and (ii) a second anti anti-SARS2-S antibody that binds to the S1_{C} subunit.

The invention provides a combination of anti-SARS2-S antibodies comprising: (i) a first anti-SARS2-S antibody that binds to the S1_{B} subunit, and (ii) a second anti anti-SARS2-S antibody that binds to the S1_{D} subunit.

The invention provides a combination of anti-SARS2-S antibodies comprising: (i) a first anti-SARS2-S antibody that binds to the S1_{B} subunit, and (ii) a second anti anti-SARS2-S antibody that binds to the S2 subunit.

The invention provides a combination of anti-SARS2-S antibodies comprising: (i) a first anti-SARS2-S antibody that binds to the S1_{B} subunit, wherein the antibody is capable of inhibiting the infection of human cells by SARS2, and wherein the antibody does not inhibit the binding of SARS2-S to human angiotensin converting enzyme 2 (ACE2), and (ii) a second anti-SARS2-S antibody that binds to the S1_{B} subunit, wherein the antibody is capable of inhibiting the infection of human cells by SARS2, and wherein the antibody inhibits the binding of SARS2-S to ACE2.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is the K347N,
   (c) SARS2-S having the sequence of SEQ ID NO: 8 with a single substitution, wherein the substitution is the E414K, and
   (d) SARS2-S having the sequence of SEQ ID NO: 8 with a single substitution, wherein the substitution is the N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A, and
   (c) SARS2-S having the sequence of SEQ ID NO: 8 with a single substitution, wherein the substitution is L298A.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y,
   (e) SARS2-S having the sequence of SEQ ID NO: 8 with a single substitution, wherein the substitution is N369K,
   (f) SARS2-S having the sequence of SEQ ID NO: 8 with a single substitution, wherein the substitution is Q423R, and
   (g) SARS2-S having the sequence of SEQ ID NO: 8 with a single substitution, wherein the substitution is S424P; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K, and
   (d) SARS2-S having the sequence of SEQ ID NO: 71 with a single substitution, wherein the substitution is N501Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the first anti-SARS2-S antibody:
(i) binds optionally with a K_{D} of 10⁻⁸M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K347N,
   (c) SARS2-S having the sequence of SEQ ID NO: 71 with a single substitution, wherein the substitution is E414K, and
   (d) SARS2-S having the sequence of SEQ ID NO: 71 with a single substitution, wherein the substitution is N431Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the first anti-SARS2-S antibody:
(i) binds optionally with a K_{D} of 10⁻⁸M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (g) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the first anti-SARS2-S antibody:
(i) binds optionally with a K_{D} of 10⁻⁸ M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (b) SARS2-S having the sequence of SEQ ID NO: 71 with a single substitution, wherein the substitution is S494P; and
(iii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the first antibody is an anti-SARS2-S antibody that binds to the same epitope as an antibody comprising a light chain variable region of the amino acid sequence of SEQ ID NO: 9 and a heavy chain variable region of the amino acid sequence of SEQ ID NO: 10.

In some embodiments, the first antibody is an anti-SARS2-S antibody that binds to the same epitope as a heavy chain-only antibody comprising a heavy chain variable region of the amino acid sequence of SEQ ID NO: 10.

In some embodiments, the first antibody is an anti-SARS2-S antibody that competes for binding to SARS2-S with an antibody comprising a light chain variable region of the amino acid sequence of SEQ ID NO: 9 and a heavy chain variable region of the amino acid sequence of SEQ ID NO: 10.

In some embodiments, the first antibody is an anti-SARS2-S antibody that competes for binding to SARS2-S with a heavy chain-only antibody comprising a heavy chain variable region of the amino acid sequence of SEQ ID NO: 10.

In some embodiments, the first antibody is an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain.

In some embodiments, the first antibody comprises a light chain variable region of the amino acid sequence of SEQ ID NO: 9.

In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 9.

In some embodiments, the first antibody comprises a heavy chain variable region of the amino acid sequence of SEQ ID NO: 10.

In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 10.

In some embodiments, the first antibody comprises a light chain variable region of the amino acid sequence of SEQ ID NO: 9 and a heavy chain variable region of the amino acid sequence of SEQ ID NO: 10.

In some embodiments, the first antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 9 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 10.

In some embodiments, the first antibody is an anti-SARS2-S antibody comprising complementarity determining regions (CDRs) with:
i. a sequence that is at least 90% identical to SEQ ID NO: 53 for CDR1 of the light chain;
ii. a sequence that is at least 90% identical to SEQ ID NO: 54 for CDR2 of the light chain;
iii. a sequence that is at least 90% identical to SEQ ID NO: 55 for CDR3 of the light chain;
iv. a sequence that is at least 90% identical to SEQ ID NO: 56 for CDR1 of the heavy chain;
v. a sequence that is at least 90% identical to SEQ ID NO: 57 for CDR2 of the heavy chain; and
vi. a sequence that is at least 90% identical to SEQ ID NO: 58 for CDR3 of the heavy chain,
   wherein the antibody competes for binding to SARS2-S with an antibody comprising a light chain variable region of the amino acid sequence of SEQ ID NO: 9 and a heavy chain variable region of the amino acid sequence of SEQ ID NO: 10.

In some embodiments, the first antibody is an anti-SARS2-S antibody comprising complementarity determining regions (CDRs) with:
i. a sequence that is at least 95% identical to SEQ ID NO: 53 for CDR1 of the light chain;
ii. a sequence that is at least 95% identical to SEQ ID NO: 54 for CDR2 of the light chain;
iii. a sequence that is at least 95% identical to SEQ ID NO: 55 for CDR3 of the light chain;
iv. a sequence that is at least 95% identical to SEQ ID NO: 56 for CDR1 of the heavy chain;
v. a sequence that is at least 95% identical to SEQ ID NO: 57 for CDR2 of the heavy chain; and
vi. a sequence that is at least 95% identical to SEQ ID NO: 58 for CDR3 of the heavy chain,
   wherein the antibody competes for binding to SARS2-S with an antibody comprising a light chain variable region of the amino acid sequence of SEQ ID NO: 9 and a heavy chain variable region of the amino acid sequence of SEQ ID NO: 10.

The invention provides a combination of anti-SARS2-S antibodies comprising: (i) a first anti-SARS2-S antibody that binds to the S1_{A} subunit, and (ii) a second anti anti-SARS2-S antibody that binds to the S1_{C} subunit.

The invention provides a combination of anti-SARS2-S antibodies comprising: (i) a first anti-SARS2-S antibody that binds to the S1_{A} subunit, and (ii) a second anti anti-SARS2-S antibody that binds to the S1_{D} subunit.

The invention provides a combination of anti-SARS2-S antibodies comprising: (i) a first anti-SARS2-S antibody that binds to the S1_{A} subunit, and (ii) a second anti anti-SARS2-S antibody that binds to the S2 subunit.

The invention provides a combination of anti-SARS2-S antibodies comprising: (i) a first anti-SARS2-S antibody that binds to the S1_{C} subunit, and (ii) a second anti anti-SARS2-S antibody that binds to the S1_{D} subunit.

The invention provides a combination of anti-SARS2-S antibodies comprising: (i) a first anti-SARS2-S antibody that binds to the S1_{C} subunit, and (ii) a second anti anti-SARS2-S antibody that binds to the S2 subunit.

The invention provides a combination of anti-SARS2-S antibodies comprising: (i) a first anti-SARS2-S antibody that binds to the S1_{D} subunit, and (ii) a second anti anti-SARS2-S antibody that binds to the S2 subunit.

The Examples show that the 49f1 antibody binds to SARS2-S1, as well as SARS-S_{ecto} and SARS-S1. The 49f1 antibody does not bind to SARS-S1_{A}.

The invention provides a combination of anti-SARS2-S antibodies comprising: (i) a first anti-SARS2-S antibody that binds to S1, and (ii) a second anti anti-SARS2-S antibody that binds to a different epitope on the SARS2-S protein. In some embodiments, the different epitopes are non-overlapping.

In some embodiments, the second antibody binds to the S1_{A} subunit. In some embodiments, the second antibody binds to the S1_{B} subunit. In some embodiments, the second antibody binds to the Sic subunit. In some embodiments, the second antibody binds to the S1_{D} subunit. In some embodiments, the second antibody binds to the S2 subunit.

In some embodiments, the first antibody binds to SARS-S1 and SARS-S_{ecto}. In some embodiments, the first antibody binds to SARS-S1 and SARS-S_{ecto}, but not to SARS-S1_{A}.

In some embodiments, the first antibody is an anti-SARS2-S antibody that binds to the same epitope as an antibody comprising a light chain variable region of the amino acid sequence of SEQ ID NO: 5 and a heavy chain variable region of the amino acid sequence of SEQ ID NO: 6.

In some embodiments, the first antibody is an anti-SARS2-S antibody that binds to the same epitope as a heavy chain-only antibody comprising a heavy chain variable region of the amino acid sequence of SEQ ID NO: 6.

In some embodiments, the first antibody is an anti-SARS2-S antibody that competes for binding to SARS2-S with an antibody comprising a light chain variable region of the amino acid sequence of SEQ ID NO: 5 and a heavy chain variable region of the amino acid sequence of SEQ ID NO: 6.

In some embodiments, the first antibody is an anti-SARS2-S antibody that competes for binding to SARS2-S with a heavy chain-only antibody comprising a heavy chain variable region of the amino acid sequence of SEQ ID NO: 6.

In some embodiments, the first antibody is an anti-SARS2-S antibody comprising complementarity determining regions (CDRs) with the sequences of:
i. SEQ ID NO: 41 for CDR1 of the light chain;
ii. SEQ ID NO: 42 for CDR2 of the light chain;
iii. SEQ ID NO: 43 for CDR3 of the light chain;
iv. SEQ ID NO: 44 for CDR1 of the heavy chain;
v. SEQ ID NO: 45 for CDR2 of the heavy chain; and
vi. SEQ ID NO: 46 for CDR3 of the heavy chain.

In some embodiments, the first antibody comprises a light chain variable region of the amino acid sequence of SEQ ID NO: 5.

In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 5.

In some embodiments, the first antibody comprises a heavy chain variable region of the amino acid sequence of SEQ ID NO: 6.

In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6.

In some embodiments, the first antibody comprises a light chain variable region of the amino acid sequence of SEQ ID NO: 5 and a heavy chain variable region of the amino acid sequence of SEQ ID NO: 6.

In some embodiments, the first antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 5 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6.

In some embodiments, the first antibody is an anti-SARS2-S antibody comprising complementarity determining regions (CDRs) with:
i. a sequence that is at least 90% identical to SEQ ID NO: 41 for CDR1 of the light chain;
ii. a sequence that is at least 90% identical to SEQ ID NO: 42 for CDR2 of the light chain;
iii. a sequence that is at least 90% identical to SEQ ID NO: 43 for CDR3 of the light chain;
iv. a sequence that is at least 90% identical to SEQ ID NO: 44 for CDR1 of the heavy chain;
v. a sequence that is at least 90% identical to SEQ ID NO: 45 for CDR2 of the heavy chain; and
vi. a sequence that is at least 90% identical to SEQ ID NO: 46 for CDR3 of the heavy chain,
   wherein the antibody competes for binding to SARS2-S with an antibody comprising a light chain variable region of the amino acid sequence of SEQ ID NO: 5 and a heavy chain variable region of the amino acid sequence of SEQ ID NO: 6.

In some embodiments, the first antibody is an anti-SARS2-S antibody comprising complementarity determining regions (CDRs) with:
i. a sequence that is at least 95% identical to SEQ ID NO: 41 for CDR1 of the light chain;
ii. a sequence that is at least 95% identical to SEQ ID NO: 42 for CDR2 of the light chain;
iii. a sequence that is at least 95% identical to SEQ ID NO: 43 for CDR3 of the light chain;
iv. a sequence that is at least 95% identical to SEQ ID NO: 44 for CDR1 of the heavy chain;
v. a sequence that is at least 95% identical to SEQ ID NO: 45 for CDR2 of the heavy chain; and
vi. a sequence that is at least 95% identical to SEQ ID NO: 46 for CDR3 of the heavy chain,
   wherein the antibody competes for binding to SARS2-S with an antibody comprising a light chain variable region of the amino acid sequence of SEQ ID NO: 5 and a heavy chain variable region of the amino acid sequence of SEQ ID NO: 6.

The V_{H} domain of the 49f1 antibody can bind to either of the alternative V_{L} domains of SEQ ID NOs: 47 and 48.

Accordingly, In some embodiments, the first antibody is an anti-SARS2-S antibody that binds to the same epitope as an antibody comprising a light chain variable region of the amino acid sequence of SEQ ID NO: 47 and a heavy chain variable region of the amino acid sequence of SEQ ID NO: 6. In some embodiments, the first antibody is an anti-SARS2-S antibody that binds to the same epitope as an antibody comprising a light chain variable region of the amino acid sequence of SEQ ID NO: 48 and a heavy chain variable region of the amino acid sequence of SEQ ID NO: 6.

In some embodiments, the first antibody is an anti-SARS2-S antibody that competes for binding to SARS2-S with an antibody comprising a light chain variable region of the amino acid sequence of SEQ ID NO: 47 and a heavy chain variable region of the amino acid sequence of SEQ ID NO: 6. The invention further provides an anti-SARS2-S antibody that competes for binding to SARS2-S with an antibody comprising a light chain variable region of the amino acid sequence of SEQ ID NO: 48 and a heavy chain variable region of the amino acid sequence of SEQ ID NO: 6.

In some embodiments, the first antibody is an anti-SARS2-S antibody comprising complementarity determining regions (CDRs) with the sequences of:
i. SEQ ID NO: 59 for CDR1 of the light chain;
ii. SEQ ID NO: 60 for CDR2 of the light chain;
iii. SEQ ID NO: 61 for CDR3 of the light chain;
iv. SEQ ID NO: 44 for CDR1 of the heavy chain;
v. SEQ ID NO: 45 for CDR2 of the heavy chain; and
vi. SEQ ID NO: 46 for CDR3 of the heavy chain.

In some embodiments, the first antibody is an anti-SARS2-S antibody comprising complementarity determining regions (CDRs) with the sequences of:
i. SEQ ID NO: 62 for CDR1 of the light chain;
ii. SEQ ID NO: 63 for CDR2 of the light chain;
iii. SEQ ID NO: 64 for CDR3 of the light chain;
iv. SEQ ID NO: 44 for CDR1 of the heavy chain;
v. SEQ ID NO: 45 for CDR2 of the heavy chain; and
vi. SEQ ID NO: 46 for CDR3 of the heavy chain.

In some embodiments, the first antibody comprises a light chain variable region of the amino acid sequence of SEQ ID NO: 47. In some embodiments, the antibody comprises a light chain variable region of the amino acid sequence of SEQ ID NO: 48.

In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 47. In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 48.

In some embodiments, the first antibody comprises a light chain variable region of the amino acid sequence of SEQ ID NO: 47 and a heavy chain variable region of the amino acid sequence of SEQ ID NO: 6. In some embodiments, the first antibody comprises a light chain variable region of the amino acid sequence of SEQ ID NO: 48 and a heavy chain variable region of the amino acid sequence of SEQ ID NO: 6.

In some embodiments, the first antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 47 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6. In some embodiments, the first antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 48 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 6.

In some embodiments, the first antibody is an anti-SARS2-S antibody comprising complementarity determining regions (CDRs) with:
i. a sequence that is at least 90% identical to SEQ ID NO: 59 for CDR1 of the light chain;
ii. a sequence that is at least 90% identical to SEQ ID NO: 60 for CDR2 of the light chain;
iii. a sequence that is at least 90% identical to SEQ ID NO: 61 for CDR3 of the light chain;
iv. a sequence that is at least 90% identical to SEQ ID NO: 44 for CDR1 of the heavy chain;
v. a sequence that is at least 90% identical to SEQ ID NO: 45 for CDR2 of the heavy chain; and
vi. a sequence that is at least 90% identical to SEQ ID NO: 46 for CDR3 of the heavy chain,
   wherein the antibody competes for binding to SARS2-S with an antibody comprising a light chain variable region of the amino acid sequence of SEQ ID NO: 47 and a heavy chain variable region of the amino acid sequence of SEQ ID NO: 6.

In some embodiments, the first antibody is an anti-SARS2-S antibody comprising complementarity determining regions (CDRs) with:
i. a sequence that is at least 90% identical to SEQ ID NO: 62 for CDR1 of the light chain;
ii. a sequence that is at least 90% identical to SEQ ID NO: 63 for CDR2 of the light chain;
iii. a sequence that is at least 90% identical to SEQ ID NO: 64 for CDR3 of the light chain;
iv. a sequence that is at least 90% identical to SEQ ID NO: 44 for CDR1 of the heavy chain;
v. a sequence that is at least 90% identical to SEQ ID NO: 45 for CDR2 of the heavy chain; and
vi. a sequence that is at least 90% identical to SEQ ID NO: 46 for CDR3 of the heavy chain,
   wherein the antibody competes for binding to SARS2-S with an antibody comprising a light chain variable region of the amino acid sequence of SEQ ID NO: 48 and a heavy chain variable region of the amino acid sequence of SEQ ID NO: 6.

In some embodiments, the first antibody is an anti-SARS2-S antibody comprising complementarity determining regions (CDRs) with:
i. a sequence that is at least 95% identical to SEQ ID NO: 59 for CDR1 of the light chain;
ii. a sequence that is at least 95% identical to SEQ ID NO: 60 for CDR2 of the light chain;
iii. a sequence that is at least 95% identical to SEQ ID NO: 61 for CDR3 of the light chain;
iv. a sequence that is at least 95% identical to SEQ ID NO: 44 for CDR1 of the heavy chain;
v. a sequence that is at least 95% identical to SEQ ID NO: 45 for CDR2 of the heavy chain; and
vi. a sequence that is at least 95% identical to SEQ ID NO: 46 for CDR3 of the heavy chain,
   wherein the antibody competes for binding to SARS2-S with an antibody comprising a light chain variable region of the amino acid sequence of SEQ ID NO: 47 and a heavy chain variable region of the amino acid sequence of SEQ ID NO: 6.

In some embodiments, the first antibody is an anti-SARS2-S antibody comprising complementarity determining regions (CDRs) with:
i. a sequence that is at least 95% identical to SEQ ID NO: 62 for CDR1 of the light chain;
ii. a sequence that is at least 95% identical to SEQ ID NO: 63 for CDR2 of the light chain;
iii. a sequence that is at least 95% identical to SEQ ID NO: 64 for CDR3 of the light chain;
iv. a sequence that is at least 95% identical to SEQ ID NO: 44 for CDR1 of the heavy chain;
v. a sequence that is at least 95% identical to SEQ ID NO: 45 for CDR2 of the heavy chain; and
vi. a sequence that is at least 95% identical to SEQ ID NO: 46 for CDR3 of the heavy chain,
   wherein the antibody competes for binding to SARS2-S with an antibody comprising a light chain variable region of the amino acid sequence of SEQ ID NO: 48 and a heavy chain variable region of the amino acid sequence of SEQ ID NO: 6.

In some embodiments, the first antibody is a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 75,
and wherein each light chain comprises the amino acid sequence of SEQ ID NO: 73. In some embodiments, the first antibody is a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 74,
and wherein each light chain is encoded by the nucleic acid sequence of SEQ ID NO: 72.

In some embodiments, the first antibody is a human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 75, with DLE modifications (Ser239Asp, Ala330Leu and Ile332Glu)
and wherein each light chain comprises the amino acid sequence of SEQ ID NO: 73. In some embodiments, the first antibody is a human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 75, with DLE modifications (Ser239Asp, Ala330Leu and Ile332Glu according to Kabat numbering)
and wherein each light chain comprises the amino acid sequence of SEQ ID NO: 73.

In some embodiments, the first antibody is a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain.

In some embodiments, the first antibody is a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9.

In some embodiments, the first antibody is a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66.

In some embodiments, the first antibody is a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69.

The section below specifies features that are combinable with each of the previous embodiments, as appropriate.

In some embodiments, the combination of antibodies is a synergistic combination of antibodies. In some embodiments, each of the anti-SARS2-S antibodies in the combination binds to SARS2-S with a K_{D} of 10⁻⁷ M or less, 10⁻⁸ M or less, 10⁻⁹M or less, or 10⁻¹⁰ M or less. In some embodiments, antibody binding affinity is determined using an Octet® RED96 system (ForteBio, Inc.). For example, a Flag-tagged S1 domain or a Flag-tagged S2 domain may be immobilized to an anti-Flag biosensor and incubated with varying concentrations of the antibody in solution, binding data are then collected. In some embodiments, antibody binding affinity is determined by surface plasmon resonance.

In some embodiments, one or more (e.g. two, three, four, five, six, seven or more) of the anti-SARS2-S antibodies in the combination is capable of inhibiting the interaction between SARS2-S and the human ACE2 protein by at least 20%, at least 30%, at least 40%, at least 50%, at least 60%, at least 70%, at least 80%, at least 90%, at least 95%, at least 99% or 100%. In some embodiments, each of the anti-SARS2-S antibodies in the combination is capable of inhibiting the interaction between SARS2-S and the human ACE2 protein by at least 20%, at least 30%, at least 40%, at least 50%, at least 60%, at least 70%, at least 80%, at least 90%, at least 95%, at least 99% or 100%. In some embodiments, the capability for inhibiting the interaction between SARS2-S and the human ACE2 protein is measured in a blocking ELISA assay.

In some embodiments, one or more (e.g. two, three, four, five, six, seven or more) of the anti-SARS2-S antibodies in the combination is capable of neutralizing SARS2 infectivity of human host cells by more than 50%, by more than 60%, by more than 70%, by more than 80%, by more than 90%, by more than 95%, by more than 99% or by 100%. In some embodiments, each of the anti-SARS2-S antibodies in the combination is capable of neutralizing SARS2 infectivity of human host cells by more than 50%, by more than 60%, by more than 70%, by more than 80%, by more than 90%, by more than 95%, by more than 99% or by 100%. In some embodiments, the combination of anti-SARS2-S antibodies is capable of neutralizing SARS2 infectivity of human host cells by more than 50%, by more than 60%, by more than 70%, by more than 80%, by more than 90%, by more than 95%, by more than 99% or by 100%. In some embodiments, the 50% inhibitory concentration (IC₅₀) value of the anti-SARS2-S antibodies for neutralizing SARS2 infectivity is less than 100 μg/ml, less than 50 μg/ml, less than 40 μg/ml, less than 30 μg/ml, less than 20 μg/ml, less than 15 μg/ml, less than 10 μg/ml, less than 5 μg/ml, less than 1 μg/ml, less than 0.1 μg/ml, less than 0.01 μg/ml, less than 0.001 μg/ml or less than 0.0001 μg/ml. In some embodiments, the neutralizing capability of anti-SARS2-S antibodies is measured in a virus-like particle (VLP) neutralization assay (Tang et al. (2012) PNAS 111(19): E2018-E2026).

In some embodiments, one or more (e.g. two, three, four, five, six, seven or more) of the anti-SARS2-S antibodies in the combination is capable of interfering with SARS2-S protein-mediated membrane fusion. The S2 domain is thought to be responsible for mediating membrane fusion. Accordingly, antibodies that bind to the S2 domain may be capable of interfering with interfering with SARS2-S protein-mediated membrane fusion. To test this property, a SARS2-S driven cell-cell fusion assay may be used (e.g. an assay which uses a GFP-tagged SARS2 spike protein that has a mutated the furin cleavage site at the S1/S2 junction). Inhibition of the formation of syncytia in this assay indicates that the test antibody is capable of interfering with SARS2 S protein-mediated membrane fusion.

In some embodiments, whether a test antibody competes with a reference antibody for binding to SARS2-S is determined using an in vitro binding competition assay. For example, a Flag-tagged S1 domain or a Flag-tagged S2 domain may be immobilized to an anti-Flag biosensor, the association of the reference antibody to the immobilized Flag-tagged S1 or S2 domain is then measured (e.g. using the Octet® RED96 system, ForteBio, Inc.) and then the degree of additional binding is assessed by exposing the immobilized Flag-tagged S1 or S2 domain to the test antibody in the presence of the reference antibody.

In some embodiments, one or more (e.g. two, three, four, five, six, seven or more) of the anti-SARS2-S antibodies in the combination recognizes SARS2 and one or more additional beta coronaviruses. For example, in some embodiments the anti-SARS2-S antibody recognizes: (i) SARS2 and MERS-CoV, (ii) SARS2 and mouse hepatitis virus (MHV), (iii) SARS2 and SARS1, (iv) SARS2, MERS-CoV and MHV, (v) SARS2, MERS-CoV and SARS1, (vi) SARS2, MHV and SARS1, or (iv) SARS2, MERS-CoV, MHV and SARS1.

In some embodiments, each of the anti-SARS2-S antibodies in the combination recognizes SARS2 and one or more additional beta coronaviruses. For example, in some embodiments the anti-SARS2-S antibody recognizes: (i) SARS2 and MERS-CoV, (ii) SARS2 and mouse hepatitis virus (MHV), (iii) SARS2 and SARS1, (iv) SARS2, MERS-CoV and MHV, (v) SARS2, MERS-CoV and SARS1, (vi) SARS2, MHV and SARS1, or (iv) SARS2, MERS-CoV, MHV and SARS1.

In some embodiments, one or more (e.g. two, three, four, five, six, seven or more) of the anti-SARS2-S antibodies in the combination is a heavy chain-only antibody. In some embodiments, each of the anti-SARS2-S antibodies in the combination is a heavy chain-only antibody.

### Antibodies

In some embodiments, the antibody of the invention is a polyclonal, monoclonal, multispecific, mouse, human, humanized, primatized or chimeric antibody or a single-chain antibody. The term “antibody” encompasses entire tetrameric antibodies and antigen-binding fragments thereof. In some embodiments, the antigen-binding fragment thereof is selected from a VH domain, Fab, Fab′, F(ab′)2, Fd, Fv, a single-chain Fv (scFv) and a disulfide-linked Fv (sdFv).

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a VH domain associated with a V_{L} domain, the VH and V_{L} domains may be situated relative to one another in any suitable arrangement. For example, the variable region may be dimeric and contain VH-VH, VH-V_{L} or V_{L}-V_{L} dimers. Alternatively, the antigen-binding fragment of an antibody may contain a monomeric VH or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-C_{H}1; (ii) VH-C_{H}2; (iii) VH-C_{H}3; (iv) VH-C_{H}1-C_{H}2; (V) VH-C_{H}1-C_{H}2-C_{H}3; (vi) VH-C_{H}2-C_{H}3; (vii) VH-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}.

In any configuration of variable and constant domains, including any of the exemplary configurations listed above, the variable and constant domains may be either directly linked to one another or may be linked by a full or partial hinge or linker region. A hinge or linker region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or V_{L} domain (e.g., by disulfide bond(s)).

As with full antibody molecules, antigen-binding fragments may be mono-specific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antibody format, including the exemplary bi-specific antibody formats disclosed herein, may be adapted for use in the context of an antigen-binding fragment of an antibody of the present invention using routine techniques available in the art.

In some embodiments, the antibody contains an Fc domain or a portion thereof that binds to the FcRn receptor. As a non-limiting example, a suitable Fc domain may be derived from an immunoglobulin subclass such as IgA, IgE, IgG or IgM. In some embodiments, a suitable Fc domain is derived from IgG1, IgG2, IgG3, or IgG4. Particularly suitable Fc domains include those derived from human antibodies.

In a preferred embodiment, the antibody is an IgG (e.g. an IgG1) that comprises two heavy and two light chains.

In some embodiments, the antibody is a human antibody.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In certain embodiments, the antibodies or antigen-binding fragments of the present invention are bispecific comprising a first binding specificity to a first epitope in the SARS2 spike protein and a second binding specificity to a second epitope in the SARS2 spike protein wherein the first and second epitopes are distinct and non-overlapping.

In certain embodiments, the antibodies or antigen-binding fragments of the present invention are tri-specific comprising a first binding specificity to a first epitope in the SARS2 spike protein, a second binding specificity to a second epitope in the receptor binding domain of SARS2 spike protein and a third binding specificity to a third epitope in the SARS2 spike protein, wherein the first, second and third epitopes are distinct and non-overlapping.

In certain embodiments, the antibodies or antigen-binding fragments of the present invention are quadri-specific comprising a first binding specificity to a first epitope in the SARS2 spike protein, a second binding specificity to a second epitope in the SARS2 spike protein, a third binding specificity to a third epitope in the SARS2 spike protein, wherein the first, second, third and fourth epitopes are distinct and non-overlapping.

In certain embodiments, the antibodies or antigen-binding fragments of the present invention are multispecific comprising multiple binding specificities for epitopes in the SARS2 spike protein that are distinct and non-overlapping.

In certain embodiments, the antibodies or antigen-binding fragments of the present invention are bispecific comprising a first binding specificity to a first epitope in the receptor binding domain of SARS2 spike protein and a second binding specificity to a second epitope in the receptor binding domain of SARS2 spike protein wherein the first and second epitopes are distinct and non-overlapping.

In certain embodiments, the antibodies or antigen-binding fragments of the present invention are tri-specific comprising a first binding specificity to a first epitope in the receptor binding domain of SARS2 spike protein, a second binding specificity to a second epitope in the receptor binding domain of SARS2 spike protein and a third binding specificity to a third epitope in the receptor binding domain of SARS2 spike protein, wherein the first, second and third epitopes are distinct and non-overlapping.

In certain embodiments, the antibodies or antigen-binding fragments of the present invention are quadri-specific comprising a first binding specificity to a first epitope in the receptor binding domain of SARS2 spike protein, a second binding specificity to a second epitope in the receptor binding domain of SARS2 spike protein, a third binding specificity to a third epitope in the receptor binding domain of SARS2 spike protein, a fourth binding specificity to a fourth epitope in the receptor binding domain of SARS2 spike protein, wherein the first, second, third and fourth epitopes are distinct and non-overlapping.

In certain embodiments, the antibodies or antigen-binding fragments of the present invention are multispecific comprising multiple binding specificities for epitopes in the receptor binding domain of SARS2 spike protein that are distinct and non-overlapping.

In certain embodiments, the antibodies or antigen-binding fragments of the present invention are multispecific comprising a binding specificity for an epitope in the SARS2 spike protein and one or more binding specificities for epitopes in spike proteins from other Coronaviruses (e.g. MERS-CoV, SARS-1, OC43, HKU1 or NL63). In some embodiments, the epitopes are distinct and non-overlapping.

In certain embodiments, the antibodies or antigen-binding fragments of the present invention are bispecific comprising a first binding specificity to a first epitope in the SARS2 spike protein and a second binding specificity to a second epitope in the spike protein of another Coronavirus (e.g. MERS-CoV, SARS-1, OC43, HKU1 or NL63), wherein the first and second epitopes are distinct and non-overlapping. In some embodiments, the first epitope is in the receptor binding domain of SARS2 spike protein.

In certain embodiments, the antibodies or antigen-binding fragments of the present invention are tri-specific comprising a first binding specificity to a first epitope in the SARS2 spike protein, a second binding specificity to a second epitope in the spike protein of another Coronavirus (e.g. MERS-CoV, SARS-1, OC43, HKU1 or NL63), and a third binding specificity to a third epitope in the spike protein of another Coronavirus (e.g. MERS-CoV, SARS-1, OC43, HKU1 or NL63), wherein the first, second and third epitopes are distinct and non-overlapping. In some embodiments, the first epitope is in the receptor binding domain of SARS2 spike protein.

In certain embodiments, the antibodies or antigen-binding fragments of the present invention are quadri-specific comprising a first binding specificity to a first epitope in the receptor binding domain of SARS2 spike protein, a second binding specificity to a second epitope in the spike protein of another Coronavirus (e.g. MERS-CoV, SARS-1, OC43, HKU1 or NL63), and a third binding specificity to a third epitope in the spike protein of another Coronavirus (e.g. MERS-CoV, SARS-1, OC43, HKU1 or NL63), a fourth binding specificity to a fourth epitope in the spike protein of another Coronavirus (e.g. MERS-CoV, SARS-1, OC43, HKU1 or NL63), wherein the first, second, third and fourth epitopes are distinct and non-overlapping.

The invention encompasses a human anti-SARS2-S monoclonal antibody conjugated to a therapeutic moiety (“immunoconjugate”), such as a toxoid or an anti-viral drug to treat SARS2 infection. As used herein, the term “immunoconjugate” refers to an antibody which is chemically or biologically linked to a radioactive agent, a cytokine, an interferon, a target or reporter moiety, an enzyme, a peptide or protein or a therapeutic agent. The antibody may be linked to the radioactive agent, cytokine, interferon, target or reporter moiety, enzyme, peptide or therapeutic agent at any location along the molecule so long as it is able to bind its target.

Examples of immunoconjugates include antibody drug conjugates and antibody-toxin fusion proteins. In one embodiment, the agent may be a second different antibody to SARS2 spike protein. In certain embodiments, the antibody may be conjugated to an agent specific for a virally infected cell. The type of therapeutic moiety that may be conjugated to the anti-SARS2-S antibody and will take into account the condition to be treated and the desired therapeutic effect to be achieved. Examples of suitable agents for forming immunoconjugates are known in the art; see for example, WO 05/103081.

### Nucleic Acids

The term “polynucleotide” or “nucleic acid” includes both single-stranded and double-stranded nucleotide polymers. The nucleotides comprising the nucleic acid can be ribonucleotides or deoxyribonucleotides or a modified form of either type of nucleotide. Said modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phoshoraniladate and phosphoroamidate.

The invention provides nucleic acids encoding anti-SARS2-S antibodies or portions thereof.

For example, the invention provides a nucleic acid molecule that comprises the light chain-encoding sequence of SEQ ID NO: 69. The invention also provides nucleic acid molecules that are at least 90%, at least 95%, at least 98% or at least 99% identical to a nucleic acid comprising the light chain-encoding sequence of SEQ ID NO: 69.

For example, the invention provides a nucleic acid molecule that comprises the heavy chain-encoding sequence of SEQ ID NO: 70. The invention also provides nucleic acid molecules that are at least 90%, at least 95%, at least 98% or at least 99% identical to a nucleic acid comprising the heavy chain-encoding sequence of SEQ ID NO: 70.

For example, the invention provides nucleic acid molecules comprising: (i) the light chain-encoding sequence of SEQ ID NO: 69 and (ii) the heavy chain-encoding sequence of SEQ ID NO: 70.

For example, the invention provides nucleic acid molecules that are at least 90%, at least 95%, at least 98% or at least 99% identical to nucleic acids comprising: (i) the light chain-encoding sequence of SEQ ID NO: 69 and (ii) the heavy chain-encoding sequence of SEQ ID NO: 70.

For example, the invention provides a nucleic acid molecule that comprises the light chain-encoding sequence of SEQ ID NO: 72. The invention also provides nucleic acid molecules that are at least 90%, at least 95%, at least 98% or at least 99% identical to a nucleic acid comprising the light chain-encoding sequence of SEQ ID NO: 74.

For example, the invention provides a nucleic acid molecule that comprises the heavy chain-encoding sequence of SEQ ID NO: 72. The invention also provides nucleic acid molecules that are at least 90%, at least 95%, at least 98% or at least 99% identical to a nucleic acid comprising the heavy chain-encoding sequence of SEQ ID NO: 74.

For example, the invention provides nucleic acid molecules comprising: (i) the light chain-encoding sequence of SEQ ID NO: 72 and (ii) the heavy chain-encoding sequence of SEQ ID NO: 74.

For example, the invention provides nucleic acid molecules that are at least 90%, at least 95%, at least 98% or at least 99% identical to nucleic acids comprising: (i) the light chain-encoding sequence of SEQ ID NO: 72 and (ii) the heavy chain-encoding sequence of SEQ ID NO: 74.

For example, the invention provides nucleic acid molecules that comprise any one of the following light chain variable region-encoding sequences: SEQ ID NOs: 15, 17, 19, 23, 49 and 50. The invention also provides nucleic acid molecules that are at least 90%, at least 95%, at least 98% or at least 99% identical to nucleic acids comprising any one of the following light chain variable region-encoding sequences: SEQ ID NOs: 15, 17, 19, 23, 49 and 50.

For example, the invention provides a nucleic acid molecule comprising the sequence of SEQ ID NO: 23. The invention also provides nucleic acid molecules that are at least 90%, at least 95%, at least 98% or at least 99% identical to a nucleic acid comprising the sequence of SEQ ID NO: 23.

For example, the invention provides nucleic acid molecules that comprise any one of the following heavy chain variable region-encoding sequences: SEQ ID NOs: 16, 18, 20 and 24. The invention also provides nucleic acid molecules that are at least 90%, at least 95%, at least 98% or at least 99% identical to nucleic acids comprising any one of the following heavy chain variable region-encoding sequences: SEQ ID NOs: 16, 18, 20 and 24.

For example, the invention provides a nucleic acid molecule having the sequence of SEQ ID NO: 24. The invention also provides nucleic acid molecules that are at least 90%, at least 95%, at least 98% or at least 99% identical to a nucleic acid having the sequence of SEQ ID NO: 24.

For example, the invention provides nucleic acid molecules comprising: (i) any one of the following light chain variable region-encoding sequences: SEQ ID NOs: SEQ ID NOs: 15, 17, 19, 23, 49 and 50 and (ii) any one of the following heavy chain variable region-encoding sequences: SEQ ID NOs: 16, 18, 20 and 24. The invention also provides nucleic acid molecules that are at least 90%, at least 95%, at least 98% or at least 99% identical to nucleic acids comprising: (i) any one of the following light chain variable region-encoding sequences: SEQ ID NOs: SEQ ID NOs: 15, 17, 19, 23, 49 and 50 and (ii) any one of the following heavy chain variable region-encoding sequences: SEQ ID NOs: 16, 18, 20 and 24.

For example, the invention provides nucleic acid molecules comprising: (i) the sequence of SEQ ID NO: 23 and (ii) the sequence of SEQ ID NO: 24. The invention also provides nucleic acid molecules that are at least 90%, at least 95%, at least 98% or at least 99% identical to nucleic acids comprising: (i) the sequence of SEQ ID NO: 23 and (ii) the sequence of SEQ ID NO: 24.

For example, the invention provides nucleic acid molecules encoding any one of the following heavy chain variable region sequences: SEQ ID NOs: 2, 4, 6, 7 and 10. The invention also provides nucleic acid molecules that are at least 90%, at least 95%, at least 98% or at least 99% identical to nucleic acids encoding any one of the following heavy chain variable region sequences: SEQ ID NOs: 2, 4, 6, 7 and 10.

For example, the invention provides a nucleic acid molecule encoding the heavy chain variable region sequence of SEQ ID NO: 10. The invention also provides nucleic acid molecules that are at least 90%, at least 95%, at least 98% or at least 99% identical to nucleic acids encoding the heavy chain variable region sequence of SEQ ID NO: 10.

For example, the invention provides nucleic acid molecules encoding any one of the following light chain variable region sequences: SEQ ID NOs: 1, 3, 5, 9, 47 and 48. The invention also provides nucleic acid molecules that are at least 90%, at least 95%, at least 98% or at least 99% identical to nucleic acids encoding any one of the following light chain variable region sequences: SEQ ID NOs: 1, 3, 5, 9, 47 and 48.

For example, the invention provides a nucleic acid molecule encoding the light chain variable region sequence of SEQ ID NO: 9. The invention also provides nucleic acid molecules that are at least 90%, at least 95%, at least 98% or at least 99% identical to nucleic acids encoding the light chain variable region sequence of SEQ ID NO: 9.

For example, the invention provides nucleic acid molecules encoding: (i) any one of the following heavy chain variable region sequences: SEQ ID NOs: 2, 4, 6, 7 and 10 and (ii) any one of the following light chain variable region sequences: SEQ ID NOs: 1, 3, 5, 9, 47 and 48. The invention also provides nucleic acid molecules that are at least 90%, at least 95%, at least 98% or at least 99% identical to nucleic acids encoding: (i) any one of the following heavy chain variable region sequences: SEQ ID NOs: 2, 4, 6, 7 and 10 and (ii) any one of the following light chain variable region sequences: SEQ ID NOs: 1, 3, 5, 9, 47 and 48.

For example, the invention provides nucleic acid molecules encoding: (i) the heavy chain variable region sequence of SEQ ID NO: 10 and (ii) the light chain variable region sequence of SEQ ID NO: 9. The invention also provides nucleic acid molecules that are at least 90%, at least 95%, at least 98% or at least 99% identical to nucleic acids encoding: (i) the heavy chain variable region sequence of SEQ ID NO: 10 and (ii) the light chain variable region sequence of SEQ ID NO: 9.

For example, the invention provides nucleic acid molecules encoding a heavy chain variable region sequence that comprises the CDR sequences of any one of the following heavy chain variable region sequences: SEQ ID NOs: 2, 4, 6, 7 and 10.

For example, the invention provides a nucleic acid molecule encoding a heavy chain variable region sequence that comprises the CDR sequences of the heavy chain variable region sequence of SEQ ID NO: 10.

In some embodiments, the invention provides nucleic acid molecules encoding a heavy chain variable region sequence that comprises any one of the following groups of three CDR sequences: SEQ ID NOs: 32-34, 38-40, 44-46 and 56-58.

In some embodiments, the invention provides nucleic acid molecules encoding a heavy chain variable region sequence that comprises the following group of three CDR sequences: SEQ ID NOs: 56-58.

The invention also provides nucleic acid molecules that encode a heavy chain variable region sequence that comprises CDR sequences that are at least 90%, at least 95%, at least 98% or at least 99% identical to the CDR sequences of any one of the following heavy chain variable region sequences: SEQ ID NOs: 2, 4, 6, 7 and 10.

For example, the invention provides nucleic acid molecules that encode a heavy chain variable region sequence that comprises CDR sequences that are at least 90%, at least 95%, at least 98% or at least 99% identical to the CDR sequences of the heavy chain variable region sequence of SEQ ID NO: 10.

In some embodiments, the invention provides nucleic acid molecules that encode a heavy chain variable region sequence that comprises CDR1, CDR2 and CDR3 sequences that are at least 90%, at least 95%, at least 98% or at least 99% identical to the CDR1, CDR2 and CDR3, respectively, of any one of the following groups of three CDR sequences: SEQ ID NOs: 32-34, 38-40, 44-46 and 56-58.

In some embodiments, the invention provides nucleic acid molecules that encode a heavy chain variable region sequence that comprises CDR1, CDR2 and CDR3 sequences that are at least 90%, at least 95%, at least 98% or at least 99% identical to the CDR1, CDR2 and CDR3, respectively, of the following group of three CDR sequences: SEQ ID NOs: 56-58.

For example, the invention provides nucleic acid molecules encoding a light chain variable region sequence that comprises the CDR sequences of any one of the following light chain variable region sequences: SEQ ID NOs: 1, 3, 5, 9, 47 and 48.

For example, the invention provides nucleic acid molecules encoding a light chain variable region sequence that comprises the CDR sequences of the light chain variable region sequence of SEQ ID NO: 9.

In some embodiments, the invention provides nucleic acid molecules encoding a light chain variable region sequence that comprises any one of the following groups of three CDR sequences: SEQ ID NOs: 29-31, 35-37, 41-43, 53-55, 59-61 and 62-64.

In some embodiments, the invention provides nucleic acid molecules encoding a light chain variable region sequence that comprises the following group of three CDR sequences: SEQ ID NOs: 53-55.

The invention also provides nucleic acid molecules that encode a light chain variable region sequence that comprises CDR sequences that are at least 90%, at least 95%, at least 98% or at least 99% identical to the CDR sequences of any one of the following light chain variable region sequences: SEQ ID NOs: 1, 3, 5, 9, 47 and 48.

The invention also provides nucleic acid molecules that encode a light chain variable region sequence that comprises CDR sequences that are at least 90%, at least 95%, at least 98% or at least 99% identical to the CDR sequences of the light chain variable region sequence of SEQ ID NO: 9.

In some embodiments, the invention provides nucleic acid molecules that encode a light chain variable region sequence that comprises CDR1, CDR2 and CDR3 sequences that are at least 90%, at least 95%, at least 98% or at least 99% identical to the CDR1, CDR2 and CDR3, respectively, of any one of the following groups of three CDR sequences: SEQ ID NOs: 29-31, 35-37, 41-43, 53-55, 59-61 and 62-64.

In some embodiments, the invention provides nucleic acid molecules that encode a light chain variable region sequence that comprises CDR1, CDR2 and CDR3 sequences that are at least 90%, at least 95%, at least 98% or at least 99% identical to the CDR1, CDR2 and CDR3, respectively, of the following group of three CDR sequences: SEQ ID NOs: 53-55.

For example, the invention provides nucleic acid molecules encoding: (i) a heavy chain variable region sequence that comprises the CDR sequences of any one of the following heavy chain variable region sequences: SEQ ID NOs: 2, 4, 6, 7 and 10 and (ii) a light chain variable region sequence that comprises the CDR sequences of any one of the following light chain variable region sequences: SEQ ID NOs: 1, 3, 5, 9, 47 and 48. In some embodiments, the invention provides nucleic acid molecules encoding (i) a heavy chain variable region sequence that comprises any one of the following groups of three CDR sequences: SEQ ID NOs: 32-34, 38-40, 44-46 and 56-58 and (ii) a light chain variable region sequence that comprises any one of the following groups of three CDR sequences: SEQ ID NOs: 29-31, 35-37, 41-43, 53-55, 59-61 and 62-64. The invention also provides nucleic acid molecules that encode: (i) a heavy chain variable region sequence that comprises CDR sequences that are at least 90%, at least 95%, at least 98% or at least 99% identical to the CDR sequences of any one of the following heavy chain variable region sequences: SEQ ID NOs: 2, 4, 6, 7 and 10 and (ii) a light chain variable region sequence that comprises CDR sequences that are at least 90%, at least 95%, at least 98% or at least 99% identical to the CDR sequences of any one of the following light chain variable region sequences: SEQ ID NOs: 1, 3, 5, 9, 47 and 48. In some embodiments, the invention provides nucleic acid molecules that encode (i) a heavy chain variable region sequence that comprises CDR1, CDR2 and CDR3 sequences that are at least 90%, at least 95%, at least 98% or at least 99% identical to the CDR1, CDR2 and CDR3, respectively, of any one of the following groups of three CDR sequences: SEQ ID NOs: 32-34, 38-40, 44-46 and 56-58 and (ii) a light chain variable region sequence that comprises CDR1, CDR2 and CDR3 sequences that are at least 90%, at least 95%, at least 98% or at least 99% identical to the CDR1, CDR2 and CDR3, respectively, of any one of the following groups of three CDR sequences: SEQ ID NOs: 29-31, 35-37, 41-43, 53-55, 59-61 and 62-64.

For example, the invention provides nucleic acid molecules encoding: (i) a heavy chain variable region sequence that comprises the CDR sequences of the heavy chain variable region sequence of SEQ ID NO: 10 and (ii) a light chain variable region sequence that comprises the CDR sequences of the light chain variable region sequence of SEQ ID NO: 9. In some embodiments, the invention provides nucleic acid molecules encoding (i) a heavy chain variable region sequence that comprises the following group of three CDR sequences: SEQ ID NOs: 56-58 and (ii) a light chain variable region sequence that comprises the following group of three CDR sequences: SEQ ID NOs: 53-55. The invention also provides nucleic acid molecules that encode: (i) a heavy chain variable region sequence that comprises CDR sequences that are at least 90%, at least 95%, at least 98% or at least 99% identical to the CDR sequences of the heavy chain variable region sequence of SEQ ID NO: 10 and (ii) a light chain variable region sequence that comprises CDR sequences that are at least 90%, at least 95%, at least 98% or at least 99% identical to the CDR sequences of the light chain variable region sequence of SEQ ID NO: 9. In some embodiments, the invention provides nucleic acid molecules that encode (i) a heavy chain variable region sequence that comprises CDR1, CDR2 and CDR3 sequences that are at least 90%, at least 95%, at least 98% or at least 99% identical to the CDR1, CDR2 and CDR3, respectively, of the following group of three CDR sequences: SEQ ID NOs: 56-58 and (ii) a light chain variable region sequence that comprises CDR1, CDR2 and CDR3 sequences that are at least 90%, at least 95%, at least 98% or at least 99% identical to the CDR1, CDR2 and CDR3, respectively, of the following group of three CDR sequences: SEQ ID NOs: 53-55.

The invention further provides recombinant expression vectors capable of expressing a polypeptide comprising a heavy or light chain variable region of an anti-SARS2-S antibody. For example, the invention provides recombinant expression vectors comprising any of the nucleic acid molecules mentioned above.

The invention further provides host cells into which any of the vectors mentioned above have been introduced. The invention further provides methods of producing the antibodies and antibody fragments of the invention by culturing the host cells under conditions permitting production of the antibodies or antibody fragments, and recovering the antibodies and antibody fragments so produced.

### Pharmaceutical Compositions

The invention provides pharmaceutical composition comprising an antibody of the invention. In some embodiments, the pharmaceutical composition further comprises a pharmaceutically acceptable carrier. The term “pharmaceutically acceptable carrier” includes any and all solvents, buffers, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e. g. by injection or infusion). For example, in some embodiments, a composition for intravenous administration typically is a solution in sterile isotonic aqueous buffer.

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

The invention provides therapeutic compositions comprising the anti-SARS2-S antibodies or antigen-binding fragments thereof of the present invention. Therapeutic compositions in accordance with the invention will be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of antibody may vary depending upon the age and the size of a subject to be administered, target disease, conditions, route of administration, and the like. When an antibody of the present invention is used for treating a disease or disorder in an adult patient, or for preventing such a disease, it is advantageous to administer the antibody of the present invention normally at a single dose of about 0.1 to about 60 mg/kg body weight, more preferably about 5 to about 60, about 10 to about 50, or about 20 to about 50 mg/kg body weight. Depending on the severity of the condition, the frequency and the duration of the treatment can be adjusted. In certain embodiments, the antibody or antigen-binding fragment thereof of the invention can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the antibody or antigen-binding fragment thereof in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

### Methods of Production

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103) Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies. (See Kozbor, J. Immunol., 133:3001 (1984); Brodeur et al., Monoclonal Antibody Production Techniques and Applications, Marcel Dekker, Inc., New York, (1987) pp. 51-63)).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Moreover, in therapeutic applications of monoclonal antibodies, it is important to identify antibodies having a high degree of specificity and a high binding affinity for the target antigen.

After the desired hybridoma cells are identified, the clones can be subcloned by limiting dilution procedures and grown by standard methods. (See Goding, Monoclonal Antibodies: Principles and Practice, Academic Press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

Monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (see U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Fully human antibodies are antibody molecules in which the entire sequence of both the light chain and the heavy chain, including the CDRs, arise from human genes. Such antibodies are termed “humanized antibodies”, “human antibodies”, or “fully human antibodies” herein.

Human monoclonal antibodies can be prepared by using trioma technique; the human B-cell hybridoma technique (see Kozbor, et al., 1983 Immunol Today 4: 72); and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, humanized antibodies can be produced in transgenic plants, as an inexpensive production alternative to existing mammalian systems. For example, the transgenic plant may be a tobacco plant, i.e., Nicotiania benthamiana, and Nicotiana tabaccum. The antibodies are purified from the plant leaves. Stable transformation of the plants can be achieved through the use of Agrobacterium tumefaciens or particle bombardment. For example, nucleic acid expression vectors containing at least the heavy and light chain sequences are expressed in bacterial cultures, i.e., A. tumefaciens strain BLA4404, via transformation. Infiltration of the plants can be accomplished via injection. Soluble leaf extracts can be prepared by grinding leaf tissue in a mortar and by centrifugation. Isolation and purification of the antibodies can be readily be performed by many of the methods known to the skilled artisan in the art. Other methods for antibody production in plants are described in, for example, Fischer et al., Vaccine, 2003, 21:820-5; and Ko et al, Current Topics in Microbiology and Immunology, Vol. 332, 2009, pp. 55-78. As such, the present invention further provides any cell or plant comprising a vector that encodes the antibody of the present invention, or produces the antibody of the present invention.

In addition, human antibodies can also be produced using additional techniques, including phage display libraries. (See Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in WO 2006/008548, WO 2007/096779, WO 2010/109165, WO 2010/070263, WO 2014/141189 and WO 2014/141192.

One method for producing an antibody of interest, such as a human antibody, is disclosed in U.S. Pat. No. 5,916,771. This method includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen and a correlative method for selecting an antibody that binds immunospecifically to the relevant epitope with high affinity, are disclosed in PCT publication WO 99/53049.

The antibody can be expressed by a vector containing a DNA segment encoding the single chain antibody described above.

These can include vectors, liposomes, naked DNA, adjuvant-assisted DNA, gene gun, catheters, etc. Vectors include chemical conjugates such as described in WO 93/64701, which has targeting moiety (e.g. a ligand to a cellular surface receptor), and a nucleic acid binding moiety (e.g. polylysine), viral vector (e.g. a DNA or RNA viral vector), fusion proteins such as described in PCT/US 95/02140 (WO 95/22618) which is a fusion protein containing a target moiety (e.g. an antibody specific for a target cell) and a nucleic acid binding moiety (e.g. a protamine), plasmids, phage, etc. The vectors can be chromosomal, non-chromosomal or synthetic.

Preferred vectors include viral vectors, fusion proteins and chemical conjugates. Retroviral vectors include Moloney murine leukemia viruses. DNA viral vectors are preferred. These vectors include pox vectors such as orthopox or avipox vectors, herpesvirus vectors such as a herpes simplex I virus (HSV) vector (see Geller, A. I. et al., J. Neurochem, 64:487 (1995); Lim, F., et al., in DNA Cloning: Mammalian Systems, D. Glover, Ed. (Oxford Univ. Press, Oxford England) (1995); Geller, A. I. et al., Proc Natl. Acad. Sci.: U.S.A. 90:7603 (1993); Geller, A. I., et al., Proc Natl. Acad. Sci USA 87:1149 (1990), Adenovirus Vectors (see LeGal LaSalle et al., Science, 259:988 (1993); Davidson, et al., Nat. Genet 3:219 (1993); Yang, et al., J. Virol. 69:2004 (1995) and Adeno-associated Virus Vectors (see Kaplitt, M. G. et al., Nat. Genet. 8:148 (1994).

Pox viral vectors introduce the gene into the cells cytoplasm. Avipox virus vectors result in only a short term expression of the nucleic acid. Adenovirus vectors, adeno-associated virus vectors and herpes simplex virus (HSV) vectors are preferred for introducing the nucleic acid into neural cells. The adenovirus vector results in a shorter term expression (about 2 months) than adeno-associated virus (about 4 months), which in turn is shorter than HSV vectors. The particular vector chosen will depend upon the target cell and the condition being treated. The introduction can be by standard techniques, e.g. infection, transfection, transduction or transformation. Examples of modes of gene transfer include e.g., naked DNA, CaPO4 precipitation, DEAE dextran, electroporation, protoplast fusion, lipofection, cell microinjection, and viral vectors.

The vector can be employed to target essentially any desired target cell. For example, stereotaxic injection can be used to direct the vectors (e.g. adenovirus, HSV) to a desired location. Additionally, the particles can be delivered by intracerebroventricular (icy) infusion using a minipump infusion system, such as a SynchroMed Infusion System. A method based on bulk flow, termed convection, has also proven effective at delivering large molecules to extended areas of the brain and may be useful in delivering the vector to the target cell. (See Bobo et al., Proc. Natl. Acad. Sci. USA 91:2076-2080 (1994); Morrison et al., Am. J. Physiol. 266:292-305 (1994)). Other methods that can be used include catheters, intravenous, parenteral, intraperitoneal and subcutaneous injection, and oral or other known routes of administration.

These vectors can be used to express large quantities of antibodies that can be used in a variety of ways. For example, to detect the presence of SARS2 in a sample. The antibody can also be used to try to bind to and disrupt SARS2.

In a preferred embodiment, the antibodies of the present invention are full-length antibodies, containing an Fc region similar to wild-type Fc regions that bind to Fc receptors.

Heteroconjugate antibodies are also within the scope of the present invention. Heteroconjugate antibodies are composed of two covalently joined antibodies. It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

It can be desirable to modify the antibody of the invention with respect to effector function, so as to enhance, e.g., the effectiveness of the antibody in neutralizing or preventing viral infection. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). (See Caron et al., J. Exp Med., 176: 1191-1195 (1992) and Shopes, J. Immunol., 148: 2918-2922 (1992)). Alternatively, an antibody can be engineered that has dual Fc regions and can thereby have enhanced complement lysis and ADCC capabilities. (See Stevenson et al., Anti-Cancer Drug Design, 3: 219-230 (1989)). In a preferred embodiment, the antibody of the present invention has modifications of the Fc region, such that the Fc region does not bind to the Fc receptors. Preferably, the Fc receptor is Fcγ receptor. Particularly preferred are antibodies with modification of the Fc region such that the Fc region does not bind to Fcγ, but still binds to neonatal Fc receptor.

The invention also pertains to immunoconjugates comprising an antibody conjugated to a cytotoxic agent such as a toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include 212Bi, 131I 131In, 90Y, and 186Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. (See WO94/11026).

Those of ordinary skill in the art will recognize that a large variety of possible moieties can be coupled to the resultant antibodies or to other molecules of the invention. (See, for example, “Conjugate Vaccines”, Contributions to Microbiology and Immunology, J. M. Cruse and R. E. Lewis, Jr (eds), Carger Press, New York, (1989), the entire contents of which are incorporated herein by reference).

Coupling may be accomplished by any chemical reaction that will bind the two molecules so long as the antibody and the other moiety retain their respective activities. This linkage can include many chemical mechanisms, for instance covalent binding, affinity binding, intercalation, coordinate binding and complexation. The preferred binding is, however, covalent binding. Covalent binding can be achieved either by direct condensation of existing side chains or by the incorporation of external bridging molecules. Many bivalent or polyvalent linking agents are useful in coupling protein molecules, such as the antibodies of the present invention, to other molecules. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents. (See Killen and Lindstrom, Jour. Immun 133:1335-2549 (1984); Jansen et al, Immunological Reviews 62:185-216 (1982); and Vitetta et al., Science 238:1098 (1987)). Preferred linkers are described in the literature. (See, for example, Ramakrishnan, S. et al., Cancer Res. 44:201-208 (1984) describing use of MBS (M-maleimidobenzoyl-N-hydroxysuccinimide ester). See also, U.S. Pat. No. 5,030,719, describing use of halogenated acetyl hydrazide derivative coupled to an antibody by way of an oligopeptide linker. Particularly preferred linkers include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide] hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to conjugates with differing physio-chemical properties. For example, sulfo-NHS esters of alkyl carboxylates are more stable than sulfo-NHS esters of aromatic carboxylates. NETS-ester containing linkers are less soluble than sulfo-NHS esters. Further, the linker SMPT contains a sterically hindered disulfide bond, and can form conjugates with increased stability. Disulfide linkages, are in general, less stable than other linkages because the disulfide linkage is cleaved in vitro, resulting in less conjugate available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

Methods for the screening of antibodies that possess the desired specificity include, but are not limited to, enzyme linked immunosorbent assay (ELISA) and other immunologically mediated techniques known within the art.

### Uses of Anti-SARS2-S Antibodies

Antibodies directed against the spike protein of SARS2 may be used in methods known within the art relating to the localization and/or quantitation of SARS2 (e.g., for use in measuring levels of the SARS2-S protein within appropriate physiological samples, for use in diagnostic methods, for use in imaging the protein, and the like). In a given embodiment, antibodies specific to SARS2, or derivative, fragment, analogue or homolog thereof, that contain the antibody derived antigen binding domain, are utilized as pharmacologically active compounds (referred to hereinafter as “Therapeutics”).

An antibody specific for SARS2 can be used to isolate a SARS2 polypeptide by standard techniques, such as immunoaffinity, chromatography or immunoprecipitation. Antibodies directed against a SARS2 protein (or a fragment thereof) can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H. Accordingly, the anti-SARS2-S antibodies of the present invention may be used to detect and/or measure SARS2 in a sample, e.g., for diagnostic purposes. Some embodiments contemplate the use of one or more antibodies of the present invention in assays to detect a disease or disorder such as viral infection. Exemplary diagnostic assays for SARS2 may comprise, e.g., contacting a sample, obtained from a patient, with an anti-SARS2-S antibody of the invention, wherein the anti-SARS2-S antibody is labelled with a detectable label or reporter molecule or used as a capture ligand to selectively isolate SARS2 from patient samples. Alternatively, an unlabelled anti-SARS2-S antibody can be used in diagnostic applications in combination with a secondary antibody which is itself detectably labelled. The detectable label or reporter molecule can be a radioisotope, such as ³H, ¹⁴C, ³²P, ³⁵S, or ¹²⁵I; a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure SARS2 in a sample include enzyme-linked immunosorbent assay (ELISA), radioimmunoassay (RIA), and fluorescence-activated cell sorting (FACS).

Samples that can be used in SARS2 diagnostic assays according to the present invention include any tissue or fluid sample obtainable from a patient, which contains detectable quantities of either SARS2 spike protein, or fragments thereof, under normal or pathological conditions. Generally, levels of SARS2 spike protein in a particular sample obtained from a healthy patient (e.g., a patient not afflicted with a disease associated with SARS2) will be measured to initially establish a baseline, or standard, level of SARS2. This baseline level of SARS2 can then be compared against the levels of SARS2 measured in samples obtained from individuals suspected of having a SARS2-associated condition, or symptoms associated with such condition.

The antibodies specific for SARS2 spike protein may contain no additional labels or moieties, or they may contain an N-terminal or C-terminal label or moiety. In one embodiment, the label or moiety is biotin. In a binding assay, the location of a label (if any) may determine the orientation of the peptide relative to the surface upon which the peptide is bound. For example, if a surface is coated with avidin, a peptide containing an N-terminal biotin will be oriented such that the C-terminal portion of the peptide will be distal to the surface

Antibodies of the invention, including polyclonal, monoclonal, humanized and fully human antibodies, may be used as therapeutic agents. Such agents will generally be employed to treat or prevent a SARS2-related disease or pathology in a subject. An antibody preparation, preferably one having high specificity and high affinity for its target antigen, is administered to the subject and will generally have an effect due to its binding with the target. Administration of the antibody may abrogate or inhibit or interfere with the internalization of the virus into a cell. For example, the antibody may bind to the target and prevent SARS2 binding the ACE2 receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target. The amount required to be administered will furthermore depend on the binding affinity of the antibody for its specific antigen and will also depend on the rate at which an administered antibody is depleted from the free volume of the subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Antibodies specifically binding a SARS2 protein or a fragment thereof of the invention, as well as other molecules identified by the screening assays disclosed herein, can be administered for the treatment of a SARS2-related disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa., 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

Formulations can contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nanoparticles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody. The matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

An antibody according to the invention can be used as an agent for detecting the presence of a SARS2 (or a protein or a protein fragment thereof) in a sample. Preferably, the antibody contains a detectable label. Antibodies can be polyclonal, or more preferably, monoclonal. An intact antibody is preferred. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is, for example, blood and a fraction or component of blood including blood serum, blood plasma, sputum, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-analyte protein antibody. For example, the antibody can be labeled with a radioactive marker whose presence and location in a subject can be detected by standard imaging techniques.

Passive immunization has proven to be an effective and safe strategy for the prevention and treatment of viral diseases. (See Keller et al., Clin. Microbiol. Rev. 13:602-14 (2000); Casadevall, Nat. Biotechnol. 20:114 (2002); Shibata et al., Nat. Med. 5:204-10 (1999); and Igarashi et al., Nat. Med. 5:211-16 (1999)). Passive immunization using neutralizing human monoclonal antibodies could provide an immediate treatment strategy for emergency prophylaxis and treatment of SARS2 infection and related diseases and disorders while the alternative and more time-consuming development of vaccines and new drugs is underway.

The invention provides for both prophylactic and therapeutic methods of treating a subject at risk of (or susceptible to) a SARS2-related disease or disorder.

In one aspect, the invention provides methods for preventing a SARS2-related disease or disorder in a subject by administering to the subject an antibody of the invention. Optionally, two or more anti-SARS2 antibodies are co-administered.

Subjects at risk for a SARS2-related diseases or disorders include patients who have been exposed to the SARS2. For example, the subjects have travelled to regions or countries of the world in which other SARS2 infections have been reported and confirmed. Administration of a prophylactic agent can occur prior to the manifestation of symptoms characteristic of the SARS2-related disease or disorder, such that a disease or disorder is prevented or, alternatively, delayed in its progression.

In some embodiments, the antibody of the present invention can be administered with other antibodies or antibody fragments known to neutralize SARS2. Administration of said antibodies can be sequential, concurrent, or alternating.

Another aspect of the invention pertains to methods of treating a SARS2-related disease or disorder in a patient. In one embodiment, the method involves administering an agent (e.g., an agent identified by a screening assay described herein and/or monoclonal antibody identified according to the methods of the invention), or combination of agents that neutralize the SARS2 to a patient suffering from the disease or disorder.

In some embodiments, the anti-SARS2-S antibodies of the invention are for use in therapy. In some embodiments, the antibodies of the invention are for use in preventing, treating or ameliorating coronavirus infection, optionally betacoronavirus infection, such as SARS2 infection. In some embodiments, the antibodies of the invention are for use in preventing, treating or ameliorating ACE2-dependent coronavirus infection. In some embodiments, the antibodies of the invention are for use in preventing, treating or ameliorating ACE2-dependent betacoronavirus infection. In some embodiments, the antibodies of the invention are for use in preventing, treating or ameliorating ACE2-dependent SARS-like virus infection.

In some embodiments, the antibodies of the invention are for use in preventing, treating or ameliorating SARS2 virus infection, wherein the SARS2 virus has the E484K mutation in its spike protein.

In some embodiments, the antibodies of the invention are for use in preventing, treating or ameliorating SARS2 virus infection, wherein the SARS2 virus has the N501Y mutation in its spike protein.

In some embodiments, the antibodies of the invention are for use in preventing, treating or ameliorating SARS2 virus infection, wherein the SARS2 virus has the K417N mutation in its spike protein.

In some embodiments, the antibodies of the invention are for use in preventing, treating or ameliorating SARS2 virus infection, wherein the SARS2 virus has: (a) the E484K and the N501Y, (b) the E484K and the K417N, or (b) the E484K, N501Y and K417N mutations in its spike protein.

In some embodiments, the antibodies of the invention are for use in preventing, treating or ameliorating SARS2 virus infection, wherein the SARS2 virus has one or more spike protein mutations selected from N439K, Q493R and S494P.

In some embodiments, the therapy is preventing, treating or ameliorating:

(a) coronavirus-induced pneumonia, optionally severe coronavirus-induced pneumonia, and/or
(b) coronavirus-induced weight loss, and/or
(c) coronavirus-induced lung inflammation, and/or
(d) coronavirus replication, optionally coronavirus replication in the lower respiratory tract, and/or
(e) coronavirus-induced lung lesions, optionally coronavirus-induced gross lesions in the lung.

In some embodiments, the therapy is preventing, treating or ameliorating:

(a) betacoronavirus-induced pneumonia, optionally severe betacoronavirus-induced pneumonia, and/or
(b) betacoronavirus-induced weight loss, and/or
(c) betacoronavirus-induced lung inflammation, and/or
(d) betacoronavirus replication, optionally betacoronavirus replication in the lower respiratory tract, and/or
(e) betacoronavirus-induced lung lesions, optionally betacoronavirus-induced gross lesions in the lung.

In some embodiments, the therapy is preventing, treating or ameliorating:

(a) ACE2-dependent coronavirus-induced pneumonia, optionally severe coronavirus-induced pneumonia, and/or
(b) ACE2-dependent coronavirus-induced weight loss, and/or
(c) ACE2-dependent coronavirus-induced lung inflammation, and/or
(d) ACE2-dependent coronavirus replication, optionally coronavirus replication in the lower respiratory tract, and/or
(e) ACE2-dependent coronavirus-induced lung lesions, optionally coronavirus-induced gross lesions in the lung.

In some embodiments, the therapy is preventing, treating or ameliorating:

(a) ACE2-dependent betacoronavirus-induced pneumonia, optionally severe coronavirus-induced pneumonia, and/or
(b) ACE2-dependent betacoronavirus-induced weight loss, and/or
(c) ACE2-dependent betacoronavirus-induced lung inflammation, and/or
(d) ACE2-dependent betacoronavirus replication, optionally coronavirus replication in the lower respiratory tract, and/or
(e) ACE2-dependent betacoronavirus-induced lung lesions, optionally coronavirus-induced gross lesions in the lung.

In some embodiments, the therapy is preventing, treating or ameliorating:

(a) ACE2-dependent SARS-like virus-induced pneumonia, optionally severe coronavirus-induced pneumonia, and/or
(b) ACE2-dependent SARS-like virus-induced weight loss, and/or
(c) ACE2-dependent SARS-like virus-induced lung inflammation, and/or
(d) ACE2-dependent SARS-like virus replication, optionally coronavirus replication in the lower respiratory tract, and/or
(e) ACE2-dependent SARS-like virus-induced lung lesions, optionally coronavirus-induced gross lesions in the lung.

In some embodiments, the therapy is preventing, treating or ameliorating SARS2 infection in a subject.

In some embodiments, the therapy is preventing, treating or ameliorating:

(a) SARS2-induced pneumonia, optionally severe SARS2-induced pneumonia, and/or
(b) SARS2-induced weight loss, and/or
(c) SARS2-induced lung inflammation, and/or
(d) SARS2 replication, optionally SARS2 replication in the lower respiratory tract, and/or (e) SARS2-induced lung lesions, optionally SARS2-induced gross lesions in the lung.
In some embodiments, the SARS2 virus has one or more spike protein mutations selected from K417N, N439K, E484K, Q493R, S494P, and N501Y.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating coronavirus infection, optionally betacoronavirus infection, such as SARS2 infection.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating coronavirus-induced pneumonia, optionally betacoronavirus-induced pneumonia, such as SARS2-induced pneumonia.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating severe coronavirus-induced pneumonia, optionally severe betacoronavirus-induced pneumonia, such as severe SARS2-induced pneumonia.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
   vi. SEQ ID NO: 58 for CDR3 of the heavy chain, for use in preventing, treating or ameliorating coronavirus-induced weight loss, optionally betacoronavirus-induced weight loss, such as SARS2-induced weight loss.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating coronavirus-induced lung inflammation, optionally betacoronavirus-induced lung inflammation, such as SARS2-induced lung inflammation.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating coronavirus replication, optionally betacoronavirus replication, such as SARS2 replication.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating coronavirus replication in the lower respiratory tract, optionally betacoronavirus replication in the lower respiratory tract, such as SARS2 replication in the lower respiratory tract.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating coronavirus-induced lung lesions, optionally betacoronavirus-induced lung lesions, such as SARS2-induced lung lesions.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating coronavirus-induced gross lesions in the lung, optionally betacoronavirus-induced gross lesions in the lung, such as SARS2-induced gross lesions in the lung.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating ACE2-dependent coronavirus replication in the lower respiratory tract, optionally ACE2-dependent betacoronavirus replication in the lower respiratory tract, such as ACE2-dependent SARS-like virus replication in the lower respiratory tract.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating ACE2-dependent coronavirus-induced lung lesions, optionally ACE2-dependent betacoronavirus-induced lung lesions, such as ACE2-dependent SARS-like virus-induced lung lesions.

The invention further provides an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain,
   for use in preventing, treating or ameliorating ACE2-dependent coronavirus-induced gross lesions in the lung, optionally ACE2-dependent betacoronavirus-induced gross lesions in the lung, such as ACE2-dependent SARS-like virus-induced gross lesions in the lung.

The invention further provides a method for preventing, treating or ameliorating coronavirus infection, optionally betacoronavirus infection, such as SARS2 infection, wherein the method comprises administering an anti-SARS2-S antibody of the invention to a subject.

In some embodiments, the method is for preventing, treating or ameliorating:

(a) coronavirus-induced pneumonia, optionally severe coronavirus-induced pneumonia, and/or
(b) coronavirus-induced weight loss, and/or
(c) coronavirus-induced lung inflammation, and/or
(d) coronavirus replication, optionally coronavirus replication in the lower respiratory tract, and/or
(e) coronavirus-induced lung lesions, optionally coronavirus-induced gross lesions in the lung.

In some embodiments, the method is for preventing, treating or ameliorating:

(a) betacoronavirus-induced pneumonia, optionally severe betacoronavirus-induced pneumonia, and/or
(b) betacoronavirus-induced weight loss, and/or
(c) betacoronavirus-induced lung inflammation, and/or
(d) betacoronavirus replication, optionally betacoronavirus replication in the lower respiratory tract, and/or
(e) betacoronavirus-induced lung lesions, optionally betacoronavirus-induced gross lesions in the lung.

The invention further provides a method for preventing, treating or ameliorating ACE2-dependent coronavirus infection, optionally ACE2-dependent betacoronavirus infection, such as ACE2-dependent SARS-like virus infection, wherein the method comprises administering an anti-SARS2-S antibody of the invention to a subject.

In some embodiments, the method is for preventing, treating or ameliorating:

(a) ACE2-dependent coronavirus-induced pneumonia, optionally severe coronavirus-induced pneumonia, and/or
(b) ACE2-dependent coronavirus-induced weight loss, and/or
(c) ACE2-dependent coronavirus-induced lung inflammation, and/or
(d) ACE2-dependent coronavirus replication, optionally coronavirus replication in the lower respiratory tract, and/or
(e) ACE2-dependent coronavirus-induced lung lesions, optionally coronavirus-induced gross lesions in the lung.

In some embodiments, the method is for preventing, treating or ameliorating:

(a) ACE2-dependent betacoronavirus-induced pneumonia, optionally severe betacoronavirus-induced pneumonia, and/or
(b) ACE2-dependent betacoronavirus-induced weight loss, and/or
(c) ACE2-dependent betacoronavirus-induced lung inflammation, and/or
(d) ACE2-dependent betacoronavirus replication, optionally betacoronavirus replication in the lower respiratory tract, and/or
(e) ACE2-dependent betacoronavirus-induced lung lesions, optionally betacoronavirus-induced gross lesions in the lung.

In some embodiments, the method is for preventing, treating or ameliorating:

(a) ACE2-dependent SARS-like virus-induced pneumonia, optionally severe SARS-like virus-induced pneumonia, and/or
(b) ACE2-dependent SARS-like virus-induced weight loss, and/or
(c) ACE2-dependent SARS-like virus-induced lung inflammation, and/or
(d) ACE2-dependent SARS-like virus replication, optionally SARS-like virus replication in the lower respiratory tract, and/or
(e) ACE2-dependent SARS-like virus-induced lung lesions, optionally SARS-like virus-induced gross lesions in the lung.

In some embodiments, the method is for preventing, treating or ameliorating SARS2 infection in a subject, wherein the method comprises administering an anti-SARS2-S antibody of the invention to the subject.

In some embodiments, the method is for preventing, treating or ameliorating:

(a) SARS2-induced pneumonia, optionally severe SARS2-induced pneumonia, and/or
(b) SARS2-induced weight loss, and/or
(c) SARS2-induced lung inflammation, and/or
(d) SARS2 replication, optionally SARS2 replication in the lower respiratory tract, and/or
(e) SARS2-induced lung lesions, optionally SARS2-induced gross lesions in the lung.

In preferred embodiments, the subject is human.

In some embodiments, the anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is the K347N,
   (c) SARS2-S having the sequence of SEQ ID NO: 8 with a single substitution, wherein the substitution is the E414K, and
   (d) SARS2-S having the sequence of SEQ ID NO: 8 with a single substitution, wherein the substitution is the N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A, and
   (c) SARS2-S having the sequence of SEQ ID NO: 8 with a single substitution, wherein the substitution is L298A.

In some embodiments, the anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y,
   (e) SARS2-S having the sequence of SEQ ID NO: 8 with a single substitution, wherein the substitution is N369K,
   (f) SARS2-S having the sequence of SEQ ID NO: 8 with a single substitution, wherein the substitution is Q423R, and
   (g) SARS2-S having the sequence of SEQ ID NO: 8 with a single substitution, wherein the substitution is S424P; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the anti-SARS2-S antibody:
(i) binds, optionally with a K_{D} of 10⁻⁸ M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K, and
   (d) SARS2-S having the sequence of SEQ ID NO: 71 with a single substitution, wherein the substitution is N501Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the anti-SARS2-S antibody:
(i) binds optionally with a K_{D} of 10⁻⁸ M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K347N,
   (c) SARS2-S having the sequence of SEQ ID NO: 71 with a single substitution, wherein the substitution is E414K, and
   (d) SARS2-S having the sequence of SEQ ID NO: 71 with a single substitution, wherein the substitution is N431Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the anti-SARS2-S antibody:
(i) binds optionally with a K_{D} of 10⁻⁸M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (g) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the anti-SARS2-S antibody:
(i) binds optionally with a K_{D} of 10⁻⁸M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (c) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the anti-SARS2-S antibody comprises complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain.

In some embodiments, the anti-SARS2-S antibody comprises a light chain variable region of the amino acid sequence of SEQ ID NO: 9 and a heavy chain variable region of the amino acid sequence of SEQ ID NO: 10.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus infection, optionally betacoronavirus infection, such as SARS2 infection.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus-induced pneumonia, optionally betacoronavirus-induced pneumonia, such as SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating severe coronavirus-induced pneumonia, optionally severe betacoronavirus-induced pneumonia, such as severe SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus-induced weight loss, optionally betacoronavirus-induced weight loss, such as SARS2-induced weight loss.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus-induced lung inflammation, optionally betacoronavirus-induced lung inflammation, such as SARS2-induced lung inflammation.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus replication, optionally betacoronavirus replication, such as SARS2 replication.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus replication in the lower respiratory tract, optionally betacoronavirus replication in the lower respiratory tract, such as SARS2 replication in the lower respiratory tract.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus-induced lung lesions, optionally betacoronavirus-induced lung lesions, such as SARS2-induced lung lesions.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating coronavirus-induced gross lesions in the lung, optionally betacoronavirus-induced gross lesions in the lung, such as SARS2-induced gross lesions in the lung.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in reducing coronavirus load in the lungs, optionally betacoronavirus load in the lungs, such as SARS2 load in the lungs.

In preferred embodiments, the subject is human.

In preferred embodiments, the (beta)coronavirus is an ACE2-dependent (beta)coronavirus, such as SARS2.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus infection, optionally betacoronavirus infection, such as SARS2 infection.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus-induced pneumonia, optionally betacoronavirus-induced pneumonia, such as SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating severe coronavirus-induced pneumonia, optionally severe betacoronavirus-induced pneumonia, such as severe SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus-induced weight loss, optionally betacoronavirus-induced weight loss, such as SARS2-induced weight loss.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus-induced lung inflammation, optionally betacoronavirus-induced lung inflammation, such as SARS2-induced lung inflammation.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus replication, optionally betacoronavirus replication, such as SARS2 replication.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus replication in the lower respiratory tract, optionally betacoronavirus replication in the lower respiratory tract, such as SARS2 replication in the lower respiratory tract.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus-induced lung lesions, optionally betacoronavirus-induced lung lesions, such as SARS2-induced lung lesions.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating coronavirus-induced gross lesions in the lung, optionally betacoronavirus-induced gross lesions in the lung, such as SARS2-induced gross lesions in the lung.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in reducing coronavirus load in the lungs, optionally betacoronavirus load in the lungs, such as SARS2 load in the lungs.

In preferred embodiments, the subject is human.

In preferred embodiments, the (beta)coronavirus is an ACE2-dependent (beta)coronavirus, such as SARS2.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66,
   for use in preventing, treating or ameliorating coronavirus infection, optionally betacoronavirus infection, such as SARS2 infection.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating coronavirus-induced pneumonia, optionally betacoronavirus-induced pneumonia, such as SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating severe coronavirus-induced pneumonia, optionally severe betacoronavirus-induced pneumonia, such as severe SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating coronavirus-induced weight loss, optionally betacoronavirus-induced weight loss, such as SARS2-induced weight loss.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating coronavirus-induced lung inflammation, optionally betacoronavirus-induced lung inflammation, such as SARS2-induced lung inflammation.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating coronavirus replication, optionally betacoronavirus replication, such as SARS2 replication.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating coronavirus replication in the lower respiratory tract, optionally betacoronavirus replication in the lower respiratory tract, such as SARS2 replication in the lower respiratory tract.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating coronavirus-induced lung lesions, optionally betacoronavirus-induced lung lesions, such as SARS2-induced lung lesions.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating coronavirus-induced gross lesions in the lung, optionally betacoronavirus-induced gross lesions in the lung, such as SARS2-induced gross lesions in the lung.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in reducing coronavirus load in the lungs, optionally betacoronavirus load in the lungs, such as SARS2 load in the lungs.

In preferred embodiments, the subject is human.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69,
   for use in preventing, treating or ameliorating coronavirus infection, optionally betacoronavirus infection, such as SARS2 infection.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69, for use in preventing, treating or ameliorating coronavirus-induced pneumonia, optionally betacoronavirus-induced pneumonia, such as SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69,
   for use in preventing, treating or ameliorating severe coronavirus-induced pneumonia, optionally severe betacoronavirus-induced pneumonia, such as severe SARS2-induced pneumonia.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69, for use in preventing, treating or ameliorating coronavirus-induced weight loss, optionally betacoronavirus-induced weight loss, such as SARS2-induced weight loss.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69, for use in preventing, treating or ameliorating coronavirus-induced lung inflammation, optionally betacoronavirus-induced lung inflammation, such as SARS2-induced lung inflammation.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69, for use in preventing, treating or ameliorating coronavirus replication, optionally betacoronavirus replication, such as SARS2 replication.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69, for use in preventing, treating or ameliorating coronavirus replication in the lower respiratory tract, optionally betacoronavirus replication in the lower respiratory tract, such as SARS2 replication in the lower respiratory tract.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69, for use in preventing, treating or ameliorating coronavirus-induced lung lesions, optionally betacoronavirus-induced lung lesions, such as SARS2-induced lung lesions.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69,
   for use in preventing, treating or ameliorating coronavirus-induced gross lesions in the lung, optionally betacoronavirus-induced gross lesions in the lung, such as SARS2-induced gross lesions in the lung.

The invention further provides a fully human monoclonal IgG1 antibody that binds to SARS-Cov-2 coronavirus (SARS2) spike protein (SARS2-S), wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69,
   for use in reducing coronavirus load in the lungs, optionally betacoronavirus load in the lungs, such as SARS2 load in the lungs.

In preferred embodiments, the subject is human.

In preferred embodiments, the (beta)coronavirus is an ACE2-dependent (beta)coronavirus, such as SARS2.

The invention provides an anti-SARS2-S antibody that binds to the closed conformation SARS2-SIB for use in a method of treating SARS2 infection in a human subject by stabilising the partially open form of the SARS2-S trimer, and thereby reducing SARS2 infection of cells in the subject.

The invention provides an anti-SARS2-S antibody that binds to the closed conformation of SARS2-S1_{B} for use in a method of treating SARS2 infection in a human subject by stabilising the partially open form of the SARS2-S trimer, and thereby reducing SARS2 infection of cells in the subject,
wherein the method comprises administering:
(i) the anti-SARS2-S antibody that binds to the closed conformation of SARS2-S1_{B} and
(ii) an anti-SARS2-S antibody that binds to the open conformation of SARS2-S1_{B}.

The invention provides an anti-SARS2-S antibody that binds to the closed conformation of SARS2-S1_{B} for use in a method of treating SARS2 infection in a human subject by stabilising the partially open form of the SARS2-S trimer, and thereby reducing SARS2 infection of cells in the subject,
wherein the method comprises administering:
(i) the anti-SARS2-S antibody that binds to the closed conformation of SARS2-S1_{B} and
(ii) an anti-SARS2-S antibody that binds to the open conformation of SARS2-S1_{B},
   and wherein the combination of antibodies provides a synergistic effect relative to the administration of the anti-SARS2-S antibody that binds to the closed conformation of SARS2-S1_{B} or the anti-SARS2-S antibody that binds to the open conformation of SARS2-S1_{B} as a monotherapy.

In some embodiments, the anti-SARS2-S antibody that binds to the closed conformation of SARS2-S1_{B} is a fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain.

In some embodiments, the anti-SARS2-S antibody that binds to the closed conformation of SARS2-S1_{B} is a fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9.

In some embodiments, the anti-SARS2-S antibody that binds to the closed conformation of SARS2-S1_{B} is a fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66.

In some embodiments, the anti-SARS2-S antibody that binds to the closed conformation of SARS2-S1_{B} is a fully human monoclonal IgG1 antibody that binds to SARS-Cov-2 coronavirus (SARS2) spike protein (SARS2-S), wherein the antibody comprises two heavy and two light chains, wherein each heavy chain is encoded by the nucleic acid sequence of SEQ ID NO: 70,
and wherein each light chain is encoded by the nucleic acid sequence of SEQ ID NO: 69

Cross-reactive anti-SARS2-S antibodies of the invention, which bind to SARS2 and to a spike protein of one or more other betacoronaviruses (e.g. SARS-CoV-1, MERS-CoV, MHV), may be used in the diagnosis and treatment of infections by the one or more other betacoronaviruses (e.g. SARS-CoV-1, MERS-CoV, MHV). Accordingly, each of the above statements relating to the uses of anti-SARS2-S antibodies in the context of SARS2 diagnosis and treatment are also applicable to the context of the diagnosis and treatment of the one or more other betacoronaviruses (e.g. SARS-CoV-1, MERS-CoV, MHV) recognized by the cross-reactive anti-SARS2-S antibodies of the invention.

Each of the above statements relating to uses of anti-SARS2-S antibodies of the invention are also applicable to combinations of anti-SARS2-S antibodies of the invention.

Thus, the invention provides a combination of anti-SARS2-S antibodies for use in therapy.

The invention further provides a synergistic combination of anti-SARS2-S antibodies for use in therapy (e.g. for use in treating a SARS2-related disease or disorder).

The invention further provides a combination of two or more (e.g. three or more, four or more, or five or more, six or more, or seven or more) anti-SARS2-S antibodies, wherein the antibodies bind to different epitopes on the SARS2-S protein for use in therapy (e.g. for use in treating a SARS2-related disease or disorder). In some embodiments, the antibodies in the combination target non-overlapping epitopes.

The invention further provides a synergstic combination of two or more (e.g. three or more, four or more, or five or more, six or more, or seven or more) anti-SARS2-S antibodies, wherein the antibodies bind to different epitopes on the SARS2-S protein for use in therapy (e.g. for use in treating a SARS2-related disease or disorder). In some embodiments, the antibodies in the combination target non-overlapping epitopes.

The invention further provides a combination of anti-SARS2-S antibodies for use in therapy (e.g. for use in treating a SARS2-related disease or disorder), wherein the combination comprises: (i) a first anti-SARS2-S antibody that binds to the S1_{B} subunit, and (ii) a second anti anti-SARS2-S antibody that binds to the S1_{A} subunit.

The invention further provides a combination of anti-SARS2-S antibodies for use in therapy (e.g. for use in treating a SARS2-related disease or disorder), wherein the combination comprises: (i) a first anti-SARS2-S antibody that binds to the S1_{B} subunit, and (ii) a second anti anti-SARS2-S antibody that binds to the S1_{C} subunit.

The invention further provides a combination of anti-SARS2-S antibodies for use in therapy (e.g. for use in treating a SARS2-related disease or disorder), wherein the combination comprises: (i) a first anti-SARS2-S antibody that binds to the S1_{B} subunit, and (ii) a second anti anti-SARS2-S antibody that binds to the S1_{D} subunit.

The invention further provides a combination of anti-SARS2-S antibodies for use in therapy (e.g. for use in treating a SARS2-related disease or disorder), wherein the combination comprises: (i) a first anti-SARS2-S antibody that binds to the S1_{B} subunit, and (ii) a second anti anti-SARS2-S antibody that binds to the S2 subunit.

The invention further provides a combination of anti-SARS2-S antibodies for use in therapy (e.g. for use in treating a SARS2-related disease or disorder), wherein the combination comprises: (i) a first anti-SARS2-S antibody that binds to the S1_{B} subunit, wherein the antibody is capable of inhibiting the infection of human cells by SARS2, and wherein the antibody does not inhibit the binding of SARS2-S to human angiotensin converting enzyme 2 (ACE2), and (ii) a second anti-SARS2-S antibody that binds to the S1_{B} subunit, wherein the antibody is capable of inhibiting the infection of human cells by SARS2, and wherein the antibody inhibits the binding of SARS2-S to ACE2.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is the K347N,
   (c) SARS2-S having the sequence of SEQ ID NO: 8 with a single substitution, wherein the substitution is the E414K, and
   (d) SARS2-S having the sequence of SEQ ID NO: 8 with a single substitution, wherein the substitution is the N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A, and
   (c) SARS2-S having the sequence of SEQ ID NO: 8 with a single substitution, wherein the substitution is L298A.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 8,
   (b) SARS2-S having the sequence of SEQ ID NO: 8 with a single substitution, wherein the substitution is K347N,
   (c) SARS2-S having the sequence of SEQ ID NO: 8 with a single substitution, wherein the substitution is E414K, and
   (d) SARS2-S having the sequence of SEQ ID NO: 8 with a single substitution, wherein the substitution is N431Y,
   (e) SARS2-S having the sequence of SEQ ID NO: 8 with a single substitution, wherein the substitution is N369K,
   (f) SARS2-S having the sequence of SEQ ID NO: 8 with a single substitution, wherein the substitution is Q423R, and
   (g) SARS2-S having the sequence of SEQ ID NO: 8 with a single substitution, wherein the substitution is S424P; and
(ii) does not bind to any of:
   (a) SARS2-S having the sequence of SEQ ID NO: 8 with a single substitution, wherein the substitution is F268A,
   (b) SARS2-S having the sequence of SEQ ID NO: 8 with a single substitution, wherein the substitution is F272A,
   (c) SARS2-S having the sequence of SEQ ID NO: 8 with a single substitution, wherein the substitution is N273,
   (d) SARS2-S having the sequence of SEQ ID NO: 8 with a single substitution, wherein the substitution is Y295A,
   (e) SARS2-S having the sequence of SEQ ID NO: 8 with a single substitution, wherein the substitution is L298A,
   (f) SARS2-S having the sequence of SEQ ID NO: 8 with a single substitution, wherein the substitution is F304A and
   (g) SARS2-S having the sequence of SEQ ID NO: 8 with a single substitution, wherein the substitution is W366A.

In some embodiments, the first anti-SARS2-S antibody:
(i) binds, optionally with a K_{D} of 10⁻⁸ M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K, and
   (d) SARS2-S having the sequence of SEQ ID NO: 71 with a single substitution, wherein the substitution is N501Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the first anti-SARS2-S antibody:
(i) binds optionally with a K_{D} of 10⁻⁸ M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K347N,
   (c) SARS2-S having the sequence of SEQ ID NO: 71 with a single substitution, wherein the substitution is E414K, and
   (d) SARS2-S having the sequence of SEQ ID NO: 71 with a single substitution, wherein the substitution is N431Y; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the first anti-SARS2-S antibody:
(i) binds optionally with a K_{D} of 10⁻⁸M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (g) SARS2-S having the sequence of SEQ ID NO: 71 with a single substitution, wherein the substitution is S494P; and
(ii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A, and
   (c) SARS2-S having the sequence of SEQ ID NO: 71 with a single substitution, wherein the substitution is L368A.

In some embodiments, the first anti-SARS2-S antibody:
(i) binds optionally with a K_{D} of 10⁻⁸M or less (as determined by surface plasmon resonance), to:
   (a) SARS2-S having the sequence of SEQ ID NO: 71,
   (b) SARS2-S having the sequence of SEQ ID NO: 71 with a single substitution, wherein the substitution is K417N,
   (c) SARS2-S having the sequence of SEQ ID NO: 71 with a single substitution, wherein the substitution is E484K,
   (d) SARS2-S having the sequence of SEQ ID NO: 71 with a single substitution, wherein the substitution is N501Y,
   (e) SARS2-S having the sequence of SEQ ID NO: 71 with a single substitution, wherein the substitution is N439K,
   (f) SARS2-S having the sequence of SEQ ID NO: 71 with a single substitution, wherein the substitution is Q493R, and
   (d) SARS2-S having the sequence of SEQ ID NO: 71 with a single substitution, wherein the substitution is S494P; and
(iii) does not bind to any of:
   (a) SARS2-S having the sequence of SEQ ID NO: 71 with a single substitution, wherein the substitution is F338A,
   (b) SARS2-S having the sequence of SEQ ID NO: 71 with a single substitution, wherein the substitution is F342A,
   (c) SARS2-S having the sequence of SEQ ID NO: 71 with a single substitution, wherein the substitution is N343,
   (d) SARS2-S having the sequence of SEQ ID NO: 71 with a single substitution, wherein the substitution is Y365A,
   (e) SARS2-S having the sequence of SEQ ID NO: 71 with a single substitution, wherein the substitution is L368A,
   (f) SARS2-S having the sequence of SEQ ID NO: 71 with a single substitution, wherein the substitution is F374A, and
   (g) SARS2-S having the sequence of SEQ ID NO: 71 with a single substitution, wherein the substitution is W436A.

In some embodiments, the first antibody is an anti-SARS2-S antibody that binds to the same epitope as an antibody comprising a light chain variable region of the amino acid sequence of SEQ ID NO: 9 and a heavy chain variable region of the amino acid sequence of SEQ ID NO: 10.

In some embodiments, the first antibody is an anti-SARS2-S antibody that binds to the same epitope as a heavy chain-only antibody comprising a heavy chain variable region of the amino acid sequence of SEQ ID NO: 10.

In some embodiments, the first antibody is an anti-SARS2-S antibody that competes for binding to SARS2-S with an antibody comprising a light chain variable region of the amino acid sequence of SEQ ID NO: 9 and a heavy chain variable region of the amino acid sequence of SEQ ID NO: 10.

In some embodiments, the first antibody is an anti-SARS2-S antibody that competes for binding to SARS2-S with a heavy chain-only antibody comprising a heavy chain variable region of the amino acid sequence of SEQ ID NO: 10.

In some embodiments, the first antibody is an anti-SARS2-S antibody comprising complementarity determining regions (CDRs) with the sequences of:
i. SEQ ID NO: 53 for CDR1 of the light chain;
ii. SEQ ID NO: 54 for CDR2 of the light chain;
iii. SEQ ID NO: 55 for CDR3 of the light chain;
iv. SEQ ID NO: 56 for CDR1 of the heavy chain;
v. SEQ ID NO: 57 for CDR2 of the heavy chain; and
vi. SEQ ID NO: 58 for CDR3 of the heavy chain.

In some embodiments, the first antibody comprises a light chain variable region of the amino acid sequence of SEQ ID NO: 9.

In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 9.

In some embodiments, the first antibody comprises a heavy chain variable region of the amino acid sequence of SEQ ID NO: 10.

In some embodiments, the first antibody comprises an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 10.

In some embodiments, the first antibody comprises a light chain variable region of the amino acid sequence of SEQ ID NO: 9 and a heavy chain variable region of the amino acid sequence of SEQ ID NO: 10.

In some embodiments, the first antibody comprises: (i) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a light chain variable region of the amino acid sequence of SEQ ID NO: 9 and (ii) an amino acid sequence that is at least 80%, 81%, 82%, 83%, 84%, 85%, 86%, 87%, 88%, 89%, 90%, 91%, 92%, 93%, 94%, 95%, 96%, 97%, 98%, or 99% identical to a heavy chain variable region of the amino acid sequence of SEQ ID NO: 10.

In some embodiments, the first antibody is an anti-SARS2-S antibody comprising complementarity determining regions (CDRs) with:
i. a sequence that is at least 90% identical to SEQ ID NO: 53 for CDR1 of the light chain;
ii. a sequence that is at least 90% identical to SEQ ID NO: 54 for CDR2 of the light chain;
iii. a sequence that is at least 90% identical to SEQ ID NO: 55 for CDR3 of the light chain;
iv. a sequence that is at least 90% identical to SEQ ID NO: 56 for CDR1 of the heavy chain;
v. a sequence that is at least 90% identical to SEQ ID NO: 57 for CDR2 of the heavy chain; and
vi. a sequence that is at least 90% identical to SEQ ID NO: 58 for CDR3 of the heavy chain,
   wherein the antibody competes for binding to SARS2-S with an antibody comprising a light chain variable region of the amino acid sequence of SEQ ID NO: 9 and a heavy chain variable region of the amino acid sequence of SEQ ID NO: 10.

In some embodiments, the first antibody is an anti-SARS2-S antibody comprising complementarity determining regions (CDRs) with:
i. a sequence that is at least 95% identical to SEQ ID NO: 53 for CDR1 of the light chain;
ii. a sequence that is at least 95% identical to SEQ ID NO: 54 for CDR2 of the light chain;
iii. a sequence that is at least 95% identical to SEQ ID NO: 55 for CDR3 of the light chain;
iv. a sequence that is at least 95% identical to SEQ ID NO: 56 for CDR1 of the heavy chain;
v. a sequence that is at least 95% identical to SEQ ID NO: 57 for CDR2 of the heavy chain; and
vi. a sequence that is at least 95% identical to SEQ ID NO: 58 for CDR3 of the heavy chain,
   wherein the antibody competes for binding to SARS2-S with an antibody comprising a light chain variable region of the amino acid sequence of SEQ ID NO: 9 and a heavy chain variable region of the amino acid sequence of SEQ ID NO: 10.

In some embodiments, the first antibody is a fully human monoclonal IgG1 antibody that binds to SARS2-S, wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain.

In some embodiments, the first antibody is a fully human monoclonal IgG1 antibody that binds to SARS2-S, wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9.

In some embodiments, the first antibody is a fully human monoclonal IgG1 antibody that binds to SARS2-S, wherein the antibody comprises two heavy and two light chains,
wherein each heavy chain comprises the amino acid sequence of SEQ ID NO: 65,
and wherein each light chain comprises the amino acid sequence of SEQ ID NO: 66.

In some embodiments, the first antibody is a fully human monoclonal IgG1 antibody that binds to SARS2-S, wherein the antibody comprises two heavy and two light chains,
wherein each heavy chain is encoded by the nucleic acid sequence of SEQ ID NO: 70,
and wherein each light chain is encoded by the nucleic acid sequence of SEQ ID NO: 69.

### Definitions

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3.

However, the term “human antibody”, as used herein, is not intended to include mAbs in which CDR sequences derived from the germline of another mammalian species (e.g., mouse), have been grafted onto human FR sequences. The term includes antibodies recombinantly produced in a non-human mammal, or in cells of a non-human mammal. The term is not intended to include antibodies isolated from or generated in a human subject.

The term “recombinant”, as used herein, refers to antibodies or antigen-binding fragments thereof of the invention created, expressed, isolated or obtained by technologies or methods known in the art as recombinant DNA technology which include, e.g., DNA splicing and transgenic expression. The term refers to antibodies expressed in a non-human mammal (including transgenic non-human mammals, e.g., transgenic mice), or a cell (e.g., CHO cells) expression system or isolated from a recombinant combinatorial human antibody library.

An “isolated antibody”, as used herein, is intended to refer to an antibody that is substantially free of other antibodies (Abs) having different antigenic specificities (e.g., an isolated antibody that specifically binds SARS2-S, or a fragment thereof, is substantially free of Abs that specifically bind antigens other than SARS2-S.

A “blocking antibody” or a “neutralizing antibody”, as used herein (or an “antibody that neutralizes SARS2-S activity” or “antagonist antibody”), is intended to refer to an antibody whose binding to SARS2-S results in inhibition of at least one biological activity of SARS2. For example, an antibody of the invention may prevent or block SARS2 binding to ACE2. For example, an antibody of the invention may prevent or block SARS2 infection of human cells.

For example, an antibody of the invention may bind to SARS2-S and thereby inactivate SARS2-S.

The term “surface plasmon resonance”, as used herein, refers to an optical phenomenon that allows for the analysis of real-time biomolecular interactions by detection of alterations in protein concentrations within a biosensor matrix, for example using a ForteBio Octet instrument or the BIACORE™ system (Pharmacia Biosensor AB, Uppsala, Sweden and Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be defined as structural or functional. Functional epitopes are generally a subset of the structural epitopes and have those residues that directly contribute to the affinity of the interaction. Epitopes may also be conformational, that is, composed of nonlinear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics.

The term “competes”, as used herein, means an antibody or antigen-binding fragment thereof binds to an antigen and inhibits or blocks the binding of another antibody or antigen-binding fragment thereof. The term also includes competition between two antibodies in both orientations, i.e., a first antibody that binds and blocks binding of second antibody and vice-versa. In certain embodiments, the first antibody and second antibody may bind to the same epitope. Alternatively, the first and second antibodies may bind to different, but overlapping epitopes such that binding of one inhibits or blocks the binding of the second antibody, e.g., via steric hindrance. Cross-competition between antibodies may be measured by methods known in the art, for example, by a real-time, label-free bio-layer interferometry assay. Cross-competition between two antibodies may be expressed as the binding of the second antibody that is less than the background signal due to self-self binding (wherein first and second antibodies is the same antibody). Cross-competition between 2 antibodies may be expressed, for example, as % binding of the second antibody that is less than the baseline self-self background binding (wherein first and second antibodies is the same antibody).

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutant thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402.

By the phrase “therapeutically effective amount” is meant an amount that produces the desired effect for which it is administered. The exact amount will depend on the purpose of the treatment, and will be ascertainable by one skilled in the art using known techniques (see, for example, Lloyd (1999) The Art, Science and Technology of Pharmaceutical Compounding).

As used herein, the term “subject” refers to an animal, preferably a mammal, more preferably a human, in need of amelioration, prevention and/or treatment of a disease or disorder such as viral infection. The term includes human subjects who have or are at risk of having SARS2 infection.

As used herein, the terms “treat”, “treating”, or “treatment” refer to the reduction or amelioration of the severity of at least one symptom or indication of SARS2 infection due to the administration of a therapeutic agent such as an antibody of the present invention to a subject in need thereof. The terms include inhibition of progression of disease or of worsening of infection. The terms also include positive prognosis of disease, i.e., the subject may be free of infection or may have reduced or no viral titers upon administration of a therapeutic agent such as an antibody of the present invention. The therapeutic agent may be administered at a therapeutic dose to the subject.

The terms “prevent”, “preventing” or “prevention” refer to inhibition of manifestation of viral infection (e.g. SARS2 infection) or any symptoms or indications of viral infection (e.g. SARS2 infection) upon administration of an antibody of the present invention. The term includes prevention of spread of infection in a subject exposed to the virus or at risk of having a viral infection (e.g. SARS2 infection).

As used herein, the term “anti-viral drug” refers to any anti-infective drug or therapy used to treat, prevent, or ameliorate a viral infection in a subject. The term “anti-viral drug” includes, but is not limited to ribavirin, oseltamivir, zanamivir, interferon-alpha2b, analgesics and corticosteroids. In the context of the present invention, the viral infections include infection caused by human coronaviruses, including but not limited to, SARS2, MERS-CoV, HCoV_229E, HCoV_NL63, HCoV-OC43, HCoV_HKU1, and SARS-CoV-1.

### General

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

Various aspects and embodiments of the invention are described below in more detail by way of example. It will be appreciated that modification of detail may be made without departing from the scope of the invention.

### EXAMPLES

### Example 1—Generation of Cross-Reactive Anti-CoV Antibodies

Two groups of six transgenic H2L2 mice (each carrying genes encoding the heavy and light chain human immunoglobulin repertoire and constant regions of rat origin) were sequentially immunized in two weeks intervals with purified S_{ecto} of different CoVs in the following order: MERS-CoV, SARS-CoV, HCoV-OC43, MERS-CoV, SARS-CoV and HCoV-OC43. Antigens were injected at 20-25 μg/mouse using Stimune Adjuvant (Prionics) freshly prepared according to the manufacturer instruction for first injection, while boosting was done using Ribi (Sigma) adjuvant. Injections were done subcutaneously into the left and right groin each (50 μl) and 100 μl intraperitoneally. Four days after the last injection, spleen and lymph nodes were harvested, and hybridomas made by standard method using SP 2/0 myeloma cell line (ATCC #CRL-1581) as a fusion partner. Hybridomas were screened in antigen-specific ELISA and those selected for further development, subcloned and produced on a small scale (100 ml of medium). For this purpose, hybridomas are cultured in serum- and protein-free medium for hybridoma culturing (PFHM-II (1X) Gibco) with addition of non-essential amino acids 100×NEAA, Biowhittaker Lonza Cat BE13-114E).

### Example 2—Identification of SARS2-S Antibodies

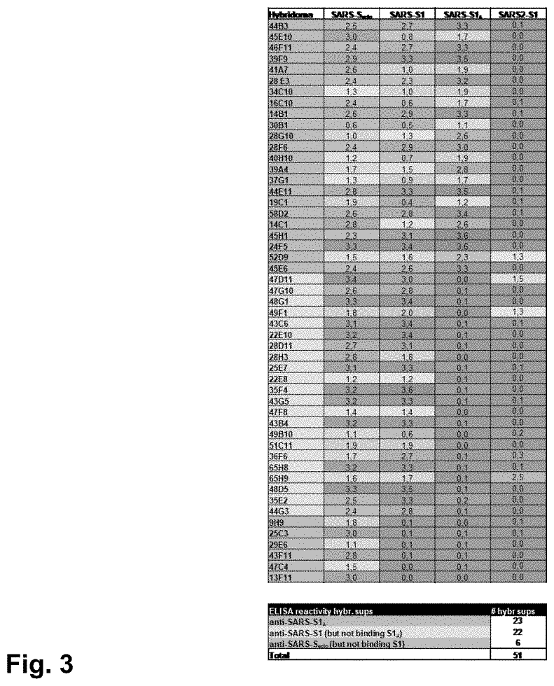

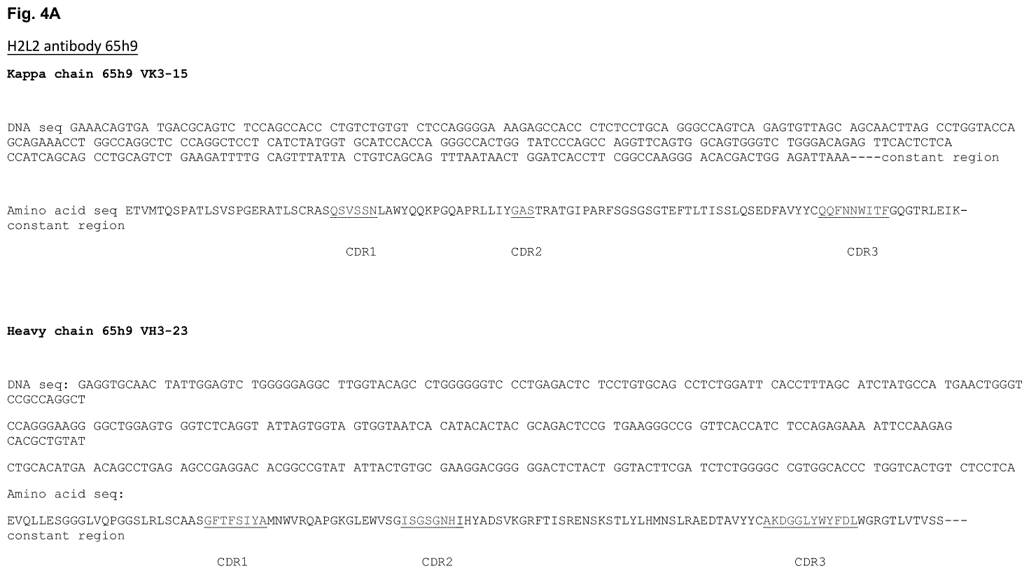

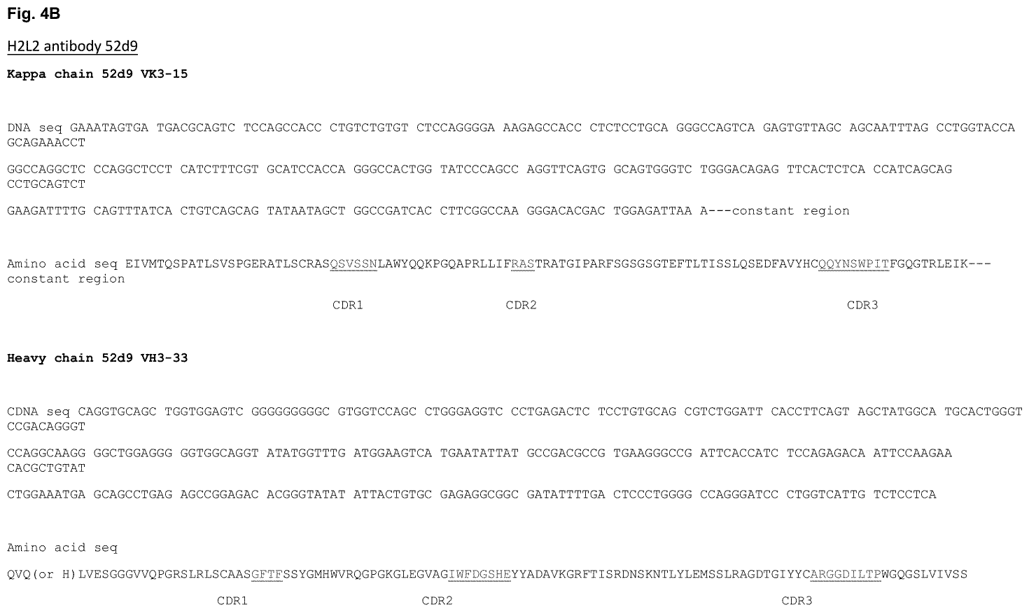

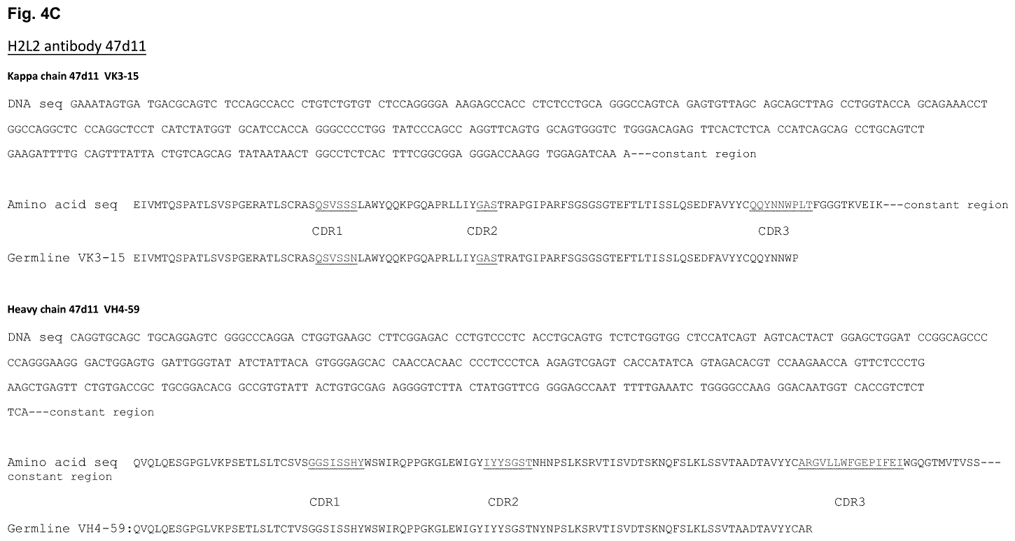

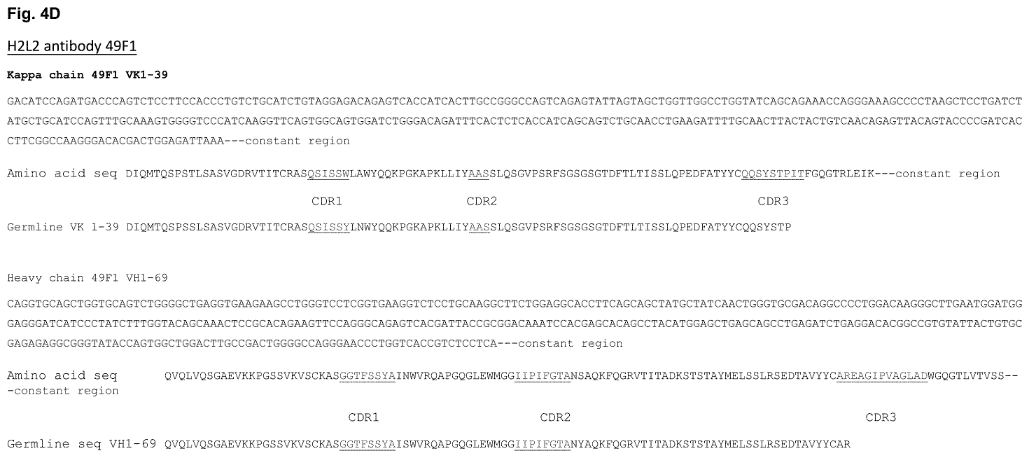

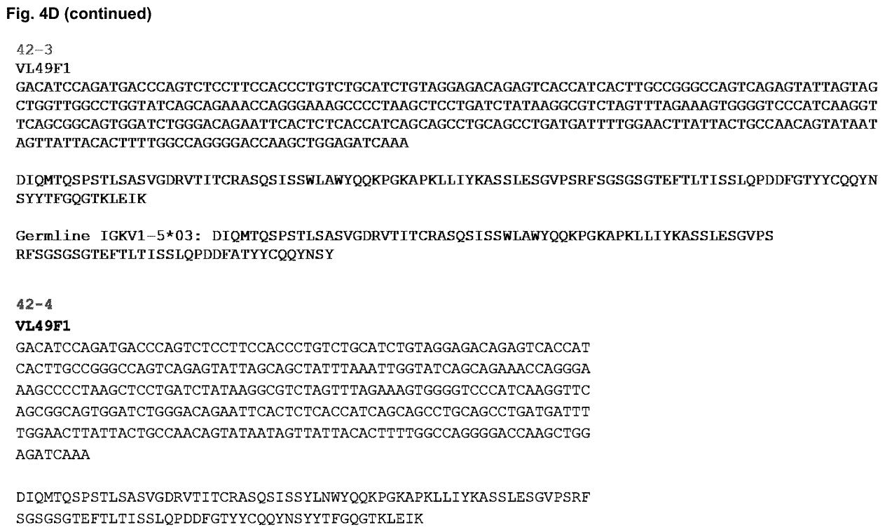

In order to identify SARS-CoV-2 neutralizing antibodies, ELISA-(cross)reactivity was assessed of antibody-containing supernatants of a collection of 51 hybridomas that had been obtained as described in Example 1. Four of 51 hybridoma supernatants (47d11, 52d9, 49f1 and 65h9) displayed ELISA-cross-reactivity with the SARS2-S1 subunit (S residues 1-681; FIG. 3).

### Example 3—the 47d11 Antibody

The 47d11 antibody identified in Example 2 exhibited cross-neutralizing activity of SARS-S and SARS2-S pseudotyped VSV infection. The chimeric 47D11 H2L2 antibody was reformatted and recombinantly expressed as a fully human IgG1 isotype antibody for further functional characterization.

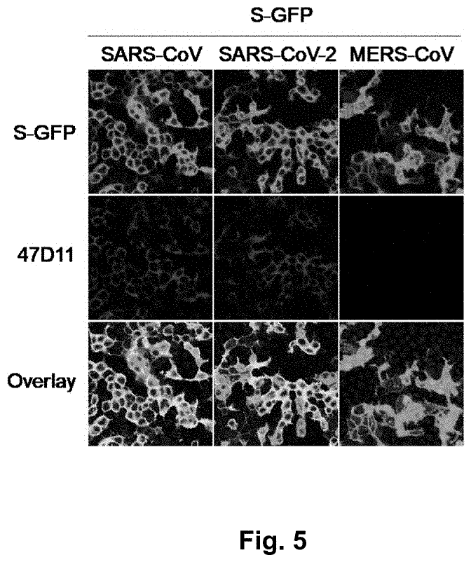

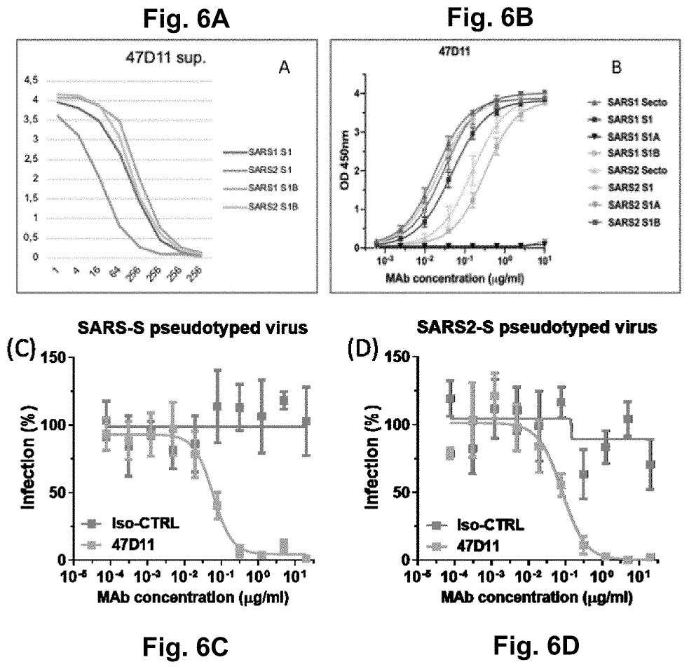

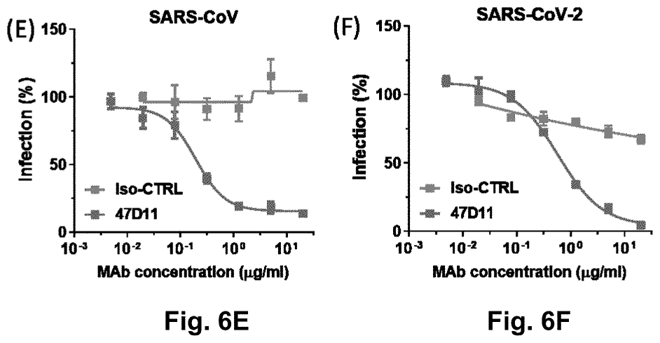

The human 47D11 antibody binds to cells expressing the full-length spike proteins of SARS-CoV and SARS-CoV-2 (FIG. 5). The 47D11 antibody was found to potently inhibit infection of VeroE6 cells with SARS-S and SARS2-S pseudotyped VSV with IC₅₀ values of 0.06 and 0.08 μg/ml (FIGS. 6C and 6D), respectively. Authentic infection of Vero cells with SARS-CoV and SARS-CoV-2 was neutralized with IC₅₀ values of 0.19 and 0.57 μg/ml (FIGS. 6C and 6D).

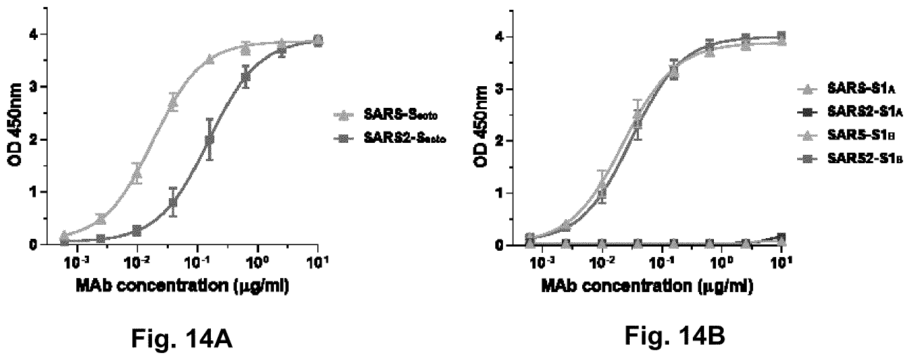

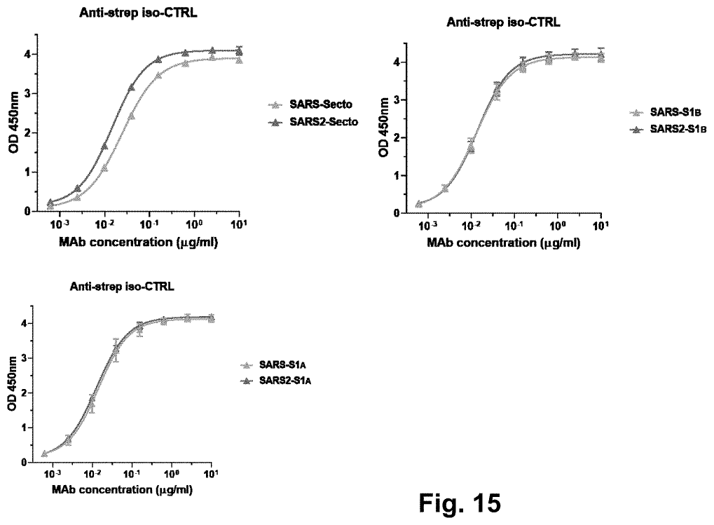

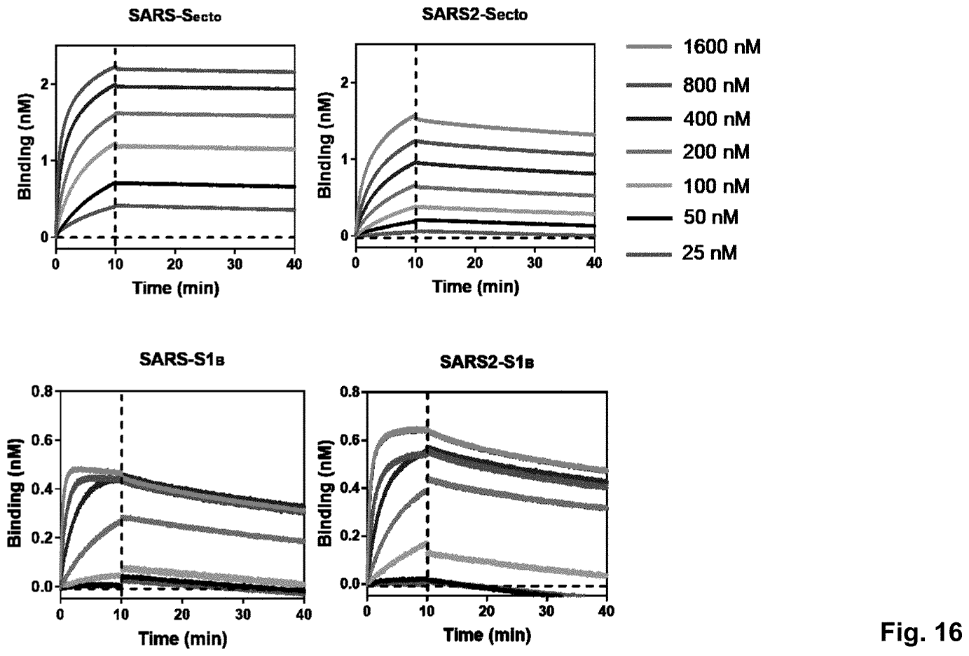

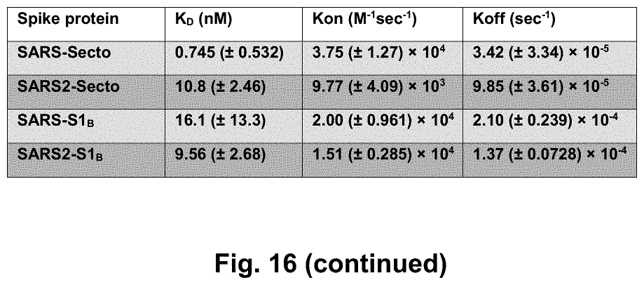

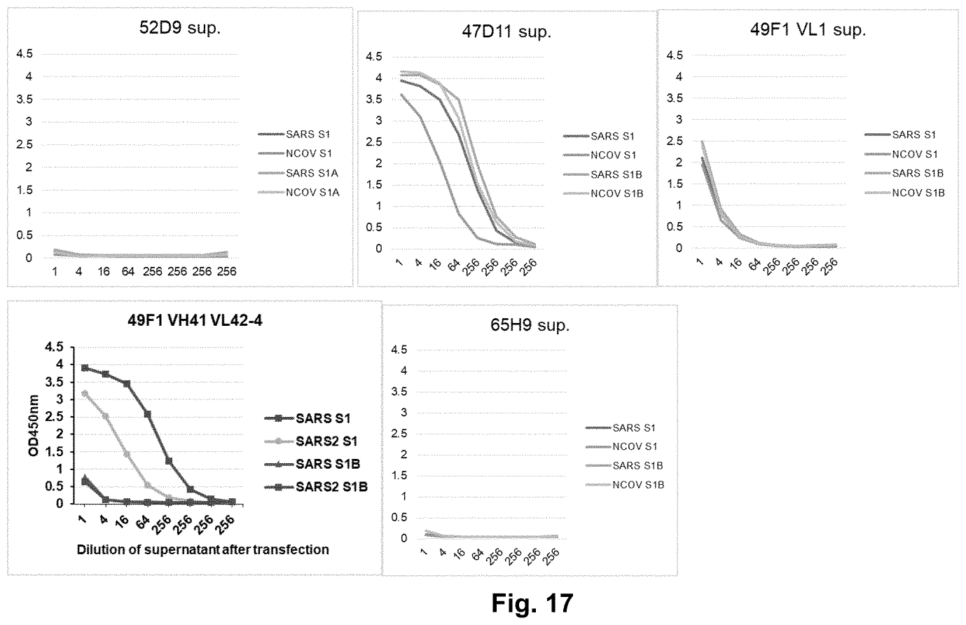

Using ELISA 47D11 was shown to target the S1_{B} receptor binding domain (RBD) of SARS-S and SARS2-S. 47D11 bound the S1_{B} of both viruses with similar affinities as shown by the ELISA-based half maximal effective concentration (EC₅₀) values (0.02 and 0.03 μg/ml, respectively; FIG. 14). ELISA-based binding affinity of 47D11 for the spike ectodomain (S_{ecto}) of SARS-CoV was higher relative to that of SARS-CoV-2 (EC₅₀ values: 0.018 and 0.15 μg/ml, respectively), despite equimolar antigen coating (FIG. 15). Congruent with the ELISA-reactivities, measurement of binding kinetics of 47D11 by biolayer interferometry showed that 47D11 binds SARS-S_{ecto} with higher affinity (equilibrium dissociation constant [K_{D}]: 0.745 nM) relative to SARS2-S_{ecto} (K_{D} 10.8 nM) whereas affinity for SARS-S1_{B} and SARS2-S1_{B} was in a similar range (16.1 and 9.6 nM, respectively, FIG. 16). This difference may originate from differences in epitope accessibility in SARS-S versus SARS2-S, as domain B can adopt a closed and open conformation in the prefusion spike homotrimer (Wrapp et al. (2020) Cryo-EM structure of the 2019-nCoV spike in the prefusion conformation. Science (2020)).

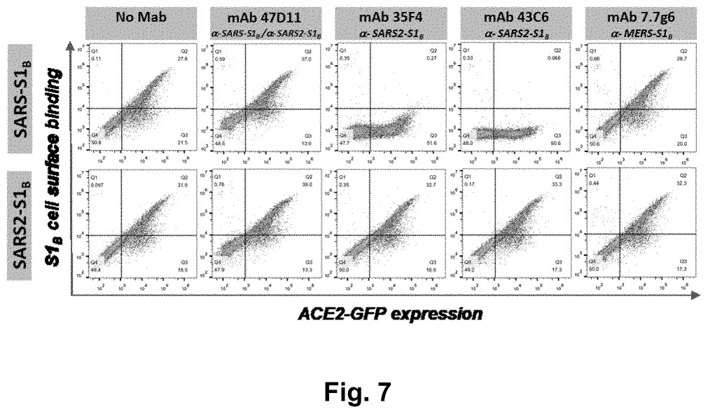

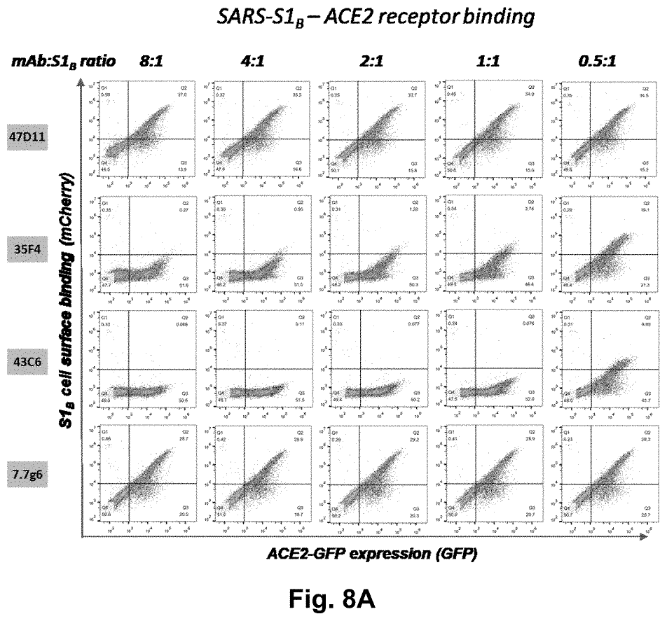

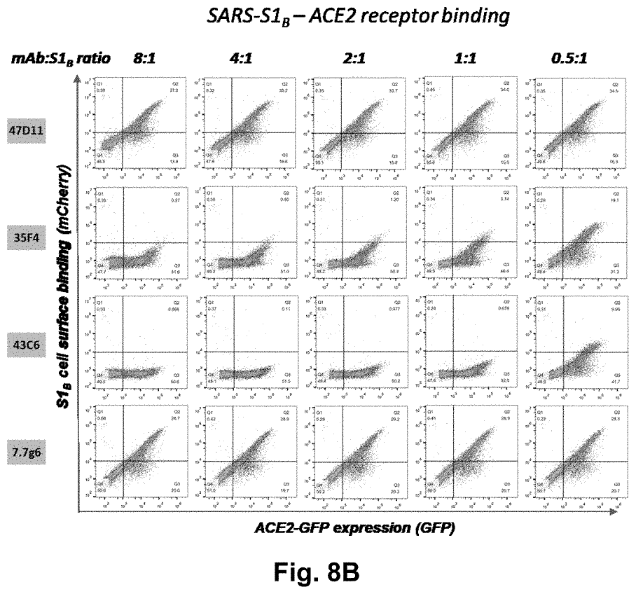

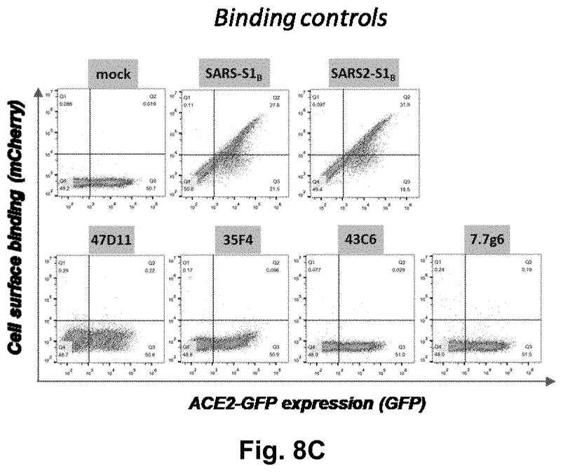

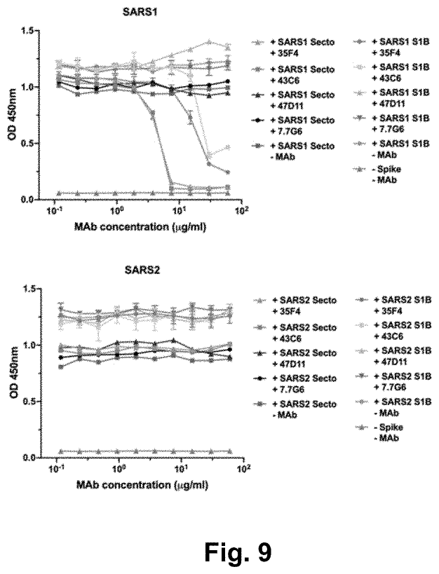

Remarkably, binding of 47D11 to SARS-S1_{B} and SARS2-S1_{B} did not compete with S1_{B} binding to the ACE2 receptor expressed at the cell surface as shown by flow cytometry (FIGS. 7 and 8) or with S_{ecto} and S1_{B} binding to soluble ACE2 in solid-phase based assay (FIG. 9).

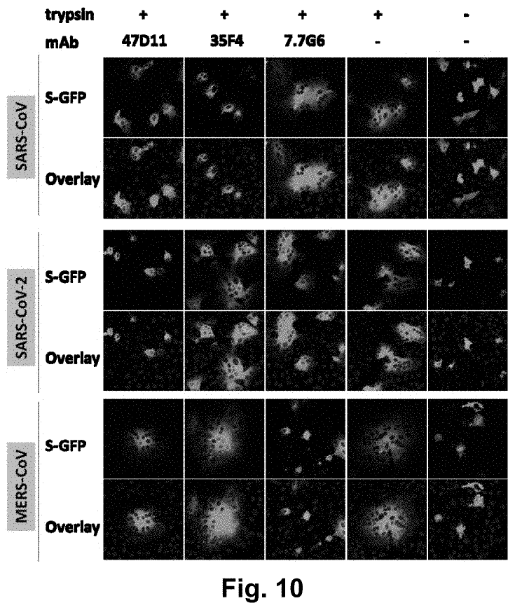

Using a trypsin-triggered cell-cell fusion assay, 47D11 was shown to impair SARS-S and SARS2-S mediated syncytia formation (FIG. 10). Our data show that 47D11 neutralizes SARS-CoV and SARS-CoV-2 through a previously unknown mechanism that is different from receptor binding interference. Alternative mechanisms of coronavirus neutralization by RBD-targeting antibodies have been reported including spike inactivation through antibody-induced destabilization of its prefusion structure (Walls et al. (2019) Cell 176(5):1026-1039), which may also apply for 47D11.

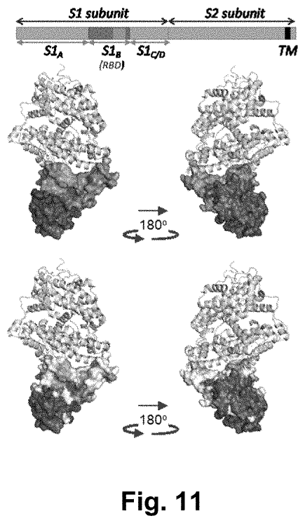

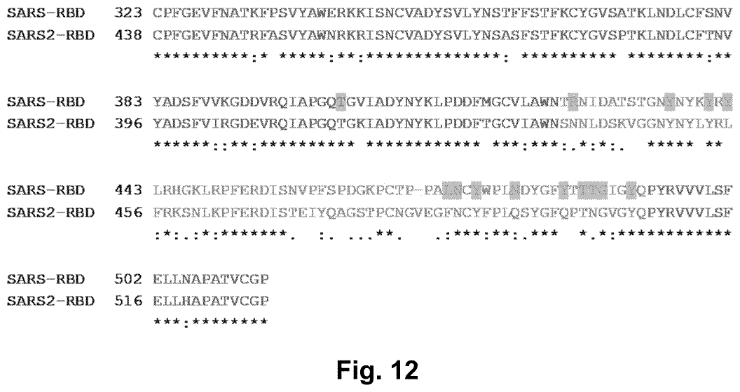

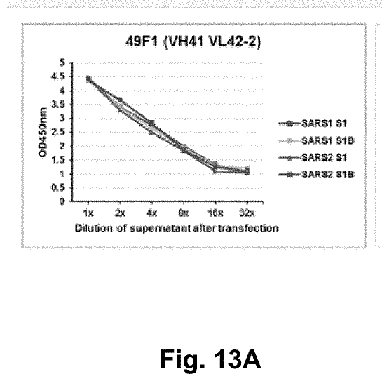

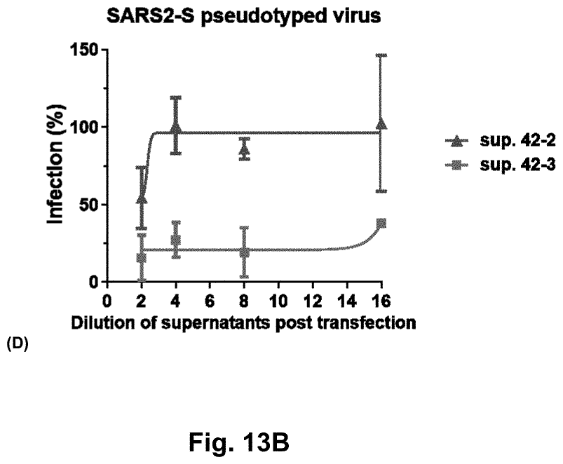

The SARS2-S1_{B} receptor binding domain (residues 338-506) consists of a core domain and a receptor binding subdomain (residues 438-498) looping out from the antiparallel betasheet core domain structure that directly engages the receptor. Compared to the S1_{B} core domain, the protein sequence identity of the S1_{B} receptor interacting subdomain of SARS-S and SARS2-S is substantially lower (46.7% versus 86.3%; FIGS. 11 and 12). Due to common variations in this subdomain, neutralizing antibodies that target this subdomain are often virus-specific and bind and neutralize related viruses poorly. The cross-reactive nature of 47D11 indicates that the antibody is more likely to target the conserved core structure of the S1_{B} receptor binding domain. S1_{B} binding by 47D11 further away from the receptor binding interface explains its inability to compromise spike-receptor interaction.

In conclusion, this is the first report on a (human) monoclonal antibody that neutralizes SARS-CoV-2. 47D11 binds a conserved epitope on the spike receptor binding domain explaining its ability to cross-neutralize SARS-CoV and SARS-CoV-2, using a mechanism that is independent of receptor binding inhibition. This antibody will be useful for development of antigen detection tests and serological assays targeting SARS-CoV-2, e.g. diagnostics. Neutralizing antibodies can alter the course of infection in the infected host supporting virus clearance or protect an uninfected host that is exposed to the virus (Prabakaran et al. (2009) Expert Opinion on Biological Therapy 9(3):355-68). Hence, this antibody offers the potential to prevent and/or treat COVID-19, and possibly also other future emerging diseases in humans caused by viruses from the Sarbecovirus subgenus.

### Example 4—Materials and Methods

Expression and purification of coronavirus spike proteins. Coronavirus spike ectodomains (S_{ecto}) of SARS-CoV (residues 1-1,182; strain CUHK-W1; GenBank: AAP13567.1) and SARS-CoV-2 (residues 1-1,213; strain Wuhan-Hu-1; GenBank: QHD43416.1) were expressed transiently in HEK-293T cells with a C-terminal trimerization motif and Strep-tag using the pCAGGS expression plasmid. Similarly, pCAGGS expression vectors encoding S1 or its subdomains of SARS-CoV (S1, residues 1-676; S1_{A}, residues 1-302; S1_{B}, residues, 325-533), and SARS-CoV-2 (S1, residues 1-682; S1_{A}, residues 1-294; SIB, residues 329-538) C-terminally tagged with Fc domain of human or mouse IgG or Strep-tag were generated as described before¹⁹. Recombinant proteins were expressed transiently in HEK-293T cells and affinity purified from the culture supernatant by protein-A sepharose beads (GE Healthcare) or streptactin beads (IBA) purification. Purity and integrity of all purified recombinant proteins was checked by coomassie stained SDS-PAGE

Generation of H2L2 mAbs. H2L2 mice were sequentially immunized in two weeks intervals with purified S_{ecto} of different CoVs in the following order: HCoV-OC43, SARS-CoV, MERS-CoV, HCoV-OC43, SARS-CoV and MERS-CoV. Antigens were injected at 20-25 μg/mouse using Stimune Adjuvant (Prionics) freshly prepared according to the manufacturer instruction for first injection, while boosting was done using Ribi (Sigma) adjuvant. Injections were done subcutaneously into the left and right groin each (50 μl) and 100 μl intraperitoneally. Four days after the last injection, spleen and lymph nodes are harvested, and hybridomas made by standard method using SP 2/0 myeloma cell line (ATCC #CRL-1581) as a fusion partner. Hybridomas were screened in antigen-specific ELISA and those selected for further development, subcloned and produced on a small scale (100 ml of medium). For this purpose, hybridomas are cultured in serum- and protein-free medium for hybridoma culturing (PFHM-II (1X), Gibco) with addition of non-essential amino acids 100×NEAA, Biowhittaker Lonza, Cat BE13-114E). H2L2 antibodies were purified from hybridoma culture supernatants using Protein-A affinity chromatography. Purified antibodies were stored at 4° C. until use.

Production of human monoclonal antibody 47D11. For recombinant human mAb production, the cDNA's encoding the 47D11 H2L2 mAb variable regions of the heavy and light chains were cloned into expression plasmids containing the human IgG1 heavy chain and Ig kappa light chain constant regions, respectively (InvivoGen). Both plasmids contain the interleukin-2 signal sequence to enable efficient secretion of recombinant antibodies. Recombinant human 47D11 mAb and previously described Isotype-control (anti-Streptag mAb) or 7.7G6 mAb²⁰ were produced in HEK-293T cells following transfection with pairs of the IgG1 heavy and light chain expression plasmids according to protocols from InvivoGen. Human antibodies were purified from cell culture supernatants using Protein-A affinity chromatography. Purified antibodies were stored at 4° C. until use.

Immunofluorescence microscopy. Antibody binding to cell surface spike proteins of SARS-CoV, SARS-CoV-2 and MERS-CoV was measured by immunofluoresence microscopy. HEK-293T cells seeded on glass slides were transfected with plasmids encoding SARS-S, SARS2-S or MERS-S-C-terminally fused to the green fluorescence protein (GFP)—using Lipofectamine 2000 (Invitrogen). Two days post transfection, cells were fixed by incubation with 2% paraformaldehyde in PBS for 20 min at room temperature and stained for nuclei with 4,6-diamidino-2-phenylindole (DAPI). Cells were subsequently incubated with mAbs at a concentration of 10 μg/ml for 1 h at room temperature, followed by incubation with Alexa Fluor 594 conjugated goat anti-human IgG antibodies (Invitrogen, Thermo Fisher Scientific) for 45 min at room temperature. The fluorescence images were recorded using a Leica SpeII confocal microscope.

Flow cytometry-based receptor binding inhibition assay. Antibody interference of S1_{B} binding to human ACE2 receptor on the cell surface was measured by flow cytometry. HEK-293T cells were seeded at a density of 2.5×10⁵ cells per ml in a T75 flask. After reaching 70˜80% confluency, cells were transfected with an expression plasmid encoding human ACE2-C-terminally fused to the GFP—using Lipofectamine 2000 (Invitrogen). Two days post transfection, cells were dissociated by cell dissociation solution (Sigma-aldrich, Merck KGaA; cat. no. C5914). 2.5 μg/ml of human Fc tagged SARS-S1_{B} and SARS2-S1_{B} was preincubated with mAb at the indicated mAb:S1_{B} molar ratios for 1 hour on ice and subjected to flow cytometry. Single cell suspensions in FACS buffer were centrifuged at 400×g for 10 min. Cells were subsequently incubated with S1_{B} and mAb mixture for 1 h on ice, followed by incubation with Alexa Fluor 594 conjugated goat anti-human IgG antibodies (Invitrogen, Thermo Fisher Scientific) for 45 min at room temperature. Cells were subjected to flow cytometric analysis with a CytoFLEX Flow Cytometer (Beckman Coulter). The results were analysed by FlowJo (version 10).

Pseudotyped virus neutralization assay. Production of VSV pseudotyped with SARS-S and SARS2-S was performed as described previously with some adaptations (Widjaja et al. (2019) Emerging Microbes & Infections 8(1):516-530). Briefly, HEK-293T cells were transfected with pCAGGS expression vectors encoding SARS-S or SARS2-S carrying a 28- or 18-a.a. cytoplasmic tail truncation, respectively. One day post transfection, cells were infected with the VSV-G pseudotyped VSVAG bearing the firefly (Photinus pyralis) luciferase reporter gene. Twenty-four hours later, supernatants containing SARS-S/SARS2-S pseudotyped VSV particles were harvested and titrated on African green monkey kidney VeroE6 cells. In the virus neutralization assay, mAbs were serially diluted at two times the desired final concentration in DMEM supplemented with 1% fetal calf serum (Bodinco), 100 U/ml Penicillin and 100 μg/ml Streptomycin. Diluted mAbs were incubated with an equal volume of pseudotyped VSV particles for 1 hour at room temperature, inoculated on confluent VeroE6 monolayers in 96-well plated, and further incubated at 37° C. for 24 hours. Luciferase activity was measured on a Berthold Centro LB 960 plate luminometer using D-luciferin as a substrate (Promega). The percentage of infectivity was calculated as ratio of luciferase readout in the presence of mAbs normalized to luciferase readout in the absence of mAb. The half maximal inhibitory concentrations (IC₅₀) were determined using 4-parameter logistic regression (GraphPad Prism v8).

Virus neutralization assay. Neutralization of authentic SARS-CoV and SARS-CoV-2 was performed using a plaque reduction neutralization test (PRNT) as described earlier (Okba et al. (2019) Emerg. Infect. Dis. 25, 1868-1877) with some modifications. In brief, mAbs were two-fold serially diluted and mixed with SARS-CoV or SARS-CoV-2 for 1 hour. The mixture was then added to VeroE6 cells and incubated for 1 hr, after which the cells were washed and further incubated in medium for 8 hrs. The cells were then fixed and stained using a rabbit anti-SARS-CoV serum (Sino Biological) and a secondary peroxidase-labelled goat anti-rabbit IgG (Dako). The signal was developed using a precipitate forming TMB substrate (True Blue, KPL) and the number of infected cells per well were counted using the ImmunoSpot® Image analyzer (CTL Europe GmbH). The half maximal inhibitory concentrations (IC₅₀) were determined using 4-parameter logistic regression (GraphPad Prism version 8).

ELISA analysis of antibody binding to CoV spike antigens. NUNC Maxisorp plates (Thermo Scientific) were coated with equimolar antigen amounts at 4° C. overnight. Plates were washed three times with Phosphate Saline Buffer (PBS) containing 0.05% Tween-20 and blocked with 3% Bovine Serum Albumin (BSA) in PBS containing 0.1% Tween-20 at room temperature for 2 hours. Four-folds serial dilutions of mAbs starting at 10 μg/ml (diluted in blocking buffer) were added and plates were incubated for 1 hour at room temperature. Plates were washed three times and incubated with HRP-conjugated goat anti-human secondary antibody (ITK Southern Biotech) diluted 1:2000 in blocking buffer for 1 hour at room temperature. An HRP-conjugated anti-StrepMAb (IBA, Cat. no: 2-1509-001) antibody was used to corroborate equimolar coating of the Strep-tagged spike antigens. HRP activity was measured at 450 nanometer using tetramethylbenzidine substrate (BioFX) and an ELISA plate reader (EL-808, Biotek). Half-maximum effective concentration (EC₅₀) binding values were calculated by non-linear regression analysis on the binding curves using GraphPad Prism (version 8).

ELISA cross-reactivity of antibody-containing supernatants of SARS-S H2L2 hybridomas towards SARS2-S1—SARS-S targeting hybridomas were developed by conventional hybridoma technology from immunized H2L2 transgenic mice (Harbour Biomed), as described before (Widjaja et al. (2019) Emerging Microbes & Infections 8(1):516-530; PCT/EP2020/054521). These mice—carrying genes encoding the heavy and light chain human immunoglobulin repertoire—were sequentially immunized with 2-week intervals with trimeric spike protein ectodomains (S_{ecto}) of three human coronaviruses from the betacoronavirus genus in the following order: 1. HCoV-OC43-S_{ecto}, 2. SARS-CoV-S_{ecto}, 3. MERS-CoV-S_{ecto}, 4. HCoV-OC43-S_{ecto}, 5. SARS-CoV-S_{ecto}, 6. MERS-CoV-S_{ecto}. Four days after the last immunization, splenocytes and lymph node lymphocytes were harvested and hybridomas were generated. Antibodies in the cell supernatants were tested for ELISA-reactivity against SARS-S_{ecto}, SARS-S1, SARS-S1_{A} and SARS2-S1. Of the 51 hybridoma supernatants that reacted with SARS-S_{ecto} only, 23 reacted with SARS-S1_{A}, 22 with SARS-S1 but not SARS-S1_{A}, 6 with SARS-S_{ecto} but not SARS-S1. Four of the 51 SARS-S_{ecto} hybridoma supernatants reacted with SARS2-S1 (see column on the right). The table displays ELISA-signal intensities (OD_{450 nm} values) of hybridoma supernatants for the different antigens.

Binding kinetics of 47D11 to the S ectodomain and SIB of SARS-CoV and SARS-CoV-2-Binding kinetics of 47D11 to immobilized recombinant SARS-S_{ecto}, SARS2-S_{ecto}, SARS-S1_{B} and SARS2-S1_{B} was measured using biolayer interferometry at 25° C., as described previously²⁰. Kinetic binding assay was performed by loading 47D11 mAb at optimal concentration (42 nM) on anti-human Fc biosensor for 10 mins. Antigen association step was performed by incubating the sensor with a range of concentrations of the recombinant spike ectodomain (1600-800-400-200-100-50-25 nM) for 10 min, followed by a dissociation step in PBS for 60 min. The kinetics constants were calculated using 1:1 Langmuir binding model on Fortebio Data Analysis 7.0 software.

Antibody capability for preventing the binding of SARS-S1_{B} and SARS2-S1_{B} to hACE2-expressing cells—Human HEK-293T cells expressing human ACE2-GFP proteins (see Methods) were detached and fixed with 2% PFA, incubated with a fixed amount of human Fc-tagged S1_{B} domain of SARS-S or SARS2-S that was preincubated for 1h with mAb (mAbs 47D11, 35F4, 43C6, 7.7G6, in H2L2 format) at the indicated mAb:S1_{B} molar ratios, and analysed by flow cytometry using a Alexa Fluor 594-conjugated secondary antibody targeting the human Fc tag. Cells are analysed for GFP expression (x-axis, GFP signal) and antibody binding (y-axis, Alexa 594 signal). Percentages of cells that scored negative, single positive, or double positive are shown in each quadrant. Binding controls include PBS-treated cells (mock), treatment of cells with SARS-S1_{B} and SARS2-S1_{B} in the absence of antibody, and cells treated with antibodies only. The experiment was performed twice, data from a representative experiment are shown in FIG. 8.

ELISA-based receptor binding inhibition assay—Recombinant soluble human ACE2 was coated on NUNC Maxisorp plates (Thermo Scientific) at 4° C. overnight. Plates were washed three times with PBS containing 0.05% Tween-20 and blocked with 3% BSA in PBS containing 0.1% Tween-20 at room temperature for 2 hours. Recombinant S_{ecto} and S1_{B} of SARS-S or SARS2-S (300 ng) and serially diluted mAbs (mAbs 47D11, 35F4, 43C6, 7.7G6, in H2L2 format) were mixed for 1 h at RT, added to the plate for 1 hour at room temperature, after which the plates were washed three times. Binding to ACE2 was detected using HRP-conjugated StrepMAb (IBA) that recognizes the C-terminal Streptag on the S_{ecto} and S1_{B} proteins.

Cell-cell fusion inhibition assay—The cell-cell-fusion inhibition assay was performed as described previously (see Widjaja et al. (2019) Emerging Microbes & Infections 8(1):516-530; PCT/EP2020/054521) with some adaptations. VeroE6 cells were seeded with density of 10⁵ cells per ml. After reaching 70˜80% confluency, cells were transfected with plasmids encoding full length SARS-S, SARS2-S and MERS-S—C-terminally fused to GFP—using Lipofectamine 2000 (Invitrogen). The furin recognition site in the SARS2-S was mutated (R⁶⁸²RAR to A⁶⁸²AAR) to inhibit cleavage of the protein by endogenous furin and allow trypsin-induced syncytia formation. Two days post transfection, cells were pretreated DMEM only or DMEM with 20 μg/ml mAbs for 1 h and subsequently treated with DMEM with 15 μg/ml trypsin (to activate the spike fusion function) in the absence or presence of 20 μg/ml mAbs (47D11 crossreactive SARS-S and SARS2-S, 35F4 reactive to SARS-S, 7.7G6 reactive to MERS-S). After incubation at 37° C. for 2 hrs, the cells were fixed with 2% PFA in PBS for 20 min at room temperature and stained for nuclei with 4,6-diamidino-2-phenylindole (DAPI). Cells expressing the S-GFP proteins were detected by fluorescence microscopy and S-mediated cell-cell fusion was observed by the formation of (fluorescent) multi-nucleated syncytia. The fluorescence images were recorded using a Leica SpeII confocal microscope. The experiment was performed twice, data from a representative experiment are shown in FIG. 10.

Protein sequence alignment of the S1_{B} receptor binding domain (RBD) of the SARS-CoV and SARS-CoV-2 spike proteins by ClustalW. Numbering denotes the residue position in the full-length spike protein of SARS-CoV (Genbank: AAP13441.1) and SARS-CoV-2 (Genbank: QHD43416.1). Asterisks (*) indicated fully conserved residues, the colon symbol (:) indicates conservation between groups of very similar properties, and the period symbol (.) indicates conservation between groups of weakly similar properties. Sequences corresponding to the S1_{B} receptor binding core domain and the receptor binding subdomain are colored in blue and orange, respectively. The fourteen residues that are involved in binding of SARS-CoV S1_{B} to human ACE2 are highlighted in grey.

### Example 5—Prophylactic SARS-CoV-2 Treatment in Hamster

The protective efficacy of neutralizing monoclonal antibody 47D11 was tested in a hamster model of severe SARS-CoV-2 pneumonia. The efficacy was compared to two doses of human convalescent plasma. Animals treated with monoclonal antibody or a high dose of human convalescent plasma were protected against weight loss, and significantly reduced pneumonia and pulmonary virus replication compared to control animals. A ten-fold lower dose of convalescent plasma showed no protective effect. These data show that prophylactic administration of high levels of neutralizing antibody prevent severe SARS-CoV-2 pneumonia in a hamster model, and could be used as an alternative or complement other antiviral treatments for COVID-19.

### Characteristics of Neutralizing Antibodies

Six convalescent plasma samples were pooled from PCR-confirmed COVID-19 patients. The samples were selected based on a minimum neutralizing antibody titer of 1:1280 (PRNT₅₀; Table 1 below). The neutralizing antibody titer of the pooled plasma as well as the 10-fold diluted pooled plasma were determined to be 1:2560 and 1:320 respectively. Ten of 115 convalescent plasma donors previously tested had a titer of 1:2560 or higher, while the 1:256 titer of the diluted plasma was still above the median titer of 1:160 of all donors tested.

TABLE 1
SARS-CoV-2 neutralizing antibody titers
in individual and pooled plasma

| Sample | PRNT₅₀ TITER |
|---|---|
| Donor 1 | 1280 |
| Donor 2 | 1280 |
| Donor 3 | 1280 |
| Donor 4 | >2560 |
| Donor 5 | 1280 |
| Donor 6 | 2560 |
| Pooled High | 2560 |
| Pooled Median | 320 |
| Pooled Negative | <20 |

In addition, human MAb 47D11 directed against SARS-CoV, which cross-reacts with SARS-CoV-2 and targets a conserved epitope in the S1 domain, was previously shown to neutralize SARS-CoV-2 with an IC50 of 0.57 μg/ml. At a concentration of 3 mg/mL the human MAb 47D11 preparation had an equivalent neutralizing antibody titer of 1:5260.

Neutralizing Antibodies Protect Against Body Weight Loss from SARS-CoV-2 Infection

To date, the Syrian golden hamster is the only animal species in which experimental SARS-CoV-2 infection results in severe pneumonia, with clinical signs, as well as shedding of virus. Therefore, the prophylactic potential of a MAb 47D11 and convalescent human plasma was evaluated in this hamster model. Twenty-four hours prior to challenge with SARS-CoV-2, animals were treated with MAb 47D11 or human convalescent plasma from COVID-19 patients. Volumes of human plasma treatment were chosen to mimic the application in humans. Animals were treated via intraperitoneal administration with either 3 mg MAb in 1 mL (equivalent of a PRNT₅₀ of 1:5260) or 500 ul human convalescent plasma (comparable to 300 mL of convalescent plasma treatment in an adult human) containing either high (PRNT₅₀ 1:2560) or median (PRNT₅₀ 1:320) levels of SARS-CoV-2 neutralizing antibodies. Therefore, animals treated with human convalescent plasma were given a 4× or 40× lower dose of neutralizing antibodies compared to MAb 47D11, respectively. Due to technical restrictions serum was not available on day 0 to determine the circulating neutralizing antibody titer. However, with a total blood volume in hamsters of 7.8 mL, we assumed that administration of MAb, and human convalescent plasma with high or median levels of neutralizing antibodies, lead to circulating neutralizing antibody titers of approximately 1:67, 1:16 and 1:2 respectively. There were three control groups, consisting of hamsters that were not treated prior to SARS-CoV-2 inoculation, and hamsters that were treated either with an irrelevant MAb or with normal healthy human plasma (not containing neutralizing antibodies to SARS-CoV-2; Table 1 above) 24 hr before SARS-CoV-2 inoculation.

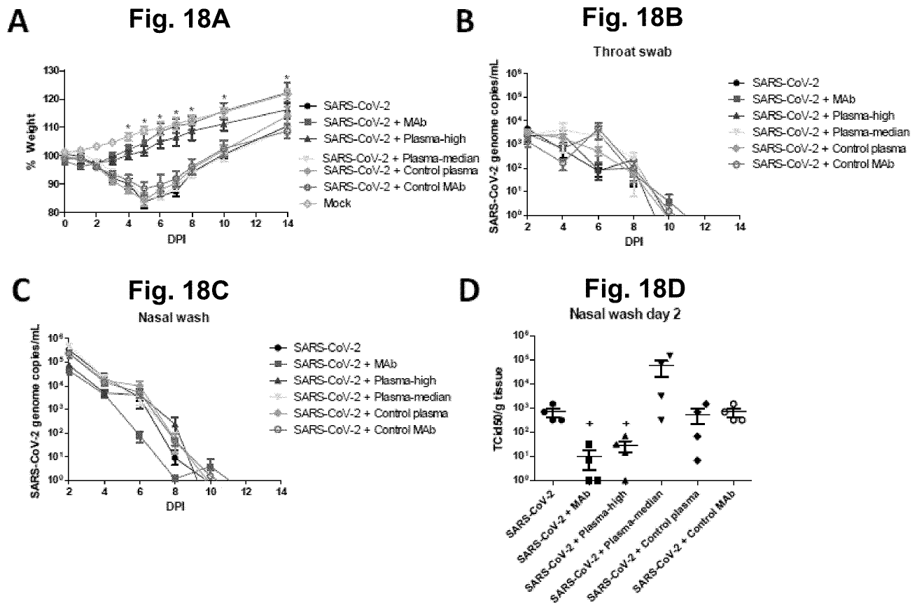

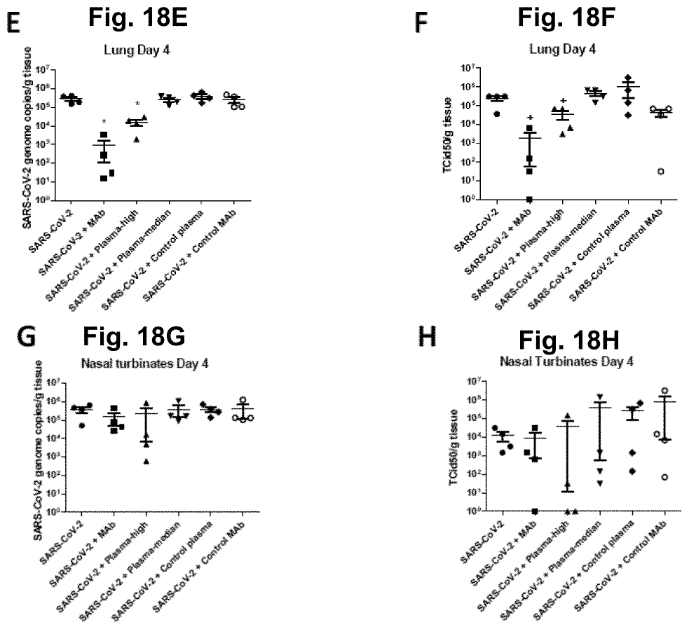

Experimental SARS-CoV-2 infection via the intranasal route resulted in a transient but significant weight loss in untreated animals as early as 2 days post inoculation (p.i.), approaching 20% weight loss by day 5 p.i. and normalizing by day 10 p.i. (FIG. 18A). No other clinical signs were observed. Treatment with MAb 47D11 or a high dose of convalescent plasma protected animals against significant weight loss (FIG. 18A). In contrast, treatment with the diluted convalescent plasma, control plasma, or control MAb did not protect against significant weight loss, with animals approaching 20% weight loss by day 5 p.i.

### Minimal Effect of Antibody Treatment on SARS-CoV-2 Shedding

SARS-CoV-2 inoculation of hamsters resulted in detection of viral RNA in throat swabs from all groups for up to 10 days p.i., with peak shedding on day 2 p.i. (FIG. 18B). There was no significant difference in virus detection in throat swabs between treated and control groups and no infectious virus could be detected at any time point.

In addition, viral RNA was detected in nasal washes for up to 10 days p.i. (FIG. 18C). Viral RNA loads were higher compared to those in throat swabs. While animals were protected against weight loss following treatment with either MAb 47D11 or high dose convalescent plasma, no significant reduction in detection of viral RNA in nasal washes was observed. Despite high levels of viral RNA in nasal washes for several days p.i., infectious virus could only be isolated on day 2 p.i. (FIG. 18D). Interestingly, while no significant effect of treatment was found on viral RNA detection, both treatment with MAb 47D11 and high dose convalescent plasma resulted in significant reduction of 1-2 logs in infectious virus on day 2 p.i. (p<0.05, ANOVA; FIG. 18D). Low levels of viral RNA were detected in rectal swabs on day 2 p.i. and occasionally at very low levels on other days in individual animals. There was no significant difference in virus detection in rectal swabs between treated and control groups and no infectious virus was detected.

### Antibody Treatment Reduced SARS-CoV-2 Replication in the Lower Respiratory Tract

Virus replication in the lungs and nasal turbinates was examined on day 4 p.i. (FIG. 18E-H). In the lungs, treatment with MAb 47D11 or plasma with high neutralizing antibodies resulted in significant reduction of viral loads (both viral RNA, p<0.01 and infectious virus, p<0.05, ANOVA) of 2 and 1 log, respectively (FIGS. 18E and F). In contrast, these treatments did not result in a significant reduction of viral load in the nasal turbinates (FIGS. 18G and H).

### Antibody Treatment Reduces Histopathological Changes in the Respiratory Tract Following SARS-CoV-2 Infection

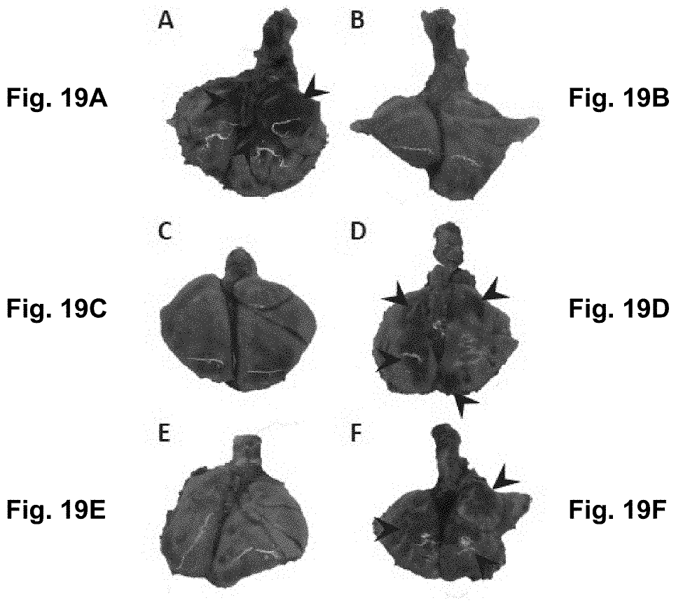

At autopsy on day 4 p.i., non-treated hamsters had single or multiple foci of pulmonary consolidation, visible as well-delimited, dark red areas, and covering 50-90% of the lung surface (FIG. 19). No gross lesions were observed in any of the animals treated with either MAb 47D11 or high dose of convalescent plasma. Lungs from animals treated with the diluted plasma, or control plasma/MAb showed similar lesions to untreated animals.

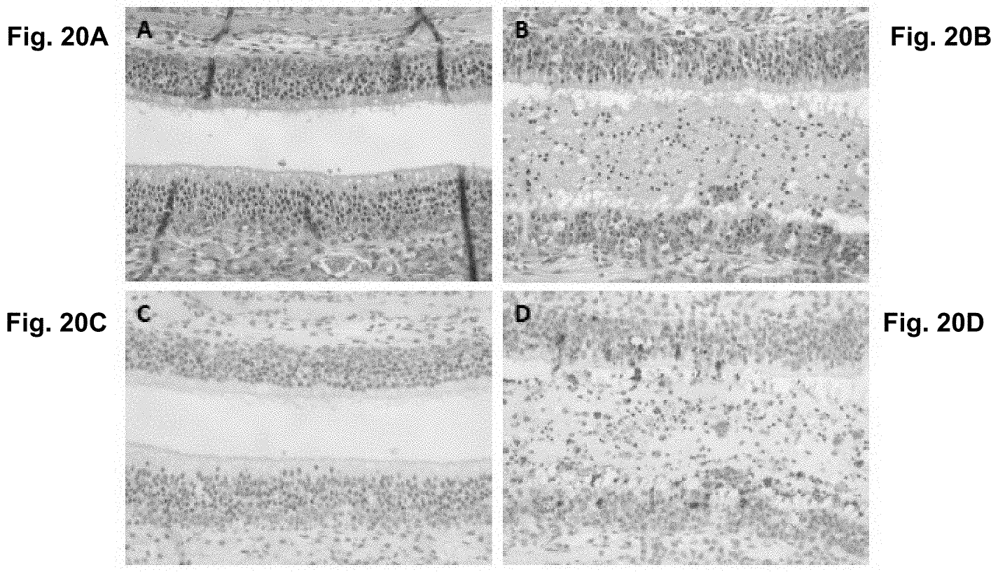

All animals, including the MAb 47D11 and high dose convalescent plasma groups, showed acute necrotizing and seropurulent rhinitis in the nasal cavity (FIG. 20). It was centred on the olfactory mucosa, where it was marked and locally extensive. There, it was characterized by edema in the lumen mixed with sloughed epithelial cells, neutrophils, and cell debris, and by the presence of a moderate number of neutrophils in the epithelium and underlying lamina propria.

Many olfactory epithelial cells in all animals expressed SARS-CoV-2 antigen, as demonstrated by immunohistochemistry. The inflammation in the undifferentiated mucosa and respiratory mucosa of the nasal cavity was mild and multifocal, and a few undifferentiated epithelial cells and ciliated columnar respiratory epithelial cells expressed virus antigen. There was no difference between treated or untreated animals inoculated with SARS-CoV-2.

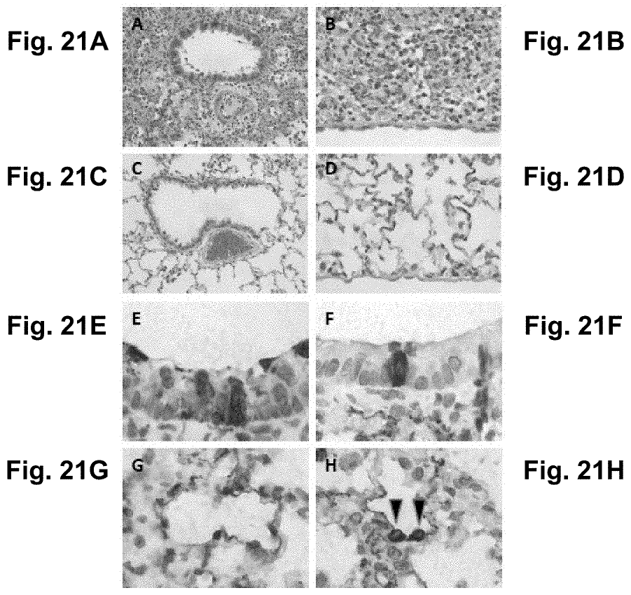

The main observation in the lungs of the non-treated animals and the animals treated with diluted convalescent plasma, control plasma, or control MAb was multifocal or coalescing diffuse alveolar damage, which was characterized by loss of histological architecture of the lung parenchyma, edema, fibrin, sloughed epithelial cells, cell debris, neutrophils, mononuclear cells, and erythrocytes (FIG. 21). By immunohistochemistry, many type I pneumocytes and fewer type II pneumocytes at the edges of the lesions expressed virus antigen. Besides diffuse alveolar damage, there also was mild multifocal necrotizing and purulent bronchiolitis, characterized by loss of bronchiolar epithelium and the presence of a few neutrophils in the bronchiolar walls and lumina. By immunohistochemistry, a few bronchiolar epithelial cells expressed virus antigen.

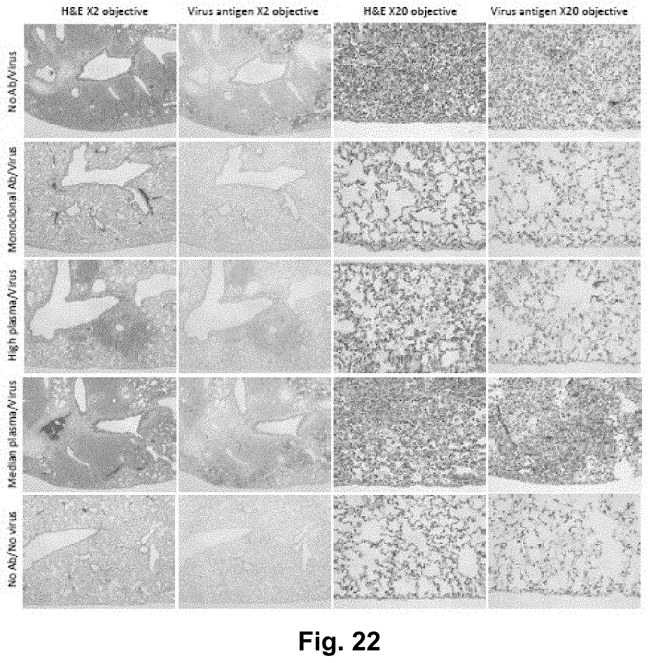

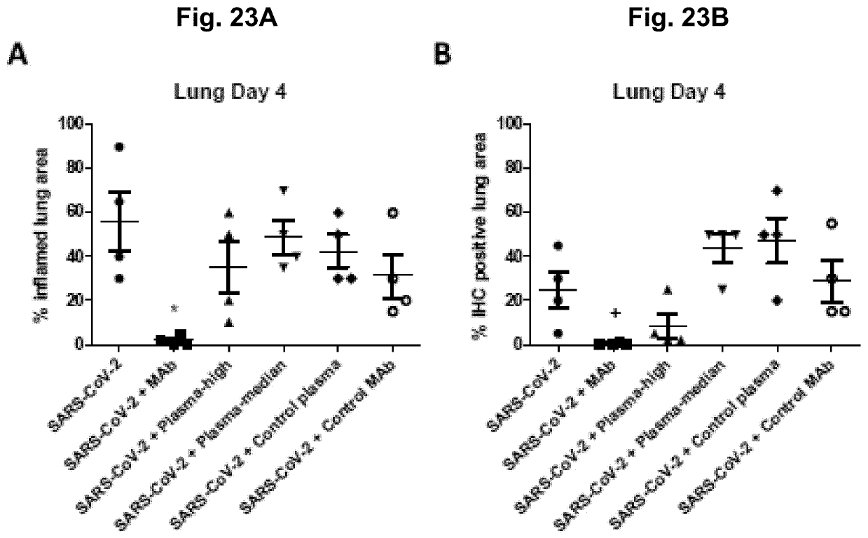

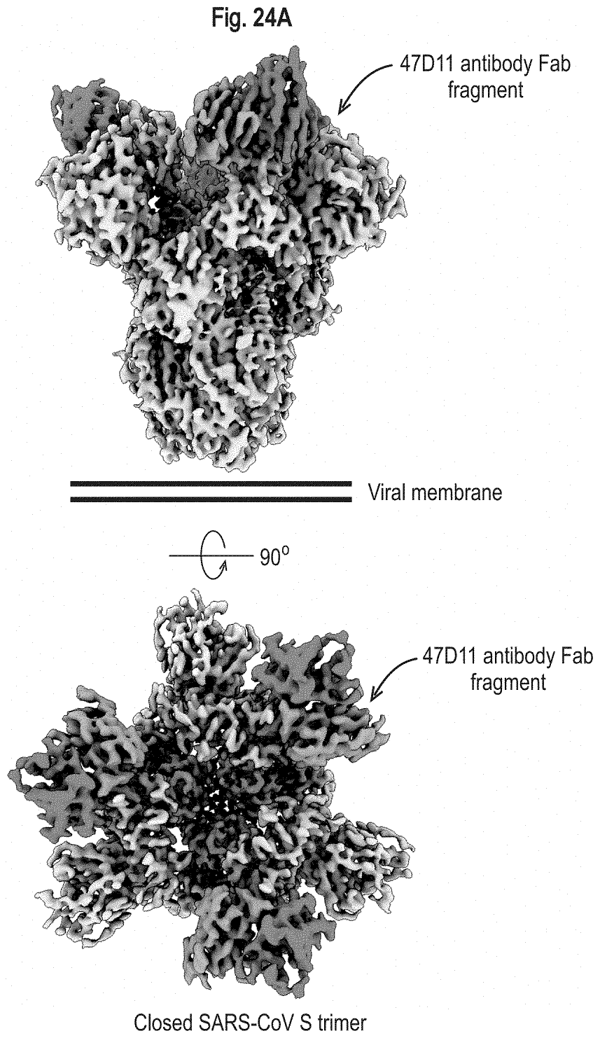

Treatment with MAb resulted in a significant reduction of inflammation in the lungs (p<0.01, ANOVA; FIGS. 22 and 23A) and viral antigen expression in the lungs (p<0.05, ANOVA; FIGS. 22 and 23B). Although a reduction in inflammation and viral antigen was observed in the lungs of animals treated with high dose convalescent plasma, this was not statistically significant.

### Antibody-Treated Animals Seroconvert Following SARS-CoV-2 Infection

Following SARS-CoV-2 inoculation, all animals seroconverted by day 22 regardless of the treatment regimen (Table 2 below). There was no significant difference in SARS-CoV-2 specific IgG titers among treatment groups with IgG titers of 1:12.800.

TABLE 2
Neutralizing antibody response (PRNT₅₀) in
hamsters following prophylactic treatment.

| | SARS- | | | | | | |
|---|---|---|---|---|---|---|---|
| | CoV-2 | | Plasma- | Plasma- | Control | Control | |
| Animal | only | MAb | high | low | plasma | MAb | Mock |
| 1 | 2560 | 2560 | 2560 | 2560 | 2560 | 2560 | <20 |
| 2 | 2560 | 2560 | 1280 | 2560 | 2560 | 640 | <20 |
| 3 | 2560 | 1280 | 2560 | 1280 | 2560 | 1280 | <20 |
| 4 | 2560 | 1280 | 640 | 2560 | 2560 | 1280 | <20 |

### DISCUSSION

This study shows that prophylactic treatment with neutralizing antibodies prevents severe SARS-CoV-2 induced pneumonia in a hamster model. Animals treated with a high dose of neutralizing antibodies were protected against significant weight loss, did not show any gross lesions in their lungs and treatment resulted in a very substantial reduction in lung inflammation and virus replication in the lungs.

Thus, this study shows that prophylactic treatment with neutralizing antibodies can protect against disease following SARS-CoV-2 infection. While hamsters infected with SARS-CoV-2 showed no overt respiratory signs, they lose significant weight. Animals treated with high titers of neutralizing antibodies were protected against significant weight loss, did not show any gross lung lesions and had significantly less histological lesions and associated virus antigen expression in the lungs.

While prophylactic treatment resulted in protection against disease and reduced SARS-CoV-2 replication in the lungs, only a limited effect was found in the upper respiratory tract. Previous studies with influenza virus have shown that serum IgG can diffuse at concentrations of 1:1 into alveolar lining fluid, thus protecting the lung parenchyma against virus infection. In contrast, the concentration of IgG on the surface of nasal mucosa is much lower. This suggests that treatment may protect against disease in the lungs but not virus transmission from the nose.

Recent studies have shown that SARS-CoV-2 can transmit between animals via both direct contact and air. Similar to our study, infectious virus was only detected in nasal washes early during infection and the period in which virus could be transmitted to naïve animals correlated with the presence of infectious virus (Sia et al. 2020 Nature). All animals treated with the MAb and convalescent plasma seroconverted, therefore, antibody based prevention of COVID-19 did not seem to prevent the development of humoral immunity after SARS-COV-2 exposure.

The use of hyperimmune globulin preparations from recovered patients has its inherent challenges, including safety, batch-to-batch variation, scalability, standardized dosing and presence of non-neutralizing antibodies. These challenges are more easily addressed by using (combinations) of recombinantly produced MAb. For example, antibody engineering allows to tweak the Fc-mediated immune effector functions and to improve MAb pharmacokinetics and reduce potential disease enhancing effects. In addition, established manufacturing pipelines allow efficient, highly controlled and scalable production.

In conclusion, these data show that prophylactic treatment with a highly neutralizing MAb not only protects against weight loss and reduces virus replication in the lungs, it also limits histopathological changes in the lungs. In addition, it is shown that while prophylactic treatment may prevent disease, animals still become infected and shed virus, indicating that transmission will not be blocked. These data highlight the importance to include virus shedding, replication in lungs as well as clinical and pathological determinants of disease in evaluating the efficacy of antibody treatment. In contrast, treatment with convalescent plasma provides only partial protection, and only at very high neutralizing titers. This protective effect is completely annulled when using the median neutralizing antibody dose found in recovered patients. It is therefore crucial to select convalescent plasma from donors with high levels of neutralizing antibody. Given the variation in antibody responses in patients, this limits the number of suitable donors for preparing immunoglobulin therapies considerably. No such limitation is present with in vitro produced MAbs and these results suggest this may be the more favorable route to develop an effective therapy.

### Materials and Methods

### Viruses and Cells

SARS-CoV-2 (isolate BetaCoV/Munich/BavPat1/2020) was obtained from a clinical case in Germany diagnosed after returning from China (European Virus Archive Global #026V-03883). The virus was propagated to passage three on Vero E6 cells in Opti-MEM I (1×)+GlutaMAX (Gibco), supplemented with penicillin (10,000 IU/mL) and streptomycin (10,000 IU/mL) at 37° C. in a humidified CO2 incubator. All work was performed in a Class II Biosafety Cabinet under BSL-3 conditions at the Erasmus Medical Center (MC).

### MAb and Convalescent Plasma

The identification and characterisation of MAb 47D11, which efficiently neutralizes SARS-CoV-2 in vitro, is described in Examples 1-4 above.

Convalescent plasma was collected from donors who had a RT-PCR confirmed SARS-CoV-2 infection and were asymptomatic for at least 14 days. Of all donors tested, only plasma with neutralizing antibodies against SARS-CoV-2 confirmed by a SARS-CoV-2 plaque reduction neutralization test (PRNT) and a PRNT50 titer of at least 1:1280 was used. Equal volumes of plasma from 6 donors was pooled and used for prophylactic treatment in hamsters (High dose). In addition, the pooled plasma was diluted 10-fold in PBS (Median dose). Normal human plasma from a healthy donor was used as a control.

### Animal Procedures SARS-CoV-2

Female Syrian golden hamsters (Mesocricetus auratus; 6-week-old hamsters from Janvier, France) were anesthetized by chamber induction (5 liters 100% 02/min and 3 to 5% isoflurane). 24-hour prior to inoculation with virus, groups of 8 animals were treated with either 3 mg of MAb in 1 mL or 500 μl human convalescent plasma via the intraperitoneal route.

Animals were inoculated with 105 TCID50 of SARS-CoV-2 or PBS (mock controls) in a 100 μl volume via the intranasal route. During the experiment the animals were monitored for general health status and behavior daily and were weighed regularly for the duration of the study (up to 22 days post inoculation; d.p.i.). Nasal washes, throat swabs and rectal swabs were collected under isoflurane anesthesia during the study. Groups of 4 animals were euthanized on day 4 or day 22 after inoculation, and serum samples, as well as lung, and nasal turbinates, were removed for virus detection and histopathology.

### Serological Analysis

To test for SARS-CoV-2 antibodies, hamster serum samples were collected at days 4 and 22. Serum samples were tested for SARS-CoV-2 antibodies using a spike S1 and nucleocapsid protein (N) ELISAs. Briefly, ELISA plates were coated overnight with SARS-CoV-2 S1. After blocking, serum samples were added and incubated for 1 h at 37° C. Bound antibodies were detected using HRP-labelled rabbit anti-human IgG (Dako) or anti-hamster IgG and TMB (Life Technologies) as a substrate. The absorbance of each sample was measured at 450 nm.

A plaque reduction neutralization test (PRNT) was used as a reference for this study as previously described (Okba et al. 2020 Emerging Infectious Diseases 1478-1488). The serum neutralization titer is the reciprocal of the highest dilution resulting in an infection reduction of >50% (PRNT50).

### Virus Detection

Samples from nasal turbinates and lungs were collected post mortem for virus detection by RT-qPCR and virus isolation as previously described (Rockx et al. 2020 Science 368:1012-1015). Briefly, tissues were homogenized 10% w/v in viral transport medium using Polytron PT2100 tissue grinders (Kinematica). After low-speed centrifugation, the homogenates were frozen at −70° C. until they were inoculated on Vero E6 cell cultures in 10-fold serial dilutions. The SARS-CoV-2 RT-qPCR was performed and quantified as copy numbers as previously published (Corman et al. 2020 Euro Surveill. 25).

### Histopathology and Immunohistochemistry

For histological examination lung and nasal turbinates were collected. Tissues for light microscope examination were fixed in 10% neutral-buffered formalin, embedded in paraffin, and 3 μm sections were stained with haematoxylin and eosin.

Sections of all tissue samples were examined for SARS-CoV-2 antigen expression by immunohistochemistry as previously described (Rockx et al. 2020 Science 368:1012-1015). Briefly, paraffin was removed from sections, and viral antigen was detected using a rabbit polyclonal antibody against SARS-CoV nucleoprotein (40143-T62, Sino Biological, Chesterbrook, Pa., USA) and horseradish peroxidase labeled goat-anti-rabbit IgG (P0448, DAKO, Agilent Technologies Netherlands B.V. Amstelveen, The Netherlands). Horseradish peroxidase activity was revealed by incubating slides in 3-amino-9-ethylcarbazole (Sigma, St Louis, Mo., USA) solution, resulting in a bright red precipitate. Sections were counterstained with haematoxylin. For quantitative assessment of SARS-CoV-2 infection-associated inflammation in the lung, each H&E-stained section was examined for inflammation by light microscopy using a 2.5× objective, and the area of visibly inflamed tissue as a percentage of the total area of the lung section was estimated. Quantitative assessment of virus antigen expression in the lung was performed according to the same method, but using lung sections stained by immunohistochemistry for SARS-CoV-2 antigen. Sections were examined without knowledge of the identity of the hamsters.

### Statistical Analysis

Statistical analyses were performed using GraphPad Prism 5 software (La Jolla, Calif., USA). Each specific test is indicated in the figure legends. P values of <0.05 were considered significant. All data are presented as means±standard error of the mean (SEM).

### Example 6—47D11 Targets a Conserved Pocket in the SARS2 Receptor-Binding Domain

In this Example, cryo-EM structures of trimeric SARS-CoV and SARS-CoV-2 spike ectodomains in complex with the 47D11 Fab. These data reveal that 47D11 binds specifically to the closed conformation of the receptor binding domain, distal to the ACE2 binding site. The CDRL3 stabilises the N343 glycan in an upright conformation, exposing a conserved and mutationally constrained hydrophobic pocket, into which the CDRH3 loop inserts two aromatic residues. Interestingly, 47D11 preferentially selects for the partially open conformation of the SARS-CoV-2 spike, suggesting that it could be used effectively in combination with other antibodies that target the exposed receptor-binding motif. Taken together, these results expose a cryptic site of vulnerability on the SARS-CoV-2 RBD and provide a structural roadmap for the development of 47D11 as a prophylactic or post-exposure therapy for COVID-19. The results also reveal a cross-protective epitope on the SARS-CoV-2 spike.

The coronavirus trimeric spike (S) glycoprotein, located on the viral envelope, is the key mediator of viral entry into host cells. The spike protein consists of two main parts: S1 is involved in receptor binding and S2 is the membrane fusion domain. The S1 domain itself is further subdivided into an N-terminal domain (NTD, or S1A) and a Receptor Binding Domain (RBD, or S1B). The spike proteins of SARS-CoV-2 (SARS2-S; 1273 residues, strain Wuhan-Hu-1) and SARS-CoV (SARS-S, 1255 residues, strain Urbani) exhibit 77.5% identity in their primary amino acid sequence and are structurally conserved. The spike protein exists in equilibrium between a closed conformation, where all three RBD are lying flat (in a “down” conformation), and a partially open conformation, where one RBD stands upright (i.e. adopts an “up” conformation) and is exposed for receptor engagement. Both viruses use the human angiotensin converting enzyme 2 (ACE2) protein as a host receptor, with binding mediated through interactions with the receptor-binding motif (RBM) located on the RBD, and the N-terminal helix of ACE2. The spike-mediated fusion of viral and cellular membranes is tightly regulated and triggered by a cascade of preceding events. The first step involves the attachment of SARS-CoV-2 to the target cell surface via the interaction between spike and ACE2. In the second step, the spike protein needs to be primed for membrane fusion by host proteases (e.g. cellular transmembrane serine protease 2 which cleaves the spike at multiple sites, enabling shedding of S1. Finally, the free S2 catalyses the fusion of the viral and the host membranes, causing the release of the viral genome into the host cell cytoplasm.

Structural and functional studies to decipher the molecular basis for 47D11 mediated neutralisation of SARS-CoV and SARS-CoV-2 were performed.

A number of recently identified SARS-CoV-2 variants harbour mutations in the RBM (K417N/T, E484K and N501Y), which could facilitate viral escape from monoclonal antibodies binding in this region, as well as some polyclonal sera dominated by this class of antibodies. The capability for 47D11 to neutralize SARS2-S pseudotyped viruses with mutations in the RBM (K417N, E484K or N501Y) observed in emerging SARS-CoV-2 variants was tested.

### Results

### 47D11 Specifically Binds to the Closed Receptor Binding Domain

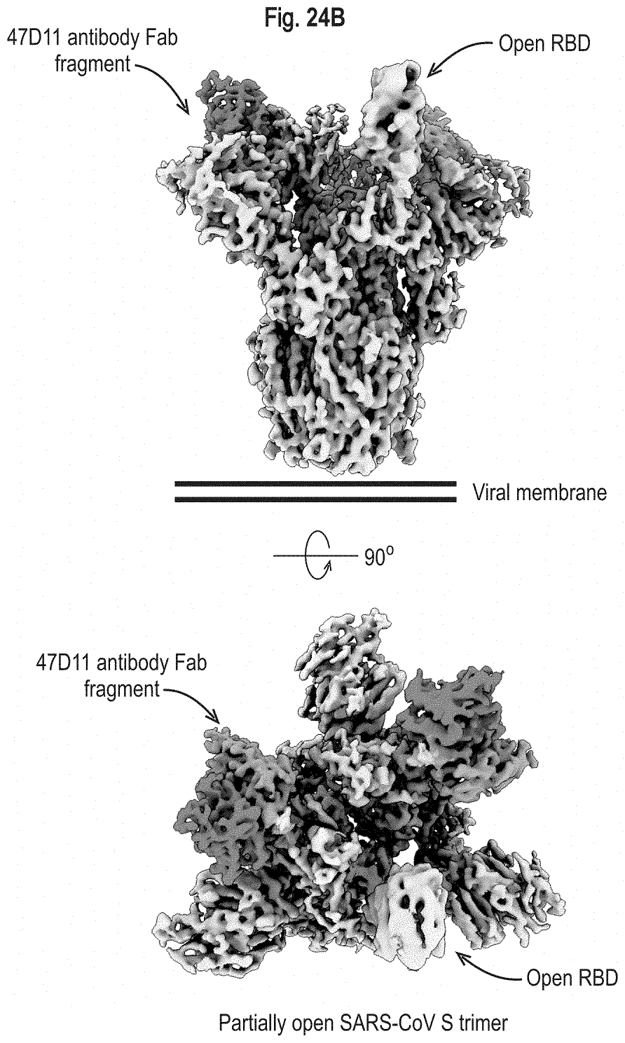

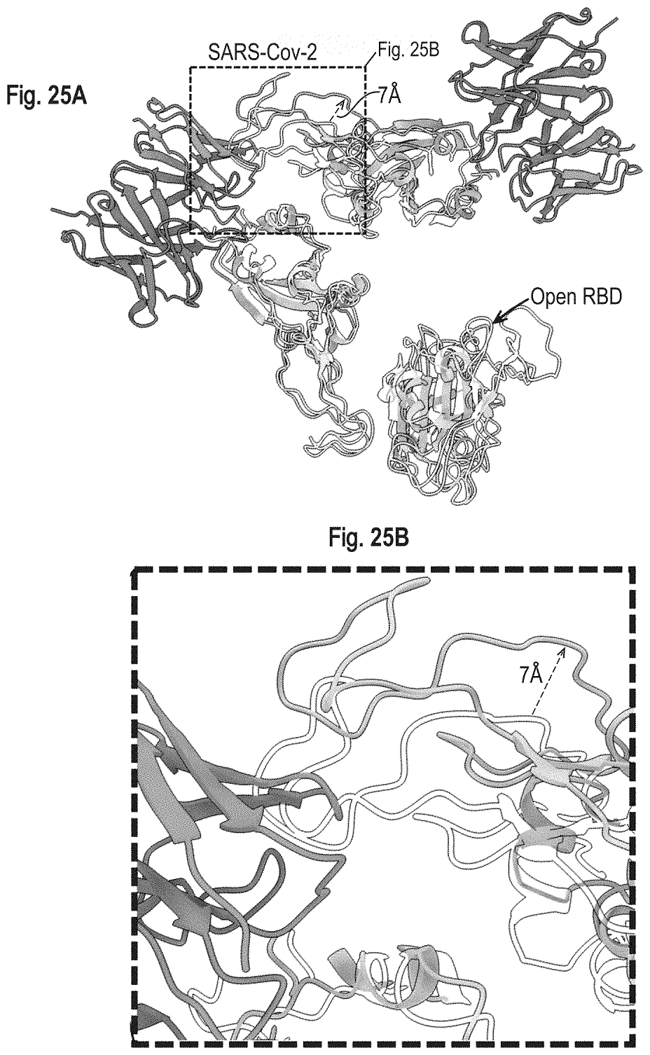

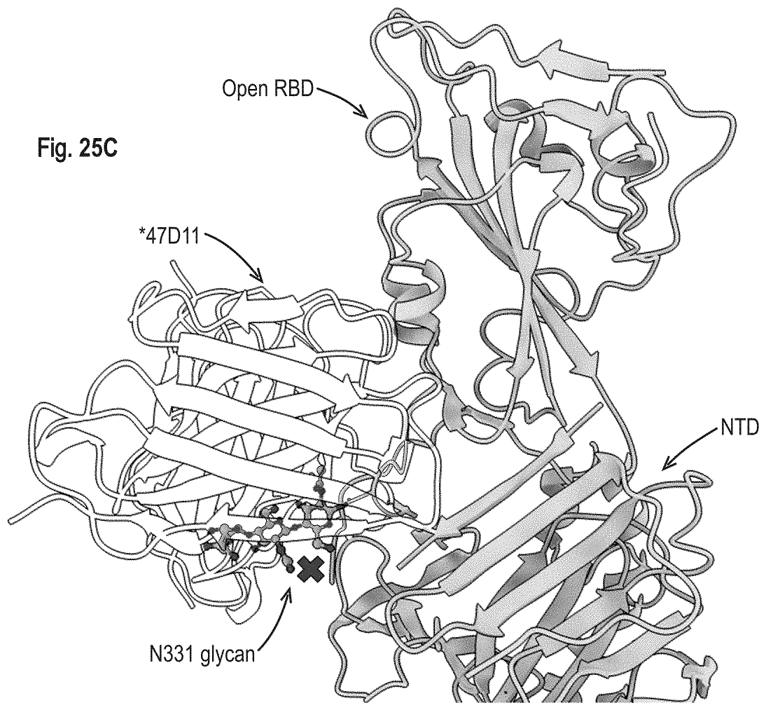

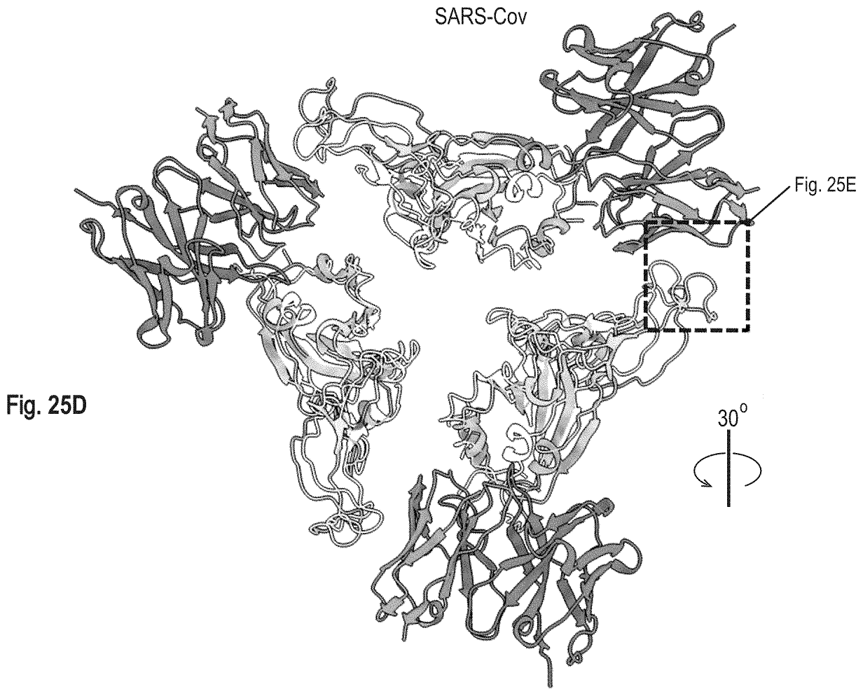

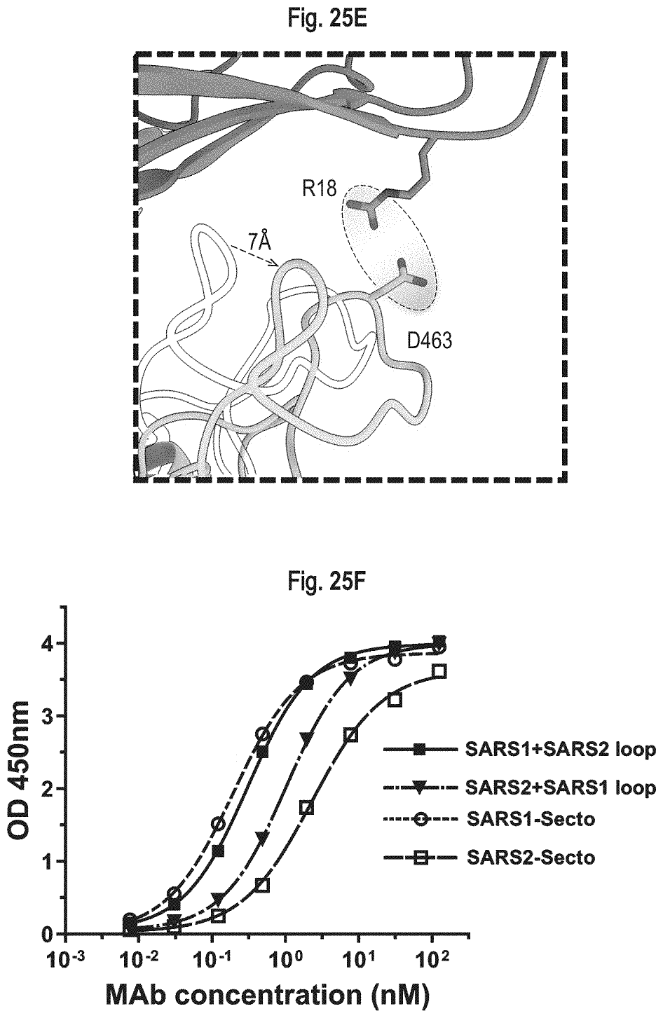

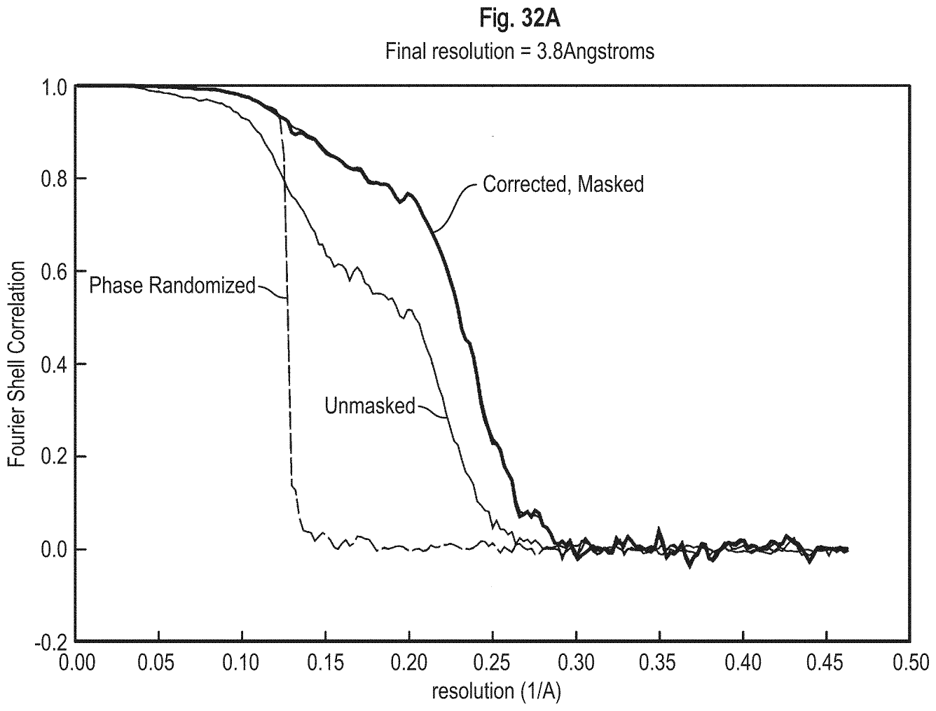

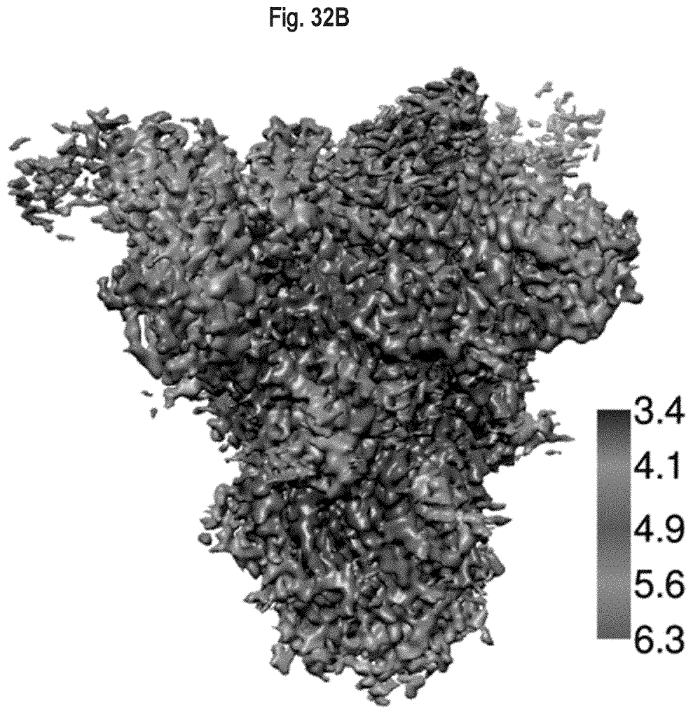

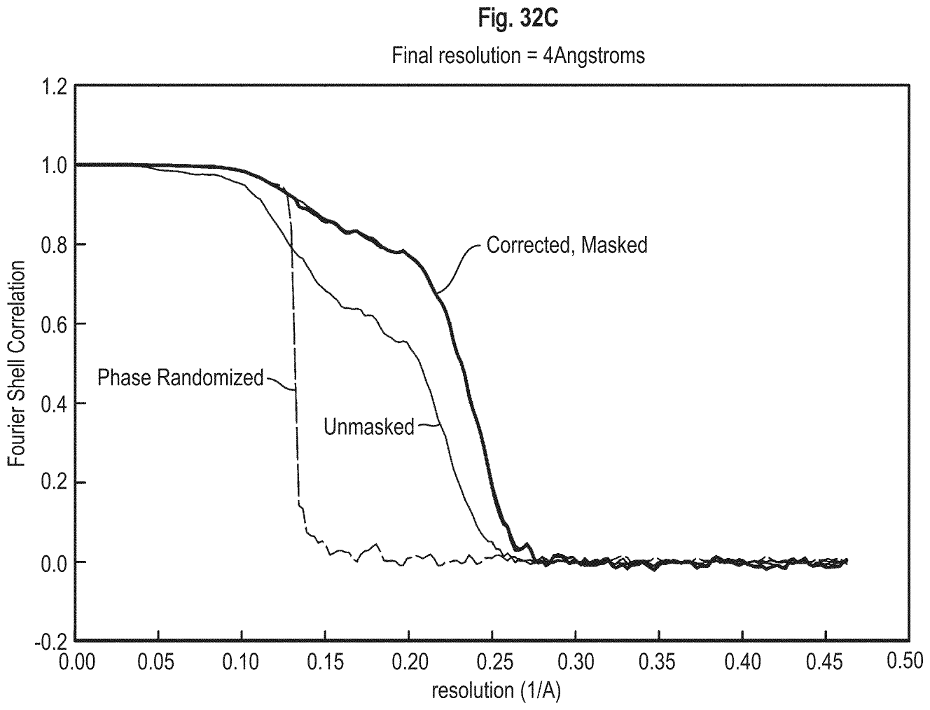

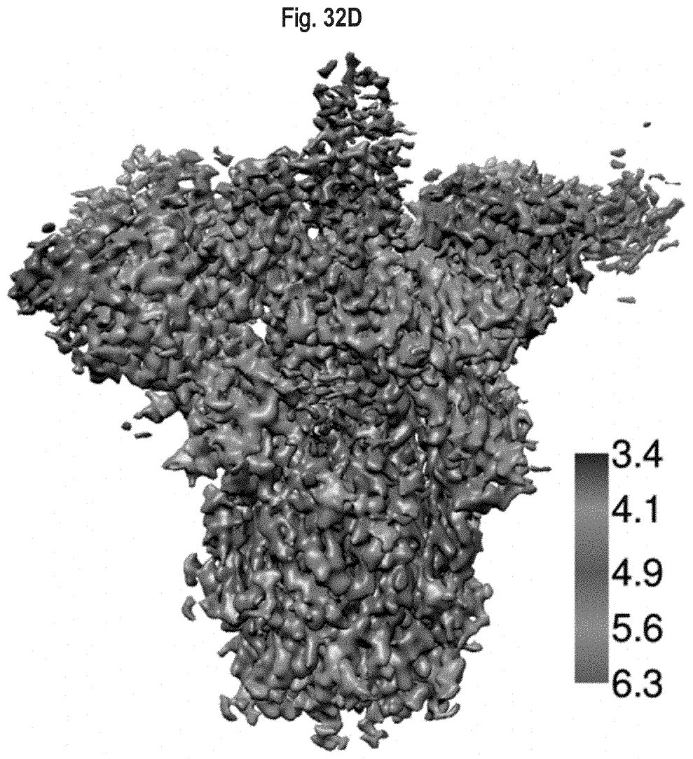

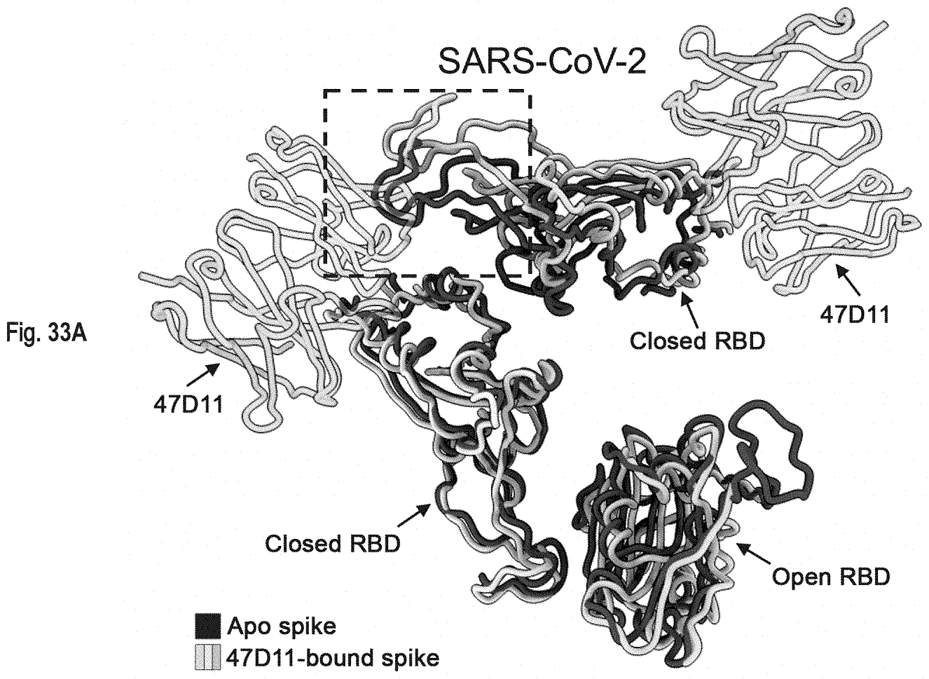

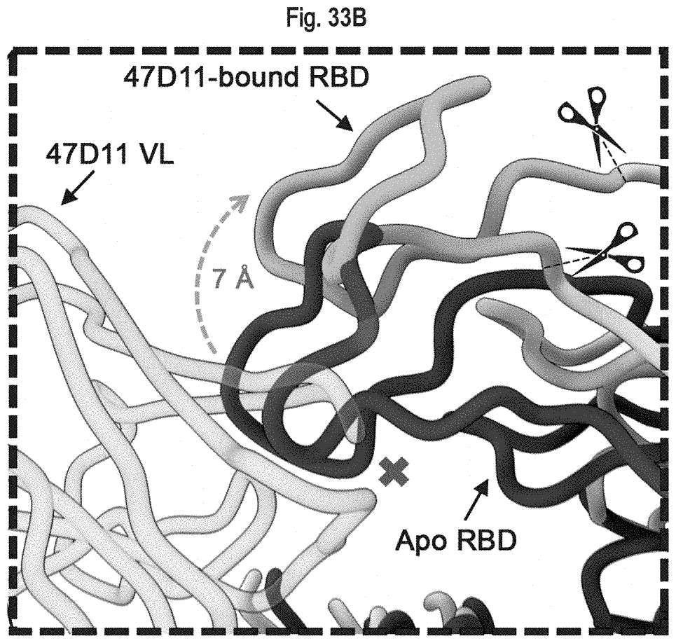

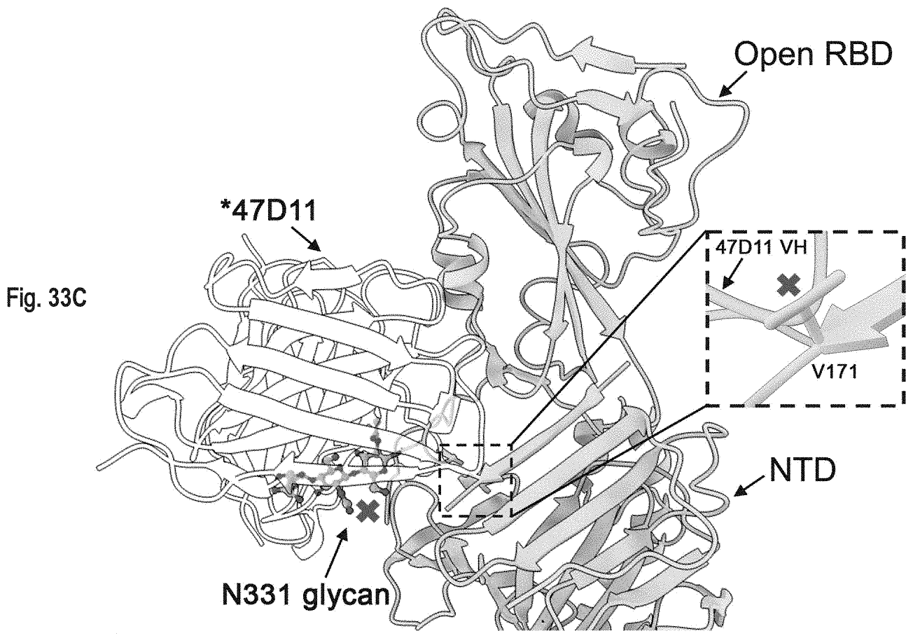

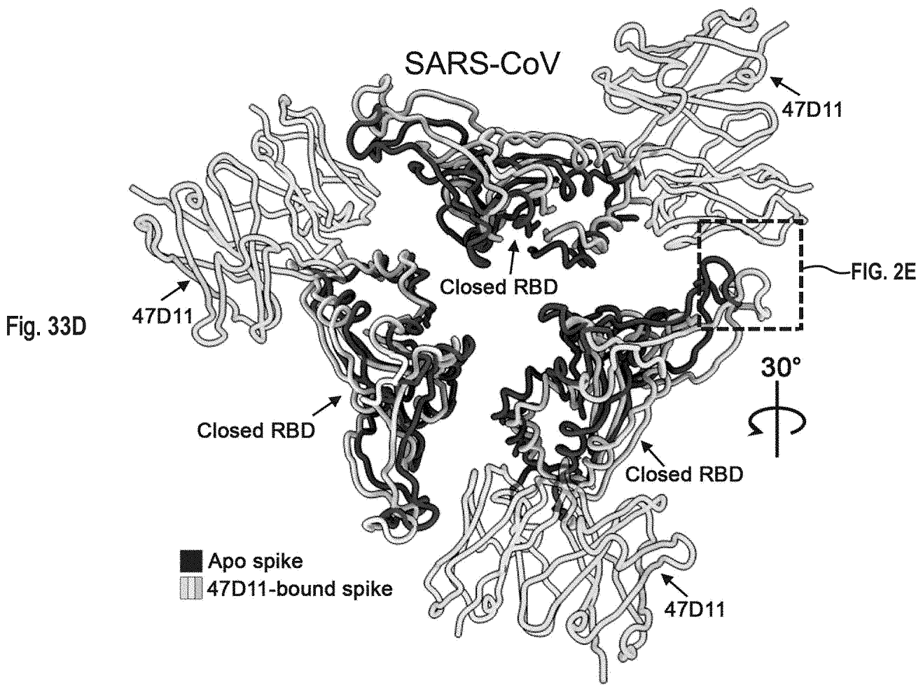

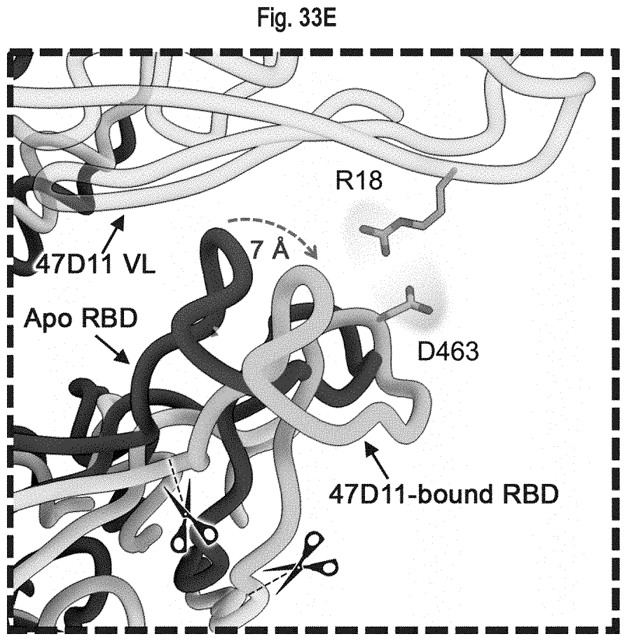

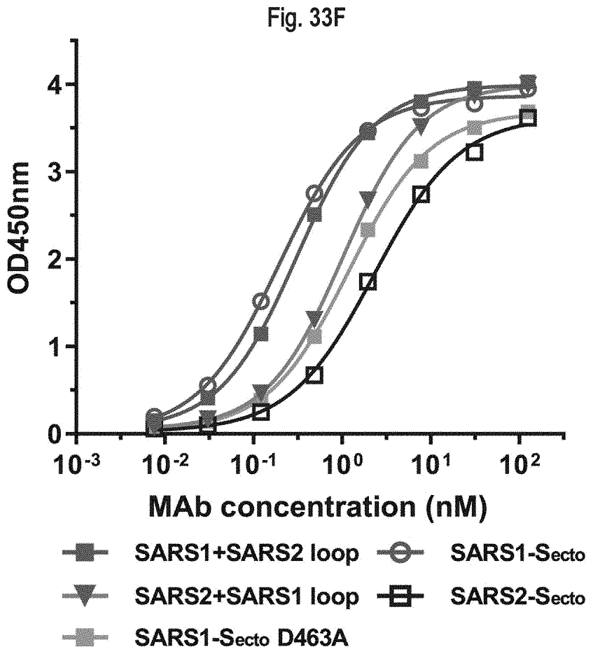

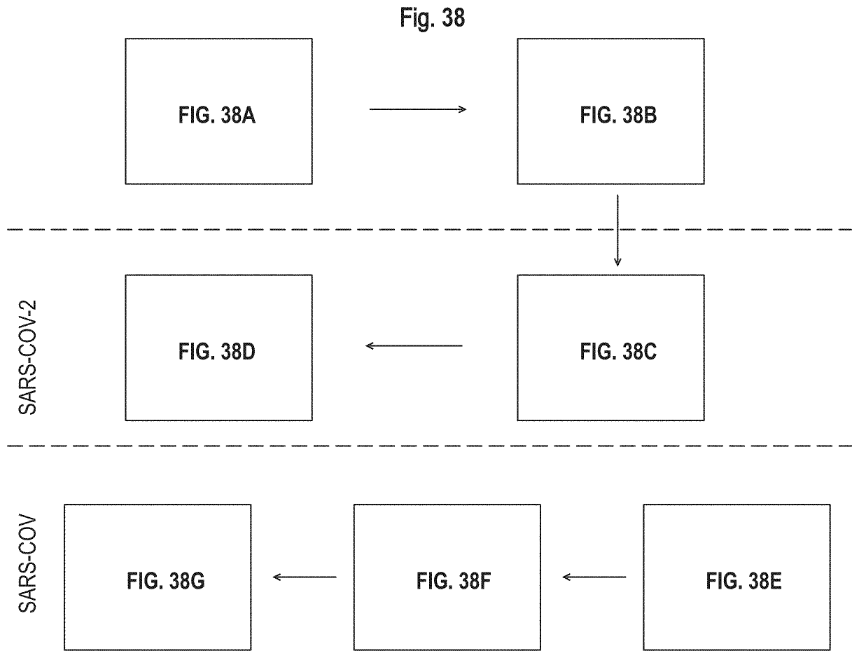

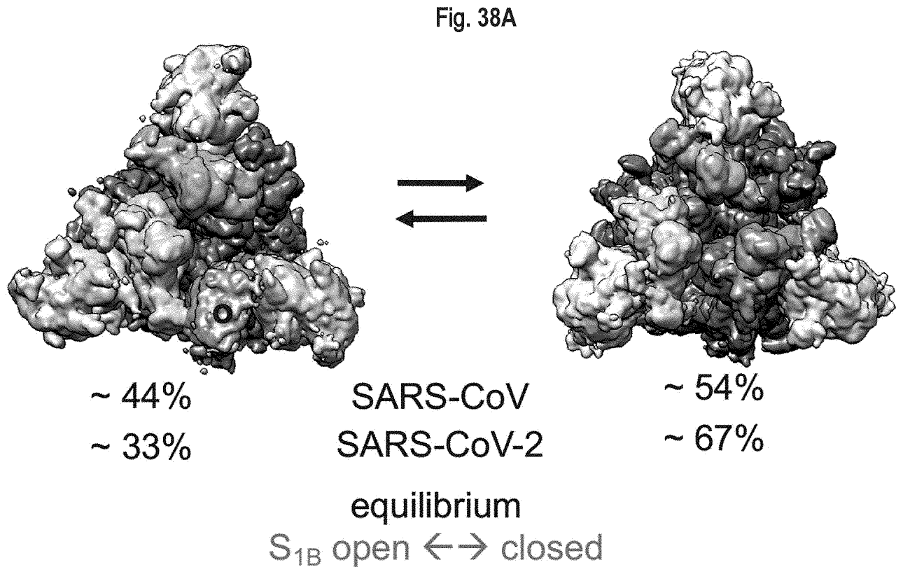

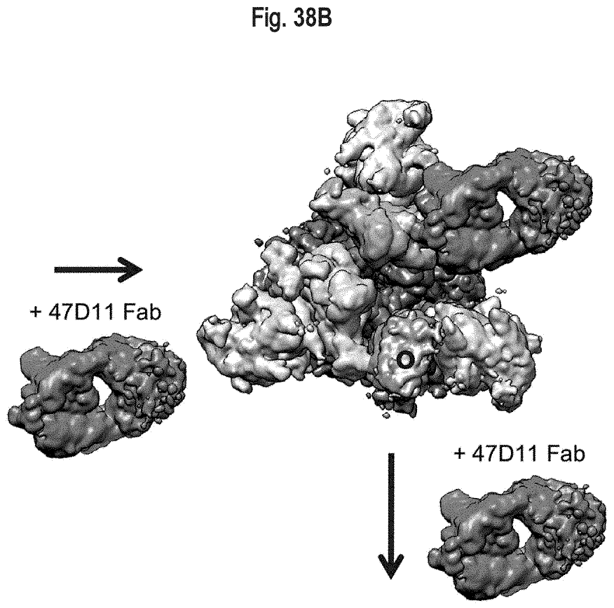

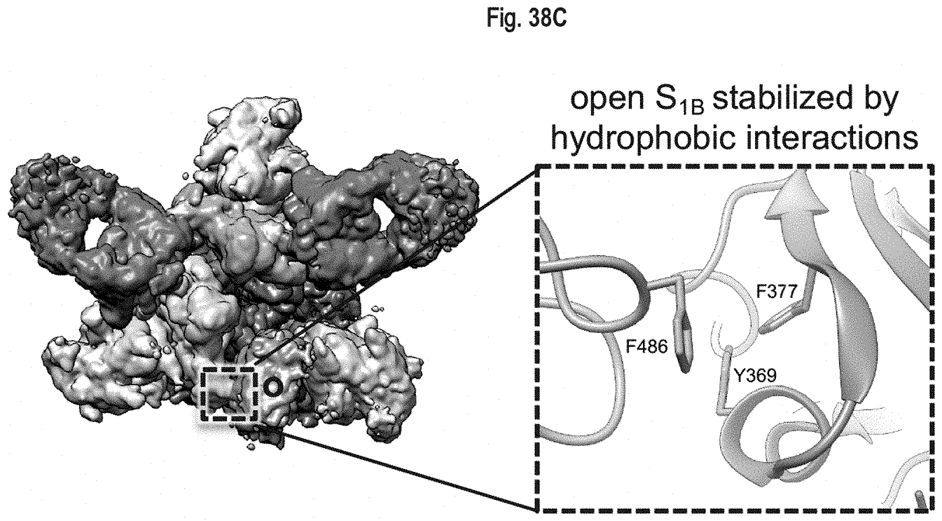

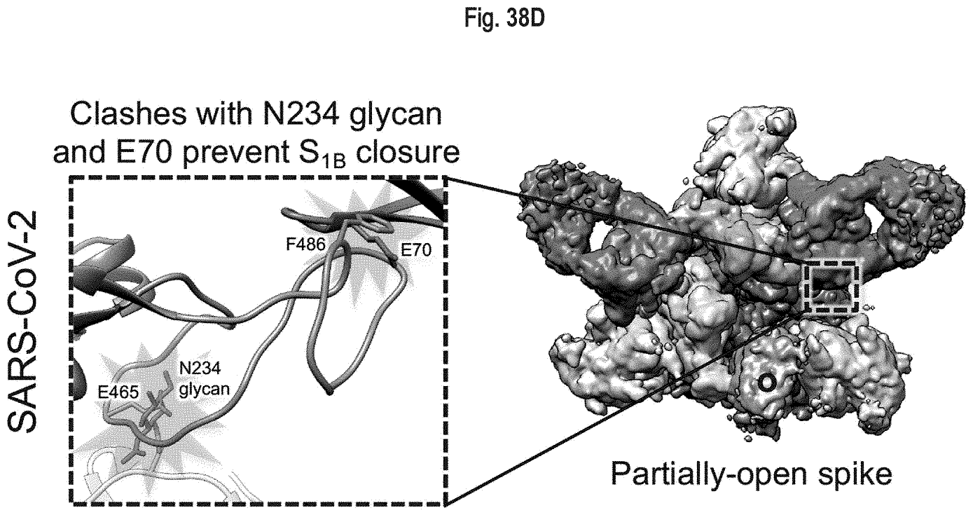

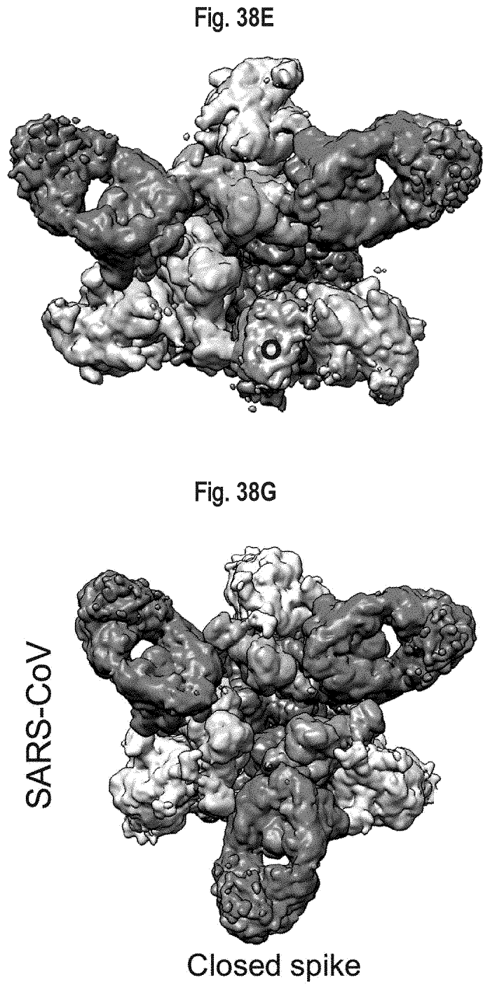

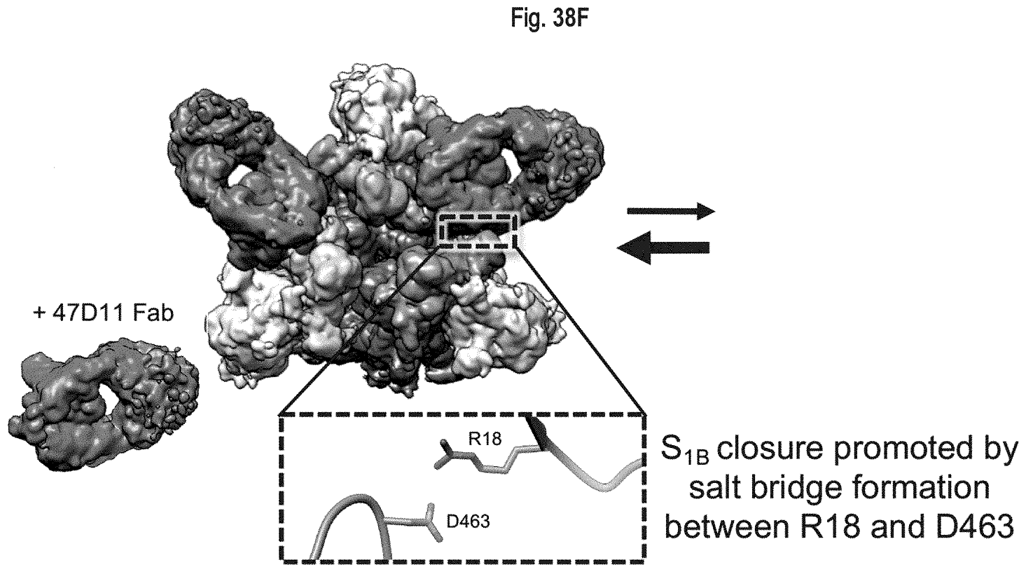

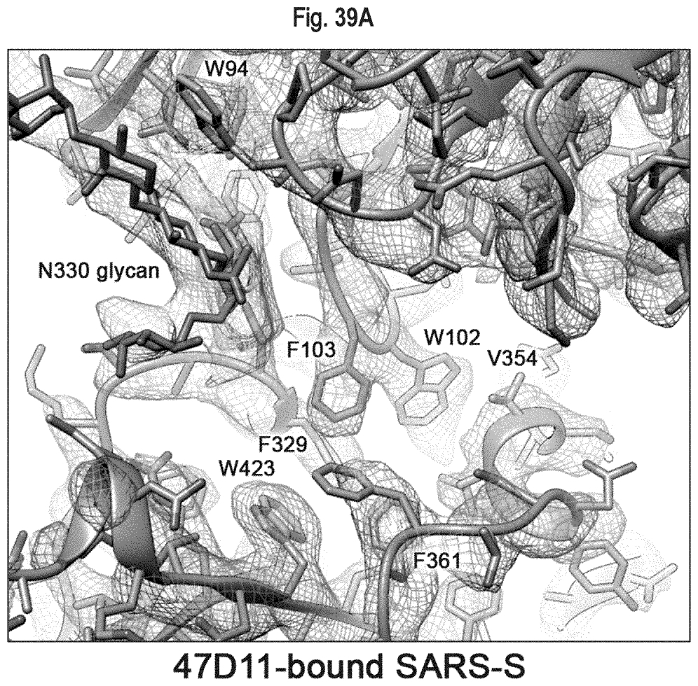

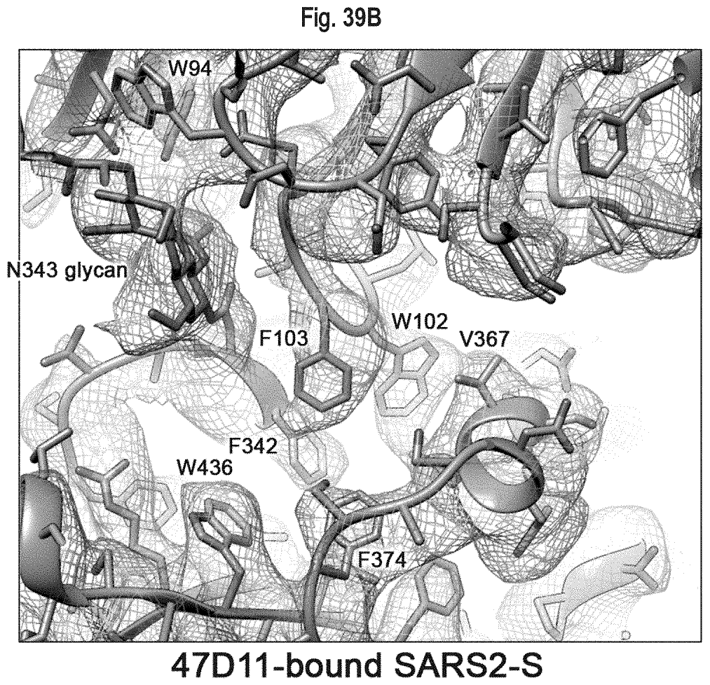

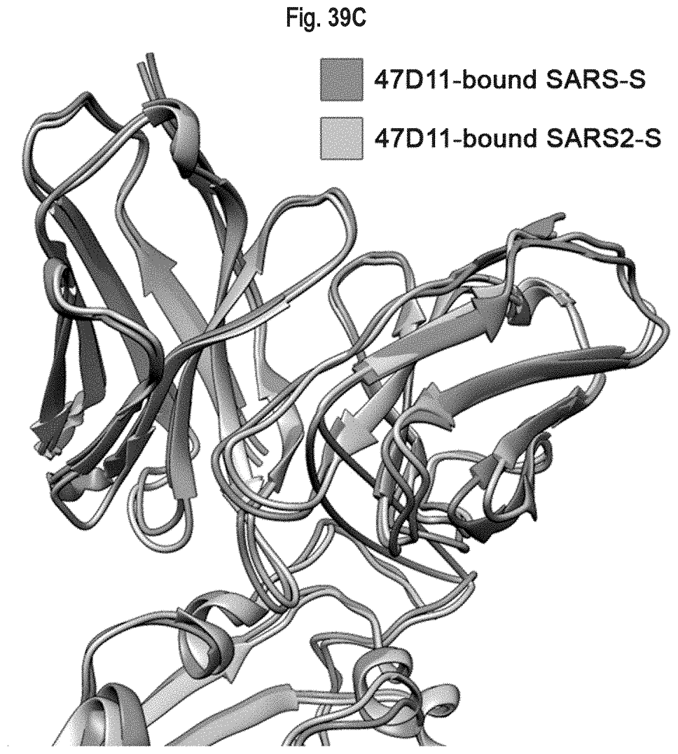

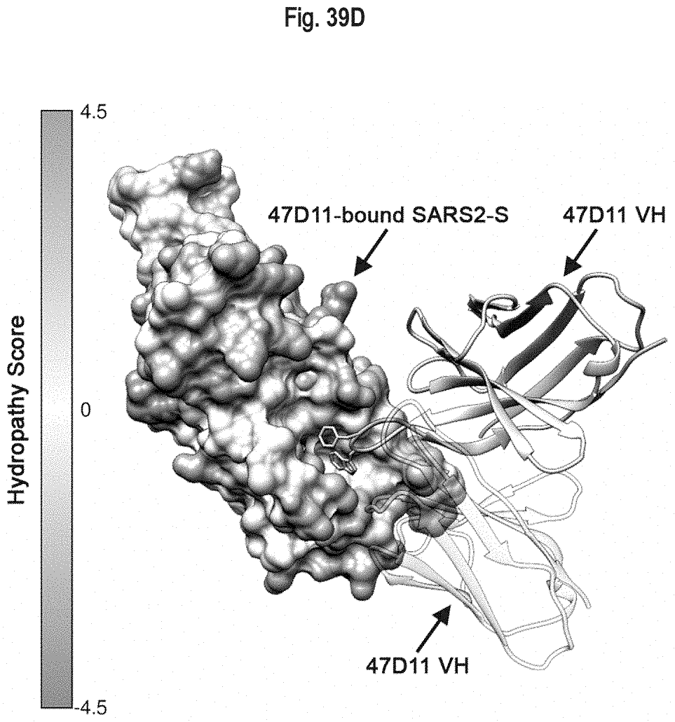

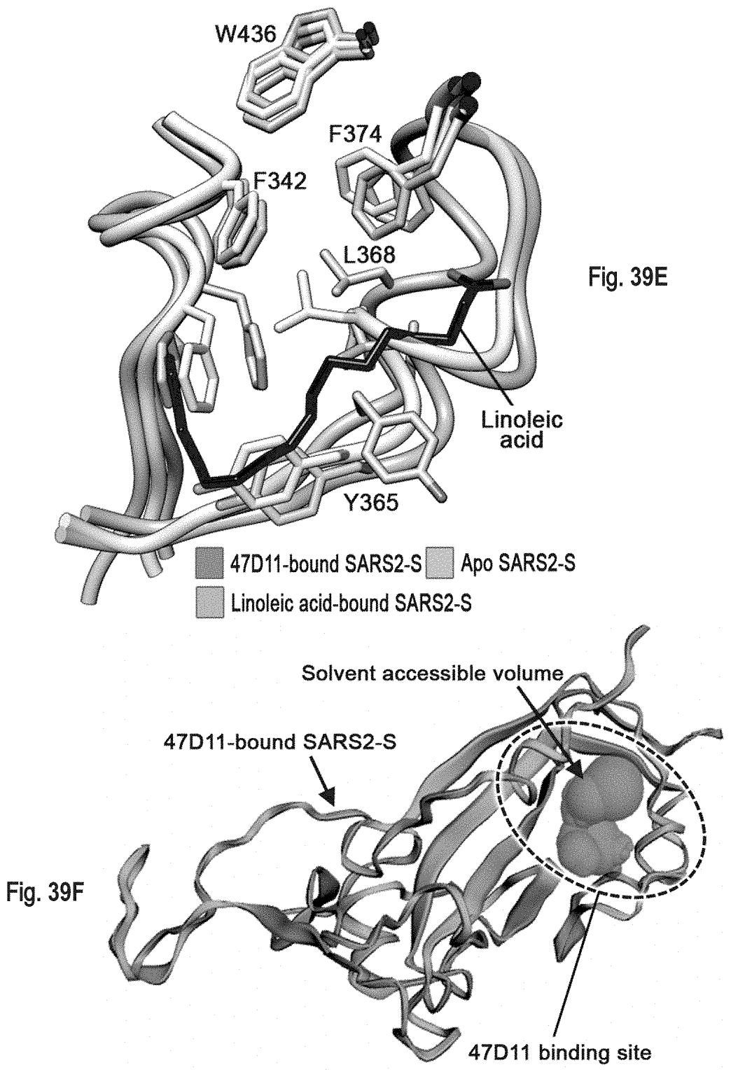

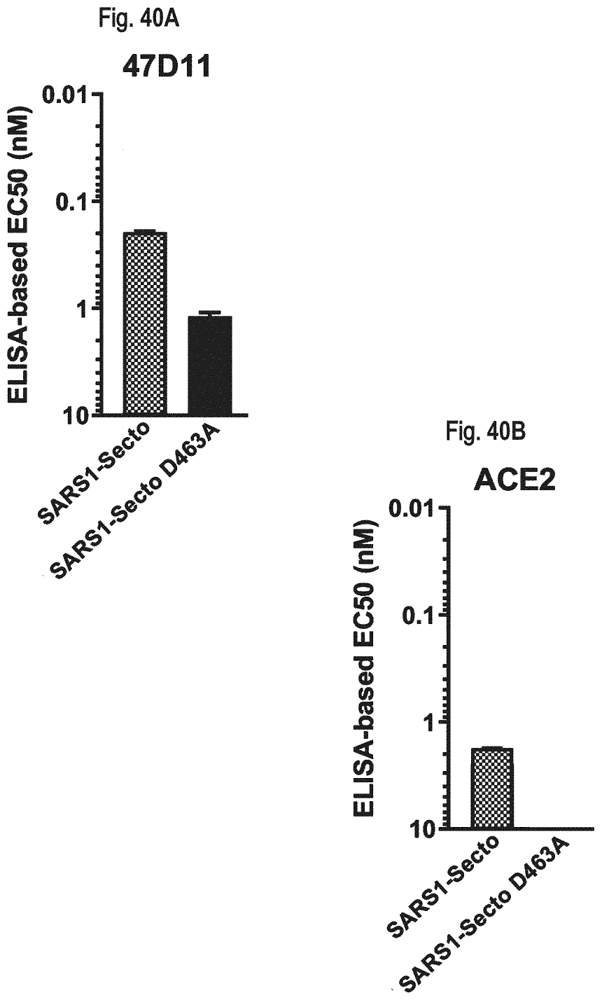

To understand the structural basis of how 47D11 binds to the SARS-CoV and SARS-CoV-2 spike proteins, cryo-electron microscopy (cryo-EM) single particle analysis was used to determine structures of prefusion stabilised ectodomain (Secto) trimers in complex with the 47D11 Fab fragment. The resulting cryo-EM maps have global resolutions of 3.8 Å and 4.0 Å resolution for SARS1-S and SARS2-S, respectively (FIGS. 32A-B and 37A). For previously reported apo S trimers, both the open and closed conformation are observed, with the latter being predominant (54% for SARS1 and 67% for SARS2). Upon incubation with 47D11, only the closed conformation of the SARS1 spike was observed, with stochiometric binding of 47D11 to each RBD (FIG. 24A). Interestingly, for SARS2, only the partially open conformation of spike was observed, with one Fab bound to each of the closed RBDs, and the remaining open RBD unoccupied and, in principle, accessible to ACE2 binding (FIG. 24B). The sub stochiometric binding observed for SARS2-S may partially explain our previous observations that 47D11 binds to the SARS-S with higher affinity than SARS2-S (equilibrium dissociation constant [K_{D}] of 0.745 nM and 10.8 nM, respectively). To understand why 47D11 favours different spike conformations for SARS1 and SARS2, the Fab bound structures were first superposed with their apo counterparts. Compared to the apo partially open SARS2-S structure, the RBDs are less compact when 47D11 is bound (FIGS. 25A and 33A). The apo conformation of the closed RBD would preclude binding of 47D11 through steric hindrance. To accommodate the bound Fab, this RBD shifts outwards by ˜7 Å (FIGS. 25B and 33B). Unlike Mabs 5309 and H014, two other RBD-core targeting cross-neutralizing antibodies, there was no indication from the cryo-EM data that 47D11 can bind to the open (up) conformation of the SARS2-S RBD. In line with this, superimposition of open and closed SARS2-S RBDs revealed that 47D11 would clash with the adjacent N-terminal domain (NTD) and the N331 glycan in the latter conformation (FIGS. 25C and 33C). Similarly, the RBDs of the 47D11 bound SARS1-S are also less compact than the reported apo fully closed structure (FIGS. 25D and 33D). However, in contrast to SARS2-S, there is a potential stabilising salt bridge between D463, located on the receptor binding ridge (RBR), and R18 on the 47D11 light chain (FIGS. 25E and 33E). Indeed, the RBR exhibits the most prominent structural differences between SARS2-S and SARS1-S (Lan et al. (2020) Nature 579:1-6). This epitope distal loop, located within the ACE2-binding region, contains an essential disulfide bridge in both viruses, but is more compact in SARS2-S. In order to test whether the epitope distal RBR impacts binding of 47D11 to the SARS1-S and SARS2-S, we swapped loop residues 470-490 (SARS2-S numbering) and produced chimeric ectodomains. A D463A mutation was also introduced into SARS-S to disrupt the observed salt bridge (FIG. 33E). In support of our hypothesis, the SARS2-S, containing the SARS1-S RBR loop, exhibited increased binding to 47D11. In contrast, the SARS-S D463A mutant displayed decreased binding to 47D11 and loss of ACE2 binding (FIGS. 40A and 40B). However, we did not observe an equivalent loss of binding for the chimeric SARS1-S, suggesting that other differences in protein sequence or quaternary structure may be involved (FIGS. 25F and 33F). Taken together, our data show that 47D11 binding to the RBD has differing outcomes for SARS-S and SARS2-S, trapping them in the fully closed and the partially open conformation, respectively (FIG. 38).

These results rationalize our previous observation that the 47D11-bound SARS2-S can still bind soluble ACE2 in a cell staining assay, given that it has one open RBD accessible to the receptor.

Without wishing to be bound by any theory, two possible explanations for how the 47D11-bound SARS1-S, stabilized with the 3 RBDs in the down conformation, can also bind ACE2 have been identified. First, the 47D11-bound RBDs may be able to adopt a semi-open conformation, too transient to be visualized by cryo-EM, which could accommodate both 47D11 and ACE2 binding. Alternatively, it is possible that 47D11 binding results in destabilization of the spike trimer leading to the separation of the protomers and exposure of the ACE2 binding site, as reported for CR3022.

Without wishing to be bound by any theory, a possible explanation for the neutralization mechanism of 47D11 has been identified. First, as viral membrane fusion is a tightly controlled process, it is possible that the perturbation of the spike conformational flexibility induced by 47D11 binding, hinders the correct and timely S1 shedding and subsequent conformational changes required for infection. It is also possible that IgG specific bivalent mechanisms like spike cross-linking, steric hindrance or virus aggregation are involved.

### 47D11 Targets a Conserved Hydrophobic Pocket in the RBD

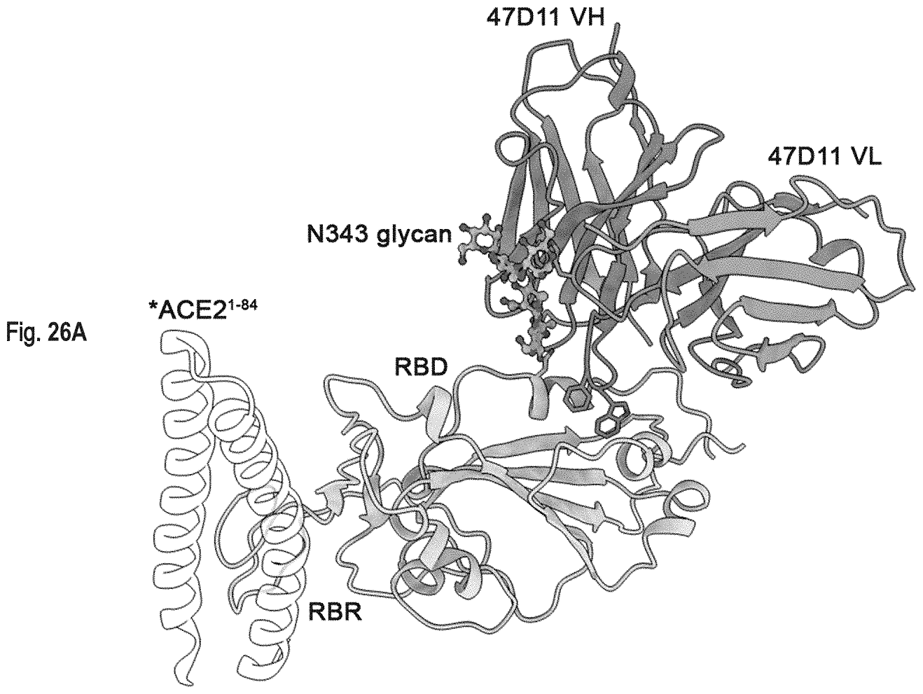

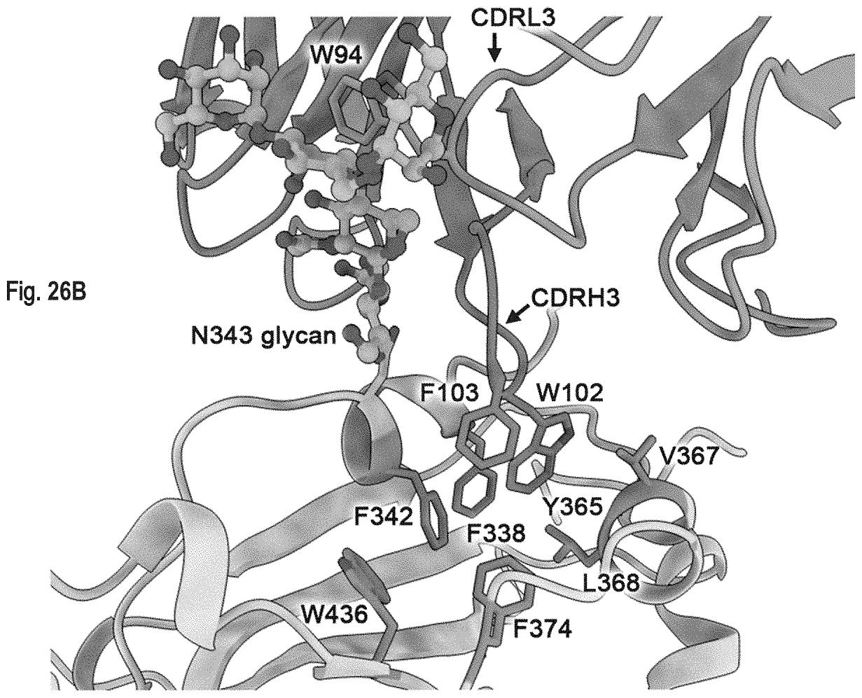

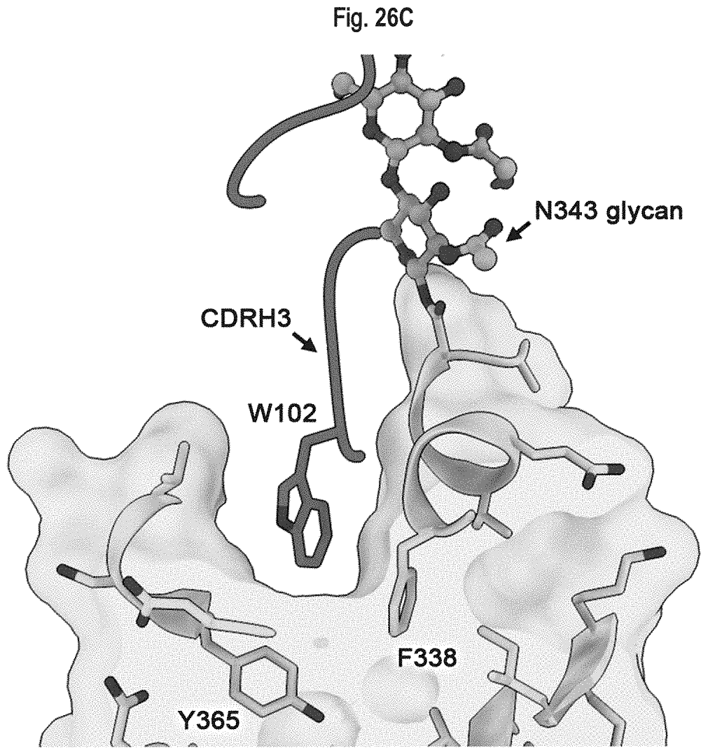

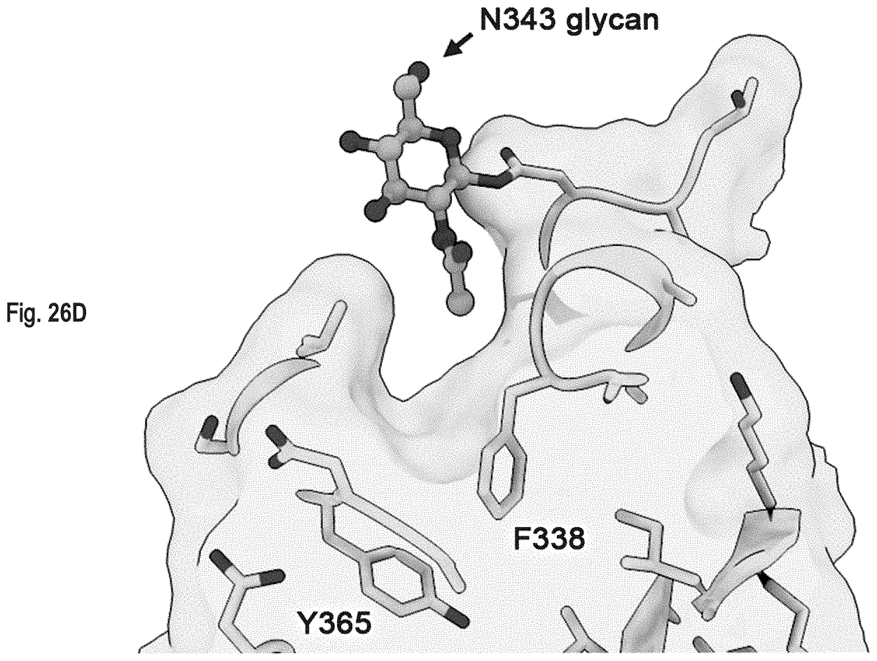

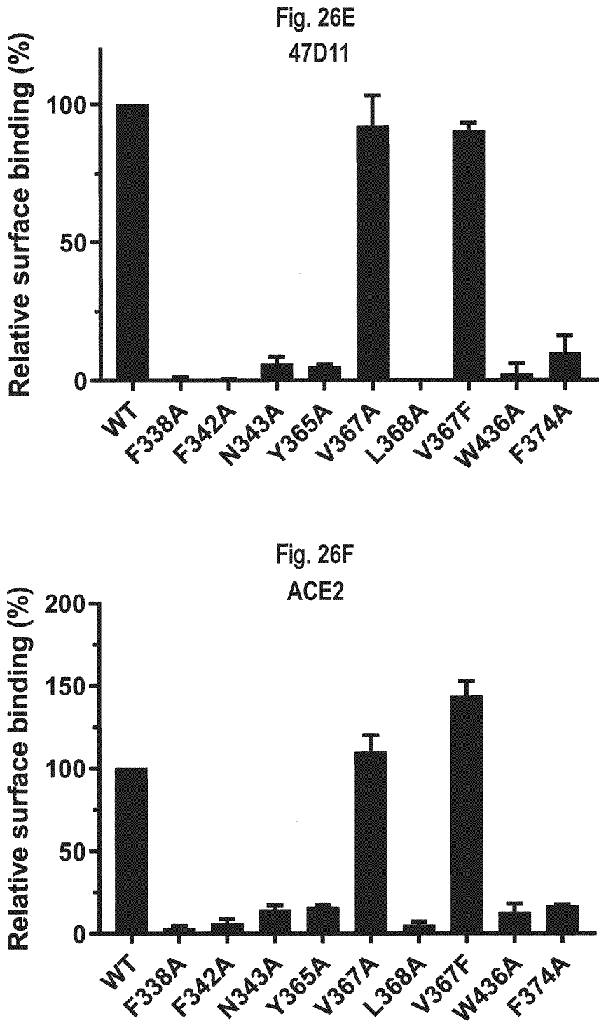

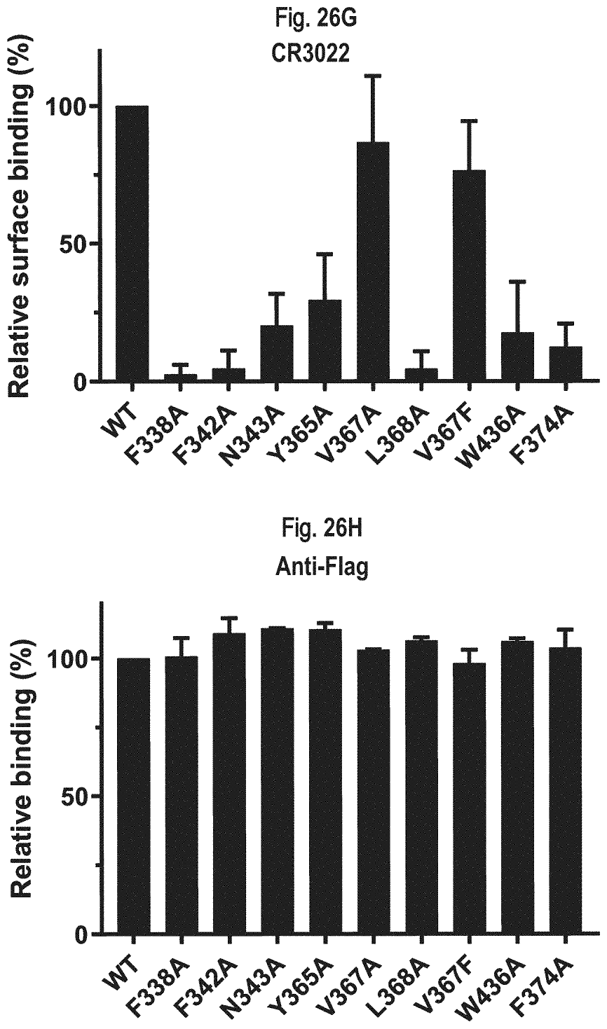

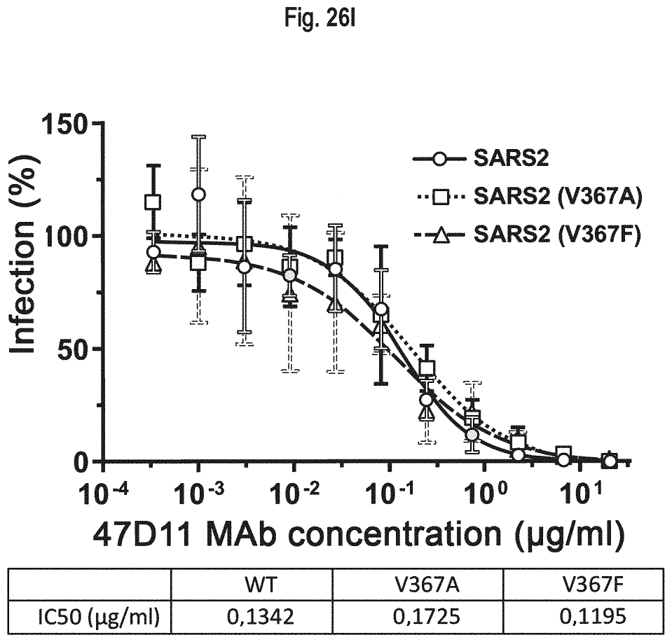

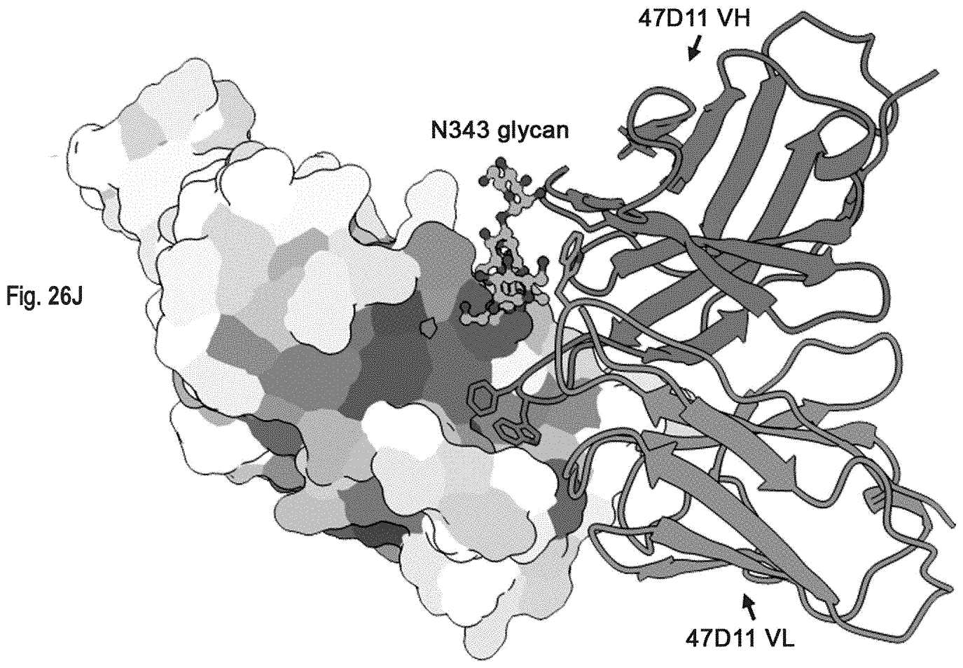

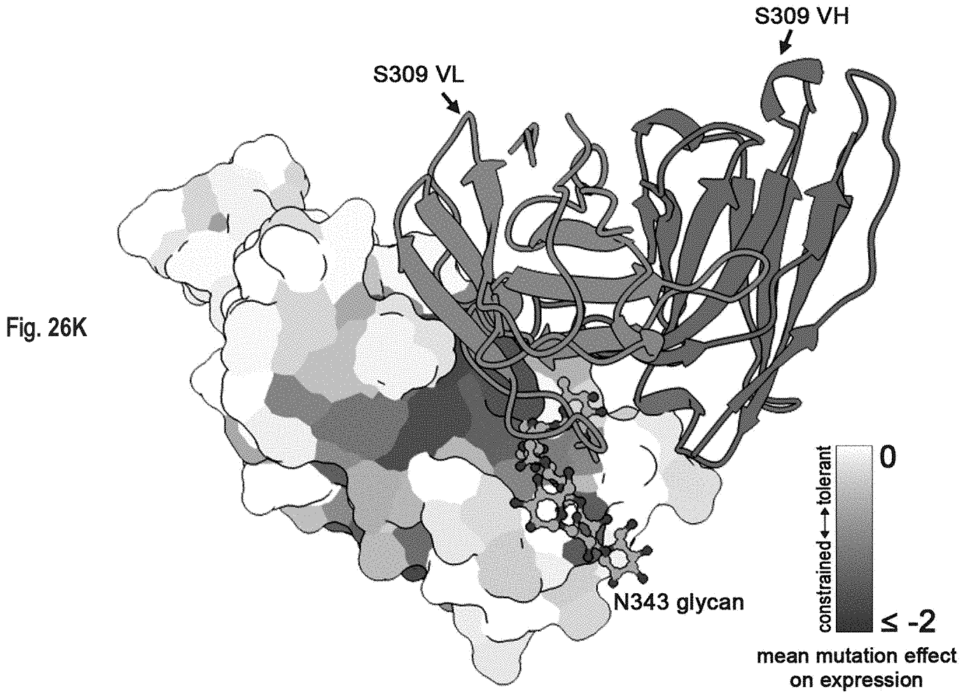

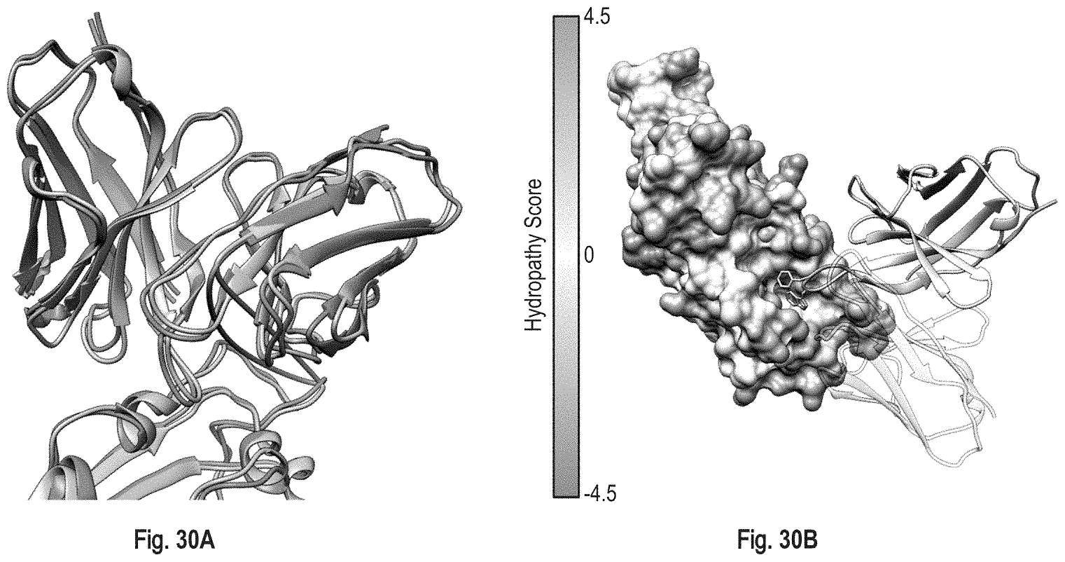

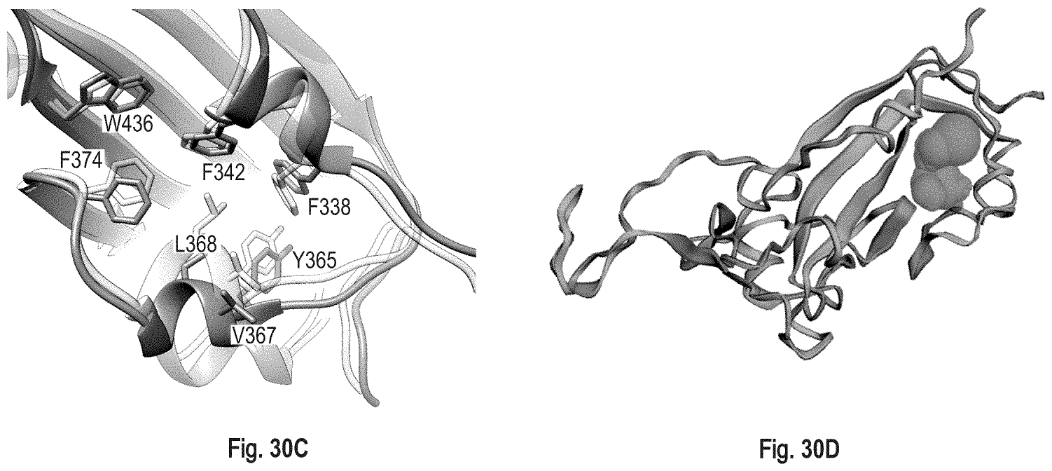

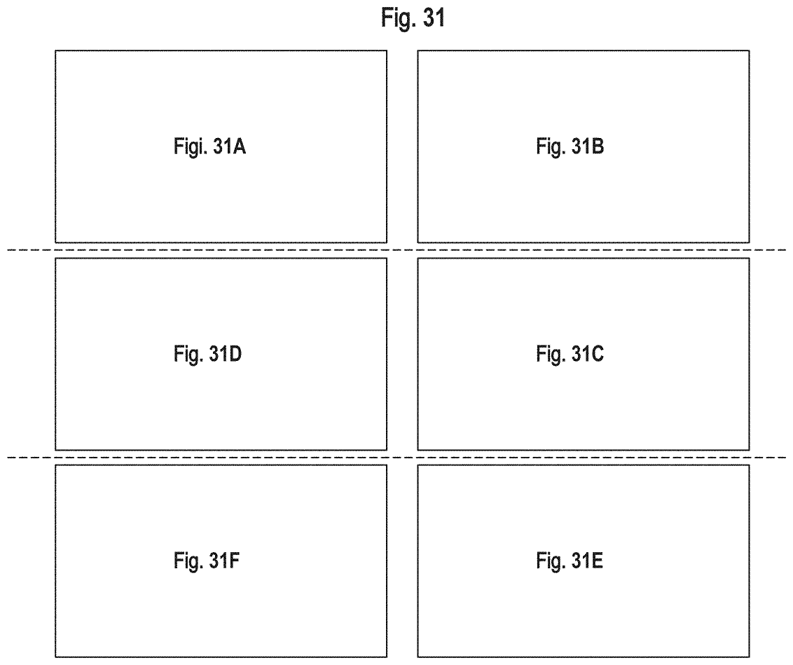

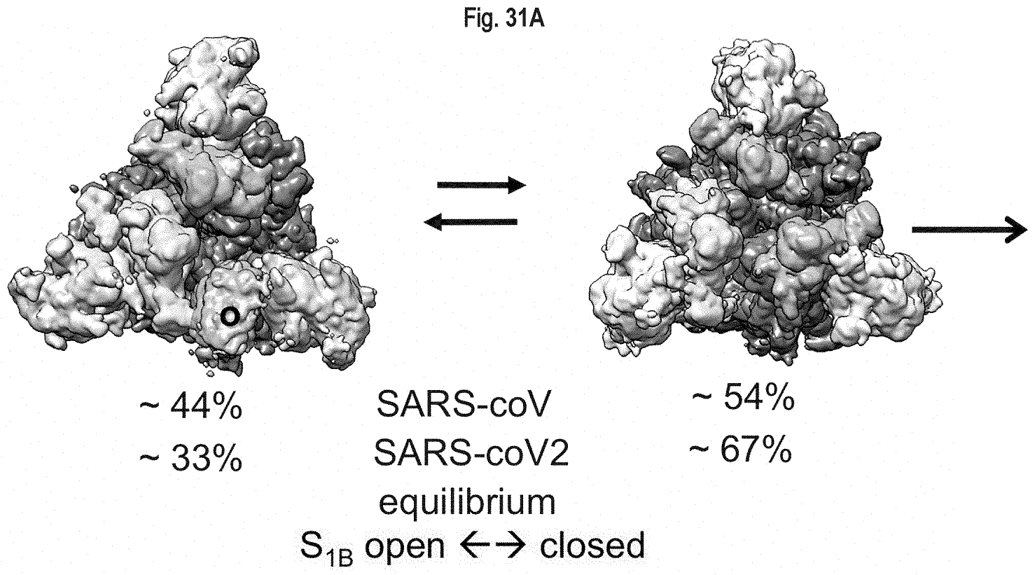

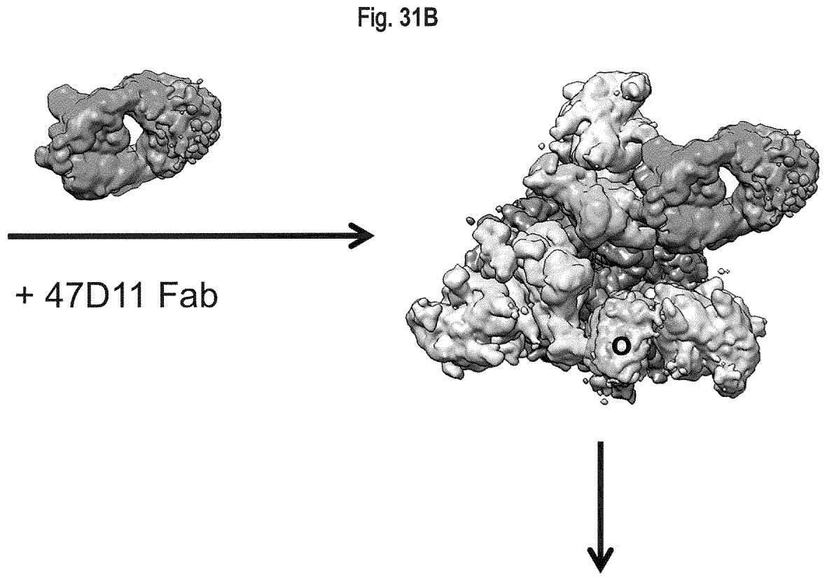

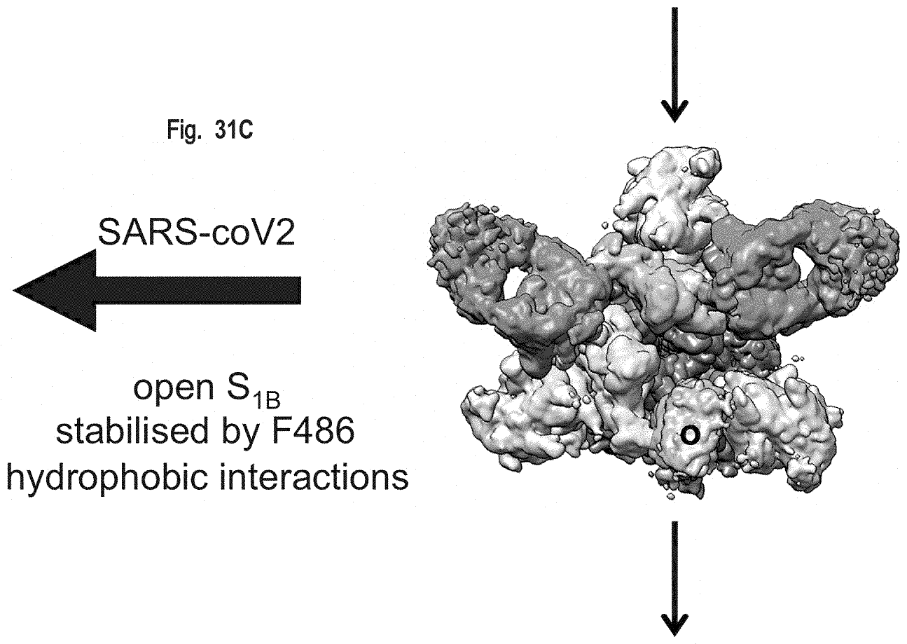

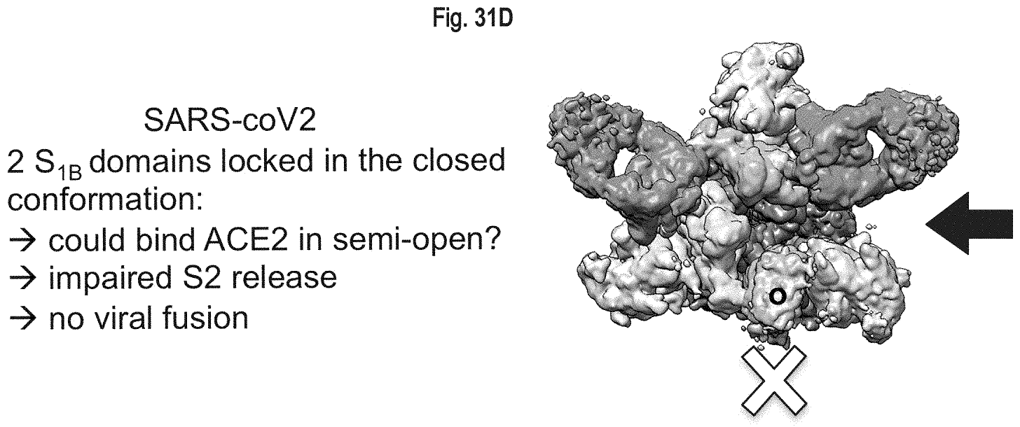

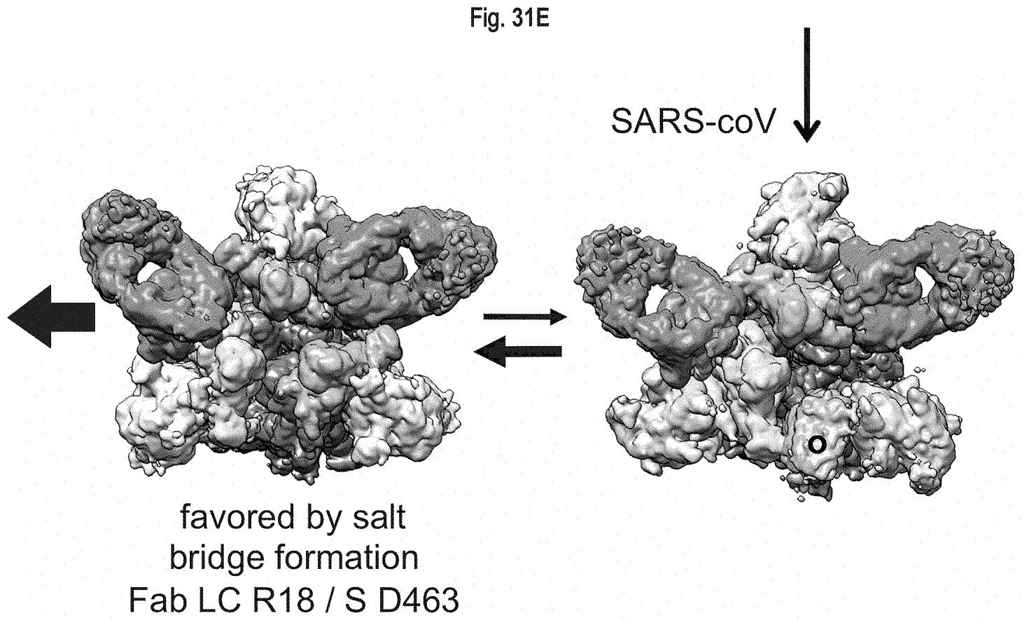

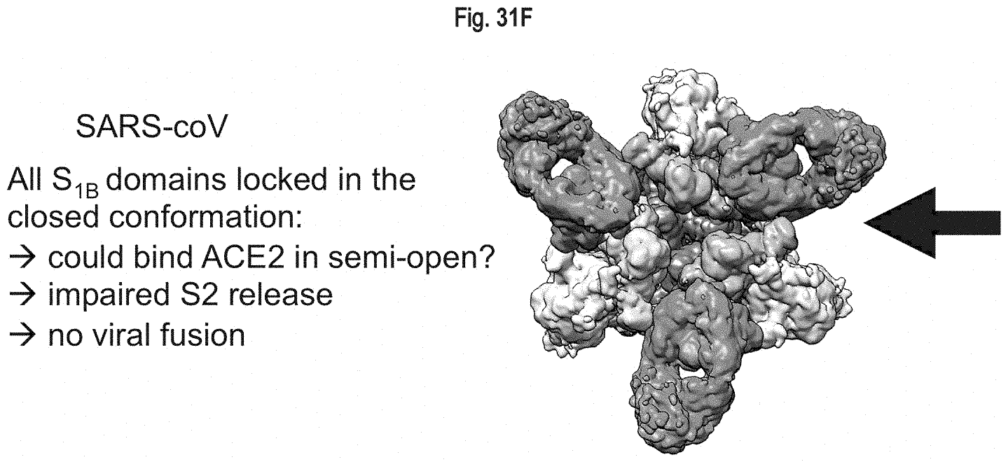

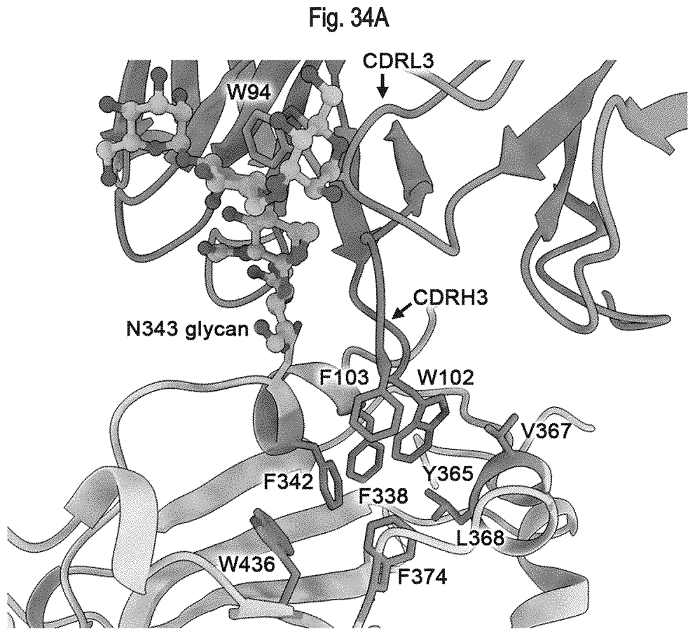

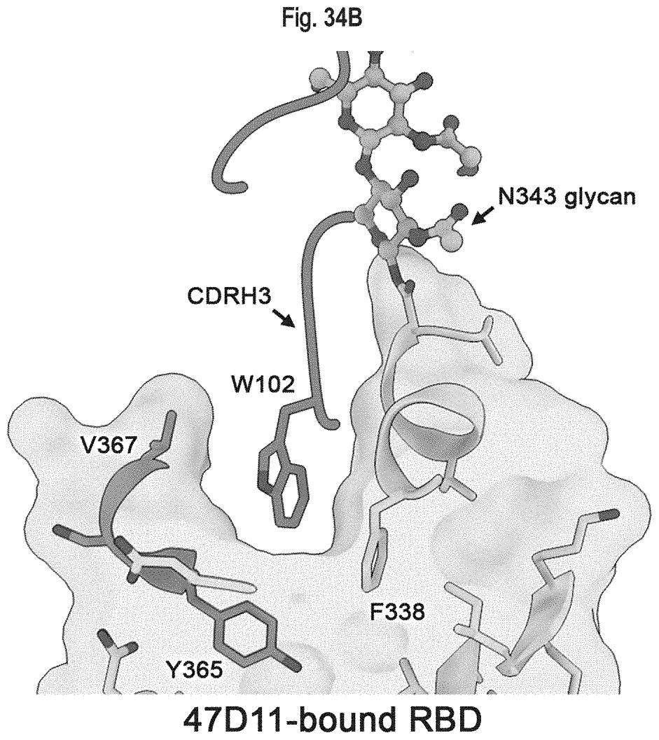

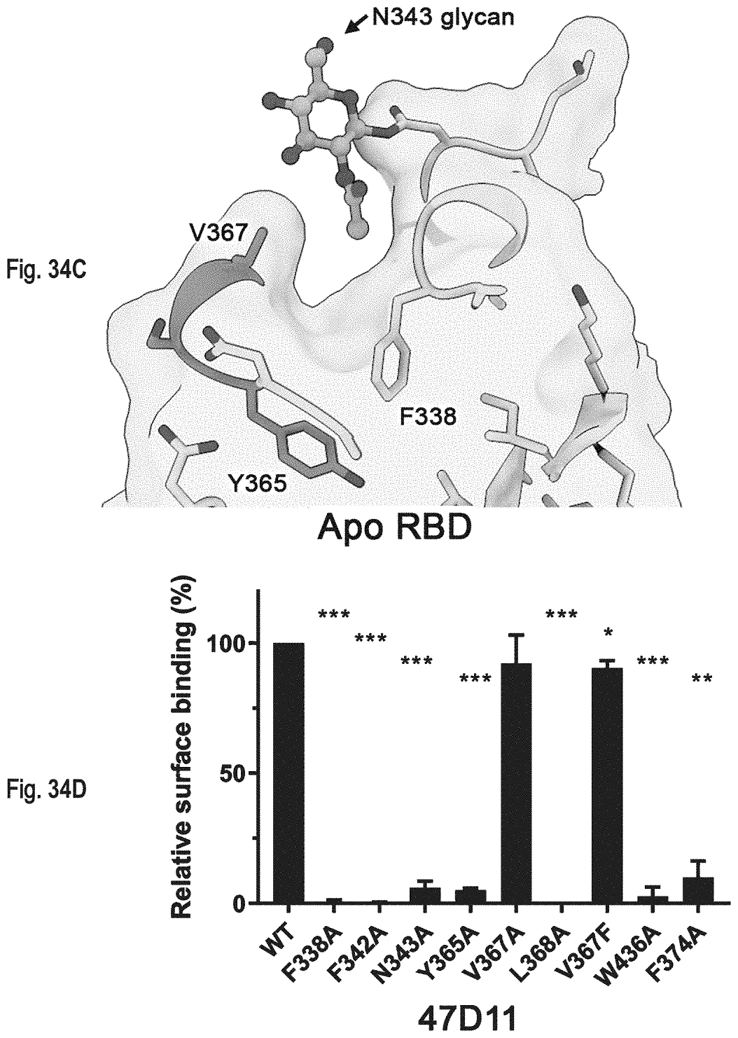

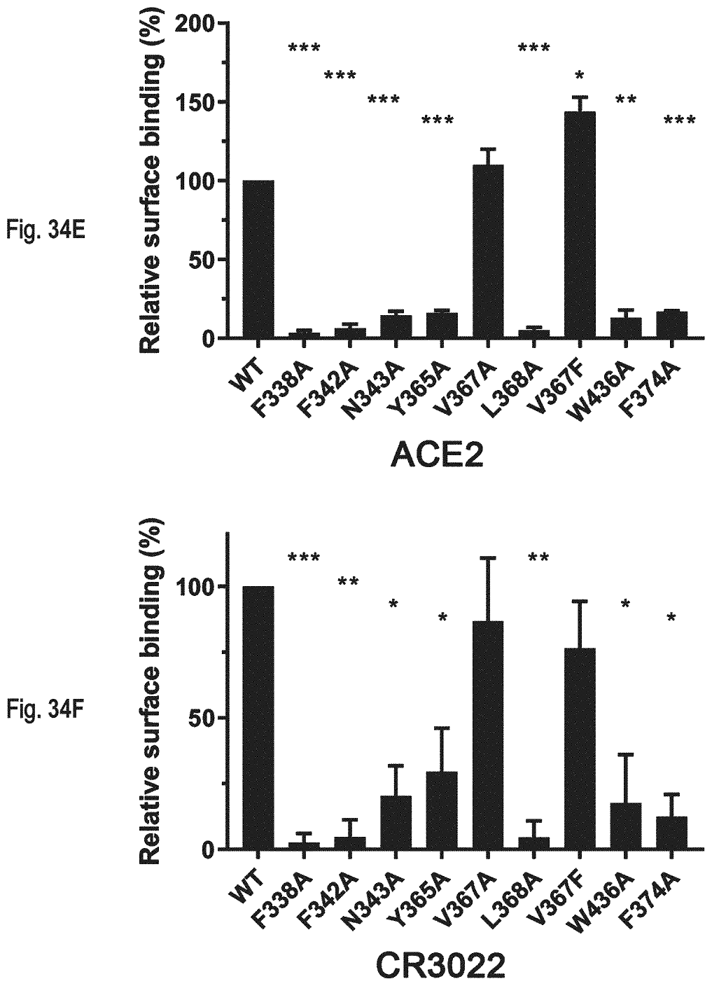

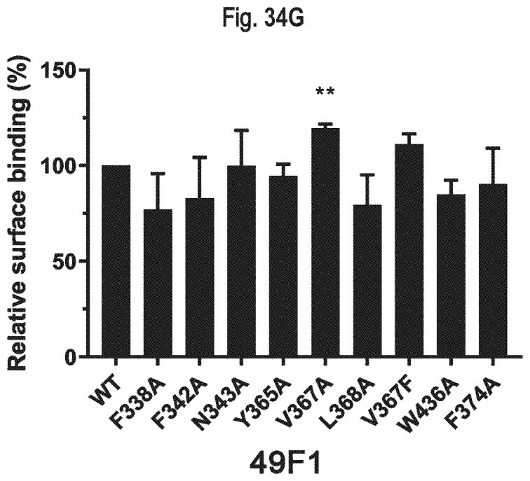

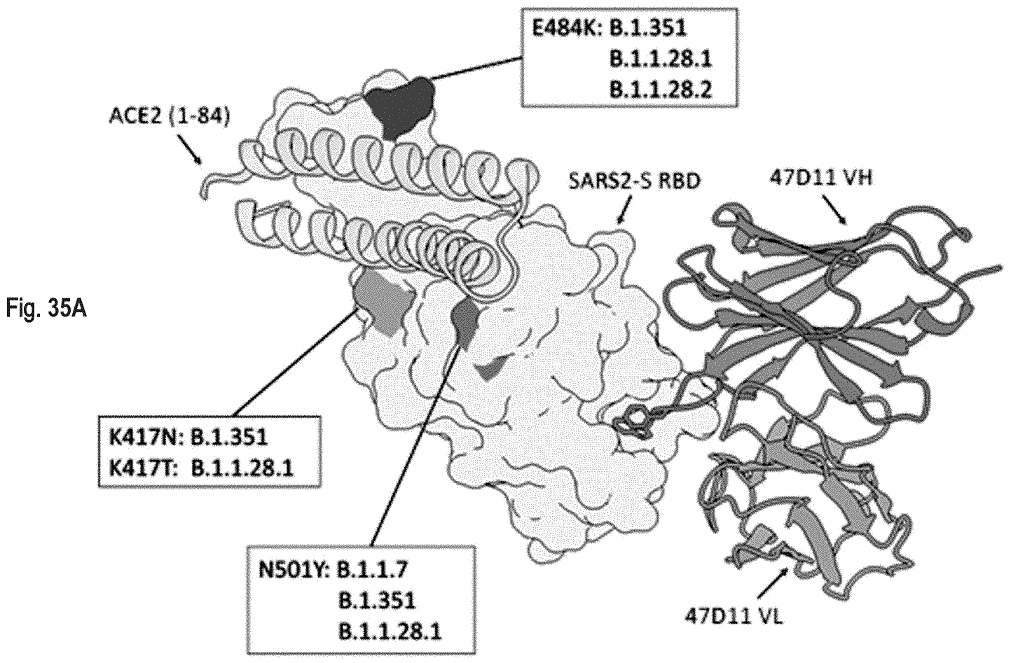

The 47D11 epitope is distinct from the ACE2 binding site (FIG. 26A), consistent with the functional data provided above. Thus, this rationalizes the ability of 47D11 to cross-neutralize SARS-CoV and SARS-CoV-2 independently of receptor-binding inhibition. The protein/glycan epitope is located on the core domain of the SARS1-S and SARS2-S RBD. The mode of binding is highly similar for SARS-S and SARS2-S (FIG. 30A-C), with the aligned 47D11:RBD complexes deviating by an RMSD value of 1.4 Å per 201 Ca atoms. The paratope is composed of CDRL3 and CDRH3 loops, that form a primarily hydrophobic interaction with the RBD surface of −830 Å² and −800 Å² for SARS1-S and SARS2-S, respectively. The side chain of 47D11 CDRL3 tryptophan W94 stacks against the N330/N343 (SARS1/SARS2) glycan tree, contributing to its stabilization in an upright conformation (FIGS. 26B and 34A). This reveals a hydrophobic pocket into which the CDRH3 loop projects, allowing Fab residues W102 and F103 to interact with RBD core residues F338, F342, Y365, V367, L368, F374 and W436 (F325, F329, Y352, V354, L355, F361 and W423 in SARS1-S)—FIGS. 26B, 34A and 27B. Interestingly, this pocket is normally shielded by the N343 glycan in previously reported apo SARS2-S structures (FIGS. 26C-D). In order to accommodate the CDRH3 loop residues, the helix encompassing residues 365-370 is displaced outwards by 2 Å (FIG. 30C), creating 55 Å³ of solvent accessible volume which is not present in the apo RBD (FIGS. 26C-D and 30E-F). Of note, the region directly below this hydrophobic pocket was recently shown bind to linoleic acid, which stabilises the closed conformation of spike by spanning two adjacent RBDs. However, the distance between the 47D11-bound RBDs is too great to be bridged by linoleic acid. In the 47D11-bound RBD, the position of the helix encompassing residues 365-370 would preclude binding of linoleic acid (FIG. 30E). Consistent with this arrangement, no density consistent with linoleic acid was present in any of our reconstructions.

To verify the 47D11 epitope, we introduced alanine mutations at each of the identified contact residues in the context of full-length spike protein. In addition, a spike mutant with the naturally occurring V367F minority variant was generated. Binding of 47D11 to surface expressed wildtype and mutant spike proteins was assessed by flow cytometry. Soluble Fc-tagged ACE2 and the RBD core binding mAb CR3022 were taken along as controls. The V367F substitution and the alanine substitute at this position only had a minor effect on 47D11 antibody binding (FIGS. 26E and 34D), consistent with data showing that this polymorphism had no effect on neutralisation of SARS2-S pseudo type virus (FIG. 26I). Collectively, this indicates that 47D11 would be effective against this SARS2 variant. In contrast, all other amino acid substitutions in the hydrophobic core not only reduced cell-surface binding by 47D11 (FIGS. 26E and 34D), but also prevented binding of ACE2 and the core targeting antibody CR3022, despite being distal to the receptor binding site (FIGS. 26F-G, 34E-F and 28). Total cellular expression of mutants was comparable to wildtype spike protein as demonstrated by an antibody targeting the C-terminal appended Flag-tag on the spike proteins (FIG. 26H), suggesting that mutations in the RBD hydrophobic core have a detrimental effect on protein folding, compromising the tertiary structure of the RBD.

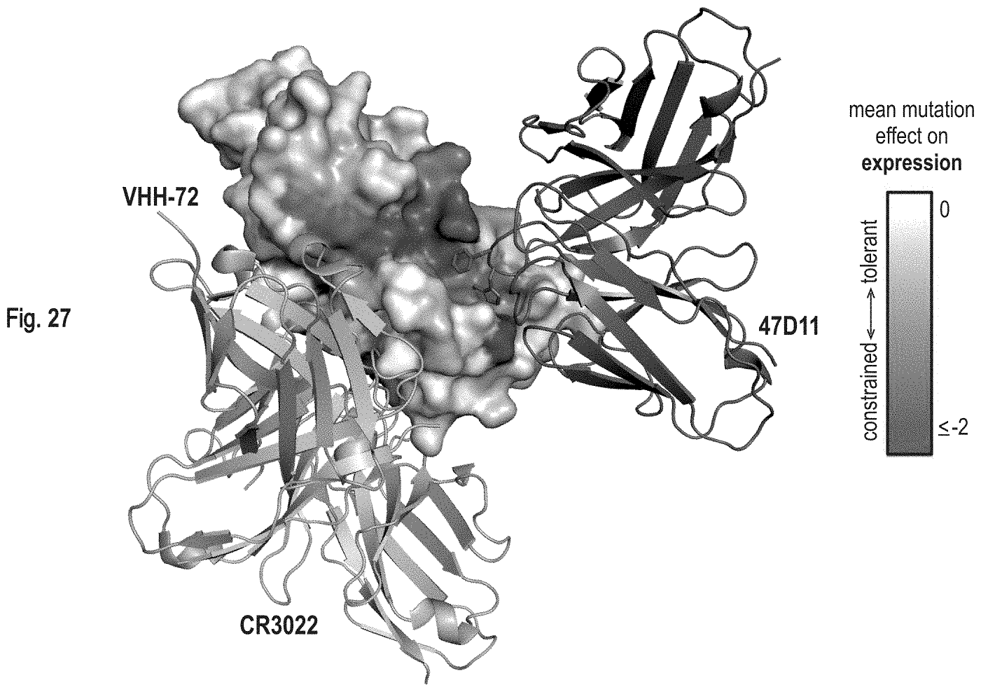

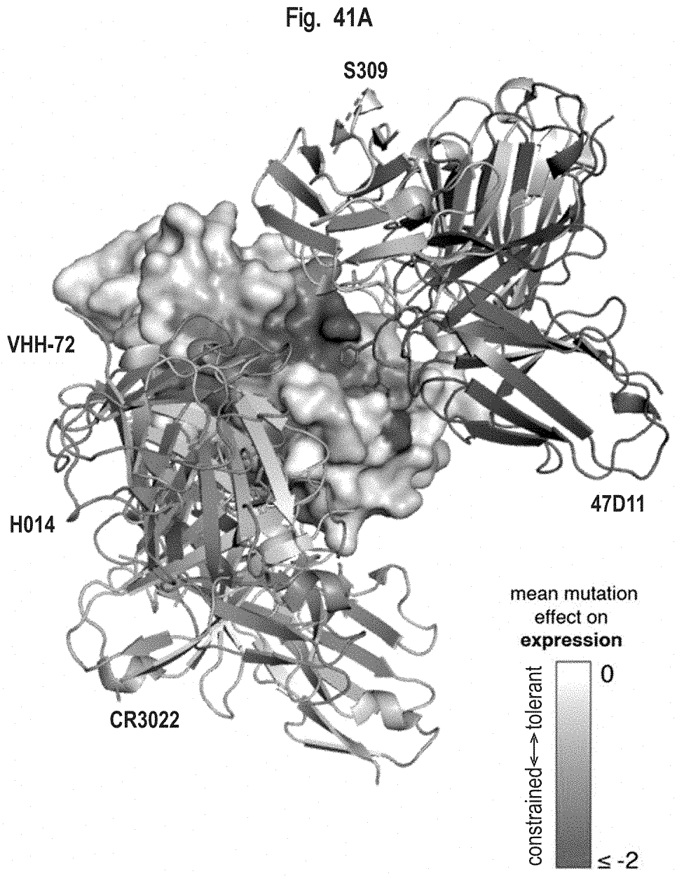

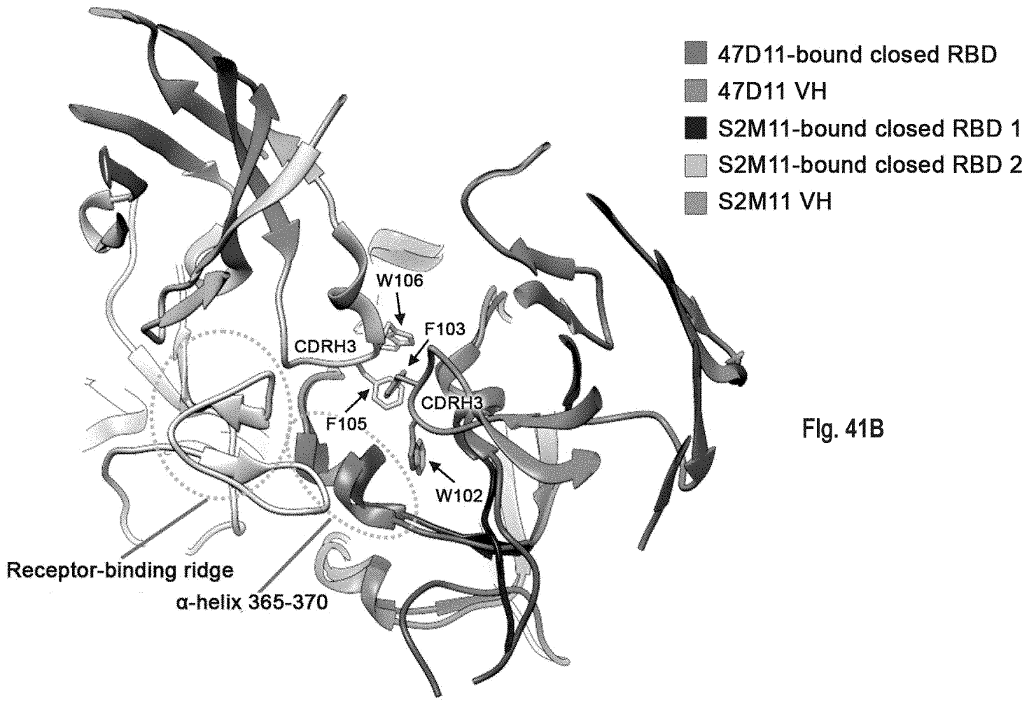

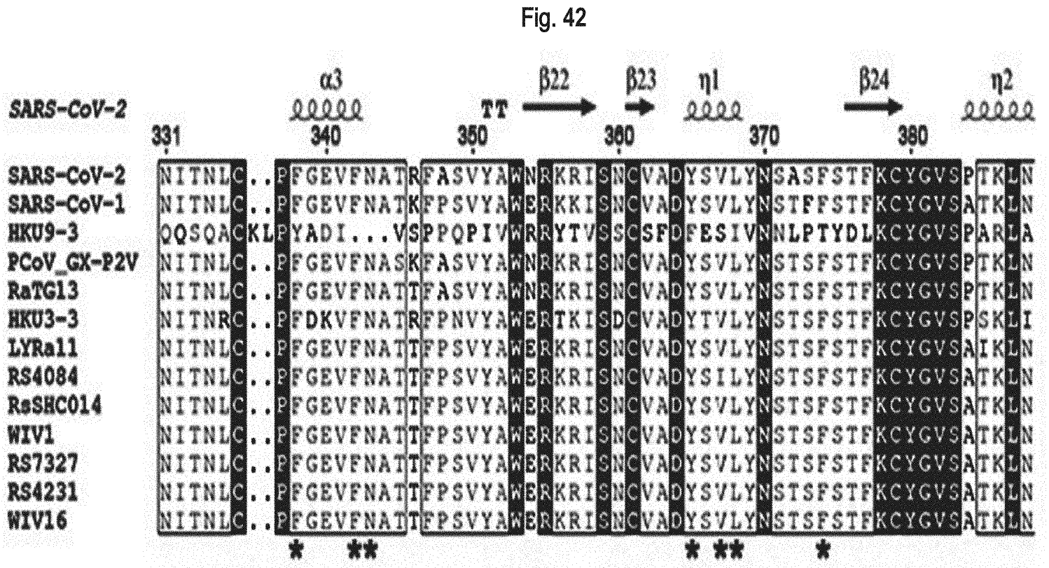

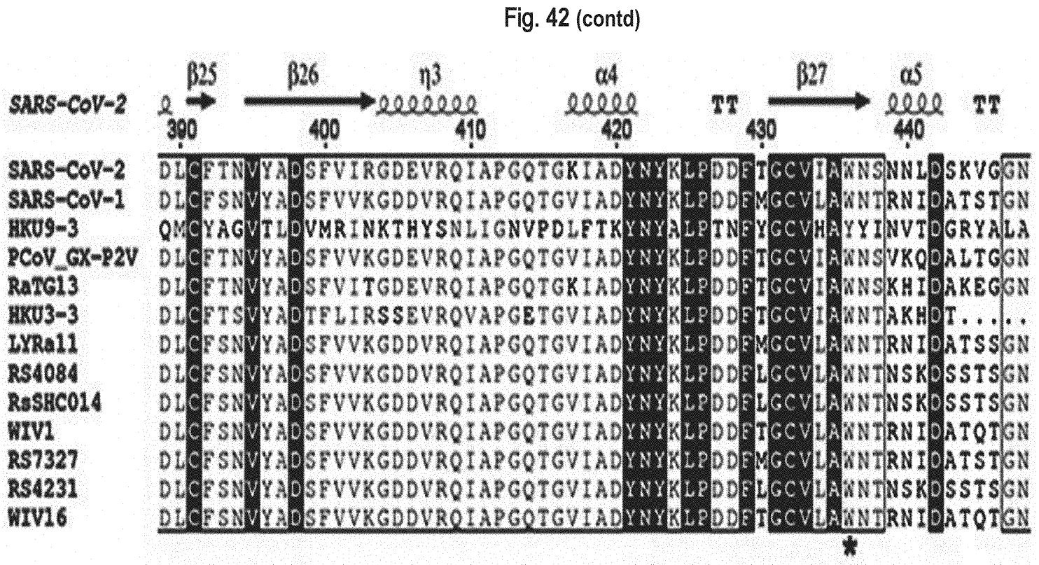

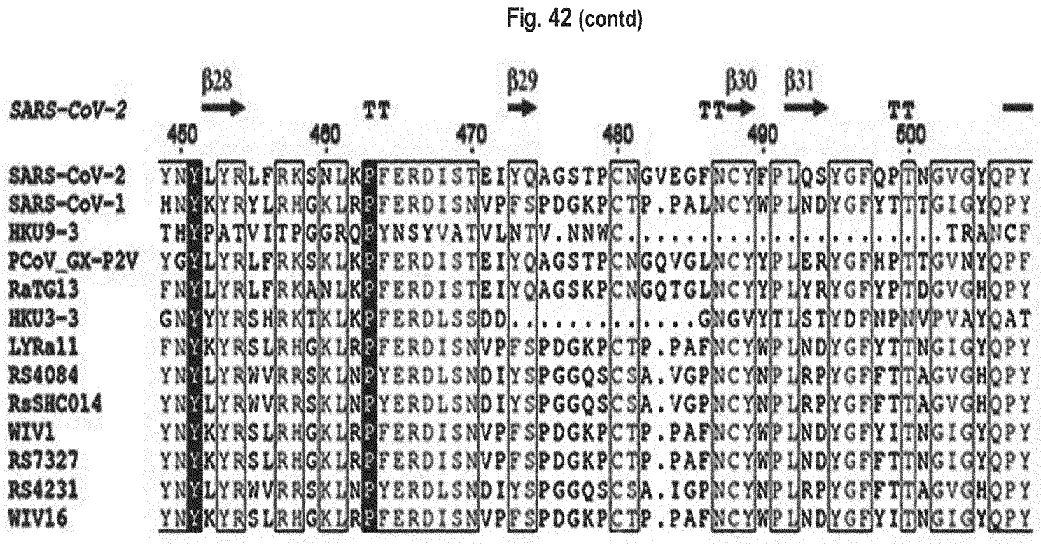

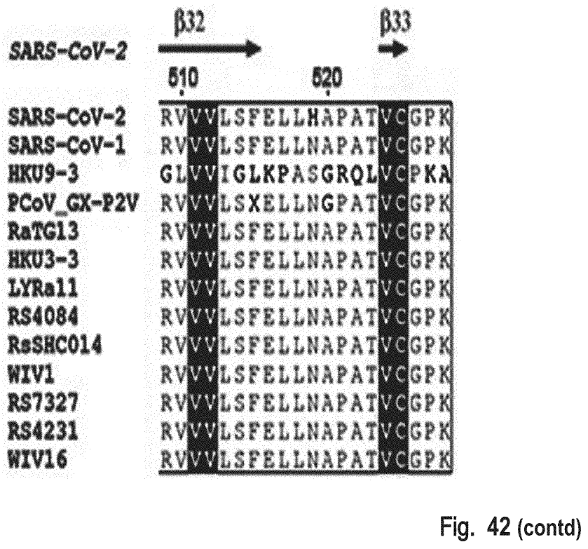

Binding of 47D11 to cell surface expressed wildtype and mutant spike proteins was assessed by flow cytometry. As controls, we used the soluble Fc-tagged ACE2, as well as the RBD core-targeting mAb CR3022 and the mAb 49F1, which binds S1 outside the RBD. Similar binding levels of the 49F1 antibody in wild type and mutant spike proteins confirmed the correct cell surface localization of the mutants (FIG. 34G). Mutations of V367 to a phenylalanine or an alanine had only a minor effect on 47D11 antibody binding (FIG. 34D), consistent with data showing that this polymorphism had no effect on neutralization of SARS2-S pseudotyped virus (FIG. 34H). Collectively, these data indicate that 47D11 would be effective against this SARS-CoV-2 variant. In contrast, all other amino acid substitutions in the hydrophobic core not only reduced 47D11 binding (FIG. 34D), but also prevented binding of ACE2 and of the core targeting antibody CR3022, despite being distal to their respective interaction sites on the RBD (FIG. 34E-F, FIG. 27A-B and FIG. 41A-B). These results suggest that these mutations have an effect on the tertiary structure of the whole RBD including the distal ACE2 binding ridge.

In line with this explanation, a recent study reported deep mutational scanning of SARS2-S RBD residues, revealing how mutation of each of the RBD residues affects expression of folded protein and its affinity for ACE2 (Starr et al. (2020) Cell; doi:10.1016/j.ce11.2020.08.012). When the mean mutation effect on expression was mapped on the 47D11 bound RBD, we observed that the hydrophobic pocket, targeted by 47D11, is highly mutationally constrained (FIG. 26J). Altogether, the mutational space in the 47D11 epitope region appears to be strongly limited by a concomitant loss of ACE2 binding, possibly lowering the risk of immune escape. The 47D11 epitope matches a region of the RBD earlier described as relatively “immune silent” (37). It is distinct from other reported RBD-core targeting antibodies/nanobodies, such as CR3022, H014 and VHH-72 (FIG. 27A and FIG. 41A). Another SARS1 and SARS2 neutralising antibody, 5309, targets a similar region to 47D11, but here the orientation of the N343 glycan prohibits access to the hydrophobic pocket, similarly to apo structures (FIG. 26K). The 47D11 epitope is distinct from other reported RBD-core targeting antibodies/nanobodies, such as CR3022 and VHH-71 (FIG. 27 and FIG. 41). The quaternary epitopes of the SARS-CoV-2 neutralizing antibodies C144 (Barnes et al. (2020) Nature 588:682-7) and S2M11 (Tortorici et al. (2020) Science 370(6519):950-7), isolated from COVID-19 recovering patients, also include the conserved hydrophobic pocket targeted by 47D11 (FIG. 41B). However, the C144 and S2M11 epitopes extend to the RBM of the adjacent RBD—which is not conserved between SARS-CoV and SARS-CoV-2. Our data show that the 47D11 epitope, being restricted to a single RBD core, accounts for its cross-neutralization ability, while C144 and S2M11 fail to neutralize SARS-CoV.

### 47D11 Neutralizes Emerging SARS-CoV-2 Variants

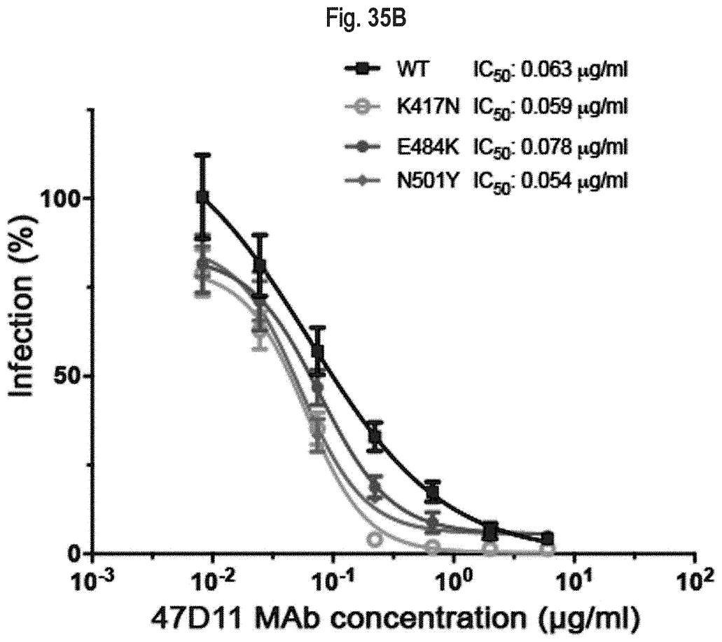

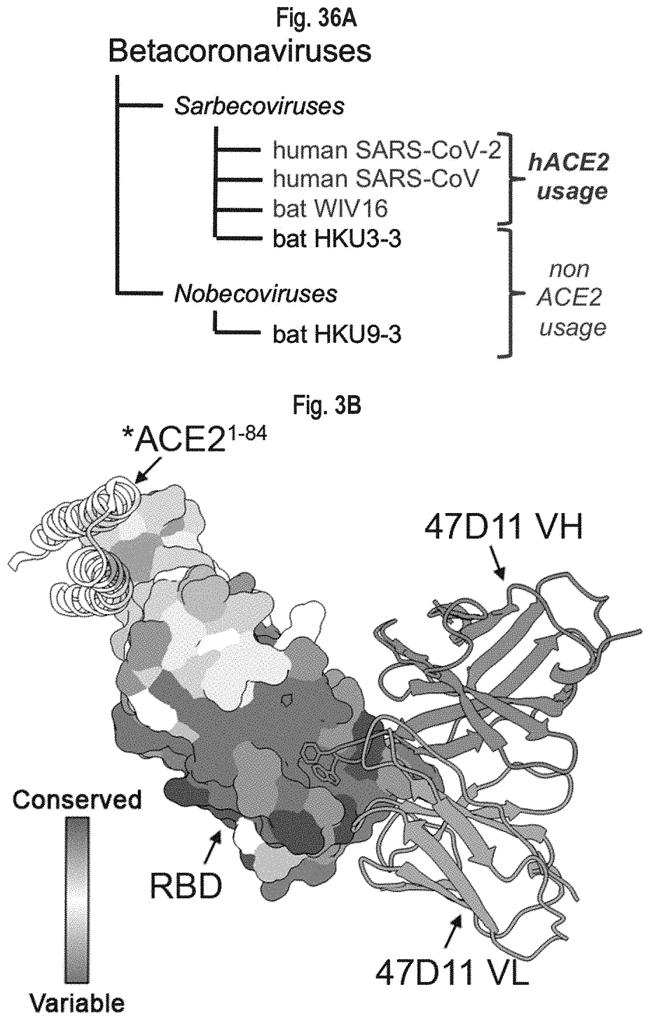

The recently emerging SARS-2 variants carry mutations in the RBD, around the ACE2 binding motif, namely K417N, E484K and N501Y (FIG. 35A). While these mutations are distal to the 47D11 epitope, our data reveals crosstalk between the RBM and the 47D11 epitope region, as mutations in the 47D11 epitope resulted in loss of ACE2 binding (FIG. 30E-F). Reciprocally, we analyzed the effect of emerging mutations in the RBM on 47D11 neutralization ability by introducing the K417N, E484K or N501Y mutation into SARS2-S pseudotyped VSV. Our data show that 47D11 neutralization efficiency is not affected by these mutations in the RBM (FIG. 35B), making 47D11 a candidate of choice in the fight against the new, fast spreading SARS-CoV-2 variants.

47D11 Displays Broader Reactivity Among at-Risk Bat Coronaviruses

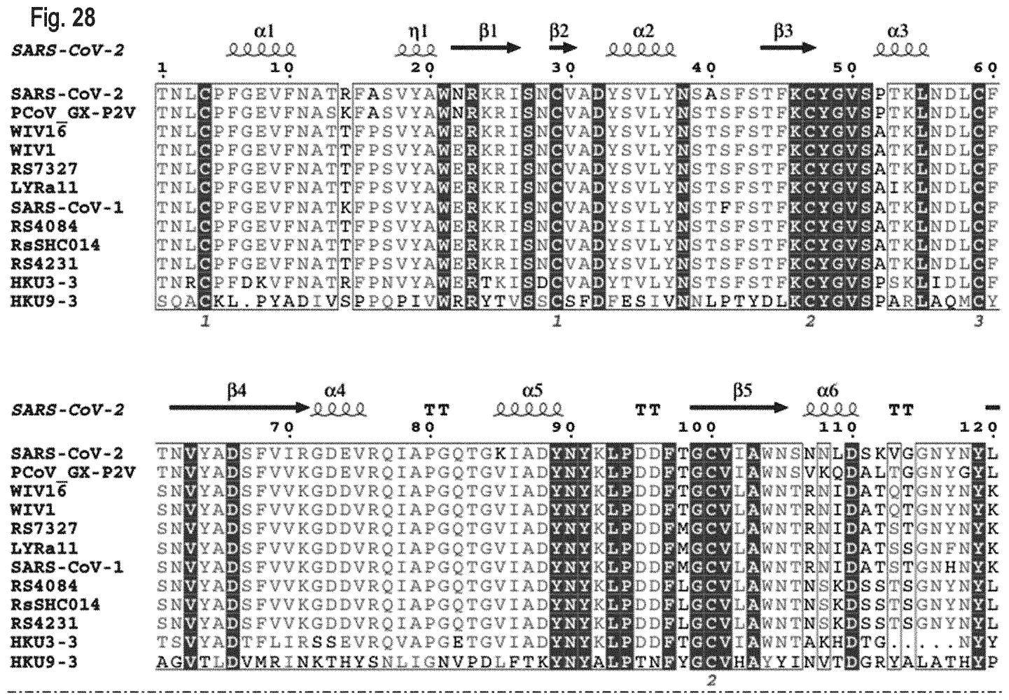

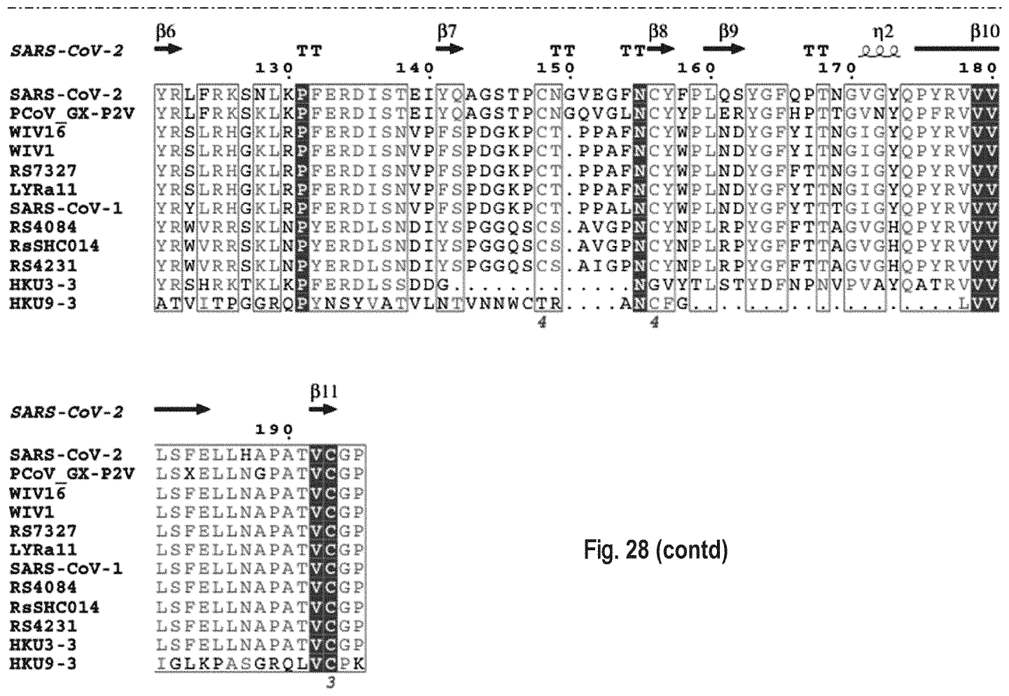

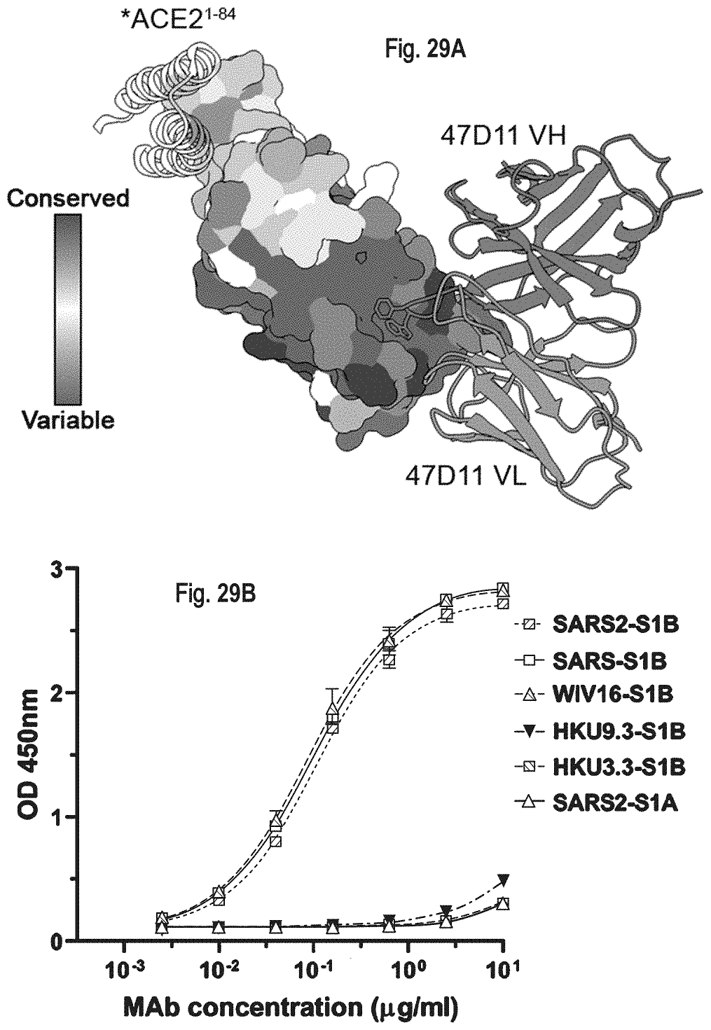

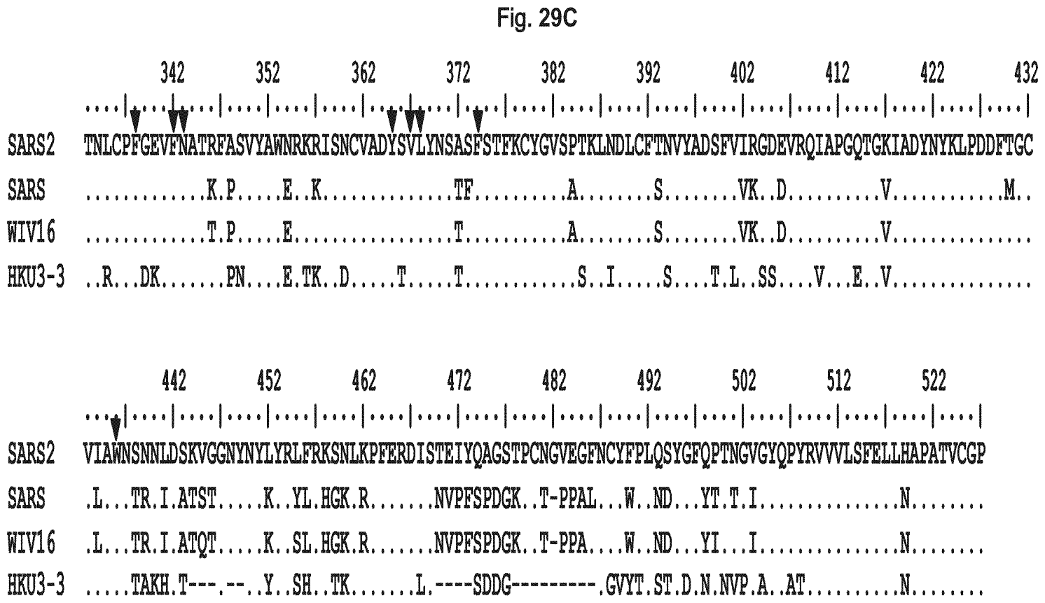

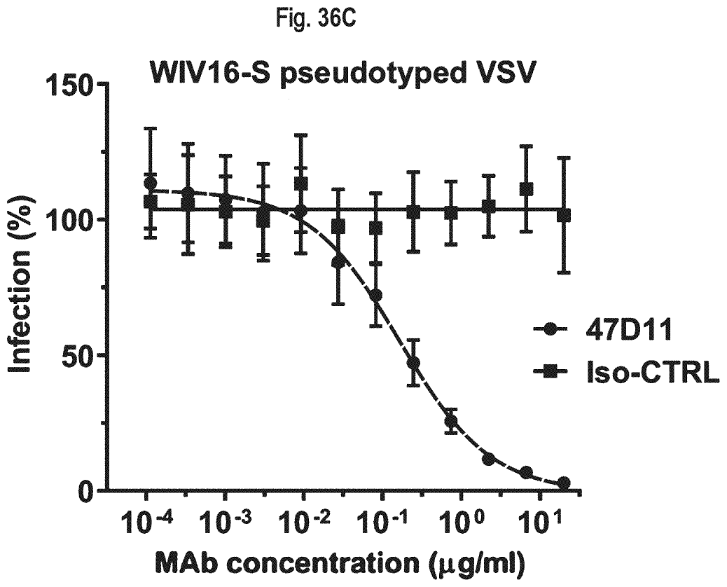

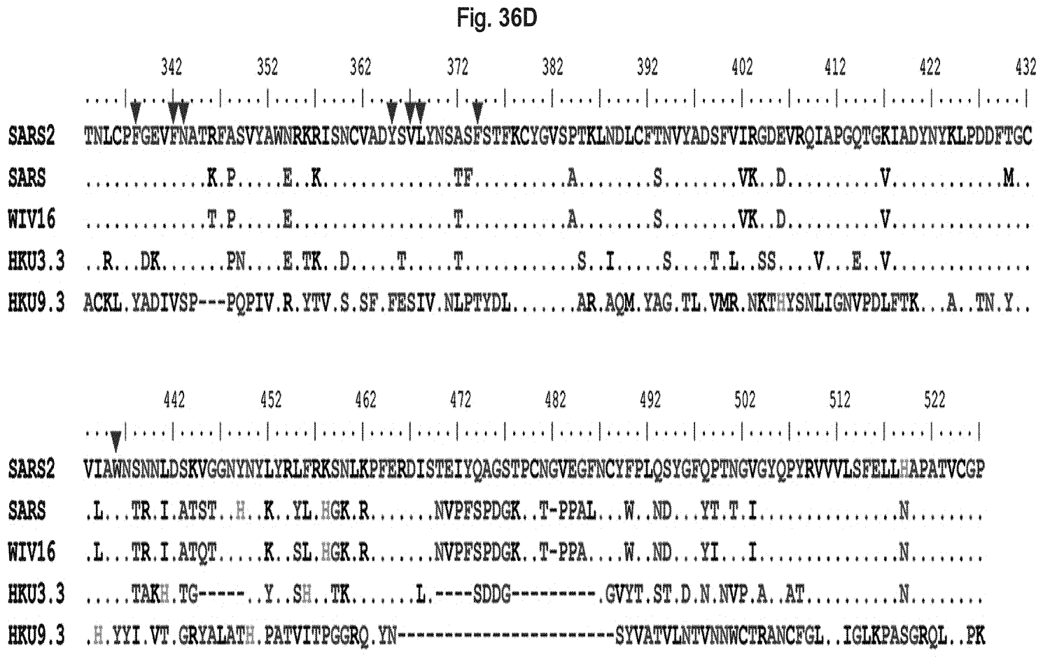

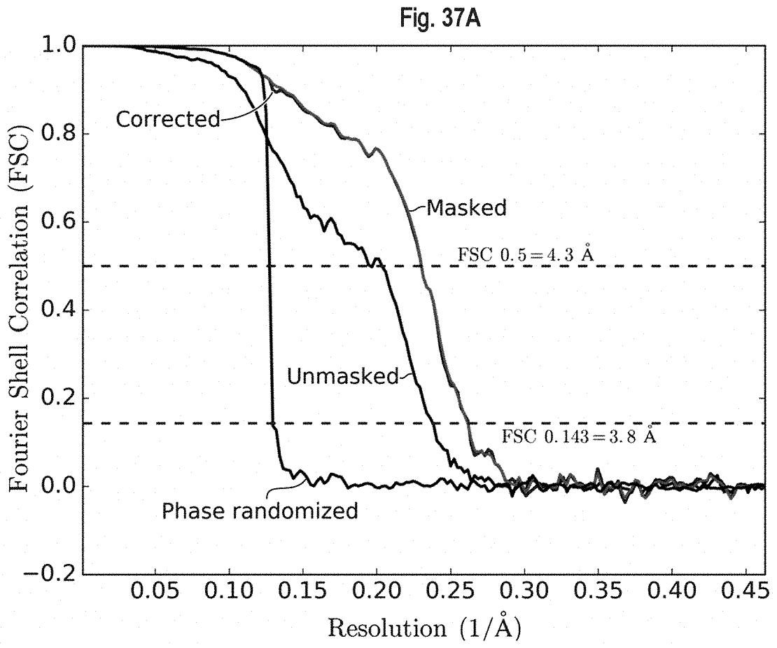

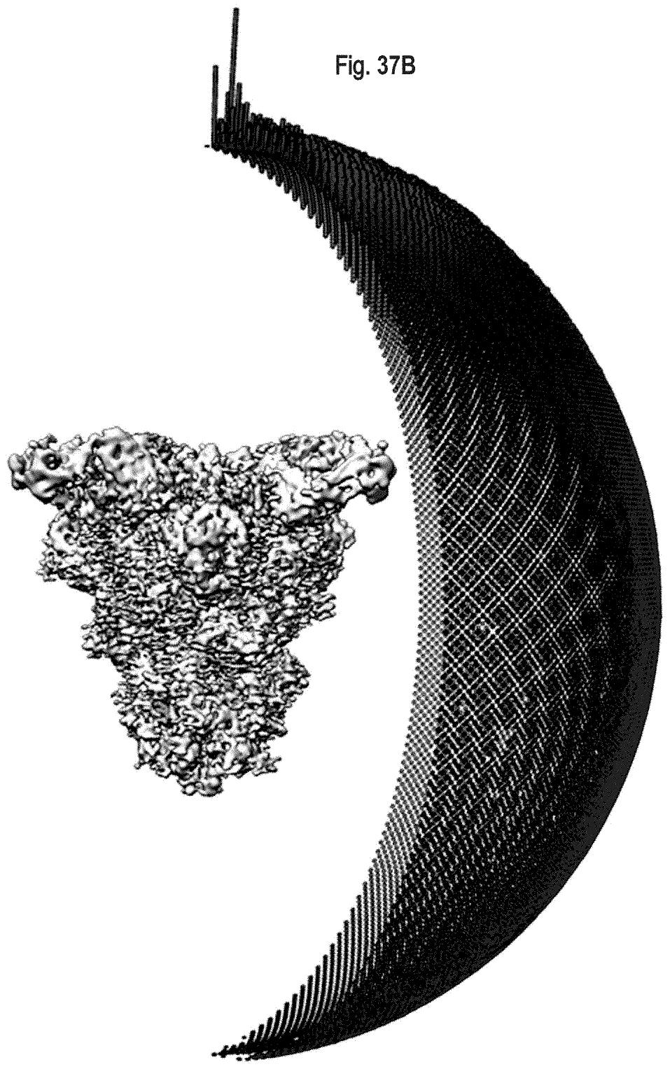

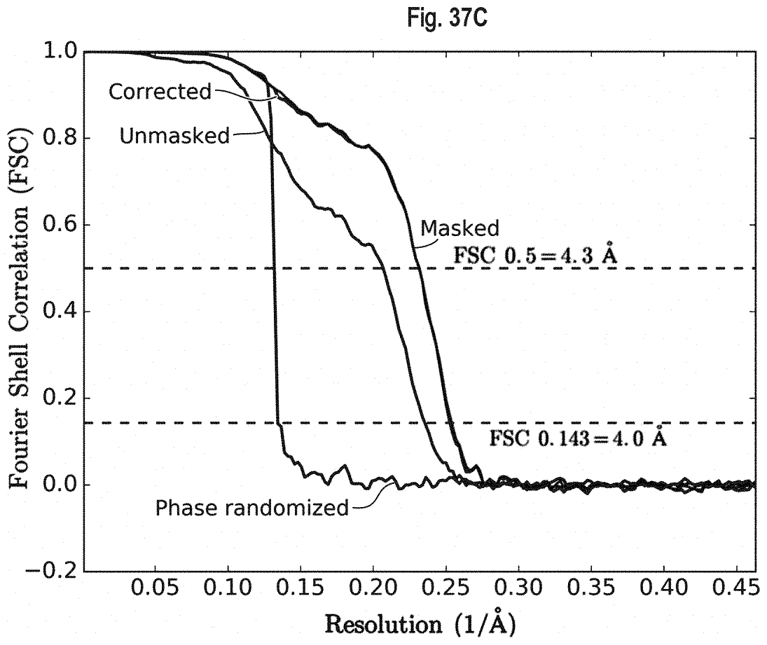

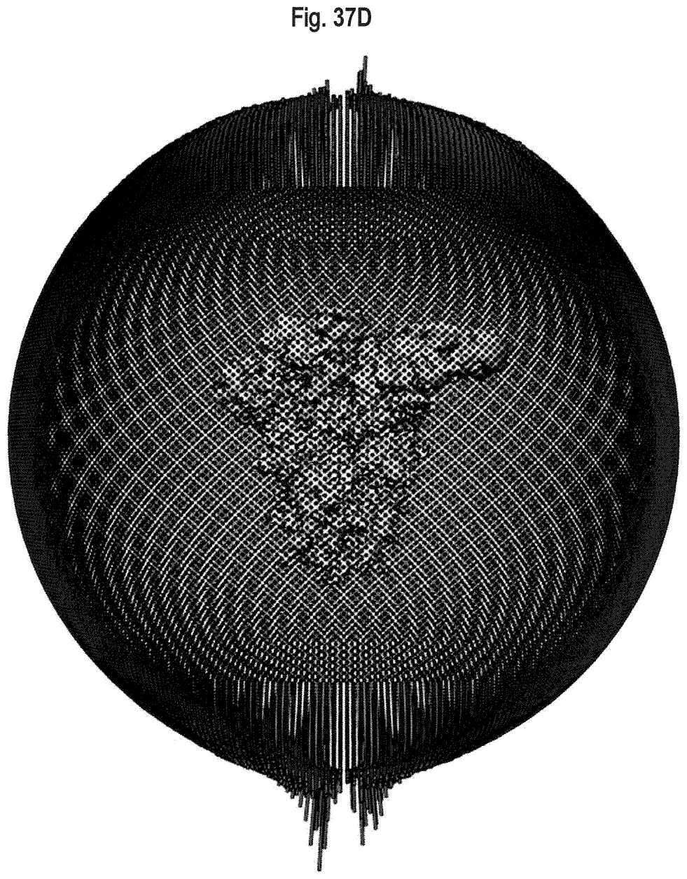

In order to assess whether 47D11 has broad reactivity, we analyzed 47D11 binding to the recombinantly expressed RBDs of 3 SARS-like bat betacoronaviruses: the sarbecoviruses WIV16 and HKU3-3, and the more distant nobecovirus HKU9-3 (FIG. 36A). Comparative sequence analysis revealed that the 47D11 epitope is highly conserved across circulating SARS-like viruses (FIGS. 28 and 29B). This is in contrast to the ACE2 binding region which exhibits the greatest sequence variability. In order to assess whether 47D11 has broad reactivity, we recombinantly expressed the RBD from WIV16, HKU3-3 and HKU9-3 in order to assess binding to these related sarbecoviruses. The results demonstrated that 47D11 can bind to the WIV16 RBD with similar affinity to SARS1-S and SARS2-S (FIG. 29B). The results also demonstrated that 47D11 neutralizes WIV16-S pseudotyped VSV with an IC₅₀ value of 0.165 μg/ml (FIG. 36C). In contrast, 47D11 does not bind the RBDs of HKU3-3 and HKU9-3 (FIG. 36B). The potent binding observed for WIV16 underscores the potential of 47D11 as a treatment for future outbreaks caused by SARS-like viruses.

HKU9-3 has the most distantly related RBD sequence to SARS-CoV-2 and the N343 glycosylation site, as well as the hydrophobic residues of the 47D11 epitope, are not conserved, explaining lack of antibody binding (FIG. 36D). In both WIV16 and HKU3-3 the 47D11 epitope is conserved but HKU3-3 displays 4 amino acid variations introducing charges in close proximity to 47D11: L335R, 339GE340 to DK and N360D, which could preclude 47D11 binding. Of note, unlike WIV16, neither HKU3-3 nor HKU9-3 is able to bind human ACE2 (Wells et al. (2020) The evolutionary history of ACE2 usage within the coronavirus subgenus Sarbecovirus bioRxiv). Sarbecoviruses (which include SARS-CoV, SARS-CoV-2, numerous bat and a few pangolin viruses) are considered to be a high-risk group for potential emergence. Both sarbecoviruses that cause human disease, SARS-CoV and SARS-CoV-2, use the human ACE2 for cell entry and hence it is thought to be a trait for particular importance in the emergence pathway of sarbecoviruses. Our results provide a proof of principle that 47D11 could contribute to treatments for future outbreaks caused by ACE2-dependent SARS-like viruses.

In conclusion, this structural and functional analyses demonstrate that 47D11 is able to unmask a conserved and mutationally constrained epitope on the SARS-CoV-2 RBD by stabilising the N343 glycan in an upright conformation. Once this site is exposed, the CDRH3 loop is able to insert two aromatic residues into the hydrophobic core of the RBD inducing conformational changes which lead to the formation of a 55 Å³ cavity. This cryptic site offers an attractive target for design of vaccines and targeted therapeutics, such as small molecule inhibitors. The 47D11 epitope is distal to the ACE2 receptor binding motif, rationalising its ability to cross-neutralize SARS-CoV and SARS-CoV-2 independently of receptor-binding inhibition. Our structural analysis also shows that 47D11 exhibits differing conformational selectivity for SARS-S and SARS2-S, providing a possible explanation for the differences in observed binding affinity. Given that 47D11 selects for the partially open conformation of the SARS2 spike protein, it is proposed that 47D11 may render the SARS2-S more susceptible to other monoclonal antibodies which target the exposed receptor-binding subdomain, making it a prime candidate for combination treatment. Antibody combinations targeting non-overlapping epitopes may act synergistically permitting a lower dosage and an increased barrier to immune escape. While genetic diversity for SARS2 is currently limited as a result of a genetic bottleneck during spill-over from animals to human, this will increase in time through antigenic and genetic evolution. The limited mutational space of the epitope targeted by 47D11, may confer the antibody sustainable applicability in neutralizing a wide range of future-emerging virus variants.

In summary, our structural and functional studies demonstrate that 47D11 achieves cross-neutralization of the sarbecoviruses SARS-CoV-2 and SARS-CoV, by targeting a glycan shielded, conserved pocket on the spike RBD. This cryptic site of vulnerability offers an attractive target for the design of cross protective vaccines and targeted therapeutics. Genetic diversity of SARS-CoV-2 has recently increased and the genetic/antigenic variation will rise further in time, as observed for the endemic human coronavirus HCoV-229E, which exhibits cumulative sequence variation in the RBD loops engaging its cellular receptor. The conserved nature of the glyco-epitope, evolutionary constrained by limited mutational space, may confer the 47D11 antibody sustainable applicability in neutralizing a wide range of future-emerging virus variants. Antibody combinations targeting non-overlapping epitopes are currently of high interest as they may act synergistically permitting a lower dosage and an increased barrier to immune escape. In this respect, and unlike C144 and S2M11, which lock the SARS2-S in its closed conformation, our structural data shows that 47D11 stabilizes the partially open conformation of the SARS2-S. This may render the spike more susceptible to other monoclonal antibodies which target epitopes only exposed in the RBD up conformation—like H014, CR3022 or antibodies targeting the ACE2 binding ridge, thus making 47D11 a prime candidate for combination treatment.

Altogether our results delineate a conserved vulnerability site on the SARS-CoV-2 spike and provide a fundamental insight to support the rational development of antibody-based interventions in the treatment of COVID-19.

### Methods

### Expression and Purification of Coronavirus Spike Proteins

To express the prefusion spike ectodomain, gene encoding residues 1-1200 of SARS2 S (GenBank: QHD43416.1) with proline substitutions at residues 986 and 987, a “AAARS” substitution at the furin cleavage site (residues 682-685) and residues 1-1160 of SARS S (GenBank: AAP13567.1) with proline substitutions at residues 956 and 957, a C-terminal T4 fibritin trimerization motif, a StrepTag was synthesized and cloned into the mammalian expression vector pCAGGS. Similarly, pCAGGS expression vectors encoding 51 or its subdomain S1B of SARS (51, residues 1-676; S1B, residues, 325-533), and SARS2 (51, residues 1-682; S1B, residues 333-527) C-terminally tagged with Fc domain of human or mouse IgG or Strep-tag were generated as described before (Wang et al. (2020) Nat. Commun. 11:2251-6). Recombinant proteins and antibody 47D11 were expressed transiently in FreeStyle™ 293-F Cells (Thermo Fisher Scientific) and affinity purified from the culture supernatant by protein-A sepharose beads (GE Healthcare) or streptactin beads (IBA) purification. Purity and integrity of all purified recombinant proteins was checked by coomassie stained SDS-PAGE.

Preparation of Fab-47D11 from IgG

47D11 Fab was digested from IgG with papain using a Pierce Fab Preparation Kit (Thermo Fisher Scientific), following the manufacturer's standard protocol.

### Pseudotyped Virus Neutralization Assay (SARS-S and SARS2-S)

Production of VSV pseudotyped with SARS-S, SARS2-S or WIV16-S was performed as described previously with some adaptations (Wang et al. (2020) Nat. Commun. 11:2251-6). Briefly, HEK-293T cells were transfected with pCAGGS expression vectors encoding SARS-S, SARS2-S or WIV16-S carrying a 28-, 18- or 19-a.a. cytoplasmic tail truncation, respectively. One day post transfection, cells were infected with the VSV-G pseudotyped VSVAG bearing the firefly (Photinus pyralis) luciferase reporter gene. Twenty-four hours later, supernatants containing SARS-S/SARS2-S/WIV16-S pseudotyped VSV particles were harvested and titrated on African green monkey kidney VeroE6 (ATCC #CRL-1586) cells. In the virus neutralization assay, mAbs were fourfold serially diluted at two times the desired final concentration in DMEM supplemented with 1% fetal calf serum (Bodinco), 100 U/ml Penicillin and 100 μg/ml Streptomycin (Lonza, Catalog #17-602E). Diluted mAbs were incubated with an equal volume of pseudotyped VSV particles for 1 hour at room temperature, inoculated on confluent VeroE6 monolayers in 96-well plate, and further incubated at 37° C. for 24 hours. Cells were then washed and lysis buffer (Promega) was added. Luciferase activity was measured on a Berthold Centro LB 960 plate luminometer using D-luciferin as a substrate (Promega). The percentage of infectivity was calculated as ratio of luciferase readout in the presence of mAbs normalized to luciferase readout in the absence of mAb. The half maximal inhibitory concentrations (IC50) were determined using 4-parameter logistic regression (GraphPad Prism version 8).

### ELISA Analysis of Antibody Binding to CoV Spike Antigens

NUNC Maxisorp plates (Thermo Scientific) were coated with equimolar antigen amounts at 4° C. overnight. Plates were washed three times with PBS containing 0.05% Tween-20 and blocked with 3% bovine serum albumin (Bio-Connect) in PBS containing 0.1% Tween-20 at room temperature for 2 hours. Fourfolds serial dilutions of mAbs starting at 10 μg/ml (diluted in blocking buffer) were added and plates were incubated for 1 hour at room temperature. Plates were washed three times and incubated with horseradish peroxidase (HRP)-conjugated goat anti-human secondary antibody (ITK Southern Biotech) diluted 1:2000 in blocking buffer for 1 hour at room temperature. An HRP-conjugated anti-StrepMAb (IBA, Catalog #2-1509-001) antibody was used to corroborate equimolar coating of the strep-tagged spike antigens. HRP activity was measured at 450 nanometer using tetramethylbenzidine substrate (BioFX) and an ELISA plate reader (EL-808, Biotek). Half-maximum effective concentration (EC50) binding values were calculated by non-linear regression analysis on the binding curves using GraphPad Prism (version 8).

### Flow Cytometry-Based Antibody Binding Assay

Antibody binding to full length SARS2-S epitope mutants on the cell surface was measured by flow cytometry. HEK-293T cells were seeded at a density of 2.5×105 cells per ml in a T25 flask. After reaching 80% confluency, cells were transfected with an expression plasmid encoding full length SARS2-S mutants with C-terminal Flag tag—using Lipofectamine 2000 (Invitrogen). 24 hours post transfection, cells were dissociated by cell dissociation solution (Sigma-Aldrich, Merck KGaA; cat. no. C5914). To detect total spike expression, cells were permeabilized by 0.2% saponin and subjected to anti-Flag tag antibody staining. For cell surface antibody binding measurement, intact (non-permeabilized) cells were incubated with 20 μg/ml of 47D11, ACE2-Fc, CR3022 (target SARS2 RBD core), 49F1 (target SARS2-S1 outside RBD) and anti-Flag (Sigma, F1804) for 1 h on ice, followed by incubation with 1:200 diluted Alexa Fluor 488 conjugated goat anti-human IgG antibodies (Invitrogen, Thermo Fisher Scientific, #A-11013) or goat anti-mouse IgG antibodies (Invitrogen, Thermo Fisher Scientific, #A28175) for 45 min at room temperature. Cells were subjected to flow cytometric analysis with a CytoFLEX Flow Cytometer (Beckman Coulter). The results were analyzed with FlowJo (version 10). FSC/SSC gates were used to select mononuclear cells. Control antibody staining was used to define positive/negative cell populations.

### CryoEM Sample Preparation and Data Collection

3 μL of SARS-CoV-2 or SARS-CoV S at 1.6 mg/mL was mixed with 0.85 μL of Fab 47D11 at 4 mg/mL and incubated for 50s at room temperature. The sample was applied onto a freshly glow discharged R1.2/1.3 Quantifoil grid in a Vitrobot Mark IV (Thermo Fisher Scientific) chamber pre-equilibrated at 4° C. and 100% humidity. The grid was immediately blotted at force 0 for 5s and plunged into liquid ethane. Data was acquired on a 200 kV Talos Arctica (Thermo Fisher Scientific) equipped with a Gatan K2 Summit direct detector and Gatan Quantum energy filter operated in zero-loss mode with a 20 eV slit width. To account for the preferred orientation exhibited by the spike ectodomains, automated data collection at tilts 0°, 20°, and 30° was carried out using EPU 2 software (Thermo Fisher Scientific), and data at tilt 40 using SerialEM. A nominal magnification of 130,000× corresponding to an effective pixel size of 1.08Å was used. Movies were acquired in counting mode with a total dose of 40e/A² distributed over 50 frames. 4,231 movies were acquired for SARS-CoV-2 and 3,247 movies for SARS-CoV, with defocus ranging between 0.5 μm and 3 μm.

### Cryo-EM Data Processing

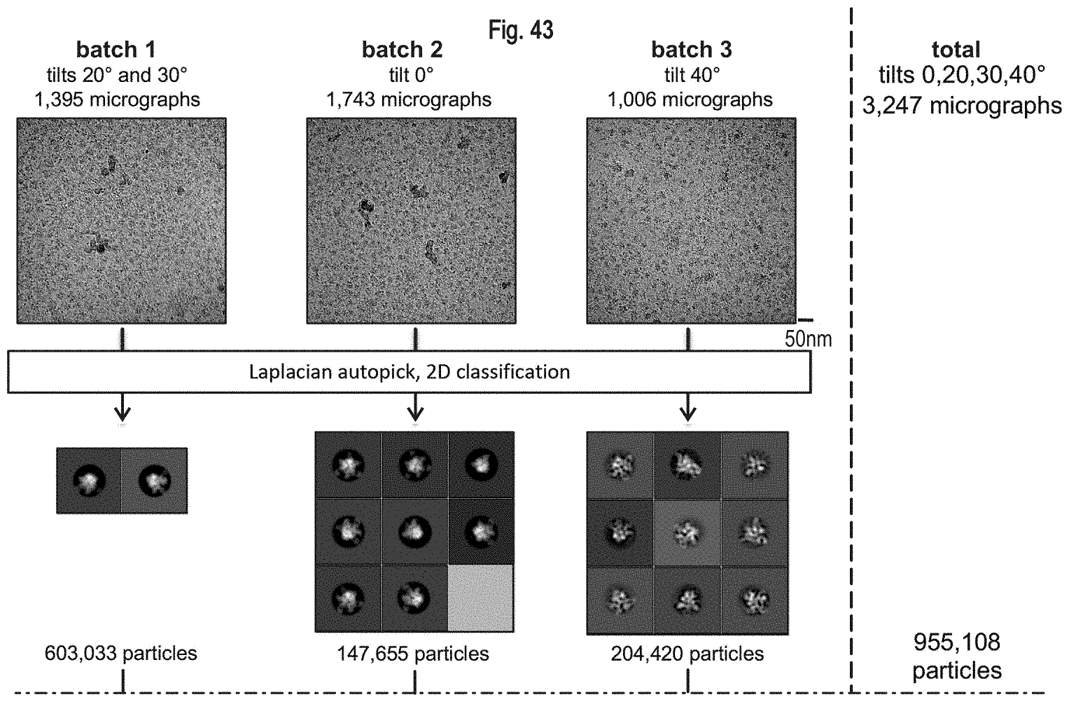

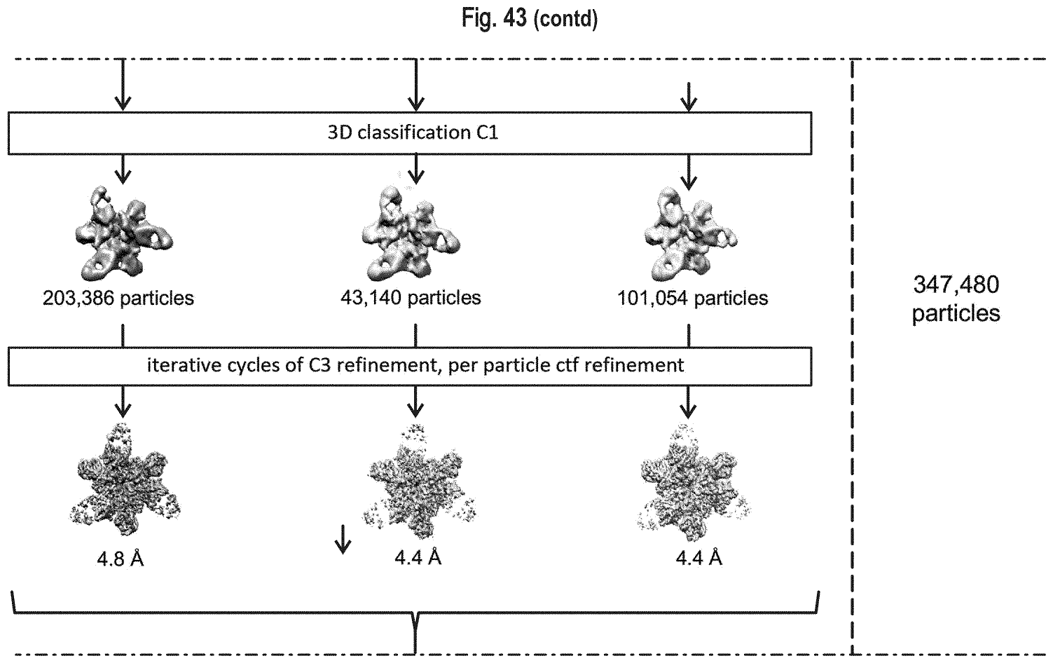

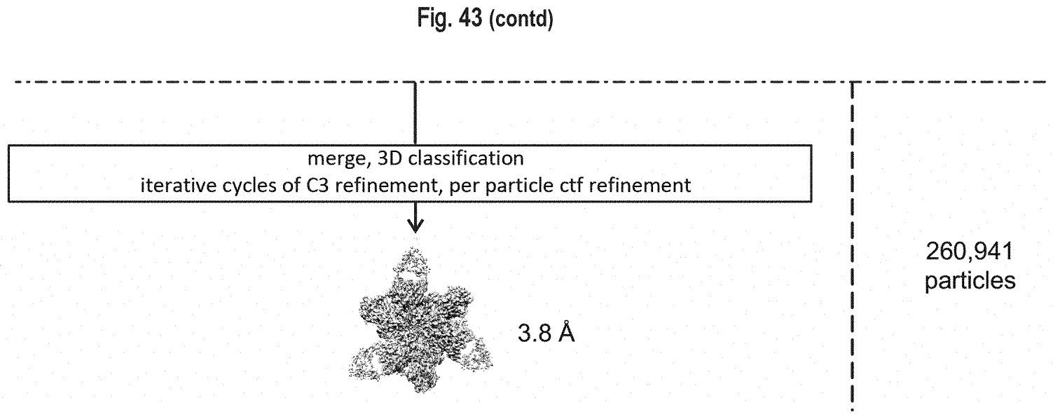

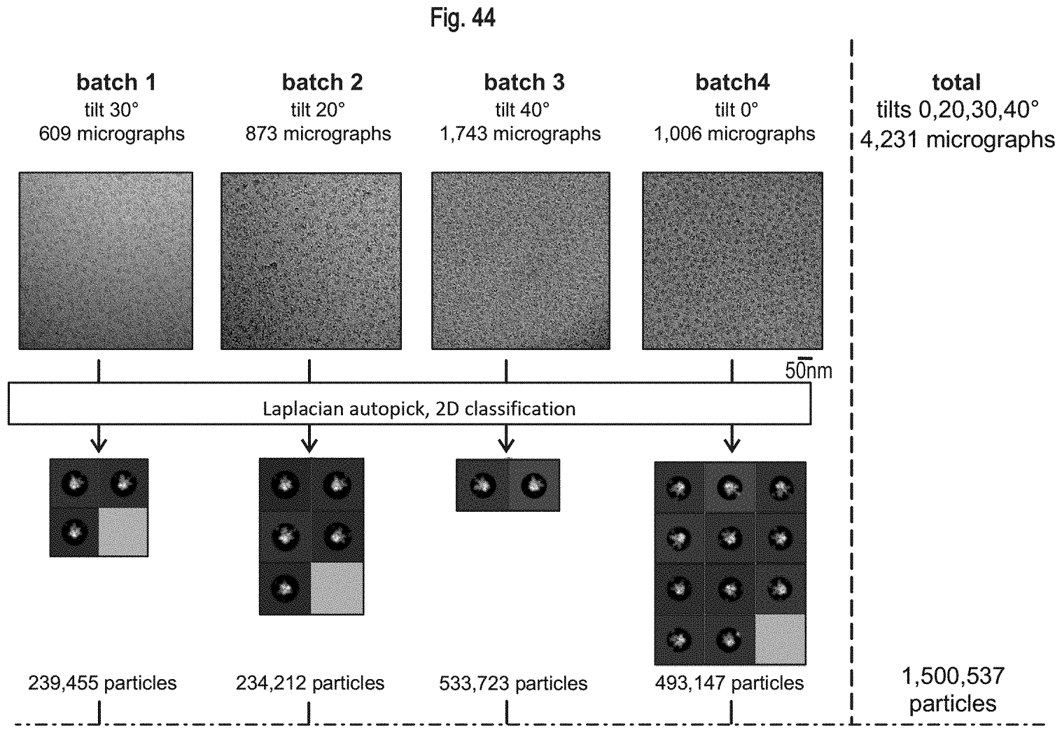

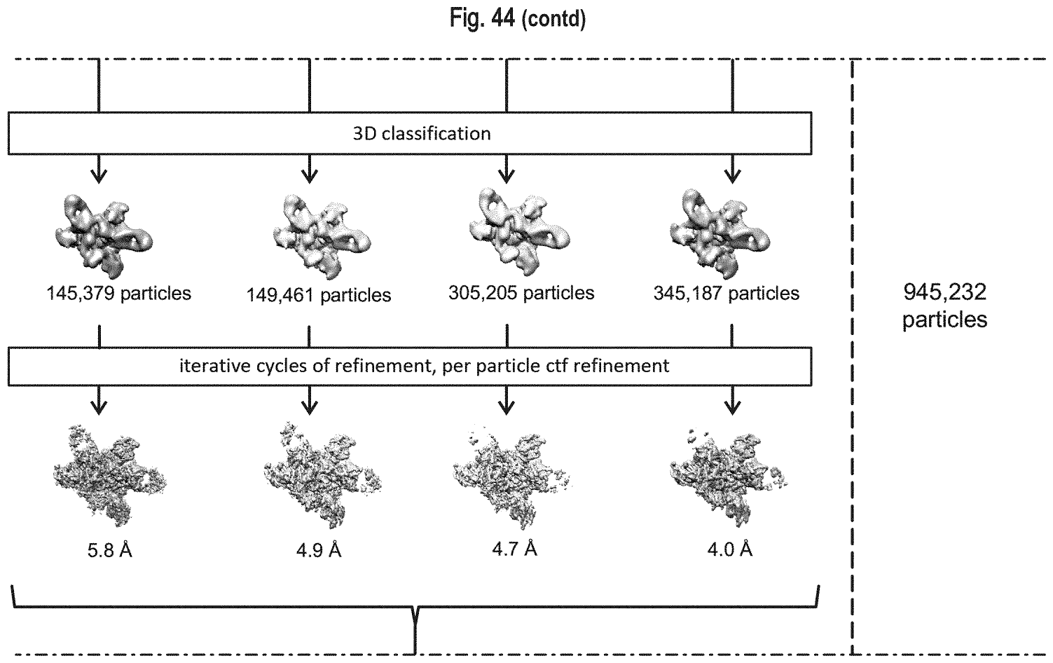

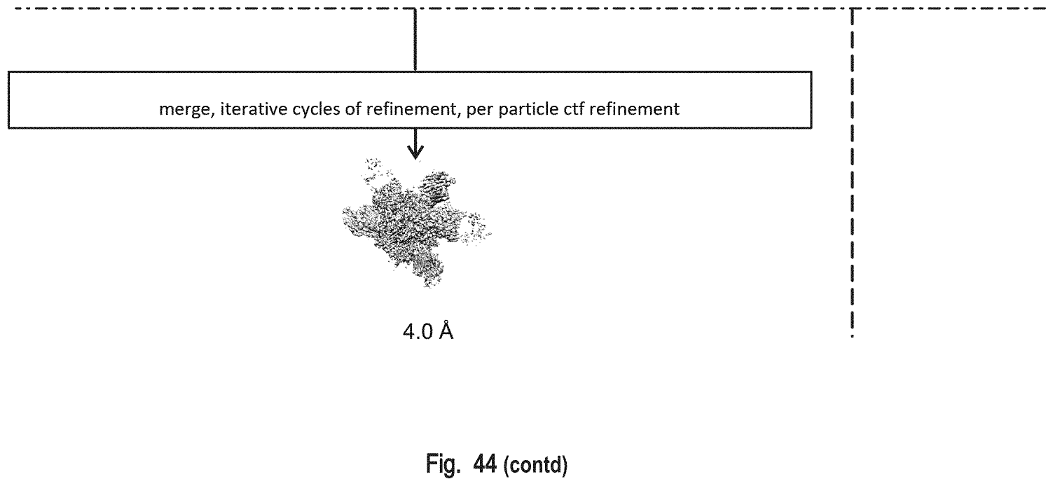

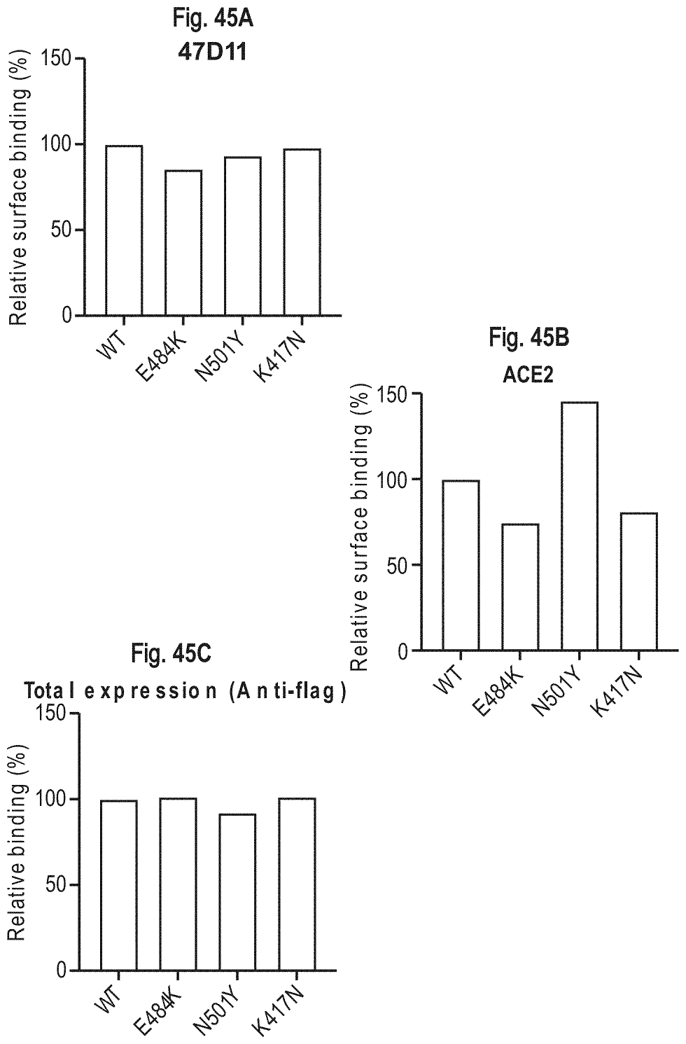

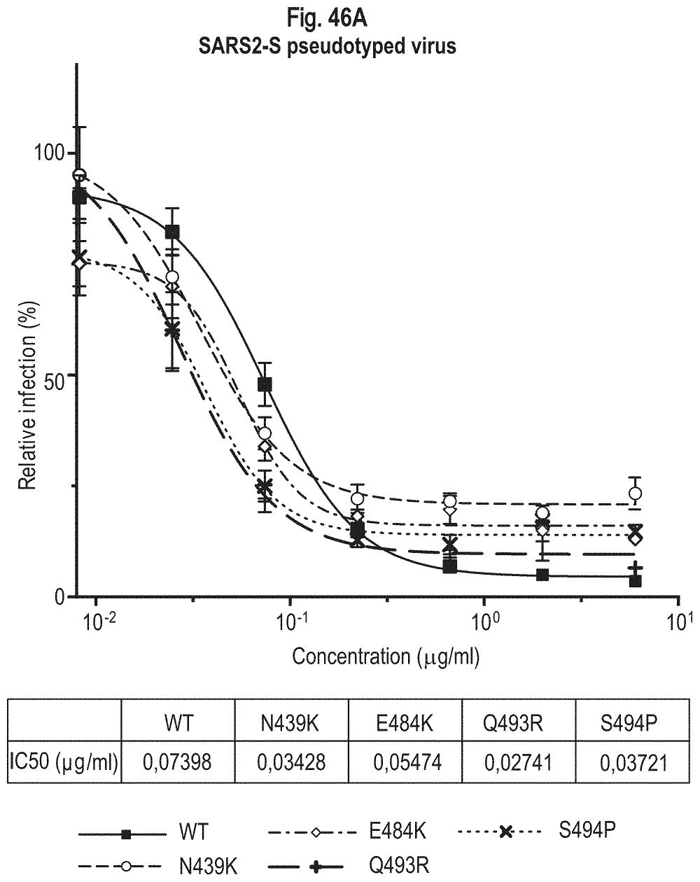

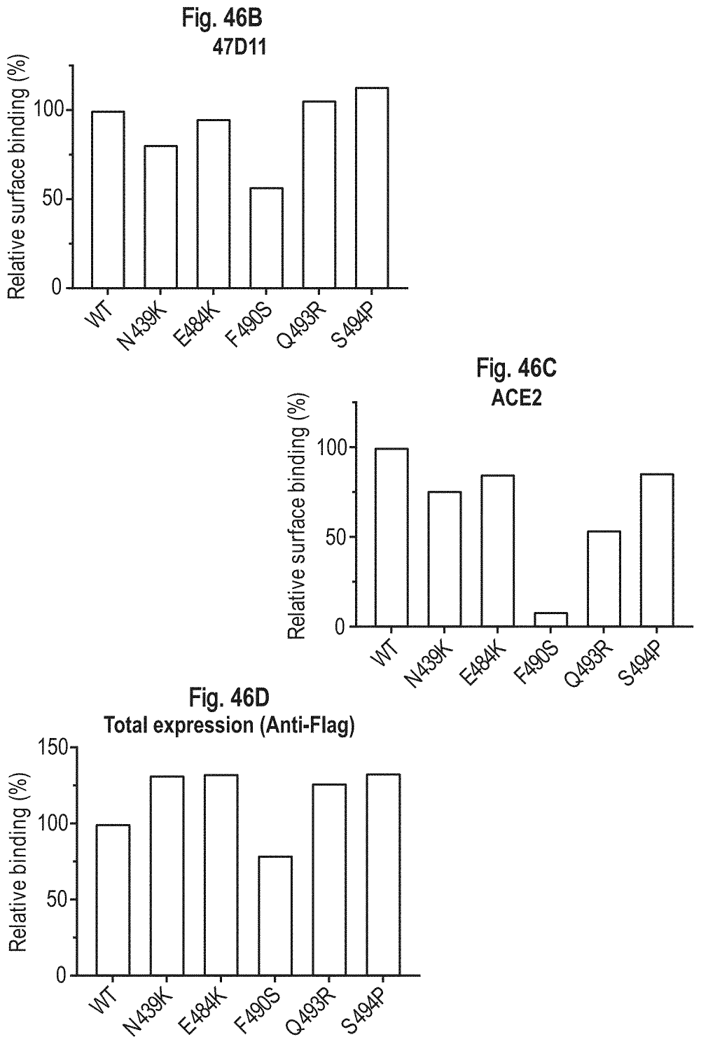

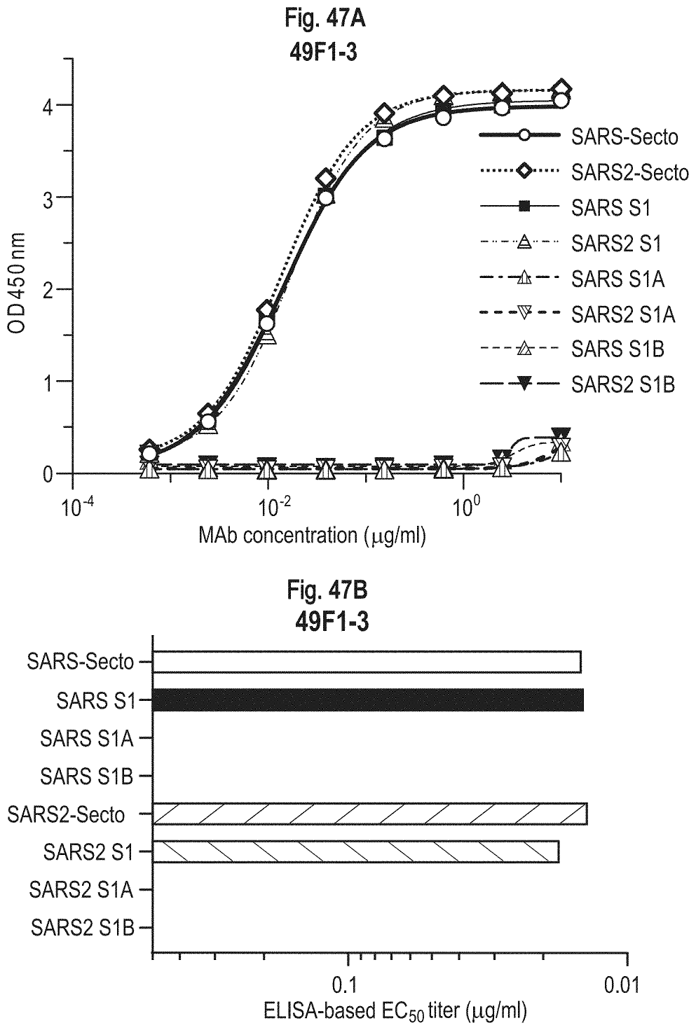

Single-particle analysis was performed in Relion version 3.1 (Zivanov et al. (2019) IUCrJ 6:5-17). The data was processed in four separate batches, corresponding to the stage tilt angle used for the acquisition. Drift and gain correction were performed with MotionCor2 (Zheng et al. (2017) Nat. Methods 14:331-332), CTF parameters were estimated using CTFFind4 (Rohou et al. (2015) Journal of Structural Biology 192:216-221) and particles were picked using the Laplacian picker in Relion (Zivanov et al. (2019) IUCrJ 6:5-17). One round of 2D classification was performed on each batch of data and particles belonging to well defined classes were retained. Subsequently, 3D classification was performed, using a 50 Å low-pass filtered partially open conformation as an initial model (EMD-21457; Walls et al. (2020) Cell 181:281-292 e286), without imposing symmetry. All particles belonging to Fab bound class were then selected for 3D auto-refinement. Before merging the different batches, iterative rounds of per particle defocus estimation, 3D auto-refinement and post-processing were used to account for the stage tilt used during data collection. The refined particle star files from each batch were then combined and subjected to a final round of 3D auto-refinement, per particle defocus estimation, 3D auto-refinement and post processing, both with and without imposed C3 symmetry. Overviews of the single-particle image processing pipelines are shown in FIGS. 43 and 44.

### Model Building and Refinement

UCSF Chimera (version 1.12.0) and Coot (version 1.0) were used for model building and analysis (Pettersen et al. (2004) J Comput Chem. 25:1605-1612; Emsley et al. (2004) Acta Crystallogr. D Biol. Crystallogr. 60:2126-2132). The SARS-CoV-2 S model, in the partially open conformation (one RBD up, pdb 6VYB), was used for the spike and fitted into our density using the UCSF Chimera ‘Fit in map’ tool (Pettersen et al. (2004) J Comput Chem. 25:1605-1612). For SARS-CoV a closed protomer of the pdb 6NB6 was used as starting model (Walls et al. (2019) Cell 176:1026-1039.e15). To build a model for the Fab the sequence of the variable regions of the HC and the LC were separately blasted against the pdb. For the HC variable region, the corresponding region of the pdb 6IEB (human monoclonal antibody R15 against RVFV Gn) was used (Wang et al. (2019) Nat Microbiol. 4:1231-1241). The LC variable region was modelled using the pdb 6FG1 as template (Fab Natalizumab) (Cassotta et al. Nat. Med. 25:1402-1407). For both chains, the query sequence of 47D11 was aligned to the template sequence. Sequence identity was particularly high (87% and 97% for the HC and LC, respectively). Phenix sculptor was used to create an initial model for the Fab chains (Bunkoczi et al. (2011) Acta Crystallogr. D Biol. Crystallogr. 67:303-312), removing the non-aligning regions (notably the CDRH3). This model was fitted into our density and the missing regions were built manually using the density map in Coot (Emsley et al. (2004) Acta Crystallogr. D Biol. Crystallogr. 60:2126-2132). Models were refined against the respective EM density maps using Phenix Real Space Refinement and Isolde (Headd et al. (2012) Acta Crystallogr D Biol Crystallogr 68, 381-390; Croll (2018) Acta Crystallogr D Struct Biol 74, 519-530) and validated with MolProbity and Privateer (glycans)(Chen et al. (2010) Acta Crystallogr. D Biol. Crystallogr. 66:12-21; Headd et al. (2012) Acta Crystallogr. D Biol. Crystallogr. 68:381-390; Agirre et al. (2015) Nat Struct. Mol. Biol. 22:833-834; Agirre et al. (2015) Nat. Chem. Biol. 11:303).

### Analysis and Visualisation

PDBePISA was used to identify spike residues interacting with 47D11 (Krissinel et al. (2007) J. Mol. Biol. 372:774-797). Surface colouring of the SARS-CoV-2 RBD using the Kyte-Doolittle hydrophobicity scale was performed in UCSF chimera (Pettersen et al. (2004) J Comput Chem. 25:1605-1612). Volume measurements were performed using CASTp 3.0, using a probe radius of 1.2 Å. In order to colour the 47D11 bound RBD surface according to each residues mean mutational effect on expression, the pdb file was populated with the mean mutation effect on expression values described by Starr et al. (Cell (2020) doi:10.1016/j.ce11.2020.08.012). The UCSF Chimera ‘MatchMaker’ tool was used to obtain RMSD values, using default settings. Figures were generated using UCSF Chimera(33) and UCSF ChimeraX (Goddard et al. (2018) Protein Sci. 27:14-15).

TABLE 3
Data collection, image processing and refinement information.

| | |
|---|---|
| SARS-coV + | SARS-coV2 + |
| 47D11 | 47D11 |

Data Collection and processing

| | | |
|---|---|---|
| Magnification | 130 000 | 130 000 |
| Voltage (kV) | 200 | 200 |
| Electron exposure (e−/Å²) | 40 | 40 |
| Defocus range (μm) | 0.5-2.5 | 0.5-2.5 |
| Pixel size (Å) | 1.08 | 1.08 |
| Symmetry imposed | C3 | C1 |
| Initial particle images (no.) | 955 108 | 1 500 537 |
| Final particle images (no.) | 260 941 | 945 232 |
| Map resolution (Å) | 3.8 | 4.0 |
| FSC threshold | 0.143 | 0.143 |
| Map resolution range (Å) | 3.4-6.3 | 3.4-6.3 |

Refinement

| | | |
|---|---|---|
| Initial model used (PDB code) | 6NB6 | 6VYB |
| Model resolution (Å) | 3.9 | 4.0 |
| FSC threshold | 0.5 | 0.5 |
| Map sharpening B factor (Å²) | −168 | −154 |
| Model composition | | |
| Amino acids | 3930 | 3404 |
| Glycans | 96 | 79 |
| mean B factors (Å²) | | |
| Protein | 42.4 | 44.1 |
| Glycans | 91.4 | 83.9 |
| R.m.s deviations | | |
| Bond lengths (Å) | 0.005 | 0.003 |
| Bond angles (°) | 0.826 | 0.624 |
| Validation | | |
| Molprobity score | 1.49 | 1.21 |
| Clashscore | 5.99 | 2.90 |
| Poor rotamers (%) | 0.09 | 0.21 |
| Ramachandran plot | | |
| Favored (%) | 97.12 | 97.31 |
| Allowed (%) | 2.88 | 2.69 |
| Disallowed (%) | 0 | 0 |

### FURTHER EMBODIMENTS OF THE INVENTION

1. An antibody that binds to SARS-Cov-2 coronavirus (SARS2) spike protein (SARS2-S).

2. The antibody of embodiment 1, wherein the antibody is capable of inhibiting the infection of human cells by SARS2.

3. The antibody of embodiment 1 or embodiment 2, wherein the antibody binds to the S1 subunit of SARS2-S.

4. The antibody of embodiment 3, wherein the antibody binds to the S1_{B} subunit of SARS2-S.

5. The antibody of embodiment 4, wherein the antibody inhibits the binding of SARS2-S to human angiotensin converting enzyme 2 (ACE2).

6. The antibody of embodiment 4, wherein the antibody does not inhibit the binding of SARS2-S to human angiotensin converting enzyme 2 (ACE2).

7. The antibody of embodiment 2, wherein the antibody binds to the S1_{B} subunit of SARS2-S, and wherein the antibody does not inhibit the binding of SARS2-S to human angiotensin converting enzyme 2 (ACE2).

8. The antibody of embodiment 4, wherein the antibody binds specifically to the closed conformation of the SARS2-S1_{B}, optionally wherein the antibody binds distal to the ACE2 binding site.

9. The antibody of embodiment 8, wherein the antibody stabilises the N343 glycan of SARS2-S in an upright conformation, thereby exposing a hydrophobic pocket to which the antibody binds via one or more aromatic residues in one or more of its CDRs.

10. The antibody of any one of embodiments 4, 8 and 9, wherein the antibody binds specifically to the partially open conformation of a SARS2-S trimer, optionally wherein the antibody also binds specifically to the closed conformation of a SARS1-S trimer.

11. The antibody of embodiment 2, wherein the antibody binds to SARS2-S1_{B} having the sequence of SEQ ID NO: 21, and wherein the antibody binds to an epitope comprising one or more (e.g. two, three, four, five, six, seven or more) of residues F3, F7, N8, Y30, V32, L33, F39 and W101 of SEQ ID NO: 21.

12. The antibody of embodiment 11, wherein the antibody binds to SARS2-S1_{B} having the sequence of SEQ ID NO: 21, and wherein the antibody binds to an epitope comprising residues F3, F7, N8, Y30, V32, L33, F39 and W101 of SEQ ID NO: 21.

13. The antibody of any one of embodiments 2, 4 and 7, wherein the antibody binds to the same epitope as an antibody comprising a heavy chain variable region of the amino acid sequence of SEQ ID NO: 10 and a light chain variable region of the amino acid sequence of SEQ ID NO: 9.

14. The antibody of any one of embodiments 2, 4 and 7, wherein the antibody competes for binding to SARS2-S with an antibody comprising a heavy chain variable region of the amino acid sequence of SEQ ID NO: 10 and a light chain variable region of the amino acid sequence of SEQ ID NO: 9.

15. The antibody of any one of embodiments 2, 4 and 7-14, wherein the antibody comprises complementarity determining regions (CDRs) with the sequences of:
i. a sequence that is at least 70%, at least 80%, at least 90%, or at least 95% identical to SEQ ID NO: 53 for CDR1 of the light chain;
ii. a sequence that is at least 70%, at least 80%, at least 90%, or at least 95% identical to SEQ ID NO: 54 for CDR2 of the light chain;
iii. a sequence comprising or consisting of X₁X₂X₃X₄X₅X₆X₇X₈X₉ for CDR3 of the light chain, wherein:
   X₁ is Q, D, E or N,
   X₂ is Q, D, E or N,
   X₃ is Y, F or W,
   X₄ is N, D, E or Q,
   X₅ is N, D, E or Q,
   X₆ is W, Y or F,
   X₇ is P,
   X₈ is L, G, A, V or I, and
   X₉ is T, S, C, U or M;
iv. a sequence that is at least 70%, at least 80%, at least 90%, or at least 95% identical to SEQ ID NO: 56 for CDR1 of the heavy chain;
v. a sequence that is at least 70%, at least 80%, at least 90%, or at least 95% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
   X₁ is A, G, V, L or I,
   X₂ is R, K or H,
   X₃ is G, A, V, L or I,
   X₄ is V, G, A, L or I,
   X₅ is L, G, A, V or I,
   X₆ is L, G, A, V or I,
   X₇ is W, F or Y,
   X₈ is F, W or Y,
   X₉ is G, A, V, L or I,
   X₁₀ is Q, D, E or N,
   X₁₁ is P,
   X₁₂ is I, G, A, V or L,
   X₁₃ is F, W or Y,
   X₁₄ is Q, D, E or N, and
   X₁₅ is I, G, A, V or L.

16. The antibody of any one of embodiments 2, 4 and 7-14, wherein the antibody comprises complementarity determining regions (CDRs) with the sequences of:
i. SEQ ID NO: 56 for CDR1 of the heavy chain;
ii. SEQ ID NO: 57 for CDR2 of the heavy chain;
iii. SEQ ID NO: 58 for CDR3 of the heavy chain;
iv. SEQ ID NO: 53 for CDR1 of the light chain;
v. SEQ ID NO: 54 for CDR2 of the light chain; and
vi. SEQ ID NO: 55 for CDR3 of the light chain.

17. The antibody of embodiment 16, wherein the antibody comprises a heavy chain variable region of the amino acid sequence of SEQ ID NO: 10 and a light chain variable region of the amino acid sequence of SEQ ID NO: 9.

18. The antibody of embodiment 3, wherein the antibody binds to the S1_{A}, S1_{C} or S1_{D} subunit of SARS2-S.

19. The antibody of embodiment 3, wherein the antibody binds to the S2 subunit of SARS2-S.

20. The antibody of embodiment 2, wherein the antibody binds to the same epitope as an antibody comprising a heavy chain variable region of the amino acid sequence of SEQ ID NO: 6 and a light chain variable region of the amino acid sequence of SEQ ID NO: 5.

21. The antibody of embodiment 2, wherein the antibody competes for binding to SARS2-S with an antibody comprising a heavy chain variable region of the amino acid sequence of SEQ ID NO: 6 and a light chain variable region of the amino acid sequence of SEQ ID NO: 5.

22. The antibody of embodiment 2, wherein the antibody comprises complementarity determining regions (CDRs) with the sequences of:
i. SEQ ID NO: 44 for CDR1 of the heavy chain;
ii. SEQ ID NO: 45 for CDR2 of the heavy chain;
iii. SEQ ID NO: 46 for CDR3 of the heavy chain;
iv. SEQ ID NO: 41 for CDR1 of the light chain;
v. SEQ ID NO: 42 for CDR2 of the light chain; and
vi. SEQ ID NO: 43 for CDR3 of the light chain.

23. The antibody of embodiment 22, wherein the antibody comprises a heavy chain variable region of the amino acid sequence of SEQ ID NO: 6 and a light chain variable region of the amino acid sequence of SEQ ID NO: 5.

24. The antibody of any one of embodiments 1-14 and 18-21, wherein the antibody is a heavy chain-only antibody.

25. The antibody of any one of embodiments 1-23, wherein the antibody is an Fab, Fab′, F(ab′)2, Fd, Fv, a single-chain Fv (scFv) or a disulfide-linked Fv (sdFv).

26. A fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain.

27. A fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9.

28. A fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66.

29. A combination of antibodies comprising:

(i) an antibody according to any one of embodiments 1-17, 20-23, and 26-28; and
(ii) an antibody according to embodiment 18 or embodiment 19.

30. A combination of antibodies comprising:

(i) an antibody according to any one of embodiments 8-17 and 26-28; and
(ii) an anti-SARS2 antibody that binds specifically to the open conformation of the SARS2-S1_{B}.

31. An isolated nucleic acid encoding the antibody of any one of embodiments 1-28.

32. A vector comprising the nucleic acid of embodiment 31.

33. A host cell comprising the vector of embodiment 32.

34. A pharmaceutical composition comprising the antibody of any one of embodiments 1-28, or the combination of antibodies of embodiment 29 or embodiment 30, and a pharmaceutically acceptable carrier.

35. The antibody of any one of embodiments 1-28, the combination of antibodies of embodiment 29 or embodiment 30, or the composition of embodiment 34, for use in therapy.

36. The antibody, combination of antibodies or composition for use of embodiment 35, wherein the therapy is preventing, treating or ameliorating coronavirus infection, optionally betacoronavirus infection, such as SARS2 infection.

37. The antibody, combination of antibodies or composition for use of embodiment 35, wherein the therapy is preventing, treating or ameliorating coronavirus infection in a human subject.

38. The antibody, combination of antibodies or composition for use of embodiment 37, wherein the therapy is preventing, treating or ameliorating:

(a) coronavirus-induced pneumonia, optionally severe coronavirus-induced pneumonia, and/or
(b) coronavirus-induced weight loss, and/or
(c) coronavirus-induced lung inflammation, and/or
(d) coronavirus replication, optionally coronavirus replication in the lower respiratory tract, and/or
(e) coronavirus-induced lung lesions, optionally coronavirus-induced gross lesions in the lung.

39. The antibody, combination of antibodies or composition for use of embodiment 37 or embodiment 38, wherein the therapy reduces coronavirus load in the lungs.

40. The antibody, combination of antibodies or composition for use of embodiment 37, wherein the therapy is preventing, treating or ameliorating:

(a) betacoronavirus-induced pneumonia, optionally severe betacoronavirus-induced pneumonia, and/or
(b) betacoronavirus-induced weight loss, and/or
(c) betacoronavirus-induced lung inflammation, and/or
(d) betacoronavirus replication, optionally betacoronavirus replication in the lower respiratory tract, and/or
(e) betacoronavirus-induced lung lesions, optionally betacoronavirus-induced gross lesions in the lung.

41. The antibody, combination of antibodies or composition for use of embodiment 40, wherein the therapy reduces betacoronavirus load in the lungs.

42. The antibody, combination of antibodies or composition for use of embodiment 37, wherein the therapy is preventing, treating or ameliorating SARS2 infection in a subject.

43. The antibody, combination of antibodies or composition for use of embodiment 42, wherein the therapy is preventing, treating or ameliorating:

(a) SARS2-induced pneumonia, optionally severe SARS2-induced pneumonia, and/or
(b) SARS2-induced weight loss, and/or
(c) SARS2-induced lung inflammation, and/or
(d) SARS2 replication, optionally SARS2 replication in the lower respiratory tract, and/or
(e) SARS2-induced lung lesions, optionally SARS2-induced gross lesions in the lung.

44. The antibody, combination of antibodies or composition for use of embodiment 42 or embodiment 43, wherein the therapy reduces SARS2 load in the lungs.

45. The antibody, combination of antibodies or composition for use of any one of embodiments 42-44, wherein the subject is human.

46. A fully human monoclonal IgG1 antibody that binds to SARS-Cov-2 coronavirus (SARS2) spike protein (SARS2-S), wherein the antibody comprises two heavy and two light chains,
wherein each heavy chain comprises:
   i. SEQ ID NO: 56 for CDR1 of the heavy chain;
   ii. SEQ ID NO: 57 for CDR2 of the heavy chain;
   iii. SEQ ID NO: 58 for CDR3 of the heavy chain;
and wherein each light chain comprises:
   iv. SEQ ID NO: 53 for CDR1 of the light chain;
   v. SEQ ID NO: 54 for CDR2 of the light chain; and
   vi. SEQ ID NO: 55 for CDR3 of the light chain,
      for use in preventing, treating or ameliorating SARS2 infection in a human subject, wherein the antibody prevents, treats or ameliorates:
      (a) SARS2-induced pneumonia, optionally severe SARS2-induced pneumonia, and/or
      (b) SARS2-induced weight loss, and/or
      (c) SARS2-induced lung inflammation, and/or
      (d) SARS2 replication, optionally SARS2 replication in the lower respiratory tract, and/or
      (e) SARS2-induced lung lesions, optionally SARS2-induced gross lesions in the lung.

47. A fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10,
and wherein each light chain comprises a light chain variable region of the amino acid sequence of SEQ ID NO: 9,
   for use in preventing, treating or ameliorating SARS2 infection in a human subject, wherein the antibody prevents, treats or ameliorates:
   (a) SARS2-induced pneumonia, optionally severe SARS2-induced pneumonia, and/or
   (b) SARS2-induced weight loss, and/or
   (c) SARS2-induced lung inflammation, and/or
   (d) SARS2 replication, optionally SARS2 replication in the lower respiratory tract, and/or
   (e) SARS2-induced lung lesions, optionally SARS2-induced gross lesions in the lung.

48. A fully human monoclonal IgG1 antibody that binds to SARS-Cov-2 coronavirus (SARS2) spike protein (SARS2-S), wherein the antibody comprises two heavy and two light chains,
wherein each heavy chain comprises the amino acid sequence of SEQ ID NO: 65,
and wherein each light chain comprises the amino acid sequence of SEQ ID NO: 66, for use in preventing, treating or ameliorating SARS2 infection in a human subject, wherein the antibody prevents, treats or ameliorates:
   (a) SARS2-induced pneumonia, optionally severe SARS2-induced pneumonia, and/or
   (b) SARS2-induced weight loss, and/or
   (c) SARS2-induced lung inflammation, and/or
   (d) SARS2 replication, optionally SARS2 replication in the lower respiratory tract, and/or
   (e) SARS2-induced lung lesions, optionally SARS2-induced gross lesions in the lung.

49. The antibody of any one of embodiments 46-48, wherein the therapy reduces SARS2 load in the lungs.

1. An antibody that binds to SARS-Cov-2 coronavirus (SARS2) spike protein (SARS2-S), wherein the antibody comprises complementarity determining regions (CDRs) with the sequences of:
i. a sequence that is at least 90% or at least 95% identical to SEQ ID NO: 53 for CDR1 of the light chain;
ii. a sequence that is at least 90% or at least 95% identical to SEQ ID NO: 54 for CDR2 of the light chain;
iii. a sequence comprising or consisting of X₁X₂X₃X₄X₅X₆X₇X₈X₉ for CDR3 of the light chain, wherein:
X₁ is Q, D, E or N,
X₂ is Q, D, E or N,
X₃ is Y, F or W,
X₄ is N, D, E or Q,
X₅ is N, D, E or Q,
X₆ is W, Y or F,
X₇ is P,
X₈ is L, G, A, V or I, and
X₉ is T, S, C, U or M;
iv. a sequence that is at least 90% or at least 95% identical to SEQ ID NO: 56 for CDR1 of the heavy chain;
v. a sequence that is at least 90% or at least 95% identical to SEQ ID NO: 57 for CDR2 of the heavy chain; and
vi. a sequence comprising or consisting of X₁X₂X₃X₄X₅X₆X₇X₈X₉X₁₀X₁₁X₁₂X₁₃X₁₄X₁₅ for CDR3 of the heavy chain, wherein:
X₁ is A, G, V, L or I,
X₂ is R, K or H,
X₃ is G, A, V, L or I,
X₄ is V, G, A, L or I,
X₅ is L, G, A, V or I,
X₆ is L, G, A, V or I,
X₇ is W, F or Y,
X₈ is F, W or Y,
X₉ is G, A, V, L or I,
X₁₀ is Q, D, E or N,
X₁₁ is P,
X₁₂ is I, G, A, V or L,
X₁₃ is F, W or Y,
X₁₄ is Q, D, E or N, and
X₁₅ is I, G, A, V or L.

2. The antibody of claim 1, wherein the antibody comprises complementarity determining regions (CDRs) with the sequences of:
i. SEQ ID NO: 56 for CDR1 of the heavy chain;
ii. SEQ ID NO: 57 for CDR2 of the heavy chain;
iii. SEQ ID NO: 58 for CDR3 of the heavy chain;
iv. SEQ ID NO: 53 for CDR1 of the light chain;
v. SEQ ID NO: 54 for CDR2 of the light chain; and
vi. SEQ ID NO: 55 for CDR3 of the light chain.

3. The antibody of claim 2, wherein the antibody comprises a heavy chain variable region of the amino acid sequence of SEQ ID NO: 10 and a light chain variable region of the amino acid sequence of SEQ ID NO: 9.

4. The antibody of claim 1, wherein the antibody is an Fab, Fab′, F(ab′)2, Fd, Fv, a single-chain Fv (scFv) or a disulfide-linked Fv (sdFv).

5. A fully human monoclonal IgG1 antibody that binds to SARS-Cov-2 coronavirus (SARS2) spike protein (SARS2-S), wherein the antibody comprises two heavy and two light chains,
wherein each heavy chain comprises:
i. SEQ ID NO: 56 for CDR1 of the heavy chain;
ii. SEQ ID NO: 57 for CDR2 of the heavy chain;
iii. SEQ ID NO: 58 for CDR3 of the heavy chain;
and wherein each light chain comprises:
iv. SEQ ID NO: 53 for CDR1 of the light chain;
v. SEQ ID NO: 54 for CDR2 of the light chain; and
vi. SEQ ID NO: 55 for CDR3 of the light chain.

6. A fully human monoclonal IgG1 antibody that binds to SARS-Cov-2 coronavirus (SARS2) spike protein (SARS2-S), wherein the antibody comprises two heavy and two light chains,
wherein each heavy chain comprises a heavy chain variable region of the amino acid sequence of SEQ ID NO: 10, and
and each light chain comprises a light chain variable region of the amino acid sequence of SEQ ID NO: 9; or
wherein each heavy chain comprises the amino acid sequence of SEQ ID NO: 65, and each light chain comprises the amino acid sequence of SEQ ID NO: 66.

7. (canceled)

8. A combination of antibodies comprising:
(i) an antibody according to claim 1; and
(ii) an antibody that binds to the S1_{A}, S1_{C}, S1_{D} or S2 subunit of SARS2-S.

9. A combination of antibodies comprising:
(i) an antibody according to claim 1; and
(ii) an anti-SARS2 antibody that binds specifically to the open conformation of the SARS2-S1_{B}.

10. An isolated nucleic acid encoding the antibody of claim 1.

11. A vector comprising the nucleic acid of claim 10.

12. A host cell comprising the vector of claim 11.

13. A pharmaceutical composition comprising the antibody of claim 1, and a pharmaceutically acceptable carrier.

14. (canceled)

15. A method of preventing, treating or ameliorating coronavirus infection, said method comprising administering the antibody of claim 1 to a human subject in need thereof.

16. (canceled)

17. The method of claim 15, wherein the method comprises preventing, treating or ameliorating:
(a) coronavirus-induced pneumonia, and/or
(b) coronavirus-induced weight loss, and/or
(c) coronavirus-induced lung inflammation, and/or
(d) coronavirus replication, and/or
(e) coronavirus-induced lung lesions, optionally coronavirus-induced gross lesions in the lung.

18. The method of claim 15 which reduces coronavirus load in the lungs.

19. The method of claim 15, wherein the method comprises preventing, treating or ameliorating:
(a) betacoronavirus-induced pneumonia, and/or
(b) betacoronavirus-induced weight loss, and/or
(c) betacoronavirus-induced lung inflammation, and/or
(d) betacoronavirus replication, optionally betacoronavirus replication in the lower respiratory tract, and/or
(e) betacoronavirus-induced lung lesions.

20. The method of claim 19, wherein the method reduces betacoronavirus load in the lungs.

21. The method for of claim 15, is for preventing, treating or ameliorating SARS2 infection in the subject.

22. The method for of claim 21, wherein the method prevents, treats, or ameliorates:
(a) SARS2-induced pneumonia, and/or
(b) SARS2-induced weight loss, and/or
(c) SARS2-induced lung inflammation, and/or
(d) SARS2 replication, and/or
(e) SARS2-induced lung lesions, optionally SARS2 induced gross lesions in the lung.

23. The a method for use of claim 21, wherein the therapy reduces SARS2 load in the lungs.

24. (canceled)

25. A method of
preventing, treating or ameliorating SARS2 infection in a human subject, said method comprising administering the antibody of claim 5 to the human subject such that the antibody prevents, treats or ameliorates:
(a) SARS2-induced pneumonia, and/or
(b) SARS2-induced weight loss, and/or
(c) SARS2-induced lung inflammation, and/or
(d) SARS2 replication, and/or
(e) SARS2-induced lung lesions.

26. A method of
preventing, treating or ameliorating SARS2 infection in a human subject, said method comprising administering the antibody of claim 6 to the human subject, such that the antibody prevents, treats or ameliorates:
(a) SARS2-induced pneumonia, and/or
(b) SARS2-induced weight loss, and/or
(c) SARS2-induced lung inflammation, and/or
(d) SARS2 replication, and/or
(e) SARS2-induced lung lesions.

27. (canceled)

28. The method of claim 25, wherein the method reduces SARS2 load in the lungs.


SEQUENCE LISTING (75 sequences)

SEQ ID NO: 1 - protein, 106 aa, Homo sapiens
ETVMTQSPATLSVSPGERATLSCRASQSVSSNLAWYQQKPGQAPRLLIYGASTRATGIPARFSGSGSGTEFTLTISSLQSEDFAVYYCQQFNNWITFGQGTRLEIK

SEQ ID NO: 2 - protein, 119 aa, Homo sapiens
EVQLLESGGGLVQPGGSLRLSCAASGFTFSIYAMNWVRQAPGKGLEWVSGISGSGNHIHYADSVKGRFTISRENSKSTLYLHMNSLRAEDTAVYYCAKDGGLYWYFDLWGRGTLVTVSS

SEQ ID NO: 3 - protein, 107 aa, Homo sapiens
EIVMTQSPATLSVSPGERATLSCRASQSVSSNLAWYQQKPGQAPRLLIFRASTRATGIPARFSGSGSGTEFTLTISSLQSEDFAVYHCQQYNSWPITFGQGTRLEIK

SEQ ID NO: 4 - protein, 116 aa, Homo sapiens
QVHLVESGGGVVQPGRSLRLSCAASGFTFSSYGMHWVRQGPGKGLEGVAGIWFDGSHEYYADAVKGRFTISRDNSKNTLYLEMSSLRAGDTGIYYCARGGDILTPWGQGSLVIVSS

SEQ ID NO: 5 - protein, 107 aa, Homo sapiens
DIQMTQSPSTLSASVGDRVTITCRASQSISSWLAWYQQKPGKAPKLLIYAASSLQSGVPSRFSGSGSGTDFTLTISSLQPEDFATYYCQQSYSTPITFGQGTRLEIK

SEQ ID NO: 6 - protein, 120 aa, Homo sapiens
QVQLVQSGAEVKKPGSSVKVSCKASGGTFSSYAINWVRQAPGQGLEWMGGIIPIFGTANSAQKFQGRVTITADKSTSTAYMELSSLRSEDTAVYYCAREAGIPVAGLADWGQGTLVTVSS

SEQ ID NO: 7 - protein, 116 aa, Homo sapiens
QVQLVESGGGVVQPGRSLRLSCAASGFTFSSYGMHWVRQGPGKGLEGVAGIWFDGSHEYYADAVKGRFTISRDNSKNTLYLEMSSLRAGDTGIYYCARGGDILTPWGQGSLVIVSS

SEQ ID NO: 8 - protein, 1203 aa, SARS-CoV-2 strain Wuhan-Hu-1
SGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - protein, 107 aa, Homo sapiens
EIVMTQSPATLSVSPGERATLSCRASQSVSSSLAWYQQKPGQAPRLLIYGASTRAPGIPARFSGSGSGTEFTLTISSLQSEDFAVYYCQQYNNWPLTFGGGTKVEIK

SEQ ID NO: 10 - protein, 121 aa, Homo sapiens
QVQLQESGPGLVKPSETLSLTCSVSGGSISSHYWSWIRQPPGKGLEWIGYIYYSGSTNHNPSLKSRVTISVDTSKNQFSLKLSSVTAADTAVYYCARGVLLWFGEPIFEIWGQGTMVTVSS

SEQ ID NO: 11 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 12 - protein, 97 aa, Homo sapiens
QVQLQESGPGLVKPSETLSLTCTVSGGSISSHYWSWIRQPPGKGLEWIGYIYYSGSTNYNPSLKSRVTISVDTSKNQFSLKLSSVTAADTAVYYCAR

SEQ ID NO: 13 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 14 - protein, 98 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAR

SEQ ID NO: 15 - DNA, 318 bp, Homo sapiens
gaaacagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtttaataactggatcaccttcggccaagggacacgactggagattaaa

SEQ ID NO: 16 - DNA, 357 bp, Homo sapiens
gaggtgcaactattggagtctgggggaggcttggtacagcctggggggtccctgagactctcctgtgcagcctctggattcacctttagcatctatgccatgaactgggtccgccaggctccagggaaggggctggagtgggtctcaggtattagtggtagtggtaatcacatacactacgcagactccgtgaagggccggttcaccatctccagagaaaattccaagagcacgctgtatctgcacatgaacagcctgagagccgaggacacggccgtatattactgtgcgaaggacgggggactctactggtacttcgatctctggggccgtggcaccctggtcactgtctcctca

SEQ ID NO: 17 - DNA, 321 bp, Homo sapiens
gaaatagtgatgacgcagtctccagccaccctgtctgtgtctccaggggaaagagccaccctctcctgcagggccagtcagagtgttagcagcaatttagcctggtaccagcagaaacctggccaggctcccaggctcctcatctttcgtgcatccaccagggccactggtatcccagccaggttcagtggcagtgggtctgggacagagttcactctcaccatcagcagcctgcagtctgaagattttgcagtttatcactgtcagcagtataatagctggccgatcaccttcggccaagggacacgactggagattaaa

SEQ ID NO: 18 - DNA, 348 bp, Homo sapiens
caggtgcagctggtggagtcgggggggggcgtggtccagcctgggaggtccctgagactctcctgtgcagcgtctggattcaccttcagtagctatggcatgcactgggtccgacagggtccaggcaaggggctggagggggtggcaggtatatggtttgatggaagtcatgaatattatgccgacgccgtgaagggccgattcaccatctccagagacaattccaagaacacgctgtatctggaaatgagcagcctgagagccggagacacgggtatatattactgtgcgagaggcggcgatattttgactccctggggccagggatccctggtcattgtctcctca

SEQ ID NO: 19 - DNA, 321 bp, Homo sapiens
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgatcaccttcggccaagggacacgactggagattaaa

SEQ ID NO: 20 - DNA, 360 bp, Homo sapiens
caggtgcagctggtgcagtctggggctgaggtgaagaagcctgggtcctcggtgaaggtctcctgcaaggcttctggaggcaccttcagcagctatgctatcaactgggtgcgacaggcccctggacaagggcttgaatggatgggagggatcatccctatctttggtacagcaaactccgcacagaagttccagggcagagtcacgattaccgcggacaaatccacgagcacagcctacatggagctgagcagcctgagatctgaggacacggccgtgtattactgtgcgagagaggcgggtataccagtggctggacttgccgactggggccagggaaccctggtcaccgtctcctca

SEQ ID NO: 21 - protein, 192 aa, SARS-CoV-2 strain Wuhan-Hu-1
CPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 22 - protein, 69 aa, SARS-CoV-2 strain Wuhan-Hu-1
SNNLDSKVGGNYNYLYRLFRKSNLKPFERDISTEIYQAGSTPCNGVEGFNCYFPLQSYGFQPTNGVGYQ

SEQ ID NO: 23 - DNA, 321 bp, Homo sapiens
gaaatagtgatgacgcagtctccagccaccctgtctgtgtctccaggggaaagagccaccctctcctgcagggccagtcagagtgttagcagcagcttagcctggtaccagcagaaacctggccaggctcccaggctcctcatctatggtgcatccaccagggcccctggtatcccagccaggttcagtggcagtgggtctgggacagagttcactctcaccatcagcagcctgcagtctgaagattttgcagtttattactgtcagcagtataataactggcctctcactttcggcggagggaccaaggtggagatcaaa

SEQ ID NO: 24 - DNA, 363 bp, Homo sapiens
caggtgcagctgcaggagtcgggcccaggactggtgaagccttcggagaccctgtccctcacctgcagtgtctctggtggctccatcagtagtcactactggagctggatccggcagcccccagggaagggactggagtggattgggtatatctattacagtgggagcaccaaccacaacccctccctcaagagtcgagtcaccatatcagtagacacgtccaagaaccagttctccctgaagctgagttctgtgaccgctgcggacacggccgtgtattactgtgcgagaggggtcttactatggttcggggagccaatttttgaaatctggggccaagggacaatggtcaccgtctcttca

SEQ ID NO: 25 - protein, 1255 aa, SARS-CoV strain Urbani
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 26 - protein, 191 aa, SARS-CoV strain Urbani
CPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGP

SEQ ID NO: 27 - protein, 68 aa, SARS-CoV strain Urbani
TRNIDATSTGNYNYKYRYLRHGKLRPFERDISNVPFSPDGKPCTPPALNCYWPLNDYGFYTTTGIGYQ

SEQ ID NO: 28 - skipped

SEQ ID NO: 29 - protein, 6 aa, Homo sapiens
QSVSSN

SEQ ID NO: 30 - protein, 3 aa, Homo sapiens
GAS

SEQ ID NO: 31 - protein, 9 aa, Homo sapiens
QQFNNWITF

SEQ ID NO: 32 - protein, 8 aa, Homo sapiens
GFTFSIYA

SEQ ID NO: 33 - protein, 8 aa, Homo sapiens
ISGSGNHI

SEQ ID NO: 34 - protein, 12 aa, Homo sapiens
AKDGGLYWYFDL

SEQ ID NO: 35 - protein, 6 aa, Homo sapiens
QSVSSN

SEQ ID NO: 36 - protein, 3 aa, Homo sapiens
RAS

SEQ ID NO: 37 - protein, 9 aa, Homo sapiens
QQYNSWPIT

SEQ ID NO: 38 - protein, 4 aa, Homo sapiens
GFTF

SEQ ID NO: 39 - protein, 8 aa, Homo sapiens
IWFDGSHE

SEQ ID NO: 40 - protein, 9 aa, Homo sapiens
ARGGDILTP

SEQ ID NO: 41 - protein, 6 aa, Homo sapiens
QSISSW

SEQ ID NO: 42 - protein, 3 aa, Homo sapiens
AAS

SEQ ID NO: 43 - protein, 9 aa, Homo sapiens
QQSYSTPIT

SEQ ID NO: 44 - protein, 8 aa, Homo sapiens
GGTFSSYA

SEQ ID NO: 45 - protein, 8 aa, Homo sapiens
IIPIFGTA

SEQ ID NO: 46 - protein, 13 aa, Homo sapiens
AREAGIPVAGLAD

SEQ ID NO: 47 - protein, 106 aa, Homo sapiens
DIQMTQSPSTLSASVGDRVTITCRASQSISSWLAWYQQKPGKAPKLLIYKASSLESGVPSRFSGSGSGTEFTLTISSLQPDDFGTYYCQQYNSYYTFGQGTKLEIK

SEQ ID NO: 48 - protein, 106 aa, Homo sapiens
DIQMTQSPSTLSASVGDRVTITCRASQSISSYLNWYQQKPGKAPKLLIYKASSLESGVPSRFSGSGSGTEFTLTISSLQPDDFGTYYCQQYNSYYTFGQGTKLEIK

SEQ ID NO: 49 - DNA, 318 bp, Homo sapiens
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttggaacttattactgccaacagtataatagttattacacttttggccaggggaccaagctggagatcaaa

SEQ ID NO: 50 - DNA, 318 bp, Homo sapiens
gacatccagatgacccagtctccttccaccctgtctgcatctgtaggagacagagtcaccatcacttgccgggccagtcagagtattagcagctatttaaattggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttggaacttattactgccaacagtataatagttattacacttttggccaggggaccaagctggagatcaaa

SEQ ID NO: 51 - skipped

SEQ ID NO: 52 - protein, 94 aa, Homo sapiens
DIQMTQSPSTLSASVGDRVTITCRASQSISSWLAWYQQKPGKAPKLLIYKASSLESGVPSRFSGSGSGTEFTLTISSLQPDDFATYYCQQYNSY

SEQ ID NO: 53 - protein, 6 aa, Homo sapiens
QSVSSS

SEQ ID NO: 54 - protein, 3 aa, Homo sapiens
GAS

SEQ ID NO: 55 - protein, 9 aa, Homo sapiens
QQYNNWPLT

SEQ ID NO: 56 - protein, 8 aa, Homo sapiens
GGSISSHY

SEQ ID NO: 57 - protein, 7 aa, Homo sapiens
IYYSGST

SEQ ID NO: 58 - protein, 15 aa, Homo sapiens
ARGVLLWFGEPIFEI

SEQ ID NO: 59 - protein, 6 aa, Homo sapiens
QSISSW

SEQ ID NO: 60 - protein, 3 aa, Homo sapiens
KAS

SEQ ID NO: 61 - protein, 8 aa, Homo sapiens
QQYNSYYT

SEQ ID NO: 62 - protein, 6 aa, Homo sapiens
QSISSY

SEQ ID NO: 63 - protein, 3 aa, Homo sapiens
KAS

SEQ ID NO: 64 - protein, 8 aa, Homo sapiens
QQYNSYYT

SEQ ID NO: 65 - protein, 451 aa, Homo sapiens
QVQLQESGPGLVKPSETLSLTCSVSGGSISSHYWSWIRQPPGKGLEWIGYIYYSGSTNHNPSLKSRVTISVDTSKNQFSLKLSSVTAADTAVYYCARGVLLWFGEPIFEIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, Homo sapiens
EIVMTQSPATLSVSPGERATLSCRASQSVSSSLAWYQQKPGQAPRLLIYGASTRAPGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 1203 aa, SARS-CoV-2 strain Wuhan-Hu-1
SGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 68 - protein, 192 aa, SARS-CoV-2 strain Wuhan-Hu-1
CPFGEVFNATRFASVYAWNRKRISNCVADYSFLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 69 - DNA, 660 bp, Homo sapiens
gaaatagtgatgacgcagtctccagccaccctgtctgtgtctccaggggaaagagccaccctctcctgcagggccagtcagagtgttagcagcagcttagcctggtaccagcagaaacctggccaggctcccaggctcctcatctatggtgcatccaccagggcccctggtatcccagccaggttcagtggcagtgggtctgggacagagttcactctcaccatcagcagcctgcagtctgaagattttgcagtttattactgtcagcagtataataactggcctctcactttcggcggagggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaatgagggcgcgccaag

SEQ ID NO: 70 - DNA, 1354 bp, Homo sapiens
caggtgcagctgcaggagtcgggcccaggactggtgaagccttcggagaccctgtccctcacctgcagtgtctctggtggctccatcagtagtcactactggagctggatccggcagcccccagggaagggactggagtggattgggtatatctattacagtgggagcaccaaccacaacccctccctcaagagtcgagtcaccatatcagtagacacgtccaagaaccagttctccctgaagctgagttctgtgaccgctgcggacacggccgtgtattactgtgcgagaggggtcttactatggttcggggagccaatttttgaaatctggggccaagggacaatggtcaccgtctcttcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaat

SEQ ID NO: 71 - protein, 1273 aa, SARS-CoV-2 strain Wuhan-Hu-1
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 72 - DNA, 639 bp, Homo sapiens
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttggaacttattactgccaacagtataatagttattacacttttggccaggggaccaagctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 73 - protein, 213 aa, Homo sapiens
DIQMTQSPSTLSASVGDRVTITCRASQSISSWLAWYQQKPGKAPKLLIYKASSLESGVPSRFSGSGSGTEFTLTISSLQPDDFGTYYCQQYNSYYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 74 - DNA, 1350 bp, Homo sapiens
caggtgcagctggtgcagtctggggctgaggtgaagaagcctgggtcctcggtgaaggtctcctgcaaggcttctggaggcaccttcagcagctatgctatcaactgggtgcgacaggcccctggacaagggcttgaatggatgggagggatcatccctatctttggtacagcaaactccgcacagaagttccagggcagagtcacgattaccgcggacaaatccacgagcacagcctacatggagctgagcagcctgagatctgaggacacggccgtgtattactgtgcgagagaggcgggtataccagtggctggacttgccgactggggccagggaaccctggtcaccgtctcctcagctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtcgtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 75 - protein, 450 aa, Homo sapiens
QVQLVQSGAEVKKPGSSVKVSCKASGGTFSSYAINWVRQAPGQGLEWMGGIIPIFGTANSAQKFQGRVTITADKSTSTAYMELSSLRSEDTAVYYCAREAGIPVAGLADWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK